SEQUENCE LISTING (187 sequences)

SEQ ID NO: 1 - DNA, 3393 bp, synthetic construct
  misc_feature 1..3393: note = L1 Construct
  source 1..3393: mol_type = other DNA
atgcgagttcctgctcagctgcttggtctgttactgctgtggttgccgggcgcacgatgtgctatctgggaattaaagaaagatgtgtacgtggtggaattggattggtacccagatgctccgggcgaaatggttgtactcacatgcgatactccggaggaagacggtatcacttggacattggatcagtcgagtgaggttctcggtagtggtaaaacactaacgatccaagtcaaagaattcggtgatgcggggcaatatacctgtcataaaggcggcgaagtactatctcatagcctcctgctgttacacaagaaggaagatggcatatggtccaccgacatccttaaggatcagaaagaacccaagaacaaaactttcttgcgttgcgaagctaagaactactccggccgcttcacatgctggtggttgacaacgatcagtacggatctaaccttctctgttaagtccagtcgggggagttcggacccccaaggcgtcacgtgtggagctgccacactttccgctgagcgcgtacgtggagataataaagagtatgaatactccgttgagtgccaggaggactccgcgtgccccgctgccgaggagagtctccccatagaggtgatggtcgacgctgttcacaaactgaaatatgagaactatacctcatccttctttatacgtgacataattaagccagatcccccgaagaacttacaattgaaaccattgaagaattcacgtcaagtcgaggtatcctgggagtatcccgacacctggtccacgccacactcatatttctctctgaccttctgtgtgcaggtacaaggcaagagcaaacgagaaaaaaaggacagagttttcacggataagactagcgccacagtgatatgtaggaaaaacgcatcgatctcagtccgcgcgcaagatcggtattactcaagcagttggtcagagtgggcatcggtgccctgctcggggggttctggtgggggcagtggaggaggatcaggtggcggcagtcgcaatctacccgtggcaacaccagacccgggaatgttcccatgtctgcaccacagtcaaaacctcttaagggccgtgtcaaatatgctacagaaggcgagacagactttagaattctacccttgtacaagcgaggagattgaccacgaggacatcaccaaagataagacgagcaccgtcgaggcttgcctgcctctagaactaacaaaaaatgaatcatgcttgaactcgagggagaccagtttcattactaacggttcatgtcttgcatcgaggaagacctcattcatgatggccctgtgcctctcgtccatttatgaagacctaaagatgtaccaggtagagtttaagaccatgaacgccaagctcctcatggatccaaaacggcaaatattcttagatcagaatatgctcgctgttatcgacgaactcatgcaggcgcttaacttcaactcagaaaccgttccccaaaagtcgagtctagaagaaccggacttttataagaccaaaattaaactgtgtatactacttcacgccttcaggataagagcagtgacgattgacagggtgatgtcctacttgaatgcatcaggatcagggggaggctcggacgcccataagagcgaagtcgcccaccgcttcaaggatttgggtgaggaaaacttcaaagccctggtcctgatagcgtttgcccaatatttgcagcagtgtccattcgaagatcacgtgaaattggtgaacgaggtaacagaatttgctaagacttgtgtggctgacgagtcggccgaaaactgtgataagagtcttcatacactgtttggcgataagctatgtactgtcgctacacttagggagacttacggtgagatggccgactgctgcgccaagcaagagccagaacgaaacgagtgttttctgcaacataaggacgacaatcccaacctgcccagattggttcgccctgaagttgatgttatgtgcaccgcatttcacgacaacgaagaaacctttcttaaaaagtatctgtacgagatagctcgacgtcacccttacttctacgcgcccgaacttctgtttttcgccaagcgatacaaagccgctttcacagagtgttgccaagctgccgacaaagccgcttgccttctaccaaagcttgacgagctcagagatgaagggaaagctagttcggcaaagcaacgattaaagtgtgcatcactgcaaaaattcggcgaacgagcctttaaagcatgggcagttgccaggttatcccaaaggttcccgaaagctgaattcgctgaggtgagcaagttagtcacggaccttacgaaggtacataccgaatgctgccacggggacctcttggagtgcgctgacgacagggcggacttagctaaatacatttgcgagaatcaggactcaatcagttctaaacttaaagaatgctgcgagaaaccgctcctggaaaaatcacattgcatcgccgaggtggaaaacgatgagatgccagcagatttaccatctctagccgccgacttcgtggaaagtaaggatgtgtgtaaaaactatgcggaagcaaaagacgtgttcctcggaatgtttctatacgaatacgctagaaggcatcctgactattctgtcgttttactgcttagactagcgaagacatatgaaacgacgttagagaaatgctgcgcggccgctgacccccacgaatgttacgcgaaggtctttgatgagttcaagcccctggttgaggagccgcaaaaccttattaaacagaattgtgagctatttgagcagttaggcgaatataaattccagaatgcacttctagtacgatacaccaaaaaggtccctcaagtgagcacccccactcttgtggaggtatccagaaatctaggaaaggtaggctctaaatgctgcaagcatcccgaagccaagagaatgccatgcgctgaagactaccttagcgttgttctgaatcagttgtgtgtccttcacgaaaagacgccggtgagtgatcgtgtcacgaagtgctgtacagagagcctcgtcaaccgtaggccatgtttctccgctctcgaggtggatgaaacatatgtacctaaggaatttaatgcggaaactttcacctttcacgcggacatctgtaccctgagcgagaaggagaggcagataaaaaagcagacggctcttgtagagttggtcaaacataagcctaaggccactaaagagcagctaaaggcagtaatggacgactttgcggctttcgttgagaagtgctgcaaggccgacgataaagagacctgtttcgcggaagaaggtaaaaagttagtggccgcctcccaggcggccctgggcctgtag

SEQ ID NO: 2 - DNA, 3393 bp, synthetic construct
  misc_feature 1..3393: note = L2 Construct
  source 1..3393: mol_type = other DNA
atgcgcgtgcccgcacaattgctcggactgctgctactgtggctgcccggggctcgctgcgcaatttgggaacttaagaaggacgtatacgttgtggaacttgactggtaccctgatgctccaggggagatggtggttttgacgtgtgacaccccggaagaagatggaattacatggaccttagaccaatcctccgaggttcttggctcgggcaaaaccttgaccattcaggtcaaggaatttggcgatgctggccaatacacctgccataaaggtggtgaagttttatctcactccctactgttgctccataagaaagaagacggcatttggtcgacagacatattgaaggatcagaaggaacctaagaataagaccttcttacgatgtgaggccaagaattattccggacgttttacgtgttggtggttgaccacgatctccactgacttaaccttctcagtgaaatcctcacgaggtagttccgatccccagggtgtgacgtgcggtgcggccacgttaagtgctgagagagtacggggcgacaataaggaatacgagtactcagttgaatgccaagaggactcggcctgtcccgcggcagaggagagtctccctatcgaagtgatggtggatgcggtgcacaagctcaagtatgaaaattacacatcatcttttttcatcagagacattataaagcccgacccaccaaagaacctccagttaaagcccttaaagaacagtagacaggttgaagtatcatgggaatacccagacacctggtccacaccccattcgtatttttccttgacgttttgcgtacaagttcagggaaagtccaaacgggaaaagaaagaccgcgtttttacagacaaaacttctgccactgtcatctgtagaaagaacgcatcaattagcgtgcgagcgcaagacagatactattcaagtagctggagcgagtgggccagtgttccatgttctggcggttcgggcggagggtccggcggtggtagcggaggcgggagcaggaacttgcccgtggctactccagaccctggcatgttcccctgtttacaccactcccagaacttattacgtgctgttagcaacatgttgcaaaaggcccgtcaaaccctcgagttttacccctgtactagtgaagaaatcgaccacgaagacataacaaaagacaagacaagcacagttgaggcatgcttacccctggagttaacaaagaacgagagctgtctgaactctcgagagacgagcttcatcaccaatgggagttgccttgcttctcgaaaaacgtcgttcatgatggccctgtgcctgtcgtctatctatgaggatctgaaaatgtatcaggttgaattcaagacaatgaatgccaaactactcatggatccaaaacggcagatattcctcgatcagaatatgctcgcagttattgacgaactaatgcaggctctgaattttaacagcgagaccgttcctcagaagtcaagtttggaagaacctgacttctacaaaactaaaataaaattatgcatcttactgcatgctttcagaattagagctgtcactattgatcgagtgatgtcatacttgaatgcttccggatcaggaggtgggagcgacgcgcacaaaagcgaggtagcgcatcgctttaaagacttaggagaggaaaactttaaggcgctggtgctcatcgcatttgcccaatacttacagcagtgtccttttgaggaccacgtaaagcttgtaaacgaagtcactgaattcgccaagacatgtgttgctgacgaaagcgcagagaactgtgacaaaagccttcataccctctttggtgacaaactctgcaccgtggcaactctaagagaaacctacggggaaatggcagactgctgcgcaaagcaggaacccgaacgcaatgagtgtttcctgcagcacaaggatgataatcccaatctgccacgacttgtacggccggaggtagatgttatgtgcactgcttttcatgacaacgaggaaactttccttaagaaatatctgtatgagatcgcaaggcggcacccttacttctacgcacccgaactgttgtttttcgcaaagaggtataaggccgcatttaccgagtgttgtcaggccgctgataaagccgcctgtcttttgccaaaattagatgaactaagggacgaaggcaaagcgagtagcgccaaacaaagattaaaatgtgcaagcctccaaaaattcggtgaaagagcatttaaggcgtgggctgtcgcccgactttcacaacgcttccccaaagctgaattcgctgaggtttcgaagctggttaccgacctaactaaagtgcatacagagtgctgtcatggggatctcttagagtgcgcggatgaccgggcagacctggctaagtacatatgtgagaaccaggacagtatatcatcaaagctgaaagagtgttgtgagaagccactactcgagaagagtcactgtattgccgaggtggaaaatgatgagatgccagccgatcttccttctttggccgctgactttgtagagagcaaggatgtctgtaagaactacgctgaggccaaggatgtctttttggggatgttcctctatgagtacgcccgacgacaccctgactatagtgtagtacttttgcttagactggctaaaacatatgagacgactctcgaaaagtgctgtgccgctgccgatccacacgagtgctacgctaaggtgtttgatgagtttaagccgctggtggaggaaccccagaacctgatcaagcagaactgtgaactattcgagcaactaggggagtacaaattccagaacgcacttttagtgcggtacaccaaaaaagtgccacaggtcagtacaccaacattagtggaagtatccaggaacctgggcaaagtgggcagcaaatgctgcaaacatccggaggctaagcggatgccctgtgcagaggactacctgtccgtggtgcttaaccagctgtgtgtgcttcacgagaaaacgcctgtgtccgaccgggtgaccaagtgctgtacggagtcactggtaaatcgacgaccgtgtttttcagcactagaagttgatgaaacttatgtaccgaaagagtttaacgcagagacctttacattccacgccgacatctgcacgctgtccgagaaggaaagacagattaaaaagcagactgccctagtcgagcttgtcaaacacaaaccgaaggcaaccaaggaacagttaaaagcagtgatggatgattttgctgcgttcgtcgaaaaatgttgcaaagcggacgacaaggagacttgcttcgcagaggaagggaagaaattggttgcggcgtcccaagcggccttagggctatag

SEQ ID NO: 3 - DNA, 3393 bp, synthetic construct
  misc_feature 1..3393: note = L3 Construct
  source 1..3393: mol_type = other DNA
atgagagttcctgctcaactactagggctgctgttgttgtggttgcccggcgcgcgatgcgcaatatgggagctcaagaaggacgtttatgtcgttgagctggattggtaccctgatgccccgggcgaaatggttgtgcttacgtgcgatacccccgaggaggatggcataacatggacgttagatcagtcttccgaggtccttggttccggtaagactcttactatccaggtgaaggagttcggcgatgccggccagtacacttgccataaaggcggtgaagttctaagccactctctactgcttttgcacaagaaggaagatggaatatggtccaccgacatcttgaaggaccagaaagaaccaaaaaataagacatttttgaggtgtgaggcaaaaaattattcgggacgcttcacctgctggtggttgacgacgatttcaaccgacctcaccttctcagtaaagagttcgagaggtagttccgatccccaaggtgtgacatgtggcgctgcgactctaagcgctgaacgcgtaagaggtgataacaaagagtacgaatacagtgtggaatgccaagaagatagcgcgtgtccagccgcagaagaatctttaccaatagaggttatggttgatgccgttcacaaattgaaatatgagaattacacctcaagctttttcattcgagacataataaagcccgaccctcctaagaatcttcagttaaaaccgctgaagaacagtagacaagttgaggttagctgggaatatcctgatacctggtcaacgccgcactcgtatttctccctgactttctgtgttcaggttcaaggaaaatctaaaagggagaagaaagaccgtgttttcaccgacaagacatctgccacagtcatatgtaggaagaacgcttcaatcagcgtgcgagcgcaggaccgatactacagctctagttggtccgaatgggccagcgtaccatgctctggcggttccggcgggggctcggggggcgggagcggtggaggcagcaggaatcttcctgtcgcgacccctgatcccggcatgtttccttgcctgcaccacagtcagaacttattacgcgccgtttccaatatgttacagaaggccagacagacattagagttttatccttgcacatcggaggagatcgaccatgaagatatcacaaaagataaaacatccaccgttgaggcctgcctgccacttgaacttactaaaaacgagagctgcctgaatagccgggaaacttctttcatcactaatggatcgtgtctagcaagccgaaaaaccagcttcatgatggctttgtgcctctcgtccatctatgaagacctgaaaatgtatcaagtagaatttaaaacgatgaatgccaaactgcttatggatcccaaacgccagatatttctagaccagaatatgctggccgtcattgatgagctaatgcaggctctcaattttaatagcgaaacagtgccccaaaaaagctctttggaagagccggatttttacaaaaccaagattaagctatgtatcctgctgcatgctttcagaattcgagctgttacaattgatcgggttatgagctacttaaacgcctcgggtagtggcggggggagcgacgcccacaagtctgaagtggcacaccggttcaaggacctcggggaggagaattttaaagccctcgtgctgatcgctttcgcgcagtacttgcagcagtgcccttttgaggaccatgtcaaattagtaaacgaagtgacggaattcgcaaagacttgcgtagcggatgagtcagcagagaattgcgacaagtcgctacacactctgttcggggataagttgtgcacagttgctaccttacgagagacctatggagagatggctgactgctgcgccaaacaagagcctgaaagaaacgagtgcttcttacaacacaaagacgataacccgaatttgccaaggcttgtaagacccgaagtagatgttatgtgcacagcttttcacgacaacgaggagacgttccttaagaaatatctgtatgaaatagcccgtcggcacccctatttttatgctcccgaactattgttcttcgccaaacgatacaaggctgcgttcactgagtgctgtcaggctgcagacaaagcagcctgcttgcttcccaaattggacgaactacgggacgaaggtaaggcgagctccgcaaaacagcggttgaaatgcgcgtcactacagaagtttggggaaagagcgttcaaagcttgggctgtggcacgattgagccaacgcttccccaaagcagagtttgcggaagtttcgaaactcgtgacagacttaacaaaggttcacaccgagtgctgtcacggcgatctgctcgaatgcgctgatgaccgcgctgacttggctaaatacatttgtgagaaccaggactctatatcgagcaaactcaaggaatgctgcgaaaagcccctgcttgagaagtcccactgcatcgctgaggtagagaatgatgagatgcctgcggatcttccgagtttagcagctgatttcgtcgagagcaaagatgtgtgcaaaaattatgccgaagcaaaagacgtatttcttgggatgttcttatacgagtatgcacggcgccacccagattactccgtagtgctactgttaagattggcgaagacctatgaaaccacattggaaaagtgctgcgccgccgccgacccccacgagtgttacgcgaaggtgttcgatgaatttaaaccgcttgttgaggagccgcaaaacctaataaagcaaaactgtgagctctttgagcaactaggtgaatacaagtttcagaatgcactcctagttcggtacaccaaaaaagtacctcaggtatctacgccaacgttagtcgaggtctcgcggaatttgggtaaagtaggttccaagtgttgcaaacacccggaagctaaacgtatgccgtgtgctgaggactatctcagcgttgtgttaaaccaactttgcgtactccacgagaaaacacctgtctcagatcgggtaaccaaatgctgcacggagtcgctagtaaatcgtcgcccatgcttttctgcgttagaggtggacgaaacttatgtaccgaaagaatttaacgcggaaacctttacattccatgcagatatctgtacactgtccgagaaagagagacagattaagaaacagacggcgctggtggagcttgtgaagcacaagcctaaagctacgaaggagcaactgaaggcagtcatggatgactttgcggcgtttgtggagaagtgctgtaaagcggacgataaggaaacatgcttcgcagaagaaggaaagaagctggtcgccgctagccaagcggctctgggcctgtag

SEQ ID NO: 4 - DNA, 3393 bp, synthetic construct
  misc_feature 1..3393: note = M1 Construct
  source 1..3393: mol_type = other DNA
atgagagtccccgcccagctgctggggcttcttttgctttggcttcctggcgcaagatgcgctatctgggagctgaaaaaggacgtgtacgtggtggaacttgactggtaccccgacgcccccggcgagatggtggtactgacctgcgacactcccgaggaagacggcattacctggaccttggaccagagcagcgaggttctgggctccggaaaaaccttgacaatccaagtgaaagaattcggcgacgctggccagtacacctgccacaagggcggcgaggtgctgtcccacagcctgctgctgctgcataagaaagaagacgggatttggagcaccgatatactgaaggatcagaaggagcccaagaacaagaccttcctgaggtgcgaggccaaaaattacagcggcagattcacctgctggtggctgaccaccattagcacagacctgactttcagcgtaaagtcttcaaggggcagctcagacccccagggagtaacttgcggagcggcaacgttgtctgccgagcgggtcagaggcgacaataaggagtacgagtattcagtagagtgtcaggaagatagcgcctgtcccgccgcggaggagagcctccccatcgaggtgatggtggacgccgtgcacaagttaaagtacgagaattacaccagctcattttttatcagagacattatcaagccggaccccccgaagaacttacagcttaaacccctaaagaacagcaggcaggttgaggtcagctgggaatatcctgacacctggtcaaccccccacagctacttctcccttactttctgtgtgcaagtgcagggcaagagcaagagagaaaagaaggaccgggtgtttaccgacaagactagcgccaccgtgatttgcagaaagaacgccagcattagtgtgagagcccaggacaggtattactccagctcatggtctgagtgggctagtgtgccttgctctggcggcagcggcggcgggagcggcggaggctccgggggaggtagccggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaagagcagcctcgaggagcccgacttctacaaaacaaagatcaaactctgcatccttctgcacgccttcagaattagagccgtgaccatcgacagagttatgagctacctgaatgccagcggcagcggcggcggatccgatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctctag

SEQ ID NO: 5 - DNA, 3393 bp, synthetic construct
  misc_feature 1..3393: note = M2 Construct
  source 1..3393: mol_type = other DNA
atgagagtccccgcccagctgctagggctgctgctgctgtggttacccggcgcccggtgtgcaatttgggagttgaagaaggacgtgtacgtggtggagctggactggtacccggatgctcccggcgagatggtggtactcacctgcgacacacctgaggaagacggcatcacctggaccctcgatcagagcagcgaggttctgggaagcggcaaaaccctgaccatccaagtgaaagagtttggcgacgccggtcagtacacctgccacaagggaggcgaggtcctgtctcactctctgctgctgctccataagaaggaggacggtatttggagcactgacatcttgaaggatcaaaaagagccaaagaataaaacgttcctgaggtgcgaagctaagaattactccgggcgttttacgtgctggtggctgaccacgatcagcaccgatctgaccttcagcgtgaagagcagccggggcagcagcgacccccaaggcgtgacttgcggcgctgcgaccctgagcgctgagcgtgtgcgcggcgacaacaaggagtatgagtattcagtggagtgtcaggaggactccgcctgtcccgcggccgaagagagtctgcctattgaggtgatggtggacgccgtgcacaagctgaagtacgagaactacacatcgtcattctttatccgcgacatcataaagcccgacccccccaagaacctgcagctgaagcctctcaagaattcccggcaagtggaggtgagctgggagtaccctgatacctggtctacccctcacagctactttagcctgaccttctgcgtccaggtgcaaggaaagtcgaagcgcgagaagaaagatagagtcttcaccgataaaaccagtgccaccgtgatttgccgcaaaaacgcctccatcagcgtgcgggctcaggatagatactactctagcagctggagcgaatgggcctcagttccttgcagcggcggcagcggtggaggaagcggcggtggcagtgggggcgggagcagaaatctgcccgtcgccactccagatcctggcatgttcccgtgcctgcatcacagccaaaacctgctgcgggcggtgtctaacatgctgcagaaggctaggcagaccttggaattctatccctgcacaagcgaggaaatagaccatgaggacatcaccaaggataagaccagcacggtcgaagcttgcctgccactggaactgacaaaaaacgagagttgcctgaactcccgcgagacatccttcatcacaaacggcagctgcctggctagcaggaagaccagcttcatgatggccctgtgcctgtcttccatctacgaggacctgaaaatgtaccaagtggagttcaagactatgaacgccaagctgctaatggatcccaagcgacagatctttctagaccagaacatgctggccgtcattgacgagctgatgcaggcactcaattttaactcagagaccgtgccacagaagtccagcctggaggagcctgacttctataagaccaagattaagctgtgcatcctgctgcatgccttccgaataagagccgtgaccattgaccgagtgatgtcatacttgaacgcaagcggctcaggcggagggagtgacgcccacaagtctgaagtggctcaccggtttaaggaccttggcgaggagaactttaaagccctggtgctgattgcctttgcccagtatttacaacaatgccctttcgaagaccacgtgaagctcgtcaatgaggtcaccgagttcgctaagacctgcgtagccgacgaaagtgccgagaactgcgacaagagcctgcacaccctgttcggggacaaactctgtaccgtggccaccctacgggagacatatggggagatggccgactgctgcgcaaaacaggagcccgagagaaatgagtgcttcctgcagcacaaggatgacaaccccaatctgcccagactggtgcgccccgaggtagacgttatgtgcaccgccttccatgacaatgaggagacgttcctgaagaaatacctgtacgagatcgcaagacgtcacccctatttctatgcacctgagctgcttttcttcgccaagagatataaggccgccttcaccgaatgctgccaggcagccgataaggcagcttgcctcctgccaaagctggacgagctgagagatgagggcaaggcctccagcgcgaagcagagactcaaatgcgcaagccttcagaagttcggagaacgcgcctttaaagcctgggccgtcgccagactgagccagcgcttccctaaagccgaattcgcagaagtgagcaagctggtaacggacctgacaaaggtgcatactgagtgctgccatggcgatctgctggagtgcgctgatgacagagcagatttggcgaaatatatttgcgaaaatcaggatagcatcagctctaagctcaaggagtgttgtgagaagcccctgctggaaaaaagccactgcattgcagaggttgagaacgatgaaatgccagccgaccttccatcattggccgccgatttcgtggagtcgaaggatgtgtgtaagaactacgccgaggccaaggacgtgttcctgggcatgttcctgtacgagtatgctagaagacatcccgattacagtgtggtgctgctattgagactggccaagacctacgaaaccaccctggagaaatgttgcgccgcggcagatcctcacgaatgttacgccaaagtgtttgacgaattcaagccactggtagaggagccccagaacttaataaagcagaattgcgagctattcgagcagttgggcgagtacaaattccagaacgcccttctggtgaggtataccaaaaaggtgccccaggtgtctacccctaccctggtggaggtcagccgaaatctgggaaaggtcggatccaagtgctgcaagcacccggaggccaagaggatgccttgcgctgaggactatctcagtgtcgtcctgaatcagctatgcgtgttgcacgagaaaaccccagtgagtgaccgcgtgactaaatgctgcaccgaaagcttggtgaatcggaggccctgtttctctgcactggaagttgacgagacttacgtcccgaaggaattcaacgccgagacattcaccttccatgctgacatatgtactctgtcagaaaaggagcgtcagatcaagaagcagacagccctggtggaactggttaagcataagcctaaagcgaccaaagagcagctgaaagccgtgatggacgattttgccgccttcgtggagaaatgttgtaaggcagacgacaaagagacatgtttcgccgaagaggggaagaaactggtggccgcaagccaggccgctctgggtctgtag

SEQ ID NO: 6 - DNA, 3393 bp, synthetic construct
  misc_feature 1..3393: note = M3 Construct
  source 1..3393: mol_type = other DNA
atgagagtcccagctcagctgctgggcctactgctgctatggctccccggcgcgcggtgcgccatttgggagctcaagaaggacgtgtacgtggtggaacttgactggtacccggacgcgcccggggaaatggtggtgctaacctgtgacacccccgaagaggacggcatcacctggaccctggaccagagcagtgaggtgctaggtagtggcaaaacgttaaccatccaggtcaaggagttcggcgacgccgggcaatacacctgtcacaaggggggggaggtactatcccactccctgctgctcctgcacaagaaagaggacgggatctggagcaccgacattctgaaagaccaaaaggagcccaaaaacaaaaccttccttagatgtgaagccaagaactacagcggccgtttcacctgctggtggctgaccaccatatctacggaccttaccttttcggtgaagagcagcagggggagttccgacccgcaaggcgtaacttgcggagccgcaaccctgagcgccgagagagtgcgcggcgacaacaaggagtacgagtatagcgtggagtgccaagaggacagcgcatgcccagccgccgaagagagcctgccaatagaggtcatggtagacgccgtgcacaagctaaaatatgaaaactacaccagcagctttttcatcagggatatcatcaaacccgacccaccaaaaaacttacagcttaagcctctgaaaaacagcagacaagttgaggtcagctgggagtaccccgacacttggagcacaccccactcctatttcagtttgacattctgcgtgcaggtgcagggtaaaagcaagagagaaaagaaggacagagtgttcacagataagacctcagccacagtgatctgccgtaagaatgccagcatcagcgtccgggctcaggacaggtactactcttcctcatggagcgagtgggcctctgtcccctgcagcggcggcagcggcggtggcagcggcggcggttctggcggcggttcaagaaacctcccagtcgctacccccgatcccggaatgttcccctgcctgcaccactcccagaatctgctccgagccgttagcaacatgctgcaaaaggcccggcagaccctggagttctacccatgcacctcggaagaaatcgatcacgaggacatcaccaaggacaagactagcaccgtcgaggcctgcctgccgctggaactaaccaagaatgaaagctgcctcaactcgcgggagacctctttcataaccaacggctcatgcctggccagccggaaaactagctttatgatggctctgtgcttaagcagcatctacgaggatctgaagatgtaccaggtagagttcaagaccatgaatgccaagctgctgatggaccccaagagacaaatcttcctggaccagaacatgctggccgtaattgatgaactgatgcaggccctgaatttcaacagcgagaccgtaccccagaaaagctcactggaggagcccgacttttataagacgaaaataaagttgtgcatccttcttcacgctttccggattagagccgtgaccatcgatagagtgatgtcatacctgaacgcatcggggagtggcggtggcagcgatgcccacaaaagcgaagtcgcacacagattcaaggacttgggtgaggagaactttaaagccctggtgctgatcgccttcgcgcagtatctccagcagtgccccttcgaagatcatgtgaaactggtgaacgaggtaaccgagttcgcgaagacatgcgttgctgatgagagcgccgaaaattgcgacaaaagcctgcatactctgttcggggacaagctgtgcacggtcgcaaccctgagagaaacctacggcgagatggcagactgctgcgccaagcaggagcctgagaggaacgagtgttttctgcagcacaaggacgataatcctaaccttcctcgtctagtgagacccgaagtggacgttatgtgtaccgcctttcacgacaatgaggaaacattcctgaaaaagtacctgtacgagatcgccagacggcacccatatttctacgcccccgagctgctcttcttcgcaaagaggtacaaggctgccttcaccgagtgctgccaggcggccgacaaggcggcgtgtttgctgcctaagctggacgaactacgtgacgaaggaaaagctagcagcgccaagcagagacttaagtgcgcgtccttacagaagtttggcgaaagagcgtttaaggcctgggccgtggcaaggctgtctcaaagattccccaaggcggagttcgccgaggtgtcaaaactggtgaccgacttaaccaaggtgcacaccgaatgctgccacggcgatctgctcgagtgcgccgacgacagagccgatctggcaaaatacatctgcgaaaaccaggatagcatcagctccaaactgaaggagtgctgtgaaaaaccactgcttgaaaaatcgcattgtatagcggaggtggagaatgacgagatgcccgccgacctgccaagcctggccgccgatttcgttgaatccaaggacgtttgcaagaactatgcagaagcgaaggacgtgttcttaggaatgttcctatacgagtacgcgagaagacatcccgactacagcgtggttctgctgttgagattagccaagacgtatgagacaaccctcgaaaagtgctgcgccgccgccgacccccacgagtgttacgcaaaggtgttcgatgagtttaaaccgctggttgaggaaccgcaaaacctgatcaagcagaactgcgagctgttcgagcagctgggtgaatacaagtttcagaatgcactgttggtgcgatataccaagaaggtgcctcaggtgagcacccctacccttgttgaggtgtcccgcaatctgggtaaggttgggagcaagtgttgcaaacaccccgaggccaagagaatgccctgcgcggaagattatctcagtgtcgtgcttaatcagttatgtgtcctgcatgagaagacccccgtgagcgacagagtgaccaagtgctgtaccgaatctctcgtgaacagacgcccgtgcttcagcgccttggaggtagacgagacctacgtgcccaaggagttcaacgcagagaccttcacctttcacgccgatatctgcaccctgtccgagaaggagagacaaatcaagaaacaaacggccctcgtggagctggtcaagcacaaacccaaggccacaaaagagcagctgaaggccgtgatggacgacttcgcagcctttgtggagaaatgctgcaaggctgacgacaaggagacatgcttcgccgaggaaggcaagaagttggtggccgccagccaggcggccctgggcctgtag

SEQ ID NO: 7 - DNA, 3393 bp, synthetic construct
  misc_feature 1..3393: note = H1 Construct
  source 1..3393: mol_type = other DNA
atgagagtgcccgcccagttgcttggcctgctgctcctatggcttcccggcgccagatgcgccatctgggagctgaagaaagacgtgtacgtggtggagctggactggtaccccgacgcccccggtgaaatggtggttctgacctgcgacacaccagaggaggacggcatcacctggaccctggaccagtccagcgaggtcctgggctctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggcagcggcggcggctccggcggcgggagtggcggcggcagcagaaatctgcccgtagccacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaagagcagcctggaggaacccgacttctacaaaaccaagatcaagctgtgcattctgctgcatgccttccgcattagagccgtgaccatcgatagggtgatgagctacctgaacgccagcggctctggcggcggcagtgacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgagtgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaagagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctttag

SEQ ID NO: 8 - DNA, 3393 bp, synthetic construct
  misc_feature 1..3393: note = H2 Construct
  source 1..3393: mol_type = other DNA
atgagagtgcccgcccagctgctgggcctgctgctcttatggctgcccggcgcccgctgtgctatttgggagctgaagaaggacgtgtacgtggtggagctggattggtatccagacgcccccggcgagatggtcgtgttgacctgcgatactcccgaggaggacggaatcacctggacacttgaccagagcagcgaggtgctgggcagcgggaagaccctgactatccaggtgaaggagtttggcgatgccggacagtacacctgccacaagggcggcgaggtgttatctcatagcctgctgctgctgcacaaaaaggaggacgggatctggagcactgacatcctgaaggaccagaaggagcccaaaaacaagaccttcctgcgttgcgaggccaagaactacagcgggagattcacctgttggtggctgaccactatcagcactgacctaaccttcagcgtgaagagcagccggggaagctctgacccccagggggtgacatgcggcgccgccacactgagcgccgagagggtgagaggggacaataaggaatacgagtatagcgtggagtgtcaggaagattccgcctgccccgccgccgaggagagcctgcctatcgaggtcatggtggacgccgtccataaactgaagtacgaaaactatacttcaagcttcttcatcagagacatcataaagcccgacccccccaagaacctgcagctaaagcccctgaagaacagcagacaggtcgaagtgagctgggagtaccccgatacctggagcaccccgcacagctacttcagcctgaccttttgcgtccaggtgcagggcaagagcaagagagagaagaaggacagggtgttcactgacaagacaagcgccactgttatctgcagaaagaacgccagtatcagcgtgcgcgcccaagacaggtattactccagcagctggtctgaatgggccagcgtgccttgcagcggggggagcggcggtggcagcgggggcggcagcggcgggggcagcagaaacctgcccgtggccacacccgatcccggcatgttcccctgtctgcaccacagccaaaacctgctgcgtgccgtgagcaacatgctgcagaaggcgcggcagaccctggagttctatccctgcaccagtgaggagattgaccacgaggatatcaccaaagacaagaccagcaccgtggaggcctgcctccccctggagctgaccaagaacgagtcctgcttgaattcaagagagaccagcttcatcaccaacggctcctgcttagccagcagaaagactagcttcatgatggccttgtgcttgtctagcatctatgaggatctgaagatgtaccaggtcgagttcaagactatgaacgccaagctgctgatggaccccaaaagacagatcttcctggaccagaacatgctggccgtcatcgacgagctgatgcaggcccttaatttcaatagcgagacagtgccccagaaatcttctctggaggagcccgatttttacaaaaccaagatcaaactatgcatcctgttgcacgccttccggatccgcgccgtgaccatcgacagagtaatgtcctacctgaacgccagcggcagcggcggcggtagcgacgcccacaagagcgaagtggcccatagattcaaggacctgggcgaggagaacttcaaggccctcgtgctgatcgccttcgcccagtacctgcagcagtgccctttcgaggaccacgttaaactggtgaatgaagtgaccgagttcgccaaaacctgcgtggccgacgagtctgccgaaaattgcgacaaaagcttacacaccctgttcggcgacaagctgtgcaccgtggccaccttaagagaaacctacggcgagatggccgactgctgcgctaagcaggagcccgagagaaacgaatgcttcctgcagcacaaggacgataacccaaatctgcctagactggtgagacccgaggtggacgtgatgtgcacagccttccacgataacgaagagacattcctgaaaaagtacctgtacgagatcgccagaagacatccttacttctatgcccccgagcttctgttcttcgccaagagatataaggccgccttcaccgagtgctgccaagccgccgacaaggcagcctgcctgctgccaaagcttgacgagctgagagacgagggcaaagccagcagcgccaagcagagactgaagtgcgcctccctgcagaagttcggcgagagagcctttaaggcctgggccgtggccagattgagtcagagattccccaaggccgagttcgccgaggtgagcaaactggtgaccgacctcactaaagtgcacacagaatgttgccatggcgatctcctggaatgcgccgatgacagggccgacctggccaagtacatctgtgagaaccaggacagcatttcgagcaagctgaaggagtgctgcgagaaacccctgctggagaagtcccactgcatcgccgaggtggagaacgacgagatgcccgccgacctgcccagcctggccgcagatttcgtggagagcaaggacgtatgcaagaactacgcagaggccaaggatgtgttcctgggcatgttcctgtacgaatacgccagaaggcaccccgactacagcgtcgtgctgttactgagactggccaagacctacgagacaaccttagagaagtgctgcgcggccgcagacccgcacgagtgctacgccaaggtgttcgacgagttcaagcccctggtggaggaacctcagaatctgattaagcagaattgtgagctgttcgagcagctgggagagtacaagttccagaacgcgctgctggtgagatataccaagaaggtgccccaggtgagcacccccaccctggtggaggtgagtcgcaacctgggcaaggtggggagcaagtgttgcaagcatcccgaggccaaaagaatgccctgcgcagaggactatctgagcgttgtgcttaaccagctgtgcgtgctgcacgagaagacccccgtgagcgacagagtgaccaagtgttgcaccgagagcctggtaaacagaagaccctgcttcagcgccctggaggtggacgagacctacgtgcccaaggagttcaacgccgagacctttaccttccacgcagacatttgcaccctgagcgagaaagagaggcagatcaagaaacagaccgctctggtcgagcttgtgaagcacaagcccaaagctaccaaggagcagctgaaggccgtgatggatgatttcgccgccttcgtagagaaatgctgcaaggccgacgataaagagacctgctttgccgaggagggcaagaaactggtggccgccagccaggcggccctgggcctgtag

SEQ ID NO: 9 - DNA, 3393 bp, synthetic construct
  misc_feature 1..3393: note = H3 Construct
  source 1..3393: mol_type = other DNA
atgagagtgcccgcccagctgctgggcctgctgttgctgtggctgcccggcgccagatgcgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaagagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggcggtggcagtggcgggggcagcagaaacctgcccgtggccacccccgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaagagcagcctggaggagcccgacttttacaagaccaagatcaagttgtgcatcctcctgcacgccttcagaatcagagcggtgacgatcgacagagtcatgagctacctcaacgctagcggcagcggtggcggcagcgacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctcttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggagaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctgtag

SEQ ID NO: 10 - DNA, 3393 bp, synthetic construct
  misc_feature 1..3393: note = CO Construct
  source 1..3393: mol_type = other DNA
atgagagtgcccgcccagctgctgggcctgctgctgctgtggctgcccggcgccagatgcgccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaagagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagcggcagcggcggcggcagcgacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 11 - DNA, 3390 bp, synthetic construct
  misc_feature 1..3390: note = CP Construct
  source 1..3390: mol_type = other DNA
atgagggtccccgctcagctcctggggctcctgctgctctggctcccaggtgcacgatgtgccatctgggagctgaagaaggatgtgtatgtggtggagctggactggtacccagatgcccctggagagatggtggtgctgacctgtgacaccccagaggaggatggcatcacctggaccctggaccagagcagcgaggtgctgggcagcggcaagaccctgaccatccaggtgaaggagtttggagatgctggccagtacacctgccacaagggcggggaggtgctgagccacagcctgctgctgctgcacaagaaggaggatggcatctggagcacagacatcctgaaggaccagaaggagcccaagaacaagaccttcctgcgctgtgaggccaagaactacagcggccgcttcacctgctggtggctgaccaccatcagcacagacctgaccttctctgtgaaaagcagccggggcagcagtgacccccagggcgtgacctgtggggccgccaccctgtctgctgagcgggtgcggggggacaacaaggagtatgagtacagcgtggagtgccaggaggacagcgcctgcccagctgctgaggagagcctgcccatcgaggtgatggtggatgctgtgcacaagctgaagtatgagaactacaccagcagcttcttcatccgggacatcatcaagccagacccccccaagaacctgcagctgaagcccctgaagaacagccggcaggtggaggtgtcctgggagtacccagacacctggagcaccccccacagctacttcagcctgaccttctgtgtgcaggtgcagggcaagagcaagcgggagaagaaggacagagtcttcacagacaagaccagcgccaccgtcatctgcaggaagaatgccagcatctctgtgcgggcccaggaccgctactacagcagctcctggagcgagtgggcctctgtgccctgcagcggcggcagcggcggcggcagcggcggcggcagcggcggcggcagcaggaacctgcctgtggccaccccagaccccggcatgttcccctgcctgcaccacagccagaacctgctgcgggctgtgagcaacatgctgcagaaggcccggcagaccctggagttctacccctgcaccagcgaggagattgaccacgaggacatcaccaaggacaagaccagcacagtggaggcctgcctgcccctggagctgaccaagaatgaaagctgcctgaacagccgggagaccagcttcatcaccaacggcagctgcctggccagcaggaagaccagcttcatgatggccctgtgcctgagcagcatctatgaggacctgaagatgtaccaggtggagttcaagaccatgaatgccaagctgctgatggaccccaagaggcagatcttcctggaccagaacatgctggccgtgattgatgagctgatgcaggccctgaacttcaacagcgagaccgtgccccagaaaagcagcctggaggagccagacttctacaagaccaagatcaagctgtgcatcctgctgcacgccttccgcatccgggctgtgaccatcgacagagtgatgagctacctgaatgccagcggcagcggcggcggcagtgatgcccacaagtctgaggtggcccaccgcttcaaggacctgggggaggagaacttcaaggccctggtgctgattgcctttgcccagtacctgcagcagtgcccctttgaggaccacgtgaagctggtgaatgaggtgacagaatttgccaagacctgtgtggctgatgaatctgctgagaactgtgacaagagcctgcacaccctgtttggagacaagctgtgcaccgtggccaccctgcgggagacctatggagagatggctgactgctgtgccaagcaggagcctgagagaaatgaatgcttcctgcagcacaaggatgacaaccccaacctgccccggctggtgcggcctgaggtggatgtgatgtgcacagccttccatgacaatgaggagaccttcctgaagaagtacctgtatgaaattgcccggcggcacccctacttctacgcccctgagctgctgttctttgccaagcgctacaaggccgccttcacagagtgctgccaggccgctgacaaggccgcctgcctgctgcccaagctggatgagctgagagatgagggcaaggccagcagcgccaagcagaggctgaagtgtgccagcctgcagaagtttggagagcgggccttcaaggcctgggccgtggcccggctgagccagcgcttccccaaggccgagtttgctgaggtgtccaagctggtgacagacctgaccaaggtgcacacagagtgctgccacggggacctgctggagtgtgctgatgacagagctgacctggccaagtacatctgtgagaaccaggacagcatcagcagcaagctgaaggagtgctgtgagaagcccctgctggaaaagagccactgcatcgccgaggtggagaatgatgagatgcctgctgacctgcccagcctggccgctgactttgtggagagcaaggatgtgtgcaagaactatgcagaggccaaggatgtgttcctgggcatgttcctgtatgaatatgcccggcggcacccagactacagcgtggtgctgctgctgcggctggccaagacctatgagaccaccctggagaagtgctgtgccgctgctgacccccatgaatgttatgccaaggtgtttgatgagttcaagcccctggtggaggagccccagaacctgatcaagcagaactgtgagctgtttgagcagctgggggagtacaagttccagaatgccctgctggtgcgctacaccaagaaggtgccccaggtgtccacccccaccctggtggaggtgtccaggaacctgggcaaggtgggcagcaagtgctgcaagcaccctgaggccaagaggatgccctgtgccgaggactacctgtctgtggtgctgaaccagctgtgtgtgctgcacgagaagacccctgtgtctgacagagtgaccaagtgctgcacagagagcctggtgaacagaagaccctgcttcagcgccctggaggtggatgagacctacgtgcccaaggagttcaatgctgagaccttcaccttccacgccgacatctgcaccctgtctgagaaggagcggcagatcaagaagcagacagccctggtggagctggtgaagcacaagcccaaggccaccaaggagcagctgaaggctgtgatggatgactttgctgcctttgtggagaagtgctgcaaggcagatgacaaggagacctgctttgctgaggagggcaagaagctggtggccgccagccaggccgccctgggcctg

SEQ ID NO: 12 - DNA, 3393 bp, synthetic construct
  misc_feature 1..3393: note = vH1 Construct
  source 1..3393: mol_type = other DNA
atgagagtgcccgcccagctgctgggcctgctgctgctgtggctgcccggcgccagatgcgccatctgggagctgaagaaggacgtgtacgtggtggagctggactggtaccccgacgcccccggcgagatggtggtgctgacctgcgacacccccgaggaggacgggatcacctggaccctggatcagagcagcgaggtgctgggcagcggcaagaccctgaccatccaggtgaaggagtttggtgacgccggccagtacacctgccacaagggcggcgaggtgctgagccactcactgctgctgctgcacaagaaggaggacggcatctggagcaccgacattctgaaggatcagaaggagcccaagaacaagaccttcctgagatgcgaggccaagaactacagcggcagattcacctgttggtggctgactactatcagcactgatctgaccttcagcgtgaagagctcaagaggcagcagcgatccccagggcgtgacctgcggcgccgccaccctgagcgccgagagagtgagaggcgacaacaaggagtacgagtacagcgtggagtgccaggaagacagcgcctgccccgctgcagaggagtctctgcccatcgaggtgatggtggacgccgtgcacaagcttaagtacgagaactacaccagctccttcttcattagagacatcatcaagcccgacccgcccaaaaacctgcagctgaagcccctgaagaacagcagacaggtggaggtcagctgggagtaccccgacacctggagcaccccccacagctacttcagcctgaccttctgcgtgcaggtgcagggcaagagcaagagagagaagaaggacagagtgttcaccgacaaaaccagcgccaccgtgatctgcagaaaaaacgccagcattagcgtgagagctcaggatagatactacagcagcagctggagtgagtgggccagcgtgccctgcagcggcggctcaggcggcggctcaggcggcggcagcggcggcggcagcagaaacctgcccgtggccacgcccgaccccggcatgtttccctgcctgcaccatagccagaatctgctgagagccgtgagcaacatgctgcagaaggccagacagacgctcgagttctacccctgcaccagcgaggagattgaccacgaggacatcaccaaggacaagaccagcaccgtggaggcctgcctgcccctcgagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagacaatgaacgccaagctgctgatggaccccaagaggcagatttttctggaccagaacatgctggccgttatcgacgagctgatgcaggccctgaatttcaatagcgaaaccgtgccccagaagagcagcctggaggagcctgacttctacaaaaccaagatcaagctttgcatcctgctgcacgccttcagaatcagagccgtgaccatcgacagagtgatgagctacctgaacgccagcggcagcggcggcggcagcgacgcccacaagagcgaggtggcccataggttcaaggacctgggcgaggagaacttcaaggccctggtactcatcgccttcgcccaatacctgcaacagtgccccttcgaggaccatgttaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgaacgtaacgagtgcttcctgcagcacaaggacgacaaccccaacctgccccgactggtcagacccgaggtggacgtgatgtgcacagccttccacgacaacgaggagaccttcctgaagaagtacctctacgagatcgccagaagacatccatacttctacgcccccgagctgctgttcttcgccaagaggtacaaggccgccttcacagagtgctgccaggccgccgacaaggccgcttgcctgctgcctaagttggacgagctgagagacgagggcaaggccagcagcgccaagcagagactgaagtgcgcaagcctgcagaaattcggcgagagagcctttaaggcctgggccgtggccagactgagccagcgcttccccaaggccgagttcgccgaggtgagcaagctggtgaccgacctgaccaaagtgcacaccgagtgctgccacggcgacctgctggagtgcgccgacgacagagccgacctggccaagtacatctgcgagaaccaggacagcatcagcagcaagttgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccggagacaccccgactacagcgtggtgctgctgctgagactggccaagacctacgagaccaccctggagaagtgctgtgccgccgccgacccccacgagtgctacgccaaggtgttcgacgagttcaagcccctggtggaggagccccagaacctgatcaagcagaactgcgagctgttcgagcagctgggcgagtataagttccagaacgccctgctggtgagatacaccaagaaggtgccccaggtcagcacccccaccctggtggaggtgagcagaaatctgggcaaggtgggcagcaaatgctgcaagcaccccgaggccaagagaatgccctgcgccgaggactacctgtcagtggtgctgaaccagctgtgtgtgctgcacgagaagacccccgtgagcgacagagtgaccaagtgctgcaccgagagtctcgtgaacagacggccctgcttcagcgccctggaggtggacgaaacatacgtgcccaaggagttcaacgcagagaccttcaccttccacgcagacatctgcaccctgagcgagaaggagagacagatcaagaagcagaccgccctggttgagcttgtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 13 - DNA, 3393 bp, synthetic construct
  misc_feature 1..3393: note = vH2 Construct
  source 1..3393: mol_type = other DNA
atgagagtgcccgcccagctgctgggcctgctgctgctgtggctgcccggcgccagatgtgccatctgggagctgaagaaggacgtgtacgtggtggagctggactggtaccccgacgcccccggagagatggtggtgctgacctgcgacacccccgaagaggacggcatcacctggaccctggaccagagcagcgaggtgctgggcagcgggaagaccctgaccatccaggtgaaggagttcggcgacgccggccagtacacctgtcacaagggcggcgaggtgctgagccacagcctgctgctgctgcacaagaaggaggacggcatatggagcaccgacatcctgaaggaccagaaggagcccaagaacaagacctttctgagatgcgaggctaagaattacagcggcagattcacctgctggtggctgaccaccatcagcaccgacctgaccttcagcgtgaagagcagcagaggcagcagcgacccccagggcgtgacatgcggcgccgccaccctgagcgccgagagagtgagaggcgacaacaaggagtacgagtacagcgtggagtgccaggaggactccgcttgccccgccgccgaggagagcttgcccatcgaggtgatggtggacgccgtgcacaagctcaagtacgaaaactacaccagcagcttcttcatcagagacatcatcaagcccgacccccccaagaacctgcagctgaagcccctgaaaaacagcagacaggtggaggtgagctgggagtaccccgacacctggagcaccccccacagctacttcagcctgaccttctgcgtgcaggtgcagggaaaaagcaagagagagaagaaggacagagtgttcaccgacaagaccagcgccaccgtgatctgcagaaagaacgccagcatctccgtgagagcccaggacagatactacagcagcagctggagcgagtgggccagcgtgccctgtagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccatgaggacatcaccaaggacaagaccagcaccgtggaggcctgcctgcccctggagctgaccaagaacgaaagctgcctgaacagcagagagaccagcttcatcaccaacggcagctgtctggcctcaagaaagaccagctttatgatggccctgtgcctgtctagcatctacgaggatctgaagatgtaccaggtggagttcaagaccatgaacgccaagctgctgatggaccccaagagacagatcttcctggaccagaacatgttagccgtgattgacgagctgatgcaggccctgaacttcaatagcgagaccgtgccccagaaaagcagcctggaggagcccgacttctacaagaccaagatcaagctgtgcatcctgctgcacgccttcagaatcagagccgtaaccatcgacagagtgatgagctacctgaacgccagtggcagcggcggtggcagcgacgctcacaagagcgaggtggcccacagattcaaggacctgggcgaggagaacttcaaggccctggtcctgatcgccttcgcccagtacctgcagcagtgccccttcgaggaccacgtgaagctggtgaacgaggtgaccgagttcgccaagacctgcgtggccgacgagtcggccgagaactgcgacaagagcctgcacaccctgttcggcgacaagctgtgcaccgtggccaccctgagagaaacctacggggagatggccgactgctgcgcaaagcaggagcccgagcgaaacgagtgcttcctgcagcacaaggacgacaaccccaacttgcccagactggtaagacccgaggtggacgtcatgtgcaccgctttccacgacaacgaggagaccttcctgaagaagtacctgtacgagatcgccagaagacacccctatttctatgcccctgagctgctgttcttcgccaagcgctacaaggccgccttcaccgagtgctgccaggccgccgacaaggccgcctgcctgctccccaagctggacgagctgagagacgaaggcaaggccagcagcgccaagcagagactgaagtgcgccagcctgcagaagttcggcgagagagccttcaaggcctgggccgtggccagactgtcgcagagattccccaaggccgagttcgccgaggtgagcaagctggttaccgacctgactaaggtgcacaccgagtgctgccacggcgacctgctggagtgcgccgacgacagagccgacctggccaagtacatctgcgagaaccaggatagcatcagcagcaagctgaaggagtgctgcgagaagccccttctggagaagtcccactgcatcgccgaggtggagaacgacgagatgcccgccgacctgcctagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagactacccttgagaagtgctgcgccgccgccgacccacatgagtgctacgccaaggtgttcgacgagttcaagcccctggtggaagagccccagaacctgatcaagcagaactgcgagctgttcgagcagctgggcgagtacaagttccagaacgcccttctggtgagatacaccaagaaggtgcctcaggtgagcacccccacccttgtggaggtgagcagaaacctcggcaaggtgggcagcaagtgctgcaagcatccagaggccaagagaatgccctgcgccgaggactacctgagcgtggtgctgaaccagctgtgcgtgctgcacgagaaaactcccgtgagcgacagagtgaccaagtgctgcaccgagagtctggtgaacagaagaccctgcttcagcgccctggaggtggacgagacctacgtgcccaaggagttcaacgccgagaccttcaccttccacgccgacatctgcaccctgagcgagaaggagcggcagatcaagaagcagaccgccctggtggagctggtgaagcacaagcccaaggccaccaaggagcagctcaaggccgtgatggacgacttcgccgcgttcgtggagaagtgctgcaaggccgacgacaaagagacctgcttcgccgaggagggcaagaagcttgtggccgccagccaggccgccctgggcctgtag

SEQ ID NO: 14 - DNA, 3393 bp, synthetic construct
  misc_feature 1..3393: note = vH3 Construct
  source 1..3393: mol_type = other DNA
atgagagtgcccgcccagcttctgggcctgctgctgctgtggctgcccggcgccagatgcgccatctgggagctgaagaaggacgtgtacgtggtggagctggactggtacccggacgccccgggcgagatggtcgtgctgacctgcgacacccccgaggaggacggcatcacctggaccctggaccagtctagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcctaagaacaagaccttcctgagatgtgaggccaagaactacagcggcagattcacctgctggtggctgaccaccatcagcaccgatctgaccttcagcgtgaagagcagcagaggcagctcagacccccagggcgtgacctgcggcgccgcgaccctgagcgccgagagagtgagaggcgacaacaaggagtacgagtacagcgtggagtgccaggaggacagcgcctgccccgcggccgaggagagcctgcccatcgaggtgatggtggacgccgtgcataagctgaagtacgagaactacaccagcagcttcttcatcagagacatcatcaagcccgatccccccaagaacctgcagctgaagccactgaagaacagcagacaggtggaggtgagctgggagtaccccgacacctggagcaccccccacagctacttcagcctgaccttctgcgtgcaggtgcagggcaagagcaagagagagaagaaggaccgggtgttcaccgacaagaccagcgccactgtgatctgcagaaagaacgccagcatcagcgtgagagcccaggacagatactacagctccagctggtcagagtgggccagcgtgccctgcagcggcggcagcggtggcgggagcggcggcgggagcggcggcggaagcagaaacctgcccgtggccacccctgaccccggcatgttcccctgcctgcaccacagccagaacctgctgagagccgtaagcaacatgctgcagaaggccagacagactctggagttctacccctgcaccagcgaggagatcgatcacgaggacatcaccaaggacaagaccagtaccgtggaggcctgcctgcccctggagctgaccaagaacgagagctgcctgaacagcagagagaccagctttataaccaacggcagctgtctggctagcagaaagaccagcttcatgatggccctgtgcctgagcagcatctacgaggacttgaagatgtaccaggtggagttcaagaccatgaacgccaagcttctgatggaccccaagagacagatctttctggaccagaacatgctggccgtgatcgatgaactgatgcaggccctgaacttcaacagcgagaccgtgccccagaagagcagtctggaggagcccgacttctacaagactaagatcaagctgtgcatcctgctgcacgccttcagaatcagagccgtgaccatcgacagagtgatgagctacctgaacgcctcaggcagcggcggcgggagcgacgcccacaagagcgaggtggcccacagattcaaggacctcggcgaggagaacttcaaggccctggtgctgatcgctttcgcccagtacctgcagcagtgccccttcgaggaccacgtgaaactggtgaacgaggtgaccgagttcgccaagacctgcgtggccgacgagagcgccgagaactgcgacaagagcctgcacacgttgttcggcgacaagctgtgcaccgtggccaccctgagagagacctacggcgagatggccgactgctgcgccaagcaggagcccgagagaaacgaatgcttcctgcagcacaaggacgacaaccccaacctgccccgcctggtgagacccgaggtggacgtgatgtgcaccgccttccacgacaacgaggagaccttcctgaagaagtacttgtacgagatcgcaagaagacacccgtacttctacgcccccgagctgctgttcttcgccaagagatacaaggccgccttcaccgagtgctgccaggccgccgacaaggccgcctgcctgctgcccaagctggacgagctcagagacgagggcaaggccagcagcgccaagcagagactgaagtgcgccagcctgcagaagttcggcgagagagcctttaaggcctgggccgtggccagactgagccagagattccccaaggccgagttcgccgaagtgagcaagctggtgaccgacctgacgaaggtgcacaccgagtgctgccacggcgacctgctggagtgcgccgacgacagagcagacctggccaagtacatctgcgagaaccaggacagcatcagcagcaagctgaaggagtgctgcgagaagcccctgctggagaagagccactgcatcgccgaggtggagaacgacgagatgcccgccgacctgcccagtctggccgcagacttcgtggagagcaaggatgtgtgcaagaactacgccgaggccaaggacgtgttcctgggcatgttcctgtacgagtacgcaagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgaactgttcgagcagcttggcgagtacaagtttcagaacgccctgctggtcagatacaccaagaaggtgccccaggtgagcacccccaccctggtggaggtgtccagaaacctgggcaaggtgggcagcaagtgctgcaagcaccccgaggcaaagagaatgccctgcgccgaggactatctgagcgtggtgctgaaccagctgtgcgtgctgcacgagaagacccccgtgagcgacagagtgaccaagtgctgcaccgagagcctggtgaatagaagaccctgcttcagcgcactggaggtggacgagacctacgtgcccaaggagttcaacgccgagacattcaccttccacgccgatatctgcaccctgagcgagaaggagagacagatcaagaagcagaccgccctggtggagctggtgaagcacaagcccaaggccaccaaggagcagttgaaggccgtgatggacgacttcgccgccttcgtggagaaatgctgcaaggccgacgacaaggagacctgcttcgccgaggagggcaagaagctggtggccgccagccaggccgccctgggcctgtag

SEQ ID NO: 15 - DNA, 1617 bp, synthetic construct
  misc_feature 1..1617: note = A1 Construct
  source 1..1617: mol_type = other DNA
atgagggtccccgctcagctcctggggctcctgctgctctggctcccaggtgcacgatgtgccatctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggaagcggcggaggctctggcggagggagtggcggaggcagcagaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagc

SEQ ID NO: 16 - DNA, 1614 bp, synthetic construct
  misc_feature 1..1614: note = A2 Construct
  source 1..1614: mol_type = other DNA
atgagggtccccgctcagctcctggggctcctgctgctctggctcccaggtgcacgatgtgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggaggtggcagtggcggaggcagcagaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgct

SEQ ID NO: 17 - DNA, 1617 bp, synthetic construct
  misc_feature 1..1617: note = A3 Construct
  source 1..1617: mol_type = other DNA
atgagggtccccgctcagctcctggggctcctgctgctctggctcccaggtgcacgatgtgccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaaaagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaaaagcagcctggaggagcccgacttctacaagaccaagatcaagctgtgcatcctgctgcacgccttcagaatcagagccgtgaccatcgacagagtgatgagctacctgaacgccagc

SEQ ID NO: 18 - DNA, 1617 bp, synthetic construct
  misc_feature 1..1617: note = A4 Construct
  source 1..1617: mol_type = other DNA
atgagggtccccgctcagctcctggggctcctgctgctctggctcccaggtgcacgatgtgccatctgggagctgaagaaggatgtgtatgtggtggagctggactggtacccagatgcccctggagagatggtggtgctgacctgtgacaccccagaggaggatggcatcacctggaccctggaccagagcagcgaggtgctgggcagcggcaagaccctgaccatccaggtgaaggagtttggagatgctggccagtacacctgccacaagggcggggaggtgctgagccacagcctgctgctgctgcacaagaaggaggatggcatctggagcacagacatcctgaaggaccagaaggagcccaagaacaagaccttcctgcgctgtgaggccaagaactacagcggccgcttcacctgctggtggctgaccaccatcagcacagacctgaccttctctgtgaaaagcagccggggcagcagtgacccccagggcgtgacctgtggggccgccaccctgtctgctgagcgggtgcggggggacaacaaggagtatgagtacagcgtggagtgccaggaggacagcgcctgcccagctgctgaggagagcctgcccatcgaggtgatggtggatgctgtgcacaagctgaagtatgagaactacaccagcagcttcttcatccgggacatcatcaagccagacccccccaagaacctgcagctgaagcccctgaagaacagccggcaggtggaggtgtcctgggagtacccagacacctggagcaccccccacagctacttcagcctgaccttctgtgtgcaggtgcagggcaagagcaagcgggagaagaaggacagagtcttcacagacaagaccagcgccaccgtcatctgcaggaagaatgccagcatctctgtgcgggcccaggaccgctactacagcagctcctggagcgagtgggcctctgtgccctgcagcggcggcagcggcggcggcagcggcggcggcagcggcggcggcagcaggaacctgcctgtggccaccccagaccccggcatgttcccctgcctgcaccacagccagaacctgctgcgggctgtgagcaacatgctgcagaaggcccggcagaccctggagttctacccctgcaccagcgaggagattgaccacgaggacatcaccaaggacaagaccagcacagtggaggcctgcctgcccctggagctgaccaagaatgaaagctgcctgaacagccgggagaccagcttcatcaccaacggcagctgcctggccagcaggaagaccagcttcatgatggccctgtgcctgagcagcatctatgaggacctgaagatgtaccaggtggagttcaagaccatgaatgccaagctgctgatggaccccaagaggcagatcttcctggaccagaacatgctggccgtgattgatgagctgatgcaggccctgaacttcaacagcgagaccgtgccccagaaaagcagcctggaggagccagacttctacaagaccaagatcaagctgtgcatcctgctgcacgccttccgcatccgggctgtgaccatcgacagagtgatgagctacctgaatgccagc

SEQ ID NO: 19 - DNA, 3390 bp, synthetic construct
  misc_feature 1..3390: note = B1 Construct
  source 1..3390: mol_type = other DNA
atgagggtccccgctcagctcctggggctcctgctgctctggctcccaggtgcacgatgtgccatctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggaagcggcggaggctctggcggagggagtggcggaggcagcagaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagcggctctggcggcggcagtgacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgaatgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaaaagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctt

SEQ ID NO: 20 - DNA, 3390 bp, synthetic construct
  misc_feature 1..3390: note = B2 Construct
  source 1..3390: mol_type = other DNA
atgagggtccccgctcagctcctggggctcctgctgctctggctcccaggtgcacgatgtgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggaggtggcagtggcggaggcagcagaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgctagcggcagcggtggcggcagcgacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctgttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggaaaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctg

SEQ ID NO: 21 - DNA, 3390 bp, synthetic construct
  misc_feature 1..3390: note = B3 Construct
  source 1..3390: mol_type = other DNA
atgagggtccccgctcagctcctggggctcctgctgctctggctcccaggtgcacgatgtgccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaaaagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagcggcggcagcggcggcggcagcggcggcggcagcggcggcggcagcagaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaaaagcagcctggaggagcccgacttctacaagaccaagatcaagctgtgcatcctgctgcacgccttcagaatcagagccgtgaccatcgacagagtgatgagctacctgaacgccagcggcagcggcggcggcagcgacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaaaagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctg

SEQ ID NO: 22 - DNA, 3390 bp, synthetic construct; identical to SEQ ID NO: 11
  misc_feature 1..3390: note = B4 Construct
  source 1..3390: mol_type = other DNA

SEQ ID NO: 23 - DNA, 4374 bp, synthetic construct
  misc_feature 1..4374: note = C1 Construct
  source 1..4374: mol_type = other DNA
atgagggtccccgctcagctcctggggctcctgctgctctggctcccaggtgcacgatgtgccatctgggagctgaaaaaggacgtgtacgtggtggaacttgactggtaccccgacgcccccggcgagatggtggtactgacctgcgacactcccgaggaagacggcattacctggaccttggaccagagcagcgaggttctgggctccggaaaaaccttgacaatccaagtgaaagaattcggcgacgctggccagtacacctgccacaagggcggcgaggtgctgtcccacagcctgctgctgctgcataagaaagaagacgggatttggagcaccgatatactgaaggatcagaaggagcccaagaacaagaccttcctgaggtgcgaggccaaaaattacagcggcagattcacctgctggtggctgaccaccattagcacagacctgactttcagcgtaaagtcttcaaggggcagctcagacccccagggagtaacttgcggagcggcaacgttgtctgccgagcgggtcagaggcgacaataaggagtacgagtattcagtagagtgtcaggaagatagcgcctgtcccgccgcggaggagagcctccccatcgaggtgatggtggacgccgtgcacaagttaaagtacgagaattacaccagctcattttttatcagagacattatcaagccggaccccccgaagaacttacagcttaaacccctaaagaacagcaggcaggttgaggtcagctgggaatatcctgacacctggtcaaccccccacagctacttctcccttactttctgtgtgcaagtgcagggcaagagcaagagagaaaagaaggaccgggtgtttaccgacaagactagcgccaccgtgatttgcagaaagaacgccagcattagtgtgagagcccaggacaggtattactccagctcatggtctgagtgggctagtgtgccttgctctggaggcagcggtggcgggagcggcggaggctccgggggaggtagccggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaaaagcagcctcgaggagcccgacttctacaaaacaaagatcaaactctgcatccttctgcacgccttcagaattagagccgtgaccatcgacagagttatgagctacctgaatgccagcggcagcggcggcggatccgatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctcggtggcgggtctggtggcggttctcagtactacgattacgacttccccctgagtatctacggtcagtcctcacctaactgcgccccagagtgtaactgccccgaaagctacccgagcgccatgtactgcgacgagctgaagttgaagtccgtgcccatggtgcccccaggcatcaagtacttatacttgaggaacaaccaaatcgatcatatcgacgagaaggccttcgaaaacgtaaccgacctgcagtggctgatactggatcacaatctgctagagaattccaagatcaagggcagagtgttctcgaagctgaaacaactgaagaagctgcacatcaaccataacaatctcaccgagagcgtgggtcccctgcccaagtcgctggaggacctgcagctgacccacaacaaaataaccaaactaggcagcttcgaggggttggtgaatctgaccttcatccatttgcagcataacagactaaaggaggatgccgtgagcgccgccttcaaaggcctcaagagccttgagtacctggacctgagcttcaaccagatcgcccggctgcccagtgggctgcccgtgagcctgctgacgctgtatctggacaataacaaaatcagcaacatccccgacgaatacttcaaaagattcaacgccttacagtacctgcgactcagccacaatgagctcgctgacagcggcatccctggcaacagcttcaacgtgtcatccctggtggagctggacctgagttacaataagctgaagaacatcccaactgtcaatgagaatttggaaaactactacctggaggtgaaccagctggagaagttcgacattaagagcttttgcaaaatcctgggcccactgtcatatagcaagatcaagcacctgcgactggacggcaaccgaatcagtgaaacttccctaccccctgacatgtacgagtgcctgagagtagcaaatgaggtgaccctgaac

SEQ ID NO: 24 - DNA, 4374 bp, synthetic construct
  misc_feature 1..4374: note = C2 Construct
  source 1..4374: mol_type = other DNA
atgagggtccccgctcagctcctggggctcctgctgctctggctcccaggtgcacgatgtgccatctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagcggtggaagcggcggaggctctggcggagggagtggcggaggcagcagaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagcggctctggcggcggcagtgacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgaatgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaaaagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggccttggtggcgggtctggtggcggttctcagtattacgactacgatttccccctgagcatctatggccagagcagccctaactgcgccccggagtgcaactgccccgaaagctacccaagcgccatgtactgcgatgagctgaagctgaagtctgtgcctatggtgcctcccggcatcaagtacctgtacctgagaaacaaccagatagaccacatcgatgagaaagccttcgagaacgtcaccgacctgcagtggctgattctggaccacaatttactggagaactccaagatcaagggcagagtgttctccaagttaaagcagctgaagaaactgcacatcaatcacaacaacctgaccgagagcgtgggcccactgcccaagagcctagaggatctgcagctcacccacaacaagatcactaagttgggcagcttcgagggcctcgtaaacttgacattcatacatctgcagcacaacagacttaaggaagacgccgtgagtgcggcctttaagggtctgaaaagcctggagtacttagacctgagcttcaaccagatcgcaaggctgcccagcggccttccggtcagtctgctgaccctgtatctggacaacaacaagatcagcaacatccccgacgagtacttcaagcggtttaacgccctccagtacctgagactgagccacaacgagttagctgactcgggcatacccggtaacagcttcaatgttagcagcctagttgagcttgacttgagctacaacaagcttaagaacatcccaaccgtgaacgagaacctcgagaattactacctggaagtcaaccagctggagaagttcgacattaagagcttctgcaaaatcctgggcccactgtcctatagcaagatcaagcacctgcgccttgacggaaacagaattagcgagaccagccttccaccagacatgtacgagtgcctgagggtggccaacgaggtgaccctgaac

SEQ ID NO: 25 - DNA, 4374 bp, synthetic construct
  misc_feature 1..4374: note = C3 Construct
  source 1..4374: mol_type = other DNA
atgagggtccccgctcagctcctggggctcctgctgctctggctcccaggtgcacgatgtgccatctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagcggcgggagcggcggcggcagcggaggtggcagtggcggaggcagcagaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgctagcggcagcggtggcggcagcgacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctgttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggaaaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctgggtggcgggtctggtggcggttctcagtactacgactacgatttccccctatccatctacgggcagagctcgcctaactgcgcccccgagtgtaactgccccgagtcgtaccccagcgccatgtactgtgacgagctgaagctgaaaagcgtgcccatggtgccccccggcatcaagtacctgtacttgagaaacaaccagatcgaccacattgacgaaaaggccttcgagaacgtaaccgacctgcagtggctgatcctggaccacaacctgcttgagaacagcaagatcaagggccgcgtgttcagcaagctgaagcagctgaagaagctgcacatcaaccacaacaacttgactgagtctgttggccccctaccaaagagcctggaggacctgcagctgacccacaataagataaccaagctgggctcattcgagggcctggtgaacttgacctttattcacctgcagcataacagactgaaggaggacgccgtgagcgccgcctttaaggggctgaaaagcctggagtacctggacctgagttttaaccagatcgccagactgccctcaggcctgcccgtgagtttgctgactctgtacctggacaacaataagatcagcaacattcctgacgagtatttcaaaagattcaatgctctgcagtacctgagactaagccacaacgagctggccgacagcggaatccccggcaacagcttcaacgtgagcagcttggtggagttggacctgagctacaacaaactgaagaacatccccaccgtcaatgagaacttggagaattactacctcgaggttaaccagcttgagaagttcgacatcaagagcttctgcaagatcctgggccccctcagctacagcaagatcaagcacttgagactggacgggaacagaatcagcgaaaccagccttcctcccgacatgtacgagtgccttagagtggcaaatgaggtgaccctgaac

SEQ ID NO: 26 - DNA, 63 bp, synthetic construct
  misc_feature 1..63: note = Leader Sequence of L1
  source 1..63: mol_type = other DNA
atgcgagttcctgctcagctgcttggtctgttactgctgtggttgccgggcgcacgatgtgct

SEQ ID NO: 27 - DNA, 63 bp, synthetic construct
  misc_feature 1..63: note = Leader Sequence of L2
  source 1..63: mol_type = other DNA
atgcgcgtgcccgcacaattgctcggactgctgctactgtggctgcccggggctcgctgcgca

SEQ ID NO: 28 - DNA, 63 bp, synthetic construct
  misc_feature 1..63: note = Leader Sequence of L3
  source 1..63: mol_type = other DNA
atgagagttcctgctcaactactagggctgctgttgttgtggttgcccggcgcgcgatgcgca

SEQ ID NO: 29 - DNA, 63 bp, synthetic construct
  misc_feature 1..63: note = Leader Sequence of M1
  source 1..63: mol_type = other DNA
atgagagtccccgcccagctgctggggcttcttttgctttggcttcctggcgcaagatgcgct

SEQ ID NO: 30 - DNA, 63 bp, synthetic construct
  misc_feature 1..63: note = Leader Sequence of M2
  source 1..63: mol_type = other DNA
atgagagtccccgcccagctgctagggctgctgctgctgtggttacccggcgcccggtgtgca

SEQ ID NO: 31 - DNA, 63 bp, synthetic construct
  misc_feature 1..63: note = Leader Sequence of M3
  source 1..63: mol_type = other DNA
atgagagtcccagctcagctgctgggcctactgctgctatggctccccggcgcgcggtgcgcc

SEQ ID NO: 32 - DNA, 65 bp, synthetic construct
  misc_feature 1..65: note = Leader Sequence of H1
  source 1..65: mol_type = other DNA
cvatgagagtgcccgcccagttgcttggcctgctgctcctatggcttcccggcgccagatgcgcc

SEQ ID NO: 33 - DNA, 63 bp, synthetic construct
  misc_feature 1..63: note = Leader Sequence of H2
  source 1..63: mol_type = other DNA
atgagagtgcccgcccagctgctgggcctgctgctcttatggctgcccggcgcccgctgtgct

SEQ ID NO: 34 - DNA, 63 bp, synthetic construct
  misc_feature 1..63: note = Leader Sequence of H3
  source 1..63: mol_type = other DNA
atgagagtgcccgcccagctgctgggcctgctgttgctgtggctgcccggcgccagatgcgcc

SEQ ID NO: 35 - DNA, 63 bp, synthetic construct
  misc_feature 1..63: note = Leader Sequence of CO
  source 1..63: mol_type = other DNA
atgagagtgcccgcccagctgctgggcctgctgctgctgtggctgcccggcgccagatgcgcc

SEQ ID NO: 36 - DNA, 63 bp, synthetic construct
  misc_feature 1..63: note = Leader Sequence of CP
  source 1..63: mol_type = other DNA
atgagggtccccgctcagctcctggggctcctgctgctctggctcccaggtgcacgatgtgcc

SEQ ID NO: 37 - DNA, 63 bp, synthetic construct; identical to SEQ ID NO: 35
  misc_feature 1..63: note = Leader Sequence of vH1
  source 1..63: mol_type = other DNA

SEQ ID NO: 38 - DNA, 63 bp, synthetic construct
  misc_feature 1..63: note = Leader Sequence of vH2
  source 1..63: mol_type = other DNA
atgagagtgcccgcccagctgctgggcctgctgctgctgtggctgcccggcgccagatgtgcc

SEQ ID NO: 39 - DNA, 63 bp, synthetic construct
  misc_feature 1..63: note = Leader Sequence of vH3
  source 1..63: mol_type = other DNA
atgagagtgcccgcccagcttctgggcctgctgctgctgtggctgcccggcgccagatgcgcc

SEQ ID NO: 40 - DNA, 63 bp, synthetic construct; identical to SEQ ID NO: 36
  misc_feature 1..63: note = Leader Sequence of A1
  source 1..63: mol_type = other DNA

SEQ ID NO: 41 - DNA, 63 bp, synthetic construct; identical to SEQ ID NO: 36
  misc_feature 1..63: note = Leader Sequence of A2
  source 1..63: mol_type = other DNA

SEQ ID NO: 42 - DNA, 63 bp, synthetic construct; identical to SEQ ID NO: 36
  misc_feature 1..63: note = Leader Sequence of A3
  source 1..63: mol_type = other DNA

SEQ ID NO: 43 - DNA, 63 bp, synthetic construct; identical to SEQ ID NO: 36
  misc_feature 1..63: note = Leader Sequence of A4
  source 1..63: mol_type = other DNA

SEQ ID NO: 44 - DNA, 63 bp, synthetic construct; identical to SEQ ID NO: 36
  misc_feature 1..63: note = Leader Sequence of B1
  source 1..63: mol_type = other DNA

SEQ ID NO: 45 - DNA, 63 bp, synthetic construct; identical to SEQ ID NO: 36
  misc_feature 1..63: note = Leader Sequence of B2
  source 1..63: mol_type = other DNA

SEQ ID NO: 46 - DNA, 63 bp, synthetic construct; identical to SEQ ID NO: 36
  misc_feature 1..63: note = Leader Sequence of B3
  source 1..63: mol_type = other DNA

SEQ ID NO: 47 - DNA, 63 bp, synthetic construct; identical to SEQ ID NO: 36
  misc_feature 1..63: note = Leader Sequence of B4
  source 1..63: mol_type = other DNA

SEQ ID NO: 48 - DNA, 63 bp, synthetic construct; identical to SEQ ID NO: 36
  misc_feature 1..63: note = Leader Sequence of C1
  source 1..63: mol_type = other DNA

SEQ ID NO: 49 - DNA, 63 bp, synthetic construct; identical to SEQ ID NO: 36
  misc_feature 1..63: note = Leader Sequence of C2
  source 1..63: mol_type = other DNA

SEQ ID NO: 50 - DNA, 63 bp, synthetic construct; identical to SEQ ID NO: 36
  misc_feature 1..63: note = Leader Sequene of C3
  source 1..63: mol_type = other DNA

SEQ ID NO: 51 - DNA, 918 bp, synthetic construct
  misc_feature 1..918: note = IL-12b of L1
  source 1..918: mol_type = other DNA
atctgggaattaaagaaagatgtgtacgtggtggaattggattggtacccagatgctccgggcgaaatggttgtactcacatgcgatactccggaggaagacggtatcacttggacattggatcagtcgagtgaggttctcggtagtggtaaaacactaacgatccaagtcaaagaattcggtgatgcggggcaatatacctgtcataaaggcggcgaagtactatctcatagcctcctgctgttacacaagaaggaagatggcatatggtccaccgacatccttaaggatcagaaagaacccaagaacaaaactttcttgcgttgcgaagctaagaactactccggccgcttcacatgctggtggttgacaacgatcagtacggatctaaccttctctgttaagtccagtcgggggagttcggacccccaaggcgtcacgtgtggagctgccacactttccgctgagcgcgtacgtggagataataaagagtatgaatactccgttgagtgccaggaggactccgcgtgccccgctgccgaggagagtctccccatagaggtgatggtcgacgctgttcacaaactgaaatatgagaactatacctcatccttctttatacgtgacataattaagccagatcccccgaagaacttacaattgaaaccattgaagaattcacgtcaagtcgaggtatcctgggagtatcccgacacctggtccacgccacactcatatttctctctgaccttctgtgtgcaggtacaaggcaagagcaaacgagaaaaaaaggacagagttttcacggataagactagcgccacagtgatatgtaggaaaaacgcatcgatctcagtccgcgcgcaagatcggtattactcaagcagttggtcagagtgggcatcggtgccctgctcg

SEQ ID NO: 52 - DNA, 918 bp, synthetic construct
  misc_feature 1..918: note = IL-12b of L2
  source 1..918: mol_type = other DNA
atttgggaacttaagaaggacgtatacgttgtggaacttgactggtaccctgatgctccaggggagatggtggttttgacgtgtgacaccccggaagaagatggaattacatggaccttagaccaatcctccgaggttcttggctcgggcaaaaccttgaccattcaggtcaaggaatttggcgatgctggccaatacacctgccataaaggtggtgaagttttatctcactccctactgttgctccataagaaagaagacggcatttggtcgacagacatattgaaggatcagaaggaacctaagaataagaccttcttacgatgtgaggccaagaattattccggacgttttacgtgttggtggttgaccacgatctccactgacttaaccttctcagtgaaatcctcacgaggtagttccgatccccagggtgtgacgtgcggtgcggccacgttaagtgctgagagagtacggggcgacaataaggaatacgagtactcagttgaatgccaagaggactcggcctgtcccgcggcagaggagagtctccctatcgaagtgatggtggatgcggtgcacaagctcaagtatgaaaattacacatcatcttttttcatcagagacattataaagcccgacccaccaaagaacctccagttaaagcccttaaagaacagtagacaggttgaagtatcatgggaatacccagacacctggtccacaccccattcgtatttttccttgacgttttgcgtacaagttcagggaaagtccaaacgggaaaagaaagaccgcgtttttacagacaaaacttctgccactgtcatctgtagaaagaacgcatcaattagcgtgcgagcgcaagacagatactattcaagtagctggagcgagtgggccagtgttccatgttct

SEQ ID NO: 53 - DNA, 918 bp, synthetic construct
  misc_feature 1..918: note = IL-12b of L3
  source 1..918: mol_type = other DNA
atatgggagctcaagaaggacgtttatgtcgttgagctggattggtaccctgatgccccgggcgaaatggttgtgcttacgtgcgatacccccgaggaggatggcataacatggacgttagatcagtcttccgaggtccttggttccggtaagactcttactatccaggtgaaggagttcggcgatgccggccagtacacttgccataaaggcggtgaagttctaagccactctctactgcttttgcacaagaaggaagatggaatatggtccaccgacatcttgaaggaccagaaagaaccaaaaaataagacatttttgaggtgtgaggcaaaaaattattcgggacgcttcacctgctggtggttgacgacgatttcaaccgacctcaccttctcagtaaagagttcgagaggtagttccgatccccaaggtgtgacatgtggcgctgcgactctaagcgctgaacgcgtaagaggtgataacaaagagtacgaatacagtgtggaatgccaagaagatagcgcgtgtccagccgcagaagaatctttaccaatagaggttatggttgatgccgttcacaaattgaaatatgagaattacacctcaagctttttcattcgagacataataaagcccgaccctcctaagaatcttcagttaaaaccgctgaagaacagtagacaagttgaggttagctgggaatatcctgatacctggtcaacgccgcactcgtatttctccctgactttctgtgttcaggttcaaggaaaatctaaaagggagaagaaagaccgtgttttcaccgacaagacatctgccacagtcatatgtaggaagaacgcttcaatcagcgtgcgagcgcaggaccgatactacagctctagttggtccgaatgggccagcgtaccatgctct

SEQ ID NO: 54 - DNA, 918 bp, synthetic construct
  misc_feature 1..918: note = IL12b of M1
  source 1..918: mol_type = other DNA
atctgggagctgaaaaaggacgtgtacgtggtggaacttgactggtaccccgacgcccccggcgagatggtggtactgacctgcgacactcccgaggaagacggcattacctggaccttggaccagagcagcgaggttctgggctccggaaaaaccttgacaatccaagtgaaagaattcggcgacgctggccagtacacctgccacaagggcggcgaggtgctgtcccacagcctgctgctgctgcataagaaagaagacgggatttggagcaccgatatactgaaggatcagaaggagcccaagaacaagaccttcctgaggtgcgaggccaaaaattacagcggcagattcacctgctggtggctgaccaccattagcacagacctgactttcagcgtaaagtcttcaaggggcagctcagacccccagggagtaacttgcggagcggcaacgttgtctgccgagcgggtcagaggcgacaataaggagtacgagtattcagtagagtgtcaggaagatagcgcctgtcccgccgcggaggagagcctccccatcgaggtgatggtggacgccgtgcacaagttaaagtacgagaattacaccagctcattttttatcagagacattatcaagccggaccccccgaagaacttacagcttaaacccctaaagaacagcaggcaggttgaggtcagctgggaatatcctgacacctggtcaaccccccacagctacttctcccttactttctgtgtgcaagtgcagggcaagagcaagagagaaaagaaggaccgggtgtttaccgacaagactagcgccaccgtgatttgcagaaagaacgccagcattagtgtgagagcccaggacaggtattactccagctcatggtctgagtgggctagtgtgccttgctct

SEQ ID NO: 55 - DNA, 918 bp, synthetic construct
  misc_feature 1..918: note = IL12b of M2
  source 1..918: mol_type = other DNA
atttgggagttgaagaaggacgtgtacgtggtggagctggactggtacccggatgctcccggcgagatggtggtactcacctgcgacacacctgaggaagacggcatcacctggaccctcgatcagagcagcgaggttctgggaagcggcaaaaccctgaccatccaagtgaaagagtttggcgacgccggtcagtacacctgccacaagggaggcgaggtcctgtctcactctctgctgctgctccataagaaggaggacggtatttggagcactgacatcttgaaggatcaaaaagagccaaagaataaaacgttcctgaggtgcgaagctaagaattactccgggcgttttacgtgctggtggctgaccacgatcagcaccgatctgaccttcagcgtgaagagcagccggggcagcagcgacccccaaggcgtgacttgcggcgctgcgaccctgagcgctgagcgtgtgcgcggcgacaacaaggagtatgagtattcagtggagtgtcaggaggactccgcctgtcccgcggccgaagagagtctgcctattgaggtgatggtggacgccgtgcacaagctgaagtacgagaactacacatcgtcattctttatccgcgacatcataaagcccgacccccccaagaacctgcagctgaagcctctcaagaattcccggcaagtggaggtgagctgggagtaccctgatacctggtctacccctcacagctactttagcctgaccttctgcgtccaggtgcaaggaaagtcgaagcgcgagaagaaagatagagtcttcaccgataaaaccagtgccaccgtgatttgccgcaaaaacgcctccatcagcgtgcgggctcaggatagatactactctagcagctggagcgaatgggcctcagttccttgcagc

SEQ ID NO: 56 - DNA, 918 bp, synthetic construct
  misc_feature 1..918: note = IL12b of M3
  source 1..918: mol_type = other DNA
atttgggagctcaagaaggacgtgtacgtggtggaacttgactggtacccggacgcgcccggggaaatggtggtgctaacctgtgacacccccgaagaggacggcatcacctggaccctggaccagagcagtgaggtgctaggtagtggcaaaacgttaaccatccaggtcaaggagttcggcgacgccgggcaatacacctgtcacaaggggggggaggtactatcccactccctgctgctcctgcacaagaaagaggacgggatctggagcaccgacattctgaaagaccaaaaggagcccaaaaacaaaaccttccttagatgtgaagccaagaactacagcggccgtttcacctgctggtggctgaccaccatatctacggaccttaccttttcggtgaagagcagcagggggagttccgacccgcaaggcgtaacttgcggagccgcaaccctgagcgccgagagagtgcgcggcgacaacaaggagtacgagtatagcgtggagtgccaagaggacagcgcatgcccagccgccgaagagagcctgccaatagaggtcatggtagacgccgtgcacaagctaaaatatgaaaactacaccagcagctttttcatcagggatatcatcaaacccgacccaccaaaaaacttacagcttaagcctctgaaaaacagcagacaagttgaggtcagctgggagtaccccgacacttggagcacaccccactcctatttcagtttgacattctgcgtgcaggtgcagggtaaaagcaagagagaaaagaaggacagagtgttcacagataagacctcagccacagtgatctgccgtaagaatgccagcatcagcgtccgggctcaggacaggtactactcttcctcatggagcgagtgggcctctgtcccctgcagc

SEQ ID NO: 57 - DNA, 918 bp, synthetic construct
  misc_feature 1..918: note = IL12b of H1
  source 1..918: mol_type = other DNA
atctgggagctgaagaaagacgtgtacgtggtggagctggactggtaccccgacgcccccggtgaaatggtggttctgacctgcgacacaccagaggaggacggcatcacctggaccctggaccagtccagcgaggtcctgggctctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagc

SEQ ID NO: 58 - DNA, 918 bp, synthetic construct
  misc_feature 1..918: note = IL12b of H2
  source 1..918: mol_type = other DNA
atttgggagctgaagaaggacgtgtacgtggtggagctggattggtatccagacgcccccggcgagatggtcgtgttgacctgcgatactcccgaggaggacggaatcacctggacacttgaccagagcagcgaggtgctgggcagcgggaagaccctgactatccaggtgaaggagtttggcgatgccggacagtacacctgccacaagggcggcgaggtgttatctcatagcctgctgctgctgcacaaaaaggaggacgggatctggagcactgacatcctgaaggaccagaaggagcccaaaaacaagaccttcctgcgttgcgaggccaagaactacagcgggagattcacctgttggtggctgaccactatcagcactgacctaaccttcagcgtgaagagcagccggggaagctctgacccccagggggtgacatgcggcgccgccacactgagcgccgagagggtgagaggggacaataaggaatacgagtatagcgtggagtgtcaggaagattccgcctgccccgccgccgaggagagcctgcctatcgaggtcatggtggacgccgtccataaactgaagtacgaaaactatacttcaagcttcttcatcagagacatcataaagcccgacccccccaagaacctgcagctaaagcccctgaagaacagcagacaggtcgaagtgagctgggagtaccccgatacctggagcaccccgcacagctacttcagcctgaccttttgcgtccaggtgcagggcaagagcaagagagagaagaaggacagggtgttcactgacaagacaagcgccactgttatctgcagaaagaacgccagtatcagcgtgcgcgcccaagacaggtattactccagcagctggtctgaatgggccagcgtgccttgcagc

SEQ ID NO: 59 - DNA, 918 bp, synthetic construct
  misc_feature 1..918: note = IL12b of H3
  source 1..918: mol_type = other DNA
atctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaagagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagc

SEQ ID NO: 60 - DNA, 918 bp, synthetic construct
  misc_feature 1..918: note = IL12b of CO
  source 1..918: mol_type = other DNA
atctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaagagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagc

SEQ ID NO: 61 - DNA, 918 bp, synthetic construct
  misc_feature 1..918: note = IL12b of CP
  source 1..918: mol_type = other DNA
atctgggagctgaagaaggatgtgtatgtggtggagctggactggtacccagatgcccctggagagatggtggtgctgacctgtgacaccccagaggaggatggcatcacctggaccctggaccagagcagcgaggtgctgggcagcggcaagaccctgaccatccaggtgaaggagtttggagatgctggccagtacacctgccacaagggcggggaggtgctgagccacagcctgctgctgctgcacaagaaggaggatggcatctggagcacagacatcctgaaggaccagaaggagcccaagaacaagaccttcctgcgctgtgaggccaagaactacagcggccgcttcacctgctggtggctgaccaccatcagcacagacctgaccttctctgtgaaaagcagccggggcagcagtgacccccagggcgtgacctgtggggccgccaccctgtctgctgagcgggtgcggggggacaacaaggagtatgagtacagcgtggagtgccaggaggacagcgcctgcccagctgctgaggagagcctgcccatcgaggtgatggtggatgctgtgcacaagctgaagtatgagaactacaccagcagcttcttcatccgggacatcatcaagccagacccccccaagaacctgcagctgaagcccctgaagaacagccggcaggtggaggtgtcctgggagtacccagacacctggagcaccccccacagctacttcagcctgaccttctgtgtgcaggtgcagggcaagagcaagcgggagaagaaggacagagtcttcacagacaagaccagcgccaccgtcatctgcaggaagaatgccagcatctctgtgcgggcccaggaccgctactacagcagctcctggagcgagtgggcctctgtgccctgcagc

SEQ ID NO: 62 - DNA, 918 bp, synthetic construct
  misc_feature 1..918: note = IL12b of vH1
  source 1..918: mol_type = other DNA
atctgggagctgaagaaggacgtgtacgtggtggagctggactggtaccccgacgcccccggcgagatggtggtgctgacctgcgacacccccgaggaggacgggatcacctggaccctggatcagagcagcgaggtgctgggcagcggcaagaccctgaccatccaggtgaaggagtttggtgacgccggccagtacacctgccacaagggcggcgaggtgctgagccactcactgctgctgctgcacaagaaggaggacggcatctggagcaccgacattctgaaggatcagaaggagcccaagaacaagaccttcctgagatgcgaggccaagaactacagcggcagattcacctgttggtggctgactactatcagcactgatctgaccttcagcgtgaagagctcaagaggcagcagcgatccccagggcgtgacctgcggcgccgccaccctgagcgccgagagagtgagaggcgacaacaaggagtacgagtacagcgtggagtgccaggaagacagcgcctgccccgctgcagaggagtctctgcccatcgaggtgatggtggacgccgtgcacaagcttaagtacgagaactacaccagctccttcttcattagagacatcatcaagcccgacccgcccaaaaacctgcagctgaagcccctgaagaacagcagacaggtggaggtcagctgggagtaccccgacacctggagcaccccccacagctacttcagcctgaccttctgcgtgcaggtgcagggcaagagcaagagagagaagaaggacagagtgttcaccgacaaaaccagcgccaccgtgatctgcagaaaaaacgccagcattagcgtgagagctcaggatagatactacagcagcagctggagtgagtgggccagcgtgccctgcagc

SEQ ID NO: 63 - DNA, 918 bp, synthetic construct
  misc_feature 1..918: note = IL12b of vH2
  source 1..918: mol_type = other DNA
atctgggagctgaagaaggacgtgtacgtggtggagctggactggtaccccgacgcccccggagagatggtggtgctgacctgcgacacccccgaagaggacggcatcacctggaccctggaccagagcagcgaggtgctgggcagcgggaagaccctgaccatccaggtgaaggagttcggcgacgccggccagtacacctgtcacaagggcggcgaggtgctgagccacagcctgctgctgctgcacaagaaggaggacggcatatggagcaccgacatcctgaaggaccagaaggagcccaagaacaagacctttctgagatgcgaggctaagaattacagcggcagattcacctgctggtggctgaccaccatcagcaccgacctgaccttcagcgtgaagagcagcagaggcagcagcgacccccagggcgtgacatgcggcgccgccaccctgagcgccgagagagtgagaggcgacaacaaggagtacgagtacagcgtggagtgccaggaggactccgcttgccccgccgccgaggagagcttgcccatcgaggtgatggtggacgccgtgcacaagctcaagtacgaaaactacaccagcagcttcttcatcagagacatcatcaagcccgacccccccaagaacctgcagctgaagcccctgaaaaacagcagacaggtggaggtgagctgggagtaccccgacacctggagcaccccccacagctacttcagcctgaccttctgcgtgcaggtgcagggaaaaagcaagagagagaagaaggacagagtgttcaccgacaagaccagcgccaccgtgatctgcagaaagaacgccagcatctccgtgagagcccaggacagatactacagcagcagctggagcgagtgggccagcgtgccctgtagc

SEQ ID NO: 64 - DNA, 918 bp, synthetic construct
  misc_feature 1..918: note = IL12b of vH3
  source 1..918: mol_type = other DNA
atctgggagctgaagaaggacgtgtacgtggtggagctggactggtacccggacgccccgggcgagatggtcgtgctgacctgcgacacccccgaggaggacggcatcacctggaccctggaccagtctagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcctaagaacaagaccttcctgagatgtgaggccaagaactacagcggcagattcacctgctggtggctgaccaccatcagcaccgatctgaccttcagcgtgaagagcagcagaggcagctcagacccccagggcgtgacctgcggcgccgcgaccctgagcgccgagagagtgagaggcgacaacaaggagtacgagtacagcgtggagtgccaggaggacagcgcctgccccgcggccgaggagagcctgcccatcgaggtgatggtggacgccgtgcataagctgaagtacgagaactacaccagcagcttcttcatcagagacatcatcaagcccgatccccccaagaacctgcagctgaagccactgaagaacagcagacaggtggaggtgagctgggagtaccccgacacctggagcaccccccacagctacttcagcctgaccttctgcgtgcaggtgcagggcaagagcaagagagagaagaaggaccgggtgttcaccgacaagaccagcgccactgtgatctgcagaaagaacgccagcatcagcgtgagagcccaggacagatactacagctccagctggtcagagtgggccagcgtgccctgcagc

SEQ ID NO: 65 - DNA, 918 bp, synthetic construct
  misc_feature 1..918: note = IL12b of A1
  source 1..918: mol_type = other DNA
atctgggagctgaagaaagacgtgtacgtggtggagcttgactggtaccccgacgcccccggtgaaatggtggttctgacctgcgacacaccagaggaggacggcatcacctggaccctggaccagtccagcgaggtcctgggatctggcaagaccctgaccatccaggttaaggaatttggcgacgccggccagtacacctgccacaaaggcggcgaggtcctttcgcacagtctgctgctgctgcataaaaaggaggacggcatttggagcaccgacattctgaaggatcagaaagagcccaagaacaagacctttctgagatgtgaggccaaaaactactctggacgcttcacctgttggtggctgaccaccatcagcacagacctgaccttctcggtgaagtctagtaggggcagcagtgacccccagggcgtaacatgcggcgccgctaccctgagcgccgagagagtgagaggcgataacaaggagtacgagtactccgtggagtgccaagaggactcagcctgccccgccgccgaggagtcgctgcccatcgaggtgatggtggatgcagtgcacaagctgaagtacgagaactacaccagtagctttttcatcagagatatcattaaacccgaccctcccaagaacctgcagctgaagcccttaaagaacagccggcaggtggaagtgtcatgggagtacccagacacctggagcactccgcacagctacttcagcctgaccttctgcgttcaggtgcagggaaaaagcaagagagagaagaaagacagagtgttcaccgacaagaccagcgcaaccgtgatctgtagaaagaacgcctcgatcagcgtgagagcccaggacagatactacagcagcagctggagcgagtgggccagtgtaccttgcagc

SEQ ID NO: 66 - DNA, 918 bp, synthetic construct
  misc_feature 1..918: note = IL12b of A2
  source 1..918: mol_type = other DNA
atctgggaactgaagaaggacgtctacgtggtggagctggattggtaccccgacgcccccggcgagatggtcgtgctgacctgtgacacccctgaggaggacggaatcacatggaccctggaccagagcagcgaagtgttgggcagcggcaagaccctgaccatccaagtgaaggaattcggcgacgcgggccagtatacttgccacaagggcggggaggttctgagccacagcctgctgctgctccacaagaaagaggatggcatctggtctaccgacatcctgaaggaccaaaaggagccaaagaacaagaccttcctaagatgcgaggccaagaactactcaggtcgtttcacctgctggtggctgaccacaatctccaccgacctgaccttcagcgtgaaaagcagccgcggcagtagcgacccacagggcgtgacctgcggggccgccactctgagcgccgagagagtgcgcggcgataataaggaatacgagtacagcgtggagtgccaggaagactcagcctgccccgccgcagaagaaagtctgccaatagaggtgatggttgacgccgtgcacaagctaaagtacgagaactacaccagcagcttctttatccgggacatcatcaagccagatccccccaagaacctgcagcttaagcccctgaagaacagcagacaggtggaggtgtcatgggaataccccgacacctggagcaccccccatagctacttctcactgaccttctgcgtgcaggtgcagggcaagagcaagagagagaagaaggatagggtattcacggacaagacctcagccaccgtgatctgccgaaagaacgccagcatcagcgtgagagcccaggacagatactatagctcctcgtggagcgagtgggccagcgtaccttgcagc

SEQ ID NO: 67 - DNA, 918 bp, synthetic construct
  misc_feature 1..918: note = IL12b of A3
  source 1..918: mol_type = other DNA
atctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaaaagcagcagaggcagcagcgacccccagggcgtgacctgcggcgccgccaccctgagcgccgagagagtgagaggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagagacatcatcaagcccgacccccccaagaacctgcagctgaagcccctgaagaacagcagacaggtggaggtgagctgggagtaccccgacacctggagcaccccccacagctacttcagcctgaccttctgcgtgcaggtgcagggcaagagcaagagagagaagaaggacagagtgttcaccgacaagaccagcgccaccgtgatctgcagaaagaacgccagcatcagcgtgagagcccaggacagatactacagcagcagctggagcgagtgggccagcgtgccctgcagc

SEQ ID NO: 68 - DNA, 918 bp, synthetic construct; identical to SEQ ID NO: 61
  misc_feature 1..918: note = IL12b of A4
  source 1..918: mol_type = other DNA

SEQ ID NO: 69 - DNA, 918 bp, synthetic construct; identical to SEQ ID NO: 65
  misc_feature 1..918: note = IL12b of B1
  source 1..918: mol_type = other DNA

SEQ ID NO: 70 - DNA, 918 bp, synthetic construct; identical to SEQ ID NO: 66
  misc_feature 1..918: note = IL12b of B2
  source 1..918: mol_type = other DNA

SEQ ID NO: 71 - DNA, 918 bp, synthetic construct; identical to SEQ ID NO: 67
  misc_feature 1..918: note = IL12b of B3
  source 1..918: mol_type = other DNA

SEQ ID NO: 72 - DNA, 918 bp, synthetic construct; identical to SEQ ID NO: 61
  misc_feature 1..918: note = IL12b of B4
  source 1..918: mol_type = other DNA

SEQ ID NO: 73 - DNA, 918 bp, synthetic construct; identical to SEQ ID NO: 54
  misc_feature 1..918: note = IL12b of C1
  source 1..918: mol_type = other DNA

SEQ ID NO: 74 - DNA, 918 bp, synthetic construct; identical to SEQ ID NO: 65
  misc_feature 1..918: note = IL12b of C2
  source 1..918: mol_type = other DNA

SEQ ID NO: 75 - DNA, 918 bp, synthetic construct; identical to SEQ ID NO: 66
  misc_feature 1..918: note = IL12b of C3
  source 1..918: mol_type = other DNA

SEQ ID NO: 76 - DNA, 45 bp, synthetic construct
  misc_feature 1..45: note = GS Linker of L1
  source 1..45: mol_type = other DNA
gggggttctggtgggggcagtggaggaggatcaggtggcggcagt

SEQ ID NO: 77 - DNA, 45 bp, synthetic construct
  misc_feature 1..45: note = GS Linker of L2
  source 1..45: mol_type = other DNA
ggcggttcgggcggagggtccggcggtggtagcggaggcgggagc

SEQ ID NO: 78 - DNA, 45 bp, synthetic construct
  misc_feature 1..45: note = GS Linker of L3
  source 1..45: mol_type = other DNA
ggcggttccggcgggggctcggggggcgggagcggtggaggcagc

SEQ ID NO: 79 - DNA, 45 bp, synthetic construct
  misc_feature 1..45: note = GS Linker of M1
  source 1..45: mol_type = other DNA
ggcggcagcggcggcgggagcggcggaggctccgggggaggtagc

SEQ ID NO: 80 - DNA, 45 bp, synthetic construct
  misc_feature 1..45: note = GS Linker of M2
  source 1..45: mol_type = other DNA
ggcggcagcggtggaggaagcggcggtggcagtgggggcgggagc

SEQ ID NO: 81 - DNA, 45 bp, synthetic construct
  misc_feature 1..45: note = GS Linker of M3
  source 1..45: mol_type = other DNA
ggcggcagcggcggtggcagcggcggcggttctggcggcggttca

SEQ ID NO: 82 - DNA, 45 bp, synthetic construct
  misc_feature 1..45: note = GS Linker of H1
  source 1..45: mol_type = other DNA
ggtggcagcggcggcggctccggcggcgggagtggcggcggcagc

SEQ ID NO: 83 - DNA, 45 bp, synthetic construct
  misc_feature 1..45: note = GS Linker of H2
  source 1..45: mol_type = other DNA
ggggggagcggcggtggcagcgggggcggcagcggcgggggcagc

SEQ ID NO: 84 - DNA, 45 bp, synthetic construct
  misc_feature 1..45: note = GS Linker of H3
  source 1..45: mol_type = other DNA
ggcgggagcggcggcggcagcggcggtggcagtggcgggggcagc

SEQ ID NO: 85 - DNA, 45 bp, synthetic construct
  misc_feature 1..45: note = GS Linker of CO
  source 1..45: mol_type = other DNA
ggcggcagcggcggcggcagcggcggcggcagcggcggcggcagc

SEQ ID NO: 86 - DNA, 45 bp, synthetic construct; identical to SEQ ID NO: 85
  misc_feature 1..45: note = GS Linker of CP
  source 1..45: mol_type = other DNA

SEQ ID NO: 87 - DNA, 45 bp, synthetic construct
  misc_feature 1..45: note = GS Linker of vH1
  source 1..45: mol_type = other DNA
ggcggctcaggcggcggctcaggcggcggcagcggcggcggcagc

SEQ ID NO: 88 - DNA, 45 bp, synthetic construct; identical to SEQ ID NO: 85
  misc_feature 1..45: note = GS Linker of vH2
  source 1..45: mol_type = other DNA

SEQ ID NO: 89 - DNA, 45 bp, synthetic construct
  misc_feature 1..45: note = GS Linker of vH3
  source 1..45: mol_type = other DNA
ggcggcagcggtggcgggagcggcggcgggagcggcggcggaagc

SEQ ID NO: 90 - DNA, 45 bp, synthetic construct
  misc_feature 1..45: note = GS Linker of A1
  source 1..45: mol_type = other DNA
ggtggaagcggcggaggctctggcggagggagtggcggaggcagc

SEQ ID NO: 91 - DNA, 45 bp, synthetic construct
  misc_feature 1..45: note = GS Linker of A2
  source 1..45: mol_type = other DNA
ggcgggagcggcggcggcagcggaggtggcagtggcggaggcagc

SEQ ID NO: 92 - DNA, 45 bp, synthetic construct; identical to SEQ ID NO: 85
  misc_feature 1..45: note = GS Linker of A3
  source 1..45: mol_type = other DNA

SEQ ID NO: 93 - DNA, 45 bp, synthetic construct; identical to SEQ ID NO: 85
  misc_feature 1..45: note = GS Linker of A4
  source 1..45: mol_type = other DNA

SEQ ID NO: 94 - DNA, 45 bp, synthetic construct; identical to SEQ ID NO: 90
  misc_feature 1..45: note = GS Linker of B1
  source 1..45: mol_type = other DNA

SEQ ID NO: 95 - DNA, 45 bp, synthetic construct; identical to SEQ ID NO: 91
  misc_feature 1..45: note = GS Linker of B2
  source 1..45: mol_type = other DNA

SEQ ID NO: 96 - DNA, 45 bp, synthetic construct; identical to SEQ ID NO: 85
  misc_feature 1..45: note = GS Linker of B3
  source 1..45: mol_type = other DNA

SEQ ID NO: 97 - DNA, 45 bp, synthetic construct; identical to SEQ ID NO: 85
  misc_feature 1..45: note = GS Linker of B4
  source 1..45: mol_type = other DNA

SEQ ID NO: 98 - DNA, 45 bp, synthetic construct
  misc_feature 1..45: note = GS Linker of C1
  source 1..45: mol_type = other DNA
ggaggcagcggtggcgggagcggcggaggctccgggggaggtagc

SEQ ID NO: 99 - DNA, 45 bp, synthetic construct; identical to SEQ ID NO: 90
  misc_feature 1..45: note = GS Linker of C2
  source 1..45: mol_type = other DNA

SEQ ID NO: 100 - DNA, 45 bp, synthetic construct; identical to SEQ ID NO: 91
  misc_feature 1..45: note = GS Linker of C3
  source 1..45: mol_type = other DNA

SEQ ID NO: 101 - DNA, 591 bp, synthetic construct
  misc_feature 1..591: note = IL12a of L1
  source 1..591: mol_type = other DNA
cgcaatctacccgtggcaacaccagacccgggaatgttcccatgtctgcaccacagtcaaaacctcttaagggccgtgtcaaatatgctacagaaggcgagacagactttagaattctacccttgtacaagcgaggagattgaccacgaggacatcaccaaagataagacgagcaccgtcgaggcttgcctgcctctagaactaacaaaaaatgaatcatgcttgaactcgagggagaccagtttcattactaacggttcatgtcttgcatcgaggaagacctcattcatgatggccctgtgcctctcgtccatttatgaagacctaaagatgtaccaggtagagtttaagaccatgaacgccaagctcctcatggatccaaaacggcaaatattcttagatcagaatatgctcgctgttatcgacgaactcatgcaggcgcttaacttcaactcagaaaccgttccccaaaagtcgagtctagaagaaccggacttttataagaccaaaattaaactgtgtatactacttcacgccttcaggataagagcagtgacgattgacagggtgatgtcctacttgaatgcatca

SEQ ID NO: 102 - DNA, 591 bp, synthetic construct
  misc_feature 1..591: note = IL12a of L2
  source 1..591: mol_type = other DNA
aggaacttgcccgtggctactccagaccctggcatgttcccctgtttacaccactcccagaacttattacgtgctgttagcaacatgttgcaaaaggcccgtcaaaccctcgagttttacccctgtactagtgaagaaatcgaccacgaagacataacaaaagacaagacaagcacagttgaggcatgcttacccctggagttaacaaagaacgagagctgtctgaactctcgagagacgagcttcatcaccaatgggagttgccttgcttctcgaaaaacgtcgttcatgatggccctgtgcctgtcgtctatctatgaggatctgaaaatgtatcaggttgaattcaagacaatgaatgccaaactactcatggatccaaaacggcagatattcctcgatcagaatatgctcgcagttattgacgaactaatgcaggctctgaattttaacagcgagaccgttcctcagaagtcaagtttggaagaacctgacttctacaaaactaaaataaaattatgcatcttactgcatgctttcagaattagagctgtcactattgatcgagtgatgtcatacttgaatgcttcc

SEQ ID NO: 103 - DNA, 591 bp, synthetic construct
  misc_feature 1..591: note = IL12a of L3
  source 1..591: mol_type = other DNA
aggaatcttcctgtcgcgacccctgatcccggcatgtttccttgcctgcaccacagtcagaacttattacgcgccgtttccaatatgttacagaaggccagacagacattagagttttatccttgcacatcggaggagatcgaccatgaagatatcacaaaagataaaacatccaccgttgaggcctgcctgccacttgaacttactaaaaacgagagctgcctgaatagccgggaaacttctttcatcactaatggatcgtgtctagcaagccgaaaaaccagcttcatgatggctttgtgcctctcgtccatctatgaagacctgaaaatgtatcaagtagaatttaaaacgatgaatgccaaactgcttatggatcccaaacgccagatatttctagaccagaatatgctggccgtcattgatgagctaatgcaggctctcaattttaatagcgaaacagtgccccaaaaaagctctttggaagagccggatttttacaaaaccaagattaagctatgtatcctgctgcatgctttcagaattcgagctgttacaattgatcgggttatgagctacttaaacgcctcg

SEQ ID NO: 104 - DNA, 591 bp, synthetic construct
  misc_feature 1..591: note = IL12a of M1
  source 1..591: mol_type = other DNA
cggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaagagcagcctcgaggagcccgacttctacaaaacaaagatcaaactctgcatccttctgcacgccttcagaattagagccgtgaccatcgacagagttatgagctacctgaatgccagc

SEQ ID NO: 105 - DNA, 591 bp, synthetic construct
  misc_feature 1..591: note = IL12a of M2
  source 1..591: mol_type = other DNA
agaaatctgcccgtcgccactccagatcctggcatgttcccgtgcctgcatcacagccaaaacctgctgcgggcggtgtctaacatgctgcagaaggctaggcagaccttggaattctatccctgcacaagcgaggaaatagaccatgaggacatcaccaaggataagaccagcacggtcgaagcttgcctgccactggaactgacaaaaaacgagagttgcctgaactcccgcgagacatccttcatcacaaacggcagctgcctggctagcaggaagaccagcttcatgatggccctgtgcctgtcttccatctacgaggacctgaaaatgtaccaagtggagttcaagactatgaacgccaagctgctaatggatcccaagcgacagatctttctagaccagaacatgctggccgtcattgacgagctgatgcaggcactcaattttaactcagagaccgtgccacagaagtccagcctggaggagcctgacttctataagaccaagattaagctgtgcatcctgctgcatgccttccgaataagagccgtgaccattgaccgagtgatgtcatacttgaacgcaagc

SEQ ID NO: 106 - DNA, 591 bp, synthetic construct
  misc_feature 1..591: note = IL12a of M3
  source 1..591: mol_type = other DNA
agaaacctcccagtcgctacccccgatcccggaatgttcccctgcctgcaccactcccagaatctgctccgagccgttagcaacatgctgcaaaaggcccggcagaccctggagttctacccatgcacctcggaagaaatcgatcacgaggacatcaccaaggacaagactagcaccgtcgaggcctgcctgccgctggaactaaccaagaatgaaagctgcctcaactcgcgggagacctctttcataaccaacggctcatgcctggccagccggaaaactagctttatgatggctctgtgcttaagcagcatctacgaggatctgaagatgtaccaggtagagttcaagaccatgaatgccaagctgctgatggaccccaagagacaaatcttcctggaccagaacatgctggccgtaattgatgaactgatgcaggccctgaatttcaacagcgagaccgtaccccagaaaagctcactggaggagcccgacttttataagacgaaaataaagttgtgcatccttcttcacgctttccggattagagccgtgaccatcgatagagtgatgtcatacctgaacgcatcg

SEQ ID NO: 107 - DNA, 591 bp, synthetic construct
  misc_feature 1..591: note = IL12a of H1
  source 1..591: mol_type = other DNA
agaaatctgcccgtagccacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaagagcagcctggaggaacccgacttctacaaaaccaagatcaagctgtgcattctgctgcatgccttccgcattagagccgtgaccatcgatagggtgatgagctacctgaacgccagc

SEQ ID NO: 108 - DNA, 591 bp, synthetic construct
  misc_feature 1..591: note = IL12a of H2
  source 1..591: mol_type = other DNA
agaaacctgcccgtggccacacccgatcccggcatgttcccctgtctgcaccacagccaaaacctgctgcgtgccgtgagcaacatgctgcagaaggcgcggcagaccctggagttctatccctgcaccagtgaggagattgaccacgaggatatcaccaaagacaagaccagcaccgtggaggcctgcctccccctggagctgaccaagaacgagtcctgcttgaattcaagagagaccagcttcatcaccaacggctcctgcttagccagcagaaagactagcttcatgatggccttgtgcttgtctagcatctatgaggatctgaagatgtaccaggtcgagttcaagactatgaacgccaagctgctgatggaccccaaaagacagatcttcctggaccagaacatgctggccgtcatcgacgagctgatgcaggcccttaatttcaatagcgagacagtgccccagaaatcttctctggaggagcccgatttttacaaaaccaagatcaaactatgcatcctgttgcacgccttccggatccgcgccgtgaccatcgacagagtaatgtcctacctgaacgccagc

SEQ ID NO: 109 - DNA, 591 bp, synthetic construct
  misc_feature 1..591: note = IL12a of H3
  source 1..591: mol_type = other DNA
agaaacctgcccgtggccacccccgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaagagcagcctggaggagcccgacttttacaagaccaagatcaagttgtgcatcctcctgcacgccttcagaatcagagcggtgacgatcgacagagtcatgagctacctcaacgctagc

SEQ ID NO: 110 - DNA, 591 bp, synthetic construct
  misc_feature 1..591: note = IL12a of CO
  source 1..591: mol_type = other DNA
agaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagc

SEQ ID NO: 111 - DNA, 591 bp, synthetic construct
  misc_feature 1..591: note = IL12a of CP
  source 1..591: mol_type = other DNA
aggaacctgcctgtggccaccccagaccccggcatgttcccctgcctgcaccacagccagaacctgctgcgggctgtgagcaacatgctgcagaaggcccggcagaccctggagttctacccctgcaccagcgaggagattgaccacgaggacatcaccaaggacaagaccagcacagtggaggcctgcctgcccctggagctgaccaagaatgaaagctgcctgaacagccgggagaccagcttcatcaccaacggcagctgcctggccagcaggaagaccagcttcatgatggccctgtgcctgagcagcatctatgaggacctgaagatgtaccaggtggagttcaagaccatgaatgccaagctgctgatggaccccaagaggcagatcttcctggaccagaacatgctggccgtgattgatgagctgatgcaggccctgaacttcaacagcgagaccgtgccccagaaaagcagcctggaggagccagacttctacaagaccaagatcaagctgtgcatcctgctgcacgccttccgcatccgggctgtgaccatcgacagagtgatgagctacctgaatgccagc

SEQ ID NO: 112 - DNA, 591 bp, synthetic construct
  misc_feature 1..591: note = IL12a of vH1
  source 1..591: mol_type = other DNA
agaaacctgcccgtggccacgcccgaccccggcatgtttccctgcctgcaccatagccagaatctgctgagagccgtgagcaacatgctgcagaaggccagacagacgctcgagttctacccctgcaccagcgaggagattgaccacgaggacatcaccaaggacaagaccagcaccgtggaggcctgcctgcccctcgagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagacaatgaacgccaagctgctgatggaccccaagaggcagatttttctggaccagaacatgctggccgttatcgacgagctgatgcaggccctgaatttcaatagcgaaaccgtgccccagaagagcagcctggaggagcctgacttctacaaaaccaagatcaagctttgcatcctgctgcacgccttcagaatcagagccgtgaccatcgacagagtgatgagctacctgaacgccagc

SEQ ID NO: 113 - DNA, 591 bp, synthetic construct
  misc_feature 1..591: note = IL12a of vH2
  source 1..591: mol_type = other DNA
agaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccatgaggacatcaccaaggacaagaccagcaccgtggaggcctgcctgcccctggagctgaccaagaacgaaagctgcctgaacagcagagagaccagcttcatcaccaacggcagctgtctggcctcaagaaagaccagctttatgatggccctgtgcctgtctagcatctacgaggatctgaagatgtaccaggtggagttcaagaccatgaacgccaagctgctgatggaccccaagagacagatcttcctggaccagaacatgttagccgtgattgacgagctgatgcaggccctgaacttcaatagcgagaccgtgccccagaaaagcagcctggaggagcccgacttctacaagaccaagatcaagctgtgcatcctgctgcacgccttcagaatcagagccgtaaccatcgacagagtgatgagctacctgaacgccagt

SEQ ID NO: 114 - DNA, 591 bp, synthetic construct
  misc_feature 1..591: note = IL12a of vH3
  source 1..591: mol_type = other DNA
agaaacctgcccgtggccacccctgaccccggcatgttcccctgcctgcaccacagccagaacctgctgagagccgtaagcaacatgctgcagaaggccagacagactctggagttctacccctgcaccagcgaggagatcgatcacgaggacatcaccaaggacaagaccagtaccgtggaggcctgcctgcccctggagctgaccaagaacgagagctgcctgaacagcagagagaccagctttataaccaacggcagctgtctggctagcagaaagaccagcttcatgatggccctgtgcctgagcagcatctacgaggacttgaagatgtaccaggtggagttcaagaccatgaacgccaagcttctgatggaccccaagagacagatctttctggaccagaacatgctggccgtgatcgatgaactgatgcaggccctgaacttcaacagcgagaccgtgccccagaagagcagtctggaggagcccgacttctacaagactaagatcaagctgtgcatcctgctgcacgccttcagaatcagagccgtgaccatcgacagagtgatgagctacctgaacgcctca

SEQ ID NO: 115 - DNA, 591 bp, synthetic construct
  misc_feature 1..591: note = IL12a of A1
  source 1..591: mol_type = other DNA
agaaatcttccagtagctacccccgaccccggcatgtttccctgtctgcaccattctcaaaacctgttacgggccgtgagcaacatgctgcagaaggccagacagacactggagttttacccctgtacctcagaggagatcgatcatgaggacattactaaggacaagaccagcaccgtggaagcctgcctgcccctagagctaaccaagaacgagagctgcctgaactctagagagacaagcttcatcacgaacggctcatgcctggccagtaggaaaaccagcttcatgatggctctgtgcctgagctccatatatgaggaccttaagatgtaccaggtggagttcaaaaccatgaacgccaagctgctgatggacccaaagagacagatcttccttgaccagaacatgctggccgttatcgatgagctgatgcaggccctgaacttcaacagcgagaccgtgccccagaaaagcagcctggaggaacccgacttctacaaaaccaagatcaagctgtgcattctgctgcatgccttccgcattagagccgtgaccatcgatagggtgatgagctacctgaacgccagc

SEQ ID NO: 116 - DNA, 588 bp, synthetic construct
  misc_feature 1..588: note = IL12a of A2
  source 1..588: mol_type = other DNA
agaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgct

SEQ ID NO: 117 - DNA, 591 bp, synthetic construct
  misc_feature 1..591: note = IL12a of A3
  source 1..591: mol_type = other DNA
agaaacctgcccgtggccacccccgaccccggcatgttcccctgcctgcaccacagccagaacctgctgagagccgtgagcaacatgctgcagaaggccagacagaccctggagttctacccctgcaccagcgaggagatcgaccacgaggacatcaccaaggacaagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagagacagatcttcctggaccagaacatgctggccgtgatcgacgagctgatgcaggccctgaacttcaacagcgagaccgtgccccagaaaagcagcctggaggagcccgacttctacaagaccaagatcaagctgtgcatcctgctgcacgccttcagaatcagagccgtgaccatcgacagagtgatgagctacctgaacgccagc

SEQ ID NO: 118 - DNA, 591 bp, synthetic construct; identical to SEQ ID NO: 111
  misc_feature 1..591: note = IL12a of A4
  source 1..591: mol_type = other DNA

SEQ ID NO: 119 - DNA, 591 bp, synthetic construct; identical to SEQ ID NO: 115
  misc_feature 1..591: note = IL12a of B1
  source 1..591: mol_type = other DNA

SEQ ID NO: 120 - DNA, 591 bp, synthetic construct
  misc_feature 1..591: note = IL12a of B2
  source 1..591: mol_type = other DNA
agaaacctccccgtggcaacccctgaccccgggatgtttccttgcctgcaccactcccagaacctcctgagagccgtgtccaacatgctgcagaaagccagacagaccctcgagttctatccctgcacaagcgaggagatcgaccacgaggacatcactaaggacaagaccagcacagtggaagcctgcctcccgctggagctgactaagaacgagagctgtctgaacagcagggagaccagcttcatcaccaacggcagctgcctggcgagcagaaaaacctcctttatgatggcgctctgcctgagctcaatctatgaggacctgaagatgtaccaggtggagtttaaaaccatgaatgccaagctgcttatggaccctaagagacagattttcctggatcagaacatgctggccgtgattgatgaattaatgcaggcgctgaactttaacagcgagaccgtgccccagaaaagcagcctggaggagcccgacttttacaagaccaagatcaagttgtgcatcctcctgcacgccttcagaatcagagcggtgacgatcgacagagtcatgagctacctcaacgctagc

SEQ ID NO: 121 - DNA, 591 bp, synthetic construct; identical to SEQ ID NO: 117
  misc_feature 1..591: note = IL12a of B3
  source 1..591: mol_type = other DNA

SEQ ID NO: 122 - DNA, 591 bp, synthetic construct; identical to SEQ ID NO: 111
  misc_feature 1..591: note = IL12a of B4
  source 1..591: mol_type = other DNA

SEQ ID NO: 123 - DNA, 591 bp, synthetic construct
  misc_feature 1..591: note = IL12a of C1
  source 1..591: mol_type = other DNA
cggaatctgcctgtcgccactccagaccccggcatgttcccatgtctgcatcattctcagaacctgctgagggccgtatccaatatgctgcagaaagccagacagaccttagagttctatccctgtacaagcgaggagatagatcacgaggatattacgaaggacaaaacttctactgttgaggcgtgtcttccattagagctgaccaagaacgaaagctgtctgaatagcagagagacttcatttatcaccaatgggagttgcttggctagcagaaagaccagcttcatgatggccctttgcttgtcttcgatatacgaagatcttaagatgtatcaagtggaatttaagacgatgaacgccaagctgcttatggatcccaagcgccaaatcttcctggatcagaacatgttggccgtgattgacgagctgatgcaagccctgaatttcaactccgagaccgtgcctcagaaaagcagcctcgaggagcccgacttctacaaaacaaagatcaaactctgcatccttctgcacgccttcagaattagagccgtgaccatcgacagagttatgagctacctgaatgccagc

SEQ ID NO: 124 - DNA, 591 bp, synthetic construct; identical to SEQ ID NO: 115
  misc_feature 1..591: note = IL12a of C2
  source 1..591: mol_type = other DNA

SEQ ID NO: 125 - DNA, 591 bp, synthetic construct; identical to SEQ ID NO: 120
  misc_feature 1..591: note = IL12a of C3
  source 1..591: mol_type = other DNA

SEQ ID NO: 126 - DNA, 18 bp, synthetic construct
  misc_feature 1..18: note = GS Linker of L1
  source 1..18: mol_type = other DNA
ggatcagggggaggctcg

SEQ ID NO: 127 - DNA, 18 bp, synthetic construct
  misc_feature 1..18: note = GS Linker of L2
  source 1..18: mol_type = other DNA
ggatcaggaggtgggagc

SEQ ID NO: 128 - DNA, 18 bp, synthetic construct
  misc_feature 1..18: note = GS Linker of L3
  source 1..18: mol_type = other DNA
ggtagtggcggggggagc

SEQ ID NO: 129 - DNA, 18 bp, synthetic construct
  misc_feature 1..18: note = GS Linker of M1
  source 1..18: mol_type = other DNA
ggcagcggcggcggatcc

SEQ ID NO: 130 - DNA, 18 bp, synthetic construct
  misc_feature 1..18: note = GS Linker of M2
  source 1..18: mol_type = other DNA
ggctcaggcggagggagt

SEQ ID NO: 131 - DNA, 18 bp, synthetic construct
  misc_feature 1..18: note = GS Linker of M3
  source 1..18: mol_type = other DNA
gggagtggcggtggcagc

SEQ ID NO: 132 - DNA, 18 bp, synthetic construct
  misc_feature 1..18: note = GS Linker of H1
  source 1..18: mol_type = other DNA
ggctctggcggcggcagt

SEQ ID NO: 133 - DNA, 18 bp, synthetic construct
  misc_feature 1..18: note = GS Linker of H2
  source 1..18: mol_type = other DNA
ggcagcggcggcggtagc

SEQ ID NO: 134 - DNA, 18 bp, synthetic construct
  misc_feature 1..18: note = GS Linker of H3
  source 1..18: mol_type = other DNA
ggcagcggtggcggcagc

SEQ ID NO: 135 - DNA, 18 bp, synthetic construct
  misc_feature 1..18: note = GS Linker of CO
  source 1..18: mol_type = other DNA
ggcagcggcggcggcagc

SEQ ID NO: 136 - DNA, 18 bp, synthetic construct
  misc_feature 1..18: note = GS Linker of CP
  source 1..18: mol_type = other DNA
ggcagcggcggcggcagt

SEQ ID NO: 137 - DNA, 18 bp, synthetic construct
  misc_feature 1..18: note = GS Linker of vH1
  source 1..18: mol_type = other DNA
ggcagcggcggcggcagc

SEQ ID NO: 138 - DNA, 18 bp, synthetic construct
  misc_feature 1..18: note = GS Linker of vH2
  source 1..18: mol_type = other DNA
ggcagcggcggtggcagc

SEQ ID NO: 139 - DNA, 18 bp, synthetic construct
  misc_feature 1..18: note = GS Linker of vH3
  source 1..18: mol_type = other DNA
ggcagcggcggcgggagc

SEQ ID NO: 140 - DNA, 18 bp, synthetic construct
  misc_feature 1..18: note = GS Linker of B1
  source 1..18: mol_type = other DNA
ggctctggcggcggcagt

SEQ ID NO: 141 - DNA, 18 bp, synthetic construct
  misc_feature 1..18: note = GS Linker of B2
  source 1..18: mol_type = other DNA
ggcagcggtggcggcagc

SEQ ID NO: 142 - DNA, 18 bp, synthetic construct
  misc_feature 1..18: note = GS Linker of B3
  source 1..18: mol_type = other DNA
ggcagcggcggcggcagc

SEQ ID NO: 143 - DNA, 18 bp, synthetic construct
  misc_feature 1..18: note = GS Linker of B4
  source 1..18: mol_type = other DNA
ggcagcggcggcggcagt

SEQ ID NO: 144 - DNA, 18 bp, synthetic construct
  misc_feature 1..18: note = GS Linker of C1
  source 1..18: mol_type = other DNA
ggcagcggcggcggatcc

SEQ ID NO: 145 - DNA, 18 bp, synthetic construct
  misc_feature 1..18: note = GS Linker of C2
  source 1..18: mol_type = other DNA
ggctctggcggcggcagt

SEQ ID NO: 146 - DNA, 18 bp, synthetic construct
  misc_feature 1..18: note = GS Linker of C3
  source 1..18: mol_type = other DNA
ggcagcggtggcggcagc

SEQ ID NO: 147 - DNA, 1758 bp, synthetic construct
  misc_feature 1..1758: note = Albumin of L1
  source 1..1758: mol_type = other DNA
gacgcccataagagcgaagtcgcccaccgcttcaaggatttgggtgaggaaaacttcaaagccctggtcctgatagcgtttgcccaatatttgcagcagtgtccattcgaagatcacgtgaaattggtgaacgaggtaacagaatttgctaagacttgtgtggctgacgagtcggccgaaaactgtgataagagtcttcatacactgtttggcgataagctatgtactgtcgctacacttagggagacttacggtgagatggccgactgctgcgccaagcaagagccagaacgaaacgagtgttttctgcaacataaggacgacaatcccaacctgcccagattggttcgccctgaagttgatgttatgtgcaccgcatttcacgacaacgaagaaacctttcttaaaaagtatctgtacgagatagctcgacgtcacccttacttctacgcgcccgaacttctgtttttcgccaagcgatacaaagccgctttcacagagtgttgccaagctgccgacaaagccgcttgccttctaccaaagcttgacgagctcagagatgaagggaaagctagttcggcaaagcaacgattaaagtgtgcatcactgcaaaaattcggcgaacgagcctttaaagcatgggcagttgccaggttatcccaaaggttcccgaaagctgaattcgctgaggtgagcaagttagtcacggaccttacgaaggtacataccgaatgctgccacggggacctcttggagtgcgctgacgacagggcggacttagctaaatacatttgcgagaatcaggactcaatcagttctaaacttaaagaatgctgcgagaaaccgctcctggaaaaatcacattgcatcgccgaggtggaaaacgatgagatgccagcagatttaccatctctagccgccgacttcgtggaaagtaaggatgtgtgtaaaaactatgcggaagcaaaagacgtgttcctcggaatgtttctatacgaatacgctagaaggcatcctgactattctgtcgttttactgcttagactagcgaagacatatgaaacgacgttagagaaatgctgcgcggccgctgacccccacgaatgttacgcgaaggtctttgatgagttcaagcccctggttgaggagccgcaaaaccttattaaacagaattgtgagctatttgagcagttaggcgaatataaattccagaatgcacttctagtacgatacaccaaaaaggtccctcaagtgagcacccccactcttgtggaggtatccagaaatctaggaaaggtaggctctaaatgctgcaagcatcccgaagccaagagaatgccatgcgctgaagactaccttagcgttgttctgaatcagttgtgtgtccttcacgaaaagacgccggtgagtgatcgtgtcacgaagtgctgtacagagagcctcgtcaaccgtaggccatgtttctccgctctcgaggtggatgaaacatatgtacctaaggaatttaatgcggaaactttcacctttcacgcggacatctgtaccctgagcgagaaggagaggcagataaaaaagcagacggctcttgtagagttggtcaaacataagcctaaggccactaaagagcagctaaaggcagtaatggacgactttgcggctttcgttgagaagtgctgcaaggccgacgataaagagacctgtttcgcggaagaaggtaaaaagttagtggccgcctcccaggcggccctgggcctgtag

SEQ ID NO: 148 - DNA, 1758 bp, synthetic construct
  misc_feature 1..1758: note = Albumin of L2
  source 1..1758: mol_type = other DNA
gacgcgcacaaaagcgaggtagcgcatcgctttaaagacttaggagaggaaaactttaaggcgctggtgctcatcgcatttgcccaatacttacagcagtgtccttttgaggaccacgtaaagcttgtaaacgaagtcactgaattcgccaagacatgtgttgctgacgaaagcgcagagaactgtgacaaaagccttcataccctctttggtgacaaactctgcaccgtggcaactctaagagaaacctacggggaaatggcagactgctgcgcaaagcaggaacccgaacgcaatgagtgtttcctgcagcacaaggatgataatcccaatctgccacgacttgtacggccggaggtagatgttatgtgcactgcttttcatgacaacgaggaaactttccttaagaaatatctgtatgagatcgcaaggcggcacccttacttctacgcacccgaactgttgtttttcgcaaagaggtataaggccgcatttaccgagtgttgtcaggccgctgataaagccgcctgtcttttgccaaaattagatgaactaagggacgaaggcaaagcgagtagcgccaaacaaagattaaaatgtgcaagcctccaaaaattcggtgaaagagcatttaaggcgtgggctgtcgcccgactttcacaacgcttccccaaagctgaattcgctgaggtttcgaagctggttaccgacctaactaaagtgcatacagagtgctgtcatggggatctcttagagtgcgcggatgaccgggcagacctggctaagtacatatgtgagaaccaggacagtatatcatcaaagctgaaagagtgttgtgagaagccactactcgagaagagtcactgtattgccgaggtggaaaatgatgagatgccagccgatcttccttctttggccgctgactttgtagagagcaaggatgtctgtaagaactacgctgaggccaaggatgtctttttggggatgttcctctatgagtacgcccgacgacaccctgactatagtgtagtacttttgcttagactggctaaaacatatgagacgactctcgaaaagtgctgtgccgctgccgatccacacgagtgctacgctaaggtgtttgatgagtttaagccgctggtggaggaaccccagaacctgatcaagcagaactgtgaactattcgagcaactaggggagtacaaattccagaacgcacttttagtgcggtacaccaaaaaagtgccacaggtcagtacaccaacattagtggaagtatccaggaacctgggcaaagtgggcagcaaatgctgcaaacatccggaggctaagcggatgccctgtgcagaggactacctgtccgtggtgcttaaccagctgtgtgtgcttcacgagaaaacgcctgtgtccgaccgggtgaccaagtgctgtacggagtcactggtaaatcgacgaccgtgtttttcagcactagaagttgatgaaacttatgtaccgaaagagtttaacgcagagacctttacattccacgccgacatctgcacgctgtccgagaaggaaagacagattaaaaagcagactgccctagtcgagcttgtcaaacacaaaccgaaggcaaccaaggaacagttaaaagcagtgatggatgattttgctgcgttcgtcgaaaaatgttgcaaagcggacgacaaggagacttgcttcgcagaggaagggaagaaattggttgcggcgtcccaagcggccttagggctatag

SEQ ID NO: 149 - DNA, 1758 bp, synthetic construct
  misc_feature 1..1758: note = Albumin of L3
  source 1..1758: mol_type = other DNA
gacgcccacaagtctgaagtggcacaccggttcaaggacctcggggaggagaattttaaagccctcgtgctgatcgctttcgcgcagtacttgcagcagtgcccttttgaggaccatgtcaaattagtaaacgaagtgacggaattcgcaaagacttgcgtagcggatgagtcagcagagaattgcgacaagtcgctacacactctgttcggggataagttgtgcacagttgctaccttacgagagacctatggagagatggctgactgctgcgccaaacaagagcctgaaagaaacgagtgcttcttacaacacaaagacgataacccgaatttgccaaggcttgtaagacccgaagtagatgttatgtgcacagcttttcacgacaacgaggagacgttccttaagaaatatctgtatgaaatagcccgtcggcacccctatttttatgctcccgaactattgttcttcgccaaacgatacaaggctgcgttcactgagtgctgtcaggctgcagacaaagcagcctgcttgcttcccaaattggacgaactacgggacgaaggtaaggcgagctccgcaaaacagcggttgaaatgcgcgtcactacagaagtttggggaaagagcgttcaaagcttgggctgtggcacgattgagccaacgcttccccaaagcagagtttgcggaagtttcgaaactcgtgacagacttaacaaaggttcacaccgagtgctgtcacggcgatctgctcgaatgcgctgatgaccgcgctgacttggctaaatacatttgtgagaaccaggactctatatcgagcaaactcaaggaatgctgcgaaaagcccctgcttgagaagtcccactgcatcgctgaggtagagaatgatgagatgcctgcggatcttccgagtttagcagctgatttcgtcgagagcaaagatgtgtgcaaaaattatgccgaagcaaaagacgtatttcttgggatgttcttatacgagtatgcacggcgccacccagattactccgtagtgctactgttaagattggcgaagacctatgaaaccacattggaaaagtgctgcgccgccgccgacccccacgagtgttacgcgaaggtgttcgatgaatttaaaccgcttgttgaggagccgcaaaacctaataaagcaaaactgtgagctctttgagcaactaggtgaatacaagtttcagaatgcactcctagttcggtacaccaaaaaagtacctcaggtatctacgccaacgttagtcgaggtctcgcggaatttgggtaaagtaggttccaagtgttgcaaacacccggaagctaaacgtatgccgtgtgctgaggactatctcagcgttgtgttaaaccaactttgcgtactccacgagaaaacacctgtctcagatcgggtaaccaaatgctgcacggagtcgctagtaaatcgtcgcccatgcttttctgcgttagaggtggacgaaacttatgtaccgaaagaatttaacgcggaaacctttacattccatgcagatatctgtacactgtccgagaaagagagacagattaagaaacagacggcgctggtggagcttgtgaagcacaagcctaaagctacgaaggagcaactgaaggcagtcatggatgactttgcggcgtttgtggagaagtgctgtaaagcggacgataaggaaacatgcttcgcagaagaaggaaagaagctggtcgccgctagccaagcggctctgggcctgtag

SEQ ID NO: 150 - DNA, 1758 bp, synthetic construct
  misc_feature 1..1758: note = Albumin of M1
  source 1..1758: mol_type = other DNA
gatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctctag

SEQ ID NO: 151 - DNA, 1758 bp, synthetic construct
  misc_feature 1..1758: note = Albumin of M2
  source 1..1758: mol_type = other DNA
gacgcccacaagtctgaagtggctcaccggtttaaggaccttggcgaggagaactttaaagccctggtgctgattgcctttgcccagtatttacaacaatgccctttcgaagaccacgtgaagctcgtcaatgaggtcaccgagttcgctaagacctgcgtagccgacgaaagtgccgagaactgcgacaagagcctgcacaccctgttcggggacaaactctgtaccgtggccaccctacgggagacatatggggagatggccgactgctgcgcaaaacaggagcccgagagaaatgagtgcttcctgcagcacaaggatgacaaccccaatctgcccagactggtgcgccccgaggtagacgttatgtgcaccgccttccatgacaatgaggagacgttcctgaagaaatacctgtacgagatcgcaagacgtcacccctatttctatgcacctgagctgcttttcttcgccaagagatataaggccgccttcaccgaatgctgccaggcagccgataaggcagcttgcctcctgccaaagctggacgagctgagagatgagggcaaggcctccagcgcgaagcagagactcaaatgcgcaagccttcagaagttcggagaacgcgcctttaaagcctgggccgtcgccagactgagccagcgcttccctaaagccgaattcgcagaagtgagcaagctggtaacggacctgacaaaggtgcatactgagtgctgccatggcgatctgctggagtgcgctgatgacagagcagatttggcgaaatatatttgcgaaaatcaggatagcatcagctctaagctcaaggagtgttgtgagaagcccctgctggaaaaaagccactgcattgcagaggttgagaacgatgaaatgccagccgaccttccatcattggccgccgatttcgtggagtcgaaggatgtgtgtaagaactacgccgaggccaaggacgtgttcctgggcatgttcctgtacgagtatgctagaagacatcccgattacagtgtggtgctgctattgagactggccaagacctacgaaaccaccctggagaaatgttgcgccgcggcagatcctcacgaatgttacgccaaagtgtttgacgaattcaagccactggtagaggagccccagaacttaataaagcagaattgcgagctattcgagcagttgggcgagtacaaattccagaacgcccttctggtgaggtataccaaaaaggtgccccaggtgtctacccctaccctggtggaggtcagccgaaatctgggaaaggtcggatccaagtgctgcaagcacccggaggccaagaggatgccttgcgctgaggactatctcagtgtcgtcctgaatcagctatgcgtgttgcacgagaaaaccccagtgagtgaccgcgtgactaaatgctgcaccgaaagcttggtgaatcggaggccctgtttctctgcactggaagttgacgagacttacgtcccgaaggaattcaacgccgagacattcaccttccatgctgacatatgtactctgtcagaaaaggagcgtcagatcaagaagcagacagccctggtggaactggttaagcataagcctaaagcgaccaaagagcagctgaaagccgtgatggacgattttgccgccttcgtggagaaatgttgtaaggcagacgacaaagagacatgtttcgccgaagaggggaagaaactggtggccgcaagccaggccgctctgggtctgtag

SEQ ID NO: 152 - DNA, 1758 bp, synthetic construct
  misc_feature 1..1758: note = Albumin of M3
  source 1..1758: mol_type = other DNA
gatgcccacaaaagcgaagtcgcacacagattcaaggacttgggtgaggagaactttaaagccctggtgctgatcgccttcgcgcagtatctccagcagtgccccttcgaagatcatgtgaaactggtgaacgaggtaaccgagttcgcgaagacatgcgttgctgatgagagcgccgaaaattgcgacaaaagcctgcatactctgttcggggacaagctgtgcacggtcgcaaccctgagagaaacctacggcgagatggcagactgctgcgccaagcaggagcctgagaggaacgagtgttttctgcagcacaaggacgataatcctaaccttcctcgtctagtgagacccgaagtggacgttatgtgtaccgcctttcacgacaatgaggaaacattcctgaaaaagtacctgtacgagatcgccagacggcacccatatttctacgcccccgagctgctcttcttcgcaaagaggtacaaggctgccttcaccgagtgctgccaggcggccgacaaggcggcgtgtttgctgcctaagctggacgaactacgtgacgaaggaaaagctagcagcgccaagcagagacttaagtgcgcgtccttacagaagtttggcgaaagagcgtttaaggcctgggccgtggcaaggctgtctcaaagattccccaaggcggagttcgccgaggtgtcaaaactggtgaccgacttaaccaaggtgcacaccgaatgctgccacggcgatctgctcgagtgcgccgacgacagagccgatctggcaaaatacatctgcgaaaaccaggatagcatcagctccaaactgaaggagtgctgtgaaaaaccactgcttgaaaaatcgcattgtatagcggaggtggagaatgacgagatgcccgccgacctgccaagcctggccgccgatttcgttgaatccaaggacgtttgcaagaactatgcagaagcgaaggacgtgttcttaggaatgttcctatacgagtacgcgagaagacatcccgactacagcgtggttctgctgttgagattagccaagacgtatgagacaaccctcgaaaagtgctgcgccgccgccgacccccacgagtgttacgcaaaggtgttcgatgagtttaaaccgctggttgaggaaccgcaaaacctgatcaagcagaactgcgagctgttcgagcagctgggtgaatacaagtttcagaatgcactgttggtgcgatataccaagaaggtgcctcaggtgagcacccctacccttgttgaggtgtcccgcaatctgggtaaggttgggagcaagtgttgcaaacaccccgaggccaagagaatgccctgcgcggaagattatctcagtgtcgtgcttaatcagttatgtgtcctgcatgagaagacccccgtgagcgacagagtgaccaagtgctgtaccgaatctctcgtgaacagacgcccgtgcttcagcgccttggaggtagacgagacctacgtgcccaaggagttcaacgcagagaccttcacctttcacgccgatatctgcaccctgtccgagaaggagagacaaatcaagaaacaaacggccctcgtggagctggtcaagcacaaacccaaggccacaaaagagcagctgaaggccgtgatggacgacttcgcagcctttgtggagaaatgctgcaaggctgacgacaaggagacatgcttcgccgaggaaggcaagaagttggtggccgccagccaggcggccctgggcctgtag

SEQ ID NO: 153 - DNA, 1758 bp, synthetic construct
  misc_feature 1..1758: note = Albumin of H1
  source 1..1758: mol_type = other DNA
gacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgagtgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaagagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctttag

SEQ ID NO: 154 - DNA, 1758 bp, synthetic construct
  misc_feature 1..1758: note = Albumin of H2
  source 1..1758: mol_type = other DNA
gacgcccacaagagcgaagtggcccatagattcaaggacctgggcgaggagaacttcaaggccctcgtgctgatcgccttcgcccagtacctgcagcagtgccctttcgaggaccacgttaaactggtgaatgaagtgaccgagttcgccaaaacctgcgtggccgacgagtctgccgaaaattgcgacaaaagcttacacaccctgttcggcgacaagctgtgcaccgtggccaccttaagagaaacctacggcgagatggccgactgctgcgctaagcaggagcccgagagaaacgaatgcttcctgcagcacaaggacgataacccaaatctgcctagactggtgagacccgaggtggacgtgatgtgcacagccttccacgataacgaagagacattcctgaaaaagtacctgtacgagatcgccagaagacatccttacttctatgcccccgagcttctgttcttcgccaagagatataaggccgccttcaccgagtgctgccaagccgccgacaaggcagcctgcctgctgccaaagcttgacgagctgagagacgagggcaaagccagcagcgccaagcagagactgaagtgcgcctccctgcagaagttcggcgagagagcctttaaggcctgggccgtggccagattgagtcagagattccccaaggccgagttcgccgaggtgagcaaactggtgaccgacctcactaaagtgcacacagaatgttgccatggcgatctcctggaatgcgccgatgacagggccgacctggccaagtacatctgtgagaaccaggacagcatttcgagcaagctgaaggagtgctgcgagaaacccctgctggagaagtcccactgcatcgccgaggtggagaacgacgagatgcccgccgacctgcccagcctggccgcagatttcgtggagagcaaggacgtatgcaagaactacgcagaggccaaggatgtgttcctgggcatgttcctgtacgaatacgccagaaggcaccccgactacagcgtcgtgctgttactgagactggccaagacctacgagacaaccttagagaagtgctgcgcggccgcagacccgcacgagtgctacgccaaggtgttcgacgagttcaagcccctggtggaggaacctcagaatctgattaagcagaattgtgagctgttcgagcagctgggagagtacaagttccagaacgcgctgctggtgagatataccaagaaggtgccccaggtgagcacccccaccctggtggaggtgagtcgcaacctgggcaaggtggggagcaagtgttgcaagcatcccgaggccaaaagaatgccctgcgcagaggactatctgagcgttgtgcttaaccagctgtgcgtgctgcacgagaagacccccgtgagcgacagagtgaccaagtgttgcaccgagagcctggtaaacagaagaccctgcttcagcgccctggaggtggacgagacctacgtgcccaaggagttcaacgccgagacctttaccttccacgcagacatttgcaccctgagcgagaaagagaggcagatcaagaaacagaccgctctggtcgagcttgtgaagcacaagcccaaagctaccaaggagcagctgaaggccgtgatggatgatttcgccgccttcgtagagaaatgctgcaaggccgacgataaagagacctgctttgccgaggagggcaagaaactggtggccgccagccaggcggccctgggcctgtag

SEQ ID NO: 155 - DNA, 1758 bp, synthetic construct
  misc_feature 1..1758: note = Albumin of H3
  source 1..1758: mol_type = other DNA
gacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctcttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggagaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctgtag

SEQ ID NO: 156 - DNA, 1758 bp, synthetic construct
  misc_feature 1..1758: note = Albumin of CO
  source 1..1758: mol_type = other DNA
gacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 157 - DNA, 1755 bp, synthetic construct
  misc_feature 1..1755: note = Albumin of CP
  source 1..1755: mol_type = other DNA
gatgcccacaagtctgaggtggcccaccgcttcaaggacctgggggaggagaacttcaaggccctggtgctgattgcctttgcccagtacctgcagcagtgcccctttgaggaccacgtgaagctggtgaatgaggtgacagaatttgccaagacctgtgtggctgatgaatctgctgagaactgtgacaagagcctgcacaccctgtttggagacaagctgtgcaccgtggccaccctgcgggagacctatggagagatggctgactgctgtgccaagcaggagcctgagagaaatgaatgcttcctgcagcacaaggatgacaaccccaacctgccccggctggtgcggcctgaggtggatgtgatgtgcacagccttccatgacaatgaggagaccttcctgaagaagtacctgtatgaaattgcccggcggcacccctacttctacgcccctgagctgctgttctttgccaagcgctacaaggccgccttcacagagtgctgccaggccgctgacaaggccgcctgcctgctgcccaagctggatgagctgagagatgagggcaaggccagcagcgccaagcagaggctgaagtgtgccagcctgcagaagtttggagagcgggccttcaaggcctgggccgtggcccggctgagccagcgcttccccaaggccgagtttgctgaggtgtccaagctggtgacagacctgaccaaggtgcacacagagtgctgccacggggacctgctggagtgtgctgatgacagagctgacctggccaagtacatctgtgagaaccaggacagcatcagcagcaagctgaaggagtgctgtgagaagcccctgctggaaaagagccactgcatcgccgaggtggagaatgatgagatgcctgctgacctgcccagcctggccgctgactttgtggagagcaaggatgtgtgcaagaactatgcagaggccaaggatgtgttcctgggcatgttcctgtatgaatatgcccggcggcacccagactacagcgtggtgctgctgctgcggctggccaagacctatgagaccaccctggagaagtgctgtgccgctgctgacccccatgaatgttatgccaaggtgtttgatgagttcaagcccctggtggaggagccccagaacctgatcaagcagaactgtgagctgtttgagcagctgggggagtacaagttccagaatgccctgctggtgcgctacaccaagaaggtgccccaggtgtccacccccaccctggtggaggtgtccaggaacctgggcaaggtgggcagcaagtgctgcaagcaccctgaggccaagaggatgccctgtgccgaggactacctgtctgtggtgctgaaccagctgtgtgtgctgcacgagaagacccctgtgtctgacagagtgaccaagtgctgcacagagagcctggtgaacagaagaccctgcttcagcgccctggaggtggatgagacctacgtgcccaaggagttcaatgctgagaccttcaccttccacgccgacatctgcaccctgtctgagaaggagcggcagatcaagaagcagacagccctggtggagctggtgaagcacaagcccaaggccaccaaggagcagctgaaggctgtgatggatgactttgctgcctttgtggagaagtgctgcaaggcagatgacaaggagacctgctttgctgaggagggcaagaagctggtggccgccagccaggccgccctgggcctg

SEQ ID NO: 158 - DNA, 1758 bp, synthetic construct
  misc_feature 1..1758: note = Albumin of vH1
  source 1..1758: mol_type = other DNA
gacgcccacaagagcgaggtggcccataggttcaaggacctgggcgaggagaacttcaaggccctggtactcatcgccttcgcccaatacctgcaacagtgccccttcgaggaccatgttaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgaacgtaacgagtgcttcctgcagcacaaggacgacaaccccaacctgccccgactggtcagacccgaggtggacgtgatgtgcacagccttccacgacaacgaggagaccttcctgaagaagtacctctacgagatcgccagaagacatccatacttctacgcccccgagctgctgttcttcgccaagaggtacaaggccgccttcacagagtgctgccaggccgccgacaaggccgcttgcctgctgcctaagttggacgagctgagagacgagggcaaggccagcagcgccaagcagagactgaagtgcgcaagcctgcagaaattcggcgagagagcctttaaggcctgggccgtggccagactgagccagcgcttccccaaggccgagttcgccgaggtgagcaagctggtgaccgacctgaccaaagtgcacaccgagtgctgccacggcgacctgctggagtgcgccgacgacagagccgacctggccaagtacatctgcgagaaccaggacagcatcagcagcaagttgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccggagacaccccgactacagcgtggtgctgctgctgagactggccaagacctacgagaccaccctggagaagtgctgtgccgccgccgacccccacgagtgctacgccaaggtgttcgacgagttcaagcccctggtggaggagccccagaacctgatcaagcagaactgcgagctgttcgagcagctgggcgagtataagttccagaacgccctgctggtgagatacaccaagaaggtgccccaggtcagcacccccaccctggtggaggtgagcagaaatctgggcaaggtgggcagcaaatgctgcaagcaccccgaggccaagagaatgccctgcgccgaggactacctgtcagtggtgctgaaccagctgtgtgtgctgcacgagaagacccccgtgagcgacagagtgaccaagtgctgcaccgagagtctcgtgaacagacggccctgcttcagcgccctggaggtggacgaaacatacgtgcccaaggagttcaacgcagagaccttcaccttccacgcagacatctgcaccctgagcgagaaggagagacagatcaagaagcagaccgccctggttgagcttgtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtag

SEQ ID NO: 159 - DNA, 1758 bp, synthetic construct
  misc_feature 1..1758: note = Albumin of vH2
  source 1..1758: mol_type = other DNA
gacgctcacaagagcgaggtggcccacagattcaaggacctgggcgaggagaacttcaaggccctggtcctgatcgccttcgcccagtacctgcagcagtgccccttcgaggaccacgtgaagctggtgaacgaggtgaccgagttcgccaagacctgcgtggccgacgagtcggccgagaactgcgacaagagcctgcacaccctgttcggcgacaagctgtgcaccgtggccaccctgagagaaacctacggggagatggccgactgctgcgcaaagcaggagcccgagcgaaacgagtgcttcctgcagcacaaggacgacaaccccaacttgcccagactggtaagacccgaggtggacgtcatgtgcaccgctttccacgacaacgaggagaccttcctgaagaagtacctgtacgagatcgccagaagacacccctatttctatgcccctgagctgctgttcttcgccaagcgctacaaggccgccttcaccgagtgctgccaggccgccgacaaggccgcctgcctgctccccaagctggacgagctgagagacgaaggcaaggccagcagcgccaagcagagactgaagtgcgccagcctgcagaagttcggcgagagagccttcaaggcctgggccgtggccagactgtcgcagagattccccaaggccgagttcgccgaggtgagcaagctggttaccgacctgactaaggtgcacaccgagtgctgccacggcgacctgctggagtgcgccgacgacagagccgacctggccaagtacatctgcgagaaccaggatagcatcagcagcaagctgaaggagtgctgcgagaagccccttctggagaagtcccactgcatcgccgaggtggagaacgacgagatgcccgccgacctgcctagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagactacccttgagaagtgctgcgccgccgccgacccacatgagtgctacgccaaggtgttcgacgagttcaagcccctggtggaagagccccagaacctgatcaagcagaactgcgagctgttcgagcagctgggcgagtacaagttccagaacgcccttctggtgagatacaccaagaaggtgcctcaggtgagcacccccacccttgtggaggtgagcagaaacctcggcaaggtgggcagcaagtgctgcaagcatccagaggccaagagaatgccctgcgccgaggactacctgagcgtggtgctgaaccagctgtgcgtgctgcacgagaaaactcccgtgagcgacagagtgaccaagtgctgcaccgagagtctggtgaacagaagaccctgcttcagcgccctggaggtggacgagacctacgtgcccaaggagttcaacgccgagaccttcaccttccacgccgacatctgcaccctgagcgagaaggagcggcagatcaagaagcagaccgccctggtggagctggtgaagcacaagcccaaggccaccaaggagcagctcaaggccgtgatggacgacttcgccgcgttcgtggagaagtgctgcaaggccgacgacaaagagacctgcttcgccgaggagggcaagaagcttgtggccgccagccaggccgccctgggcctgtag

SEQ ID NO: 160 - DNA, 1758 bp, synthetic construct
  misc_feature 1..1758: note = Albumin of vH3
  source 1..1758: mol_type = other DNA
gacgcccacaagagcgaggtggcccacagattcaaggacctcggcgaggagaacttcaaggccctggtgctgatcgctttcgcccagtacctgcagcagtgccccttcgaggaccacgtgaaactggtgaacgaggtgaccgagttcgccaagacctgcgtggccgacgagagcgccgagaactgcgacaagagcctgcacacgttgttcggcgacaagctgtgcaccgtggccaccctgagagagacctacggcgagatggccgactgctgcgccaagcaggagcccgagagaaacgaatgcttcctgcagcacaaggacgacaaccccaacctgccccgcctggtgagacccgaggtggacgtgatgtgcaccgccttccacgacaacgaggagaccttcctgaagaagtacttgtacgagatcgcaagaagacacccgtacttctacgcccccgagctgctgttcttcgccaagagatacaaggccgccttcaccgagtgctgccaggccgccgacaaggccgcctgcctgctgcccaagctggacgagctcagagacgagggcaaggccagcagcgccaagcagagactgaagtgcgccagcctgcagaagttcggcgagagagcctttaaggcctgggccgtggccagactgagccagagattccccaaggccgagttcgccgaagtgagcaagctggtgaccgacctgacgaaggtgcacaccgagtgctgccacggcgacctgctggagtgcgccgacgacagagcagacctggccaagtacatctgcgagaaccaggacagcatcagcagcaagctgaaggagtgctgcgagaagcccctgctggagaagagccactgcatcgccgaggtggagaacgacgagatgcccgccgacctgcccagtctggccgcagacttcgtggagagcaaggatgtgtgcaagaactacgccgaggccaaggacgtgttcctgggcatgttcctgtacgagtacgcaagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgaactgttcgagcagcttggcgagtacaagtttcagaacgccctgctggtcagatacaccaagaaggtgccccaggtgagcacccccaccctggtggaggtgtccagaaacctgggcaaggtgggcagcaagtgctgcaagcaccccgaggcaaagagaatgccctgcgccgaggactatctgagcgtggtgctgaaccagctgtgcgtgctgcacgagaagacccccgtgagcgacagagtgaccaagtgctgcaccgagagcctggtgaatagaagaccctgcttcagcgcactggaggtggacgagacctacgtgcccaaggagttcaacgccgagacattcaccttccacgccgatatctgcaccctgagcgagaaggagagacagatcaagaagcagaccgccctggtggagctggtgaagcacaagcccaaggccaccaaggagcagttgaaggccgtgatggacgacttcgccgccttcgtggagaaatgctgcaaggccgacgacaaggagacctgcttcgccgaggagggcaagaagctggtggccgccagccaggccgccctgggcctgtag

SEQ ID NO: 161 - DNA, 1755 bp, synthetic construct
  misc_feature 1..1755: note = Albumin of B1
  source 1..1755: mol_type = other DNA
gacgcccacaagtccgaggtcgcccacagattcaaggatttgggcgaggagaacttcaaggccctggtgctgatcgccttcgcccagtacttgcagcagtgtcccttcgaggaccatgtgaagctggtgaacgaggtgaccgagttcgccaagacctgtgtggccgacgagagcgccgagaactgcgataagtctctgcacaccctttttggcgacaaactgtgcaccgtggccaccctgagagagacctacggcgagatggccgactgctgtgcgaagcaggagcccgagcgcaatgagtgtttcctgcagcataaggacgacaaccccaacctgcccagactggtgagacccgaggtggacgtgatgtgcaccgccttccacgacaacgaggagacctttctgaaaaaatacctgtacgagatcgcaagacgccacccctacttctacgcccccgagctgctgttcttcgccaagcgctacaaggctgccttcaccgaatgctgccaggccgccgataaggccgcgtgcttactgccaaagctggacgagctgagagacgaggggaaagcctctagcgccaaacagagattgaagtgtgccagcctgcagaaattcggtgagagagccttcaaggcctgggccgtggccagattatcacagcggttccccaaggctgaattcgccgaggtgagcaaacttgtcaccgatctgacaaaagtgcacaccgagtgctgccatggcgacctgctggagtgcgccgacgaccgggccgacctggccaagtacatctgcgagaaccaggacagcatctccagcaagctgaaggagtgctgcgagaagcccctgctggagaaaagccactgcatcgccgaggtggagaatgacgaaatgcccgccgacctgcccagcctggccgccgacttcgtggaaagcaaggacgtgtgcaaaaattacgccgaagccaaggatgtgttcttgggcatgttcttgtacgagtacgccagacgccaccccgactacagcgtggtgctgctgctgcggctggccaagacctacgagaccaccctggagaagtgctgtgctgccgccgacccccacgagtgctacgccaaggtatttgacgagttcaagcccctggtggaggagcctcagaacctgattaagcagaactgtgagctgttcgagcagctgggcgagtacaagttccagaacgccctcctggtgagatacaccaaaaaggtgcctcaggtaagcactcccaccctggtggaggtgagcaggaacctcggcaaggtgggcagcaaatgctgcaagcacccagaggccaaaagaatgccctgcgcagaagactacctcagcgtggtcctgaaccagctgtgcgtgctgcacgaaaagacccctgtgagcgatagagtgacaaagtgctgcaccgagagcctggtgaacagaagaccctgttttagcgccctggaggtggacgagacctacgtgcccaaggagttcaacgccgagacgttcactttccacgcggacatctgcaccctgagcgagaaggagagacaaatcaagaagcagaccgccctagtcgagctggtaaaacacaagcccaaggccaccaaggagcagctgaaggccgtgatggacgactttgcagccttcgtggagaagtgctgcaaggctgacgacaaggagacctgcttcgccgaggagggcaagaagctcgtagccgccagccaggccgctctcggcctt

SEQ ID NO: 162 - DNA, 1755 bp, synthetic construct
  misc_feature 1..1755: note = Albumin of B2
  source 1..1755: mol_type = other DNA
gacgcccacaagagcgaggtggcccacagattcaaggatctcggggaggagaacttcaaggccctggtgctgatcgccttcgcacagtacctgcagcagtgccccttcgaggaccacgtaaaactggtgaacgaggtgacggagttcgccaagacctgtgttgccgacgagtcggccgagaattgcgacaagagcctgcataccctgttcggcgacaagctgtgcaccgtggccaccctgagagagacctacggcgagatggccgactgctgcgccaagcaagagcccgagagaaacgagtgcttcctgcagcacaaggacgacaaccccaacctgccccggctggtgagacccgaggtggacgtgatgtgcaccgccttccacgacaacgaggagaccttcctgaagaagtatctgtacgagatcgccagaagacacccttatttttacgcccccgagctgctgttcttcgctaagagatataaggcagccttcaccgagtgttgtcaggccgccgataaggccgcttgcctgctgcccaagttggacgagctcagagacgagggcaaggcgagcagcgccaagcagagactgaagtgcgccagcctgcagaagttcggcgagagggccttcaaggcctgggcggtggccagactgtcccagagatttcccaaggccgagttcgccgaggtaagcaagctggtcaccgacctgaccaaggtgcacaccgagtgttgccacggcgacctgctggaatgcgccgatgaccgtgccgacctggccaagtacatctgcgagaatcaggactccatcagcagcaaactgaaggagtgttgcgagaagcccctgctggaaaagagccattgcatcgctgaggtggaaaacgacgagatgcccgcagacctgcccagcctggccgcagactttgtggaaagtaaggacgtgtgcaagaactacgcggaggccaaagacgtgtttctgggcatgttcctatacgagtatgccagaagacaccccgactacagcgttgtgttattgctgagactggccaagacctacgagactaccttggagaagtgctgcgccgccgccgacccccacgagtgctatgccaaggtgttcgacgagttcaagcccctggtggaggagccccagaatctgattaagcagaattgcgagctttttgagcagctgggcgagtataagttccagaacgccctgctggtgagatacaccaaaaaggtacctcaggtgagcacccccaccctggtggaggtgagcagaaatctgggcaaggtgggcagcaagtgctgcaagcacccggaggccaagagaatgccctgtgccgaggattacctgtcagtggtgctgaaccagctgtgcgttctgcacgaaaagacgcccgtgtcggacagagtgaccaagtgctgcacggagagcctggtgaacagaagaccgtgcttcagcgccctagaggtggacgagacctacgtgcccaaggagttcaacgccgagaccttcaccttccacgccgacatctgtaccctgtcagagaaggagagacagatcaagaagcagaccgccttagtggagctggtgaagcacaagcccaaggccaccaaggagcagctgaaagccgtgatggacgatttcgcagccttcgtcgagaagtgctgcaaggccgacgacaaggaaacttgcttcgccgaggagggcaagaagctggtggctgcctcgcaggccgccctcggcctg

SEQ ID NO: 163 - DNA, 1755 bp, synthetic construct
  misc_feature 1..1755: note = Albumin of B3
  source 1..1755: mol_type = other DNA
gacgcccacaagagcgaggtggcccacagattcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaggagcccgagagaaacgagtgcttcctgcagcacaaggacgacaaccccaacctgcccagactggtgagacccgaggtggacgtgatgtgcaccgccttccacgacaacgaggagaccttcctgaagaagtacctgtacgagatcgccagaagacacccctacttctacgcccccgagctgctgttcttcgccaagagatacaaggccgccttcaccgagtgctgccaggccgccgacaaggccgcctgcctgctgcccaagctggacgagctgagagacgagggcaaggccagcagcgccaagcagagactgaagtgcgccagcctgcagaagttcggcgagagagccttcaaggcctgggccgtggccagactgagccagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaggacagcatcagcagcaagctgaaggagtgctgcgagaagcccctgctggagaaaagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgccagaagacaccccgactacagcgtggtgctgctgctgagactggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctg

SEQ ID NO: 164 - DNA, 1755 bp, synthetic construct; identical to SEQ ID NO: 157
  misc_feature 1..1755: note = Albumin of B4
  source 1..1755: mol_type = other DNA

SEQ ID NO: 165 - DNA, 1755 bp, synthetic construct
  misc_feature 1..1755: note = Albumin of C1
  source 1..1755: mol_type = other DNA
gatgcccataaatctgaggtggcccatagattcaaggatctgggcgaagaaaacttcaaagccttggtcttgatcgcctttgcccagtacctgcagcagtgcccctttgaggaccacgtgaagctggtgaatgaagtgaccgagtttgccaagacgtgcgtggctgatgagagcgccgaaaactgcgacaaaagcctgcacaccctgtttggcgacaagctgtgcaccgtagccaccctgagagaaacttacggcgagatggctgactgctgcgccaagcaggagcccgagagaaacgagtgctttctgcagcacaaggacgacaatcccaacctgcccagactggtgagacccgaagtggatgttatgtgcaccgctttccacgacaatgaagagacatttctcaagaagtacttgtacgagattgcaagaagacacccttacttttacgcccccgaattactgttcttcgctaagaggtataaggcagccttcactgaatgctgccaggctgccgacaaagcagcttgcctgctgccaaagctggatgaactgcgagacgaaggaaaggcgtcctccgccaagcagcgtttgaagtgcgccagccttcagaagtttggcgagcgggccttcaaggcatgggccgtggctcgacttagccagcgttttcccaaggctgaatttgcagaggtgagtaaactggttaccgatctgacaaaggtgcacaccgagtgctgtcacggtgacctcttagagtgcgccgacgacagagccgacctcgccaagtacatttgtgaaaaccaagactcaatctcttcaaagttaaaggagtgctgcgaaaagcccctgcttgaaaagagccactgcattgccgaagtcgagaatgatgagatgcctgcagacttgcccagcttggcagccgacttcgttgagtctaaggacgtgtgcaagaattacgccgaggcaaaagacgtgttcctgggcatgttcctttatgagtacgctagaagacatcccgactacagcgtggtccttctccttaggctcgctaagacttacgagacgacgttggagaagtgttgtgccgctgcggacccccacgagtgctatgccaaagtgttcgatgagtttaaacccctggtggaggaacctcagaaccttatcaagcagaattgtgagttgttcgaacagctaggcgagtacaagttccagaatgccctgctggtgagatacacaaaaaaggtgccccaggtgtcaaccccgaccttagtggaagtgtccagaaacctgggcaaggtgggcagcaagtgctgcaagcaccccgaagctaagagaatgccgtgcgcggaggattacctgagcgtggtgctcaaccagctgtgtgtgcttcacgagaaaacacccgtgagcgacagggtgacaaaatgttgcacagaaagccttgtgaaccggagaccttgtttcagcgccctggaggttgacgagacctatgttcctaaggagttcaacgctgagactttcacatttcacgctgatatatgtaccctgagcgagaaagaaagacagatcaagaagcagaccgccctggtcgagctggtgaaacacaagcctaaggccacgaaggagcagctgaaggccgtcatggacgacttcgcagccttcgtcgagaaatgctgcaaagccgacgacaaggaaacctgcttcgccgaagagggaaagaagctggtggccgcctcccaggccgcccttgggctc

SEQ ID NO: 166 - DNA, 1755 bp, synthetic construct; identical to SEQ ID NO: 161
  misc_feature 1..1755: note = Albumin of C2
  source 1..1755: mol_type = other DNA

SEQ ID NO: 167 - DNA, 1755 bp, synthetic construct; identical to SEQ ID NO: 162
  misc_feature 1..1755: note = Albumin of C3
  source 1..1755: mol_type = other DNA

SEQ ID NO: 168 - DNA, 24 bp, synthetic construct
  misc_feature 1..24: note = GS Linker of C1
  source 1..24: mol_type = other DNA
ggtggcgggtctggtggcggttct

SEQ ID NO: 169 - DNA, 24 bp, synthetic construct
  misc_feature 1..24: note = GS Linker of C2
  source 1..24: mol_type = other DNA
ggtggcgggtctggtggcggttct

SEQ ID NO: 170 - DNA, 24 bp, synthetic construct
  misc_feature 1..24: note = GS Linker of C3
  source 1..24: mol_type = other DNA
ggtggcgggtctggtggcggttct

SEQ ID NO: 171 - DNA, 960 bp, synthetic construct
  misc_feature 1..960: note = Lumican of C1
  source 1..960: mol_type = other DNA
cagtactacgattacgacttccccctgagtatctacggtcagtcctcacctaactgcgccccagagtgtaactgccccgaaagctacccgagcgccatgtactgcgacgagctgaagttgaagtccgtgcccatggtgcccccaggcatcaagtacttatacttgaggaacaaccaaatcgatcatatcgacgagaaggccttcgaaaacgtaaccgacctgcagtggctgatactggatcacaatctgctagagaattccaagatcaagggcagagtgttctcgaagctgaaacaactgaagaagctgcacatcaaccataacaatctcaccgagagcgtgggtcccctgcccaagtcgctggaggacctgcagctgacccacaacaaaataaccaaactaggcagcttcgaggggttggtgaatctgaccttcatccatttgcagcataacagactaaaggaggatgccgtgagcgccgccttcaaaggcctcaagagccttgagtacctggacctgagcttcaaccagatcgcccggctgcccagtgggctgcccgtgagcctgctgacgctgtatctggacaataacaaaatcagcaacatccccgacgaatacttcaaaagattcaacgccttacagtacctgcgactcagccacaatgagctcgctgacagcggcatccctggcaacagcttcaacgtgtcatccctggtggagctggacctgagttacaataagctgaagaacatcccaactgtcaatgagaatttggaaaactactacctggaggtgaaccagctggagaagttcgacattaagagcttttgcaaaatcctgggcccactgtcatatagcaagatcaagcacctgcgactggacggcaaccgaatcagtgaaacttccctaccccctgacatgtacgagtgcctgagagtagcaaatgaggtgaccctgaac

SEQ ID NO: 172 - DNA, 960 bp, synthetic construct
  misc_feature 1..960: note = Lumican of C2
  source 1..960: mol_type = other DNA
cagtattacgactacgatttccccctgagcatctatggccagagcagccctaactgcgccccggagtgcaactgccccgaaagctacccaagcgccatgtactgcgatgagctgaagctgaagtctgtgcctatggtgcctcccggcatcaagtacctgtacctgagaaacaaccagatagaccacatcgatgagaaagccttcgagaacgtcaccgacctgcagtggctgattctggaccacaatttactggagaactccaagatcaagggcagagtgttctccaagttaaagcagctgaagaaactgcacatcaatcacaacaacctgaccgagagcgtgggcccactgcccaagagcctagaggatctgcagctcacccacaacaagatcactaagttgggcagcttcgagggcctcgtaaacttgacattcatacatctgcagcacaacagacttaaggaagacgccgtgagtgcggcctttaagggtctgaaaagcctggagtacttagacctgagcttcaaccagatcgcaaggctgcccagcggccttccggtcagtctgctgaccctgtatctggacaacaacaagatcagcaacatccccgacgagtacttcaagcggtttaacgccctccagtacctgagactgagccacaacgagttagctgactcgggcatacccggtaacagcttcaatgttagcagcctagttgagcttgacttgagctacaacaagcttaagaacatcccaaccgtgaacgagaacctcgagaattactacctggaagtcaaccagctggagaagttcgacattaagagcttctgcaaaatcctgggcccactgtcctatagcaagatcaagcacctgcgccttgacggaaacagaattagcgagaccagccttccaccagacatgtacgagtgcctgagggtggccaacgaggtgaccctgaac

SEQ ID NO: 173 - DNA, 960 bp, synthetic construct
  misc_feature 1..960: note = Lumican of C3
  source 1..960: mol_type = other DNA
cagtactacgactacgatttccccctatccatctacgggcagagctcgcctaactgcgcccccgagtgtaactgccccgagtcgtaccccagcgccatgtactgtgacgagctgaagctgaaaagcgtgcccatggtgccccccggcatcaagtacctgtacttgagaaacaaccagatcgaccacattgacgaaaaggccttcgagaacgtaaccgacctgcagtggctgatcctggaccacaacctgcttgagaacagcaagatcaagggccgcgtgttcagcaagctgaagcagctgaagaagctgcacatcaaccacaacaacttgactgagtctgttggccccctaccaaagagcctggaggacctgcagctgacccacaataagataaccaagctgggctcattcgagggcctggtgaacttgacctttattcacctgcagcataacagactgaaggaggacgccgtgagcgccgcctttaaggggctgaaaagcctggagtacctggacctgagttttaaccagatcgccagactgccctcaggcctgcccgtgagtttgctgactctgtacctggacaacaataagatcagcaacattcctgacgagtatttcaaaagattcaatgctctgcagtacctgagactaagccacaacgagctggccgacagcggaatccccggcaacagcttcaacgtgagcagcttggtggagttggacctgagctacaacaaactgaagaacatccccaccgtcaatgagaacttggagaattactacctcgaggttaaccagcttgagaagttcgacatcaagagcttctgcaagatcctgggccccctcagctacagcaagatcaagcacttgagactggacgggaacagaatcagcgaaaccagccttcctcccgacatgtacgagtgccttagagtggcaaatgaggtgaccctgaac

SEQ ID NO: 174 - DNA, 13 bp, synthetic construct
  misc_feature 1..13: note = Kozak Sequence
  source 1..13: mol_type = other DNA
gccgccrccatgg

SEQ ID NO: 175-179 - skipped (5 entries)

SEQ ID NO: 180 - protein, 21 aa, synthetic construct
  REGION 1..21: note = Leader Sequence
MRVPAQLLGLLLLWLPGARCA

SEQ ID NO: 181 - DNA, 34 bp, synthetic construct
  misc_feature 1..34: note = Forward Primer
  source 1..34: mol_type = other DNA
taatacgactcactataatggactacgacatagt

SEQ ID NO: 182 - protein, 547 aa, synthetic construct
  REGION 1..547: note = Wild-type IL-12
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 183 - protein, 219 aa, synthetic construct
  REGION 1..219: note = Wild-type IL12a
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 184 - protein, 328 aa, synthetic construct
  REGION 1..328: note = Wild-type IL12b
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 185 - DNA, 35 bp, synthetic construct
  misc_feature 1..35: note = Reverse Primer
  source 1..35: mol_type = other DNA
gaaatattaaaaacaaaatccgattcggaaaagaa

SEQ ID NO: 186 - DNA, 10276 bp, synthetic construct
  source 1..10276: mol_type = other DNA
  misc_feature 1..10276: note = VEE replicon #1
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatgggcgcgccctcagcatcgattgaattggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctctaatacgactcactatag

SEQ ID NO: 187 - DNA, 9698 bp, synthetic construct
  source 1..9698: mol_type = other DNA; note = VEE replicon #2
gctcttctaagtaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccgatatctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaagaagagcgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

(19) United States
(12) Patent Application Publication
(10) Pub. No.: US 2025/0152737 A1
(43) Pub. Date: May 15, 2025

(54) LIPID NANOPARTICLES COMPRISING VENEZUELAN EQUINE ENCEPHALITIS (VEE) REPLICON AND USES THEREOF

(71) Applicant: Strand Therapeutics Inc., Boston, MA (US)

(72) Inventors: Ryan SOWELL, Boston, MA (US); Rio BOOTHELLO, Boston, MA (US); Wuhbet ABRAHAM, Boston, MA (US); Weiyu ZHAO, Boston, MA (US); Nandita MENON, Boston, MA (US); Aalok SHAH, Boston, MA (US); Prashant NAMBIAR, Boston, MA (US); Tasuku KITADA, Boston, MA (US)

(73) Assignee: Strand Therapeutics Inc., Boston, MA (US)

(21) Appl. No.: 18/860,535

(22) PCT Filed: Apr. 26, 2023
(86) PCT No.: PCT/US2023/066266

§ 371 Date: Oct. 25, 2024

Related U.S. Application Data
(60) Provisional application No. 63/335,092, filed on Apr. 26, 2022.

(51) Int. Cl. A61K 48/00; A61K 9/00; A61K 9/1272; A61K 9/51; A61K 45/06; B82Y 5/00; C12N 15/86; C12N 15/88

(52) U.S. Cl. CPC A61K 48/0041; A61K 9/0019; A61K 9/1272; A61K 9/5123; A61K 45/06; A61K 48/0075; C12N 15/86; C12N 15/88; B82Y 5/00; C12N 2770/36143; C12N 2830/50

(57) ABSTRACT

The present disclosure relates to lipid nanoparticles (LNPs) comprising VEE replicon, wherein the LNPs are useful for selectively reducing and/or avoiding the expression of a payload within the liver. The present disclosure also relates to the use of such LNPs to treat various diseases and disorders.

### CROSS-REFERENCE TO RELATED APPLICATION

This PCT application claims the priority benefit of U.S. Provisional Application No. 63/335,092, filed Apr. 26, 2022, which is incorporated herein by reference in its entirety.

### REFERENCE TO SEQUENCE LISTING SUBMITTED ELECTRONICALLY VIA EFS-WEB

The content of the sequence listing is submitted electronically (Name: 4597_016PC01_SequenceListing_ST26.XML; Size: 410,263 bytes; and Date of Creation: Apr. 24, 2023) and is filed with the application is herein incorporated by reference in its entirety.

### BACKGROUND OF THE DISCLOSURE

Nucleic acid-based treatment (e.g., mRNA vaccines) is becoming an increasingly important approach for the treatment of a variety of diseases. Upon administration, the encoded protein can be produced to induce an immune response (e.g., tumor antigen to induce anti-tumor immune response) or to provide important function to maintain a healthy state. However, current delivery platforms for delivering the nucleic acid molecules are ineffective or associated with undesirable side effects, limiting the full potential of nucleic acid-based therapy. For example, where mRNAs are involved, lipid nanoparticles (LNPs) are commonly used to encapsulate the mRNA. And, to improve LNP delivery, much effort has focused on identifying novel lipids or particular lipid compositions that can affect intracellular delivery and/or expression of mRNA, e.g., in various types of mammalian tissue, organs and/or cells (e.g., mammalian liver cells). However, these existing approaches are costly, time consuming and unpredictable. Additionally, LNPs have also been associated with liver toxicity when administered in vivo. Accordingly, there is a need in the art for improved LNPs that can provide safer and more effective means of delivering nucleic acids to a subject.

### BRIEF SUMMARY OF THE DISCLOSURE

Provided herein is a method of reducing or avoiding the expression of a heterologous protein in the liver of a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the heterologous protein. In some aspects, reducing the expression comprises (i) reducing the amount of heterologous protein that is expressed in the liver, (ii) reducing the duration of the expression of the heterologous protein in the liver, or (iii) both (i) and (ii), as compared to that of a reference subject (e.g., a subject who received a corresponding lipid nanoparticle but where the replicon is not a VEE replicon).

In some aspects, after the administration, the amount of heterologous protein that is expressed in the liver is reduced by at least about 5%, at least about 10%, at least about 20%, at least about 30%, at least about 40%, at least about 50%, at least about 60%, at least about 70%, at least about 80%, at least about 90%, or about 100%, as compared to that of the reference subject. In some aspects, after the administration, the liver does not express the heterologous protein.

In some aspects, after the administration, the duration of the expression of the heterologous protein in the liver is reduced by at least about 5%, at least about 10%, at least about 20%, at least about 30%, at least about 40%, at least about 50%, at least about 60%, at least about 70%, at least about 80%, at least about 90%, or about 100%, as compared to that of the reference subject.

In some aspects, after the administration, the heterologous protein is expressed in a non-liver tissue of the subject, wherein the non-liver tissue is selected from spleen, lung, tumor, or combinations thereof. In some aspects, after the administration, the amount of heterologous protein that is expressed in the non-liver tissue is at least about 90%, at least about 91%, at least about 92%, at least about 93%, at least about 94%, at least about 95%, at least about 96%, at least about 97%, at least about 98%, at least about 99%, or at least about 100% or more, as compared to that observed in the non-liver tissue of the reference subject. In some aspects, the duration of the expression of the heterologous protein in the non-liver tissue is at least about 90%, at least about 91%, at least about 92%, at least about 93%, at least about 94%, at least about 95%, at least about 96%, at least about 97%, at least about 98%, at least about 99%, or at least about 100%, as compared to that observed in the non-liver tissue of the reference subject.

Also provided herein is a method of selectively expressing a heterologous protein in a non-liver tissue of a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the heterologous protein.

In some aspects, after the administration, the amount of heterologous protein that is expressed in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold greater than the corresponding amount observed in the liver of the subject. In some aspects, after the administration, the duration of the expression of the heterologous protein in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding duration observed in the liver of the subject. In some aspects, the non-liver tissue comprises spleen, lung, or both.

Disclosed herein is a method of treating a disease or disorder in a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the heterologous protein, and wherein after the administration, the heterologous protein is preferentially expressed in a non-liver tissue of the subject.

In some aspects, after the administration, the amount of heterologous protein that is expressed in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding amount observed in the liver of the subject. In some aspects, after the administration, the duration of the expression of the heterologous protein in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding duration observed in the liver of the subject. In some aspects, the non-liver tissue comprises spleen, lung, or both.

In some aspects, the disease or disorder that can be treated with present disclosure comprises a cancer, inflammatory disorders, monogenic disorders, neurological disorders, psychiatric disorders, or combinations thereof. In some aspects, the cancer comprises a melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, head and neck cancer, or combinations thereof.

Present disclosure further provides a method of increasing tolerability of a lipid nanoparticle-based therapy in a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding a heterologous protein.

In some aspects, the tolerability of the lipid nanoparticle-based therapy is increased in the subject by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding tolerability observed in a reference subject (e.g., a subject who received a corresponding lipid nanoparticle but where the replicon is not a VEE replicon).

In some aspects, the increased tolerability of the lipid nanoparticle-based therapy is associated with decreased liver toxicity. In some aspects, after the administration, the subject exhibits decreased liver toxicity compared to the corresponding liver toxicity observed in the reference subject. In some aspects, the liver toxicity in the subject is decreased by at least about 5%, at least about 10%, at least about 20%, at least about 30%, at least about 40%, at least about 50%, at least about 60%, at least about 70%, at least about 80%, at least about 90%, or about 100%, as compared to that of the reference subject.

In some aspects, after the administration, the subject exhibits decreased expression of the heterologous protein in the liver as compared to the corresponding expression observed in the liver of the reference subject.

In some aspects, after the administration, the amount of heterologous protein that is expressed in the liver of the subject is reduced by at least about 5%, at least about 10%, at least about 20%, at least about 30%, at least about 40%, at least about 50%, at least about 60%, at least about 70%, at least about 80%, at least about 90%, or about 100%, as compared to that observed in the liver of the reference subject. In some aspects, after the administration, the duration of the expression of the heterologous protein in the liver of the subject is reduced by at least about 5%, at least about 10%, at least about 20%, at least about 30%, at least about 40%, at least about 50%, at least about 60%, at least about 70%, at least about 80%, at least about 90%, or about 100%, as compared to that observed in the liver of the reference subject. In some aspects, the amount of heterologous protein that is expressed in the non-liver tissue of the subject is at least about 90%, at least about 91%, at least about 92%, at least about 93%, at least about 94%, at least about 95%, at least about 96%, at least about 97%, at least about 98%, at least about 99%, or at least about 100% or more, as compared to that observed in the non-liver tissue of the reference subject. In some aspects, the duration of the expression of the heterologous protein in the non-liver tissue of the subject is at least about 90%, at least about 91%, at least about 92%, at least about 93%, at least about 94%, at least about 95%, at least about 96%, at least about 97%, at least about 98%, at least about 99%, or at least about 100%, as compared to that of the reference subject.

Provided herein is a method of producing a lipid nanoparticle-based therapy that is associated with decreased liver toxicity, comprising combining (i) a lipid nanoparticle, which comprises one or more types of lipids, and (ii) a replicon derived from a Venezuelan equine encephalitis (VEE) virus (“VEE replicon”), wherein the VEE replicon comprises a nucleic acid sequence encoding a heterologous protein.

In any of the methods provided herein, in some aspects, the VEE replicon has a nucleotide sequence as set forth in SEQ ID NO: 187. In some aspects, the heterologous protein comprises a cytokine, an antibody or antigen-binding fragment thereof, chimeric antigen receptor, or combination thereof. In some aspects, the cytokine comprises an interleukin (IL)-12 protein.

In some aspects, the nucleic acid sequence of the VEE replicon comprises: (i) a nucleotide sequence encoding a IL-12 beta subunit and having at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the sequence set forth in SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75; (ii) a nucleotide sequence encoding a IL-12 alpha subunit and having at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125; or (iii) both (i) and (ii).

In some aspects, the cytokine does not comprise an IL-12 protein.

In some aspects, the one or more types of lipid comprises an ionizable lipid, a cationic lipid, a lipidoid, a phospholipid, a sterol, or combinations thereof. In some aspects, the one or more types of lipid comprise TT3, 1,2-Dioleoyl-sn-glycero-3-phosphoethanolamine (DOPE), cholesterol, C14-PEG2000, or a combination thereof. In some aspects, the one or more types of lipid is TT3. In some aspects, the C14-PEG2000 comprises 1,2-dimyristoyl-rac-glycero-3-methoxypolyethylene glycol-2000 (DMG-PEG2000), 1,2-dimyristoyl-sn-glycero-3-phosphoethanolamine-N-[methoxy (polyethylene glycol)-2000] (DMPE-PEG2000), or both. In some aspects, the C14-PEG2000 is embedded in the lipid nanoparticle. In some aspects, the C14-PEG2000 has been added after the isolated polynucleotide has been encapsulated in the lipid nanoparticle.

In some aspects, the lipid nanoparticle has a diameter of about 30-500 nm. In some aspects, the lipid nanoparticle has a diameter of about 50-400 nm. In some aspects, the lipid nanoparticle has a diameter of about 70-300 nm. In some aspects, the lipid nanoparticle has a diameter of about 100-200 nm. In some aspects, the lipid nanoparticle has a diameter of about 100-175 nm. In some aspects, the lipid nanoparticle has a diameter of about 100-160 nm.

In some aspects, the one or more types of lipid and the VEE replicon have a mass ratio of about 1:2 to about 15:1. In some aspects, the one or more types of lipid and the VEE replicon have a mass ratio of 1:2, 1:1.5, 1:1.2, 1:1.1, 1:1, 1.1:1, 1.2:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, or 15:1.

In some aspects, the VEE replicon comprises a 5′-cap.

In some aspects, the VEE replicon further comprises a regulatory element. In some aspects, the regulatory element is selected from the group consisting of at least one translation enhancer element (TEE), a translation initiation sequence, at least one microRNA binding site or seed thereof, a 3′ tailing region of linked nucleosides, an AU rich element (ARE), a post transcription control modulator, a 5′ UTR, a 3′ UTR, and combinations thereof. In some aspects, the 3′ tailing region of linked nucleosides comprises a poly-A tail, a polyA-G quartet, or a stem loop sequence.

In some aspects, the VEE replicon comprises at least one modified nucleoside. In some aspects, the at least one modified nucleoside is selected from the group consisting of 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, O6-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

In some aspects, the one or more types of lipid and the VEE replicon have a mass ratio of about 10:1.

In some aspects, the lipid nanoparticle is administered to the subject via intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration.

In any of the methods provided herein, in some aspects, the method further comprises administering to the subject at least one additional therapeutic agent. In some aspects, the at least one additional therapeutic agent comprises a chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof. In some aspects, the immune checkpoint inhibitor comprises an anti-PD-1 antibody, anti-PD-L1 antibody, anti-LAG-3 antibody, anti-CTLA-4 antibody, anti-GITR antibody, anti-TIM3 antibody, or any combination thereof.

### BRIEF DESCRIPTION OF THE DRAWINGS/FIGURES

FIG. 1 provides a comparison of luminescence expression in the liver of mice after intravenous administration of one of the following: (1) RNA that without any VEE replicon (RNA-no VEE), (2) RNA with a VEE replicon (RNA-VEE), and (3) PBS. For comparison, luminescence expression in the kidney, spleen, and lung are also provided.

FIGS. 2A and 2B provide comparison of td-Tomato protein fluorescence expression in the liver of mice after a single intravenous administration of LNP1 or LNP2. Both LNP1 and LNP2 were loaded with a mRNA with a VEE replicon and encoding Cre recombinase fused to a nuclear localization signal (Cre-NLS). Control animals received an administration of PBS. In FIG. 2A, the fluorescence expression is shown as % radiant efficiency (normalized to control). In FIG. 2B, the fluorescence expression is shown as the average radiant efficiency. In both of the figures, each symbol represents one measured organ and error bars representing the standard error of the mean. The differences in fluorescent intensities between the LNP vs control groups were statistically analyzed using a two-tailed unpaired Student's t-test (N=3).

FIGS. 3A and 3B provide comparison of td-Tomato protein fluorescence expression in the liver of mice after a single intravenous administration of LNP3, LNP4, LNP5, or LNP6. Each of LNP3-LNP6 were loaded with a mRNA without any VEE replicon (RNA-no VEE) and encoding Cre recombinase fused to a nuclear localization signal (Cre-NLS). Control animals received an administration of PBS. In FIG. 3A, the fluorescence expression is shown as the % average radiant efficiency (normalized to control). In FIG. 3B, the fluorescence expression is shown as the average radiant efficiency.

FIGS. 4A and 4B provide comparison of td-Tomato protein fluorescence expression in the liver of mice after a single intravenous administration of LNP7 or LNP3. Both LNP3 and LNP7 were loaded with a mRNA without any VEE replicon (RNA-no VEE) and encoding Cre recombinase fused to a nuclear localization signal (Cre-NLS). Control animals received an administration of PBS. In FIG. 4A, the fluorescence expression is shown as the % radiant efficiency (normalized to control). In FIG. 4B, the fluorescence expression is shown as the average radiant efficiency.

FIGS. 5A and 5B provide comparison of firefly luciferase expression in the liver of mice after a single administration of LNP8, LNP9, or LNP2 all loaded with a mRNA with a VEE replicon and encoding firefly luciferase. LNP8 was administered to the animals either intravenously (IV) or intratumorally (IT). LNP9 and LNP2 were administered to the animals intravenously. Control animals received an administration of PBS. In FIG. 5A, the luciferase expression is shown as the % radiance (normalized to control). In FIG. 5B, the luciferase expression is shown as the average radiance. In both of the figures, each symbol represents one measured organ and the error bars represent the standard error of the mean.

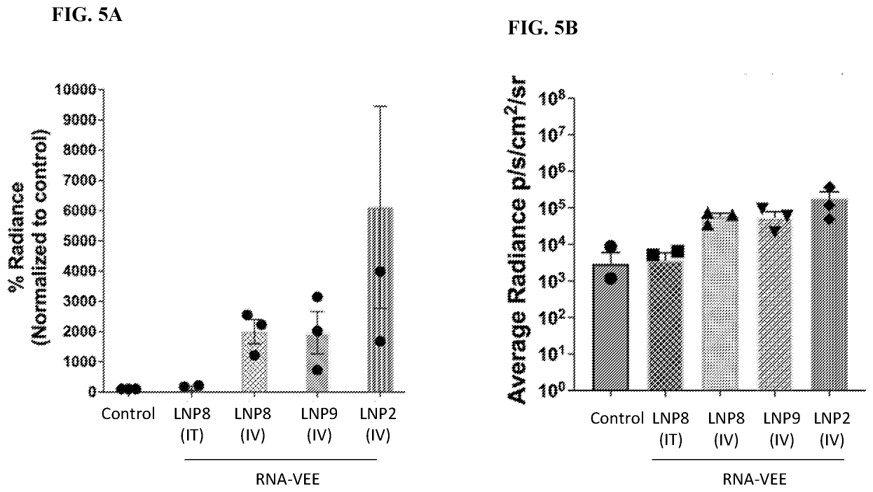

FIGS. 6A and 6B provide comparison of firefly luciferase expression in the liver of mice after a single intravenous administration of LNP8, LNP9, or LNP2 (all loaded with a mRNA without any VEE replicon (RNA-no VEE) and encoding firefly luciferase). Control animals received an administration of PBS. In FIG. 6A, the luciferase expression is shown as % radiance (normalized to control). In FIG. 6B, the luciferase expression is shown as the average radiance. In both of the figures, each symbol represents one measured organ and the error bars represent the standard error of the mean. The differences in bioluminescence intensities between the LNP vs control groups were statistically analyzed using a two-tailed unpaired Student's t-test (N=5).

FIGS. 7A and 7B provide comparison of firefly luciferase expression in the liver of mice after a single administration with either (a) LNP8 loaded with a mRNA without any VEE replicon (RNA-no VEE) and encoding firefly luciferase or (b) LNP8 loaded with a mRNA with a VEE replicon and encoding firefly luciferase. In FIG. 7A, the luciferase expression is shown as % radiance (normalized to control. In FIG. 7B, the luciferase expression is shown as the average radiance. In both of the figures, each symbol represents one measured organ and the error bars represent the standard error of the mean. The differences in bioluminescence intensities between the LNP vs control groups were statistically analyzed using a two-tailed unpaired Student's t-test.

### DETAILED DESCRIPTION OF THE DISCLOSURE

The present disclosure is directed to lipid nanoparticles (LNPs) and the use of such LNPs to selectively deliver a payload (e.g., heterologous protein) in a subject. More specifically, LNPs of the present disclosure comprise (i) one or more types of lipids, and (ii) a replicon derived from a Venezuelan equine encephalitis (VEE) virus (“VEE replicon”), wherein the VEE replicon comprises a nucleic acid sequence encoding a payload. As demonstrated herein, in some aspects, when administered to a subject, the LNPs described herein are capable of selectively avoiding and/or preventing the expression of the encoded payload within the liver of the subject. Accordingly, in some aspects, LNPs of the present disclosure are associated with decreased liver toxicity compared to other delivery platforms available in the art. Additional aspects of the present disclosure are provided throughout the present application.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

“Nucleic acid,” “nucleic acid molecule,” “nucleotide sequence,” “nucleic acid sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein. As described herein, a polynucleotide of the present disclosure comprises DNA, RNA, or both. In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

The term “replicon,” as used herein, refers to a polynucleotide comprising an origin of replication which allows for replication of the polynucleotide in a cell of interest. As further described herein, in some aspects, the replicon is a self-replicating mRNA (i.e., capable of directing its own amplification or replication within a target cell) (also referred to herein as “repRNA” or “RNA-VEE”). To direct its own amplification, the RNA molecule should encode the enzyme(s) necessary to catalyze RNA amplification (e.g., alphavirus nonstructural proteins nsP1, nsP2, nsP3, nsP4) and also contain cis RNA sequences required for replication which are recognized and utilized by the encoded enzymes(s). An alphavirus RNA vector replicon should contain the following ordered elements: 5′ viral or cellular sequences required for nonstructural protein-mediated amplification (may also be referred to as 5′CSE, or 5′ cis replication sequence, or 5′ viral sequences required in cis for replication, or 5′ sequence which is capable of initiating transcription of an alphavirus), sequences which, when expressed, code for biologically active alphavirus nonstructural proteins (e.g., nsP1, nsP2, nsP3, nsP4), and 3′ viral or cellular sequences required for nonstructural protein-mediated amplification (may also be referred as 3′CSE, or 3′ viral sequences required in cis for replication, or an alphavirus RNA polymerase recognition sequence). The alphavirus RNA vector replicon may contain a means to express one or more heterologous sequence(s), such as for example, an IRES or a viral (e.g., alphaviral) subgenomic promoter (e.g., junction region promoter) which may, in certain aspects, be modified in order to increase or reduce viral transcription of the subgenomic fragment, or to decrease homology with defective helper or structural protein expression cassettes, and one or more heterologous sequence(s) to be expressed. A replicon can also contain additional sequences, for example, one or more heterologous sequence(s) encoding one or more polypeptides (e.g., a protein-encoding gene or a 3′ proximal gene) and/or a polyadenylate tract. The replicon should not contain sequences encoding all of the alphavirus structural proteins (capsid, E1, E2). Non-limiting examples of heterologous sequences that can be expressed by replicon vectors are described, for example in U.S. Pat. No. 6,015,686, incorporated by reference in its entirety herein, and include, for example, antigens, lymphokines, cytokines, etc. As is apparent from the present disclosure, VEE replicons provided herein are repRNA. Accordingly, “VEE replicon”, “RNA-VEE”, and “repRNA” are used interchangeably in the present application. Additionally, the terms “mRNA without any VEE replicon,” “RNA without any VEE replicon,” and “RNA-no VEE” are used herein interchangeably with the term “modRNA.”

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a polynucleotide of the present disclosure, mixed or intermingled with, or suspended in one or more other chemical components, such as pharmaceutically acceptable carriers and excipients.

The terms “polypeptide,” “peptide,” and “protein” are used interchangeably herein to refer to polymers of amino acids of any length, e.g., that are encoded by a polynucleotide described herein. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multi-chain polypeptides. Most commonly disulfide linkages are found in multi-chain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, about 10, about 15, about 20, about 25, about 30, about 35, about 40, about 45, or about 50 amino acids long.

The term “coding sequence” or sequence “encoding” is used herein to mean a DNA or RNA region (the transcribed region) which “encodes” a particular protein, e.g., such as an IL-12. A coding sequence is transcribed (DNA) and translated (RNA) into a polypeptide, in vitro or in vivo, when placed under the control of an appropriate regulatory region, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotes or eukaryotes, genomic DNA from prokaryotes or eukaryotes, and synthetic DNA sequences. A transcription termination sequence can be located 3′ to the coding sequence.

A Kozak consensus sequence, Kozak consensus or Kozak sequence, is known as a sequence which occurs on eukaryotic mRNA and has the consensus (gcc)gccRccAUGG (SEQ ID NO: 174), where R is a purine (adenine or guanine) three bases upstream of the start codon (AUG), which is followed by another “G.” In some aspects, the polynucleotide comprises a nucleic acid sequence having at least about 95% or more, e.g., at least 99% sequence identity, to the Kozak consensus sequence. In some aspects, the polynucleotide comprises a Kozak consensus sequence.

The term “RNA” is used herein to mean a molecule which comprises at least one ribonucleotide residue. “Ribonucleotide” relates to a nucleotide with a hydroxyl group at the 2′-position of a β-D-ribofuranosyl group. The term comprises double-stranded RNA, single-stranded RNA, isolated RNA such as partially or completely purified RNA, essentially pure RNA, synthetic RNA, recombinantly generated RNA differs from naturally occurring RNA by addition, deletion, substitution and/or alteration of one or more nucleotides. The term “mRNA” means “messenger-RNA” and relates to a “transcript” which is generated by using a DNA template and encodes a peptide or protein. Typically, an mRNA comprises a 5′-UTR, a protein coding region and a 3′-UTR. mRNA only possesses limited half-life in cells and in vitro. In the context of the present disclosure, mRNA can be generated by in vitro transcription from a DNA template. The in vitro transcription methodology is known to the skilled person. For example, there is a variety of in vitro transcription kits commercially available. In some aspects of the disclosure, the RNA, preferably the mRNA, is modified with a 5′-cap structure.

As used herein, the term “identity” (e.g., sequence identity) refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In some aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In some aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a polynucleotide of the present disclosure, that has undergone one or more processes of purification. In some aspects, isolating or purifying as used herein is the process of removing, partially removing (e.g., a fraction) of a composition of the present disclosure from a sample containing contaminants.

The term “expression,” as used herein, refers to a process by which a polynucleotide produces a gene product, e.g., RNA or a polypeptide (e.g., therapeutic protein, e.g., coronavirus protein). It includes without limitation transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage.

“Heterologous” in reference to a polypeptide moiety or polynucleotide moiety that is part of a larger polypeptide or polynucleotide, respectively, describes a polypeptide or polynucleotide that originates from a different polypeptide or polynucleotide than the remaining part of the polypeptide or polynucleotide molecule. The additional heterologous component of the polypeptide or polynucleotide can originate from the same organism as the remaining polypeptide or polynucleotide, respectively, described herein, or the additional components can be from a different organism. For instance, a heterologous polypeptide can be synthetic, or derived from a different species, different cell type of an individual, or the same or different type of cell of distinct individuals. As described herein, a protein (or polypeptide) encoded by an ORF of a polynucleotide described herein is heterologous to the polynucleotide.

The term “half-life” relates to the period of time which is needed to eliminate half of the activity, amount, or number of molecules. In the context of the present disclosure, the half-life of an RNA is indicative for the stability of said RNA. “Development” or “progression” of a disease means initial manifestations and/or ensuing progression of the disease. Development of the disease can be detectable and assessed using standard clinical techniques as well known in the art. However, development also refers to progression that can be unpredictable. As used herein, development or progression refers to the biological course of symptoms. Development includes occurrence, recurrence, and onset. As used herein, onset or occurrence of a target disease or disorder includes initial onset and/or recurrence.

### II. Methods of the Disclosure

### II.A. Methods of Selective Expression

The present disclosure generally relates to the delivery of a payload (also referred to herein as “biologically active molecule”) to cells using lipid nanoparticles (LNPs). LNPs useful for the present disclosure have been modified or engineered, such that the LNPs exhibit tissue-specific tropism. As used herein, “tissue-specific tropism” refers to the ability of the LNPs to target specific tissues while not targeting other tissues. For instance, as demonstrated herein, LNPs described herein are capable of inducing the expression of a payload in a non-liver tissue (e.g., spleen and lung) but not in the liver. Not to be bound by any one theory, Applicant has identified that modifying or engineering LNPs to comprise a replicon derived from a Venezuelan equine encephalitis (VEE) virus (“VEE replicon”) allows for the selective delivery of a payload, such that the expression of the payload is avoided or reduced in the liver.

Accordingly, in some aspects, provided herein is a method of reducing or avoiding the expression of a payload (e.g., heterologous protein) in the liver of a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the payload.

In some aspects, the expression of the payload is avoided entirely (i.e., the payload is not expressed in the liver after the administration). In some aspects, the expression of the payload in the liver is reduced compared to a non-liver tissue (e.g., spleen and lung) within the subject. For instance, in some aspects, after administration, the expression of the payload in the liver is reduced by at least about 5%, at least about 10%, at least about 20%, at least about 30%, at least about 40%, at least about 50%, at least about 60%, at least about 70%, at least about 80%, at least about 90%, or about 100%, as compared to the corresponding expression in the non-liver tissue. In some aspects, the expression of the payload in the liver is reduced compared to the expression of the payload in the liver of a reference subject (e.g., a subject who received a corresponding lipid nanoparticle that does not comprise the VEE replicon). In some aspects, after the administration, the expression of the payload in the liver is reduced by at least about 5%, at least about 10%, at least about 20%, at least about 30%, at least about 40%, at least about 50%, at least about 60%, at least about 70%, at least about 80%, at least about 90%, or about 100%, as compared to the corresponding expression in the liver of the reference subject.

As used herein, “reducing the expression” refers to (i) a decrease in the total amount of the payload that is expressed in a tissue (e.g., liver); (ii) a decrease in how long (i.e., duration) the payload is expressed in a tissue (e.g., liver); or (iii) both (i) and (ii).

Accordingly, in some aspects, after administration of a LNP described herein (e.g., comprising a VEE replicon encoding a payload), the total amount of payload that is expressed in the liver is reduced compared to a non-liver tissue (e.g., spleen and lung). In some aspects, the total amount of payload that is expressed in the liver is reduced by at least about 5%, at least about 10%, at least about 20%, at least about 30%, at least about 40%, at least about 50%, at least about 60%, at least about 70%, at least about 80%, at least about 90%, or about 100%, as compared to the corresponding amount in the non-liver tissue. In some aspects, the total amount of payload that is expressed in the liver is reduced compared to the total amount of the payload that is expressed in the liver of a reference subject (e.g., a subject who received a corresponding lipid nanoparticle that does not comprise the VEE replicon). In some aspects, after the administration of a LNP described herein (e.g., comprising a VEE replicon comprising a nucleic acid sequence encoding a payload), the total amount of payload that is expressed in the liver is reduced by at least about 5%, at least about 10%, at least about 20%, at least about 30%, at least about 40%, at least about 50%, at least about 60%, at least about 70%, at least about 80%, at least about 90%, or about 100%, as compared to the corresponding amount in the liver of the reference subject.

As described herein, in some aspects, after administration of a LNP described herein (e.g., comprising a VEE replicon comprising a nucleic acid sequence encoding a payload), the payload is only transiently expressed in the liver, e.g., as compared to the corresponding expression in a non-liver tissue and/or as compared to the corresponding expression in the liver of a reference subject (e.g., a subject who received a corresponding lipid nanoparticle that does not comprise the VEE replicon). Accordingly, in some aspects, administering a LNP of the present disclosure results in reduced duration of payload expression in the liver as compared to a non-liver tissue (e.g., spleen and lung). In some aspects, the duration of payload expression in the liver is decreased by at least about 5%, at least about 10%, at least about 20%, at least about 30%, at least about 40%, at least about 50%, at least about 60%, at least about 70%, at least about 80%, at least about 90%, or about 100%, as compared to the corresponding duration in a non-liver tissue. In some aspects, after administration of a LNP described herein (e.g., comprising a VEE replicon comprising a nucleic acid sequence encoding a payload) to a subject in need thereof, the duration of payload expression in the liver of the subject is reduced as compared to the duration of the payload expression in the liver of a reference subject (e.g., a subject who received a corresponding lipid nanoparticle that does not comprise the VEE replicon). In some aspects, the duration of payload expression in the liver of the subject is reduced by at least about 5%, at least about 10%, at least about 20%, at least about 30%, at least about 40%, at least about 50%, at least about 60%, at least about 70%, at least about 80%, at least about 90%, or about 100%, as compared to the corresponding duration in the liver of a the reference subject.

In some aspects, after administration of a LNP described herein (e.g., comprising a VEE replicon comprising a nucleic acid sequence encoding a payload), both (i) the total amount of the payload that is expressed and (ii) the duration of payload expression is reduced in the liver as compared to a non-liver tissue (e.g., spleen and lung). In some aspects, both (i) the total amount of the payload that is expressed and (ii) the duration of payload expression is reduced by at least about 5%, at least about 10%, at least about 20%, at least about 30%, at least about 40%, at least about 50%, at least about 60%, at least about 70%, at least about 80%, at least about 90%, or about 100%, as compared to the corresponding value in the non-liver tissue. In some aspects, after administration of a LNP described herein (e.g., comprising a VEE replicon comprising a nucleic acid sequence encoding a payload), both (i) the total amount of the payload that is expressed and (ii) the duration of payload expression is reduced in the liver as compared to the corresponding value in the liver of a reference subject (e.g., a subject who received a corresponding lipid nanoparticle that does not comprise the VEE replicon). In some aspects, both (i) the total amount of the payload that is expressed and (ii) the duration of payload expression is reduced by at least about 5%, at least about 10%, at least about 20%, at least about 30%, at least about 40%, at least about 50%, at least about 60%, at least about 70%, at least about 80%, at least about 90%, or about 100%, as compared to the corresponding value in the liver of the reference subject.

As is apparent from the present disclosure, in some aspects, LNPs described herein have minimal effect on the expression of the payload in non-lymphoid tissues as compared to the corresponding expression in a reference subject (e.g., a subject who received a corresponding lipid nanoparticle that does not comprise the VEE replicon). Accordingly, in some aspects, after administration, the total amount of payload that is expressed in the non-liver tissue of the subject is at least about 90%, at least about 91%, at least about 92%, at least about 93%, at least about 94%, at least about 95%, at least about 96%, at least about 97%, at least about 98%, at least about 99%, or at least about 100% or more, as compared to that observed in the non-liver tissue of the reference subject. In some aspects, after administering an LNP described herein (e.g., a subject who received a corresponding lipid nanoparticle that does not comprise the VEE replicon), the duration of the payload expression in the non-liver tissue is at least about 90%, at least about 91%, at least about 92%, at least about 93%, at least about 94%, at least about 95%, at least about 96%, at least about 97%, at least about 98%, at least about 99%, or at least about 100%, as compared to that observed in the non-liver tissue of the reference subject.

As is apparent from at least the above disclosure, some aspects of the present disclosure is directed to methods of selectively expressing a payload (e.g., heterologous protein) in a non-liver tissue of a subject in need thereof. In some aspects, such methods comprise administering to a subject in need thereof a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the payload, and wherein after the administration, the payload expression in the liver is reduced, as compared to the corresponding expression in a non-liver tissue. As further described herein, non-liver tissue can comprise any suitable tissue within a subject other than the liver. Non-limiting examples of such non-liver tissues include the spleen, lung, or both. As used herein, “selective expression” or “preferential expression,” or grammatical variants thereof, refers to an increased expression (e.g., of a payload) in a first tissue (e.g., non-liver tissue) as compared to a second tissue (e.g., liver). In some aspects, the payload is selectively or preferentially expressed in a non-liver tissue, where the expression (e.g., total amount of the payload and/or duration of payload expression) in the non-liver tissue is increased compared to the corresponding expression in the liver. In some aspects, the expression of the payload in the non-liver tissue is the same as the corresponding expression in other non-liver tissues of the subject. In some aspects, the expression of the payload in the non-liver tissue is increased compared to the corresponding expression in other non-liver tissues of the subject. In some aspects, the expression of the payload in the non-liver tissue is decreased compared to other non-liver tissues in the subject but is increased compared to the corresponding expression in the liver.

As demonstrated herein, in some aspects, LNPs of the present disclosure are useful in inducing the selective (or preferential) expression of a payload in the spleen as compared to the liver. Accordingly, in some aspects, the total amount of the payload expressed in the spleen is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold greater, at least about 75-fold greater, or at least about 100-fold greater or more, as compared to the corresponding amount observed in the liver. In some aspects, the duration of payload expression in the spleen is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold greater, at least about 75-fold greater, or at least about 100-fold greater or more, as compared to the corresponding duration observed in the liver.

As also demonstrated herein, in some aspects, LNPs of the present disclosure are useful in inducing the selective expression of a payload in the lung as compared to the liver. Accordingly, in some aspects, the total amount of the payload expressed in the lung is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold greater, at least about 75-fold greater, or at least about 100-fold greater or more, as compared to the corresponding amount observed in the liver. In some aspects, the duration of payload expression in the lung is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold greater, at least about 75-fold greater, or at least about 100-fold greater or more, as compared to the corresponding duration observed in the liver.

### II.B. Methods of Treating

As is apparent from the present disclosure, LNPs described herein (e.g., comprising a VEE replicon which comprises a nucleic acid sequence encoding a payload) can be useful in treating a wide range of diseases or disorders. Accordingly, in some aspects, provided herein is a method of treating a disease or disorder in a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding a payload, and wherein after the administration, the payload is preferentially expressed in a non-liver tissue of the subject. In some aspects, after the administration of the LNP, the amount of payload that is expressed in the non-liver tissue is at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, or at least about 100-fold or greater than the corresponding amount observed in the liver of the subject. In some aspects, after the administration of the LNP, the duration of payload expression in the non-liver tissue is at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, or at least about 100-fold or greater than the corresponding duration observed in the liver of the subject.

The LNPs described herein (e.g., comprising a VEE replicon, which comprises a nucleic acid sequence encoding a payload) can be used to treat any suitable diseases or disorders known in the art. Non-limiting examples of diseases and disorders that can be treated include cancer, inflammatory disorders, monogenic disorders, neurological disorders, psychiatric disorders, or combinations thereof. As is apparent from the present disclosure, in some aspects, the LNPs described herein can be used to regulate (e.g., increase or decrease) an immune response. In some aspects, the LNPs described herein can be used as a vaccine. In some aspects, the disease or disorder comprises a cancer. Non-limiting examples of cancers include: a melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, head and neck cancer, or combinations thereof.

As further described elsewhere in the present disclosure, in some aspects, compared to other delivery platforms available in the art, the LNPs described herein allow for increased safety with decreased undesirable side effects. For instance, with many past LNP-based intratumoral treatment approaches, liver toxicity has been a significant problem (e.g., due to accumulation of the LNPs within the liver). See, e.g., Jain et al., Nucleic Acid Ther 28 (5): 285-296 (October 2018), which is incorporated herein by reference in its entirety. Not to be bound by any one theory, in some aspects, by avoiding or reducing expression of a payload within the liver, LNPs described herein (e.g., comprising a VEE replicon, which comprises a nucleic acid sequence encoding a payload) are associated with decreased liver toxicity. For instance, in some aspects, after the administration of a LNP provided herein, there is reduced liver toxicity in the subject compared to a reference subject (e.g., subject who received a corresponding LNP but without the VEE replicon). In some aspects, the liver toxicity in the subject is decreased by at least about 5%, at least about 10%, at least about 20%, at least about 30%, at least about 40%, at least about 50%, at least about 60%, at least about 70%, at least about 80%, at least about 90%, or about 100%, as compared to that of the reference subject. Liver toxicity can be measured using any suitable methods available in the art. In some aspects, liver toxicity can be assessed by conducting a general toxicity assessment of a subject (e.g., clinical toleration and general health status). Non-limiting criteria that can be used in making such an assessment include: activity of the subject, food consumption, body score index, hydration status, and combinations thereof. In some aspects, biological samples can be obtained from the subject (e.g., blood or serum) and hematology and/or clinical chemistry can be conducted. For instance, in some aspects, the level of liver transaminases (ALT/AST), total bilirubin, serum albumin, or combinations thereof can be measured. Such measurements can be compared to a reference (e.g., corresponding amount in a healthy subject) to assess liver toxicity. In some aspects, more invasive methods (e.g., microscopic assessment of the liver) can also be used.

As will be apparent to those skilled in the arts, in some aspects, the decreased liver toxicity could allow for greater tolerability of the administered LNP. Accordingly, in some aspects, provided herein is a method of increasing the tolerability of a LNP-based therapy in a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding a payload (e.g., heterologous protein). In some aspects, the tolerability of the lipid nanoparticle-based therapy is increased in the subject by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding tolerability observed in a reference subject (e.g., a subject who received a corresponding lipid nanoparticle but where the replicon is not a VEE replicon).

In some aspects of the disclosure, the polynucleotides, vectors, lipid nanoparticles, and/or pharmaceutical compositions described herein (also collectively referred to herein as “compositions”) are used to treat a disease or disorder. In some aspects, the disease or disorder comprises a cancer. Non-limiting examples of cancers that can be treated are provided elsewhere in the present disclosure.

In some aspects, an effective amount of any of the LNPs described herein or compositions comprising such LNPs (also collectively referred to herein as “compositions”) is administered to a subject in need thereof via a suitable route, such as intratumoral administration, intravenous administration (e.g., as a bolus or by continuous infusion over a period of time), by intramuscular, intraperitoneal, intracerebospinal, subcutaneous, intra-articular, intrasynovial, intrathecal, oral, inhalation, or topical routes. Commercially available nebulizers for liquid formulations, including jet nebulizers and ultrasonic nebulizers are useful for administration. Liquid formulations can be nebulized and lyophilized powder can be nebulized after reconstitution. In some aspects, the pharmaceutical composition described herein is aerosolized using a fluorocarbon formulation and a metered dose inhaler, or inhaled as a lyophilized and milled powder. In some aspects, the pharmaceutical composition described herein is formulated for intratumoral injection. In some aspects, the pharmaceutical composition described herein is administered to a subject via a local route, for example, injected to a local site such as a tumor site or an infectious site. In some aspects, the subject is a human.

As will be apparent from the present disclosure, in some aspects, the compositions described herein are administered to a subject in an effective amount to confer a therapeutic effect, either alone or in combination with one or more other active agents. In some aspects, the compositions are administered to a subject suffering from a cancer, and the therapeutic effect comprises reduced tumor burden, reduction of cancer cells, increased immune activity, or combinations thereof. Whether the administered composition (e.g., a lipid nanoparticle) achieved the therapeutic effect can be determined using any suitable methods known in the art (e.g., measuring tumor volume and/or T cell activity). Effective amounts vary, as recognized by those skilled in the art, depending on the particular condition being treated, the severity of the condition, the individual patient parameters including age, physical condition, size, gender and weight, the duration of the treatment, the nature of the concurrent therapy (if any), the specific route of administration and like factors within the knowledge of expertise of the health practitioner.

Empirical considerations, such as the half-life, generally will contribute to the determination of the dosage. Frequency of administration can be determined and adjusted over the course of therapy, and is generally, but not necessarily, based on treatment and/or suppression and/or amelioration and/or delay of a target disease/disorder. Alternatively, sustained continuous release formulations of a composition described herein (e.g., lipid nanoparticle) can be appropriate. Various formulations and devices for achieving sustained release are known in the art.

In some aspects of the disclosure, the treatment is a single injection of the composition disclosed herein. In some aspects, the single injection is administered intratumorally to the subject in need thereof.

In some aspects of the disclosure, dosages for a composition described herein can be determined empirically in individuals who have been given one or more administration(s) of the composition (e.g., lipid nanoparticle described herein). In some aspects, the individuals are given incremental dosages of the composition described herein. To assess efficacy of the composition herein, an indicator of disease/disorder can be followed. For repeated administrations over several days or longer, depending on the condition, in some aspects, the treatment is sustained until a desired suppression of symptoms occurs or until sufficient therapeutic levels are achieved to alleviate a target disease or disorder, or symptom thereof.

In some aspects of the disclosure, dosing frequency is about once every week, about once every 2 weeks, about once every 3 weeks, about once every 4 weeks, about once every 5 weeks, about once every 6 weeks, about once every 7 weeks, about once every 8 weeks, about once every 9 weeks, or about once every 10 weeks; or about once a month, about every 2 months, or about every 3 months, or longer. The dosing regimen (e.g., dosage and/or dosing frequency) of the composition described herein (e.g., lipid nanoparticle) used can vary over time.

In some aspects of the disclosure, the method comprises administering to a subject in need thereof one or multiple doses of a composition described herein.

The appropriate dosage of the composition (e.g., lipid nanoparticle described herein) will depend on the specific composition (e.g., lipid nanoparticle), the type and severity of the disease/disorder (e.g., cancer), whether the composition (e.g., lipid nanoparticle) is administered for preventive or therapeutic purposes, previous therapy, the subject's clinical history and response to the composition (e.g., lipid nanoparticle), and the discretion of the attending physician. In some aspects, a clinician can administer a composition disclosed herein until a dosage is reached that achieves the desired result. In some aspects, the desired result is a decrease in tumor burden, a decrease in cancer cells, or increased immune activity. Administration of one or more compositions described herein can be continuous or intermittent, depending, for example, upon the recipient's physiological condition, whether the purpose of the administration is therapeutic or prophylactic, and other factors known to skilled practitioners. The administration of the composition described herein can be essentially continuous over a preselected period of time or can be in a series of spaced doses, e.g., either before, during, or after developing a target disease or disorder.

As used herein, alleviating a target disease/disorder includes delaying the development or progression of the disease, or reducing disease severity. Alleviating the disease does not necessarily require curative results. As used herein, “delaying” the development of a target disease or disorder means to defer, hinder, slow, retard, stabilize, and/or postpone progression of the disease. This delay can be for varying lengths of time, depending on the history of the disease and/or subject being treated. A method that delays or alleviates the development of a disease, or delays the onset of the disease, is a method that reduces probability of developing one or more symptoms of the disease in a given time frame and/or reduces the extent of the symptoms in a given time frame, when compared to not using the method. Such comparisons are typically based on clinical studies, using a number of subjects sufficient to give a statistically significant result.

In some aspects, a composition described herein is administered to a subject in need thereof at an amount sufficient to reduce tumor burden or cancer cell growth in vivo by at least about 5%, at least about 10%, at least about 20%, at least about 30%, at least about 40%, at least about 50%, at least about 60%, at least about 70%, at least about 80%, at least about 90%, or greater. In some aspects, the composition described herein is administered in an amount effective in increasing immune activity by at least about 5%, at least about 10%, at least about 20%, at least about 30%, at least about 40%, at least about 50%, at least about 60%, at least about 70%, at least about 80%, at least about 90%, or greater.

In some aspects, administering the composition to a subject enhances immune activity, such as T cell activity, in the subject. In some aspects, the immune activity is enhanced or increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold or more, compared to the immune activity of a reference subject (e.g., the subject prior to the administration of the composition or a corresponding subject that did not reactive an administration of the composition).

In some aspects, the subject is a human having, suspected of having, or at risk for a cancer. In some aspects the cancer is selected from the group consisting of melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and various types of head and neck cancer, including squamous cell head and neck cancer. In some aspects, the cancer can be melanoma, lung cancer, colorectal cancer, renal-cell cancer, urothelial carcinoma, or Hodgkin's lymphoma.

A subject having a target disease or disorder can be identified by routine medical examination, e.g., laboratory tests, organ functional tests, CT scans, or ultrasounds. A subject suspected of having a target disease or disorder might show one or more symptoms of the disease or disorder. A subject at risk for the disease or disorder can be a subject having one or more of the risk factors associated with that disease or disorder. A subject at risk for a disease or disorder can also be identified by routine medical practices.

In some aspects, the composition described herein is co-administered with at least one additional suitable therapeutic agent. In some aspects, the at least one additional suitable therapeutic agent comprises an anti-cancer agent, an anti-viral agent, an anti-bacterial agent, or other agents that serve to enhance and/or complement the immunostimulatory effect of the composition (e.g., lipid nanoparticle) described herein. Further examples of additional therapeutic agents that can be used in combination with the compositions described herein include: a chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof. In some aspects, the composition described herein and the at least one additional therapeutic agent are administered to the subject in a sequential manner, i.e., each therapeutic agent is administered at a different time. In some aspects, the composition described herein and the at least one additional therapeutic agent are administered to the subject in a substantially simultaneous manner.

It will be appreciated by one of skill in the art that any combination of the composition described herein and another anti-cancer agent (e.g., a chemotherapeutic agent) can be used in any sequence for treating a cancer. The combinations described herein can be selected on the basis of a number of factors, which include, but are not limited to, the effectiveness or reducing tumor formation or tumor growth, reducing cancer cells, increasing immune activity, and/or alleviating at least one symptom associated with the cancer, or the effectiveness for mitigating the side effects of another agent of the combination. For example, a combined therapy described herein can reduce any of the side effects associated with each individual members of the combination, for example, a side effect associated with the anti-cancer agent.

In some aspects, the other anti-cancer therapeutic agent is a chemotherapy, a radiation therapy, a surgical therapy, an immunotherapy, or combinations thereof. In some aspects, the chemotherapeutic agent is carboplatin, cisplatin, docetaxel, gemcitabine, nab-paclitaxel, pemetrexed, vinorelbine, or combinations thereof. In some aspects, the radiation therapy is ionizing radiation, gamma-radiation, neutron beam radiotherapy, electron beam radiotherapy, proton therapy, brachytherapy, systemic radioactive isotopes, radiosensitizers, or combinations thereof. In some aspects, the surgical therapy is a curative surgery (e.g., tumor removal surgery), a preventative surgery, a laparoscopic surgery, a laser surgery, or combinations thereof. In some aspects, the immunotherapy is adoptive cell transfer, therapeutic cancer vaccines, or combinations thereof.

In some aspects, the chemotherapeutic agent is platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin HCl, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives); Purine antagonists (Thioguanine, Fludarabine, Cladribine, 6-Mercaptopurine, Pentostatin, clofarabine and relatives) and Pyrimidine antagonists (Cytarabine, Floxuridine, Azacitidine, Tegafur, Carmofur, Capacitabine, Gemcitabine, hydroxyurea, 5-Fluorouracil (5FU), and relatives); Alkylating agents, such as Nitrogen mustards (e.g., Cyclophosphamide, Melphalan, Chlorambucil, mechlorethamine, Ifosfamide, Trofosfamide, Prednimustine, Bendamustine, Uramustine, Estramustine, and relatives); nitrosoureas (e.g., Carmustine, Lomustine, Semustine, Fotemustine, Nimustine, Ranimustine, Streptozocin, and relatives); Triazenes (e.g., Dacarbazine, Altretamine, Temozolomide, and relatives); Alkyl sulphonates (e.g., Busulfan, Mannosulfan, Treosulfan, and relatives); Procarbazine; Mitobronitol, and Aziridines (e.g., Carboquone, Triaziquone, ThioTEPA, triethylenemalamine, and relatives); Antibiotics, such as Hydroxyurea, Anthracyclines (e.g., doxorubicin agent, daunorubicin, epirubicin and other derivatives); Anthracenediones (e.g., Mitoxantrone and relatives); Streptomyces family (e.g., Bleomycin, Mitomycin C, Actinomycin, Plicamycin); ultraviolet light; and combinations thereof.

In some aspects, the other anti-cancer therapeutic agent is an antibody. Antibodies (preferably monoclonal antibodies) achieve their therapeutic effect against cancer cells through various mechanisms. They can have direct effects in producing apoptosis or programmed cell death. They can block components of signal transduction pathways such as e.g., growth factor receptors, effectively arresting proliferation of tumor cells. In cells that express monoclonal antibodies, they can bring about anti-idiotype antibody formation. Indirect effects include recruiting cells that have cytotoxicity, such as monocytes and macrophages. This type of antibody-mediated cell kill is called antibody-dependent cell mediated cytotoxicity (ADCC). Antibodies also bind complement, leading to direct cell toxicity, known as complement dependent cytotoxicity (CDC). Combining surgical methods with immunotherapeutic drugs or methods is an successful approach, as e.g., demonstrated in Gadri et al. 2009: Synergistic effect of dendritic cell vaccination and anti-CD20 antibody treatment in the therapy of murine lymphoma. J Immunother. 32 (4): 333-40. The following list provides some non-limiting examples of anti-cancer antibodies and potential antibody targets (in brackets) which can be used in combination with the present disclosure: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Bavituximab (phosphatidylserine), Bectumomab (CD22), Belimumab (BAFF), Bevacizumab (VEGF-A), Bivatuzumab mertansine (CD44 v6), Blinatumomab (CD19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R²), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (IL-1β), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL-5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Narnatumab (RON), Necitumumab (EGFR), Nimotuzumab (EGFR), Nivolumab (IgG4), Ofatumumab (CD20), Olaratumab (PDGF-R α), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL-6), Tabalumab (BAFF), Tacatuzumab tetraxetan (alpha-fetoprotein), Taplitumomab paptox (CD19), Tenatumomab (tenascin C), Teprotumumab (CD221), Ticilimumab (CTLA-4), Tigatuzumab (TRAIL-R2), TNX-650 (IL-13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1BB), Volociximab (integrin α5β1), Votumumab (tumor antigen CTAA16.88), Zalutumumab (EGFR), Zanolimumab (CD4).

In some aspects, the other anti-cancer therapeutic agent is a cytokine, chemokine, costimulatory molecule, fusion protein, or combinations thereof. Examples of chemokines include, but are not limited to, CCR7 and its ligands CCL19 and CCL21, furthermore CCL2, CCL3, CCL5, and CCL16. Other examples are CXCR4, CXCR7 and CXCL12. Furthermore, costimulatory or regulatory molecules such as e.g., B7 ligands (B7.1 and B7.2) are useful. Also useful are other cytokines such as e.g., interleukins especially (e.g., IL-1 to IL17), interferons (e.g., IFNalpha1 to IFNalpha8, IFNalpha10, IFNalpha13, IFNalpha14, IFNalpha16, IFNalpha17, IFNalpha21, IFNbeta1, IFNW, IFNE1 and IFNK), hematopoietic factors, TGFs (e.g., TGF-α, TGF-β, and other members of the TGF family), finally members of the tumor necrosis factor family of receptors and their ligands as well as other stimulatory molecules, comprising but not limited to 41BB, 41BB-L, CD137, CD137L, CTLA-4GITR, GITRL, Fas, Fas-L, TNFR1, TRAIL-R1, TRAIL-R2, p75NGF-R, DR6, LT.beta.R, RANK, EDAR1, XEDAR, Fn114, Troy/Trade, TAJ, TNFRII, HVEM, CD27, CD30, CD40, 4-1BB, OX40, GITR, GITRL, TACI, BAFF-R, BCMA, RELT, and CD95 (Fas/APO-1), glucocorticoid-induced TNFR-related protein, TNF receptor-related apoptosis-mediating protein (TRAMP) and death receptor-6 (DR6). Especially CD40/CD40L and OX40/OX40L are important targets for combined immunotherapy because of their direct impact on T cell survival and proliferation. For a review see Lechner et al. 2011: Chemokines, costimulatory molecules and fusion proteins for the immunotherapy of solid tumors. Immunotherapy 3 (11), 1317-1340.

In some aspects, the other anti-cancer therapeutic is a bacterial treatment. Researchers have been using anaerobic bacteria, such as Clostridium novyi, to consume the interior of oxygen-poor tumors. These should then die when they come in contact with the tumor's oxygenated sides, meaning they would be harmless to the rest of the body. Another strategy is to use anaerobic bacteria that have been transformed with an enzyme that can convert a non-toxic prodrug into a toxic drug. With the proliferation of the bacteria in the necrotic and hypoxic areas of the tumor, the enzyme is expressed solely in the tumor. Thus, a systemically applied prodrug is metabolized to the toxic drug only in the tumor. This has been demonstrated to be effective with the nonpathogenic anaerobe Clostridium sporogenes.

In some aspects, the other anti-cancer therapeutic agent is a kinase inhibitor. The growth and survival of cancer cells is closely interlocked with the deregulation of kinase activity. To restore normal kinase activity and therefor reduce tumor growth a broad range of inhibitors is in used. The group of targeted kinases comprises receptor tyrosine kinases e.g., BCR-ABL, B-Raf, EGFR, HER-2/ErbB2, IGF-IR, PDGFR-α, PDGFR-β, c-Kit, Flt-4, Flt3, FGFR1, FGFR3, FGFR4, CSFIR, c-Met, RON, c-Ret, ALK, cytoplasmic tyrosine kinases e.g., c-SRC, c-YES, Abl, JAK-2, serine/threonine kinases e.g., ATM, Aurora A & B, CDKs, mTOR, PKCi, PLKs, b-Raf, S6K, STK11/LKB1 and lipid kinases e.g., PI3K, SK1. Small molecule kinase inhibitors are e.g., PHA-739358, Nilotinib, Dasatinib, and PD166326, NSC 743411, Lapatinib (GW-572016), Canertinib (CI-1033), Semaxinib (SU5416), Vatalanib (PTK787/ZK222584), Sutent (SU11248), Sorafenib (BAY 43-9006) and Leflunomide (SU101). For more information see e.g., Zhang et al. 2009: Targeting cancer with small molecule kinase inhibitors. Nature Reviews Cancer 9, 28-39.

In some aspects, the other anti-cancer therapeutic agent is a toll-like receptor. The members of the Toll-like receptor (TLRs) family are an important link between innate and adaptive immunity and the effect of many adjuvants rely on the activation of TLRs. A large number of established vaccines against cancer incorporate ligands for TLRs for boosting vaccine responses. Besides TLR2, TLR3, TLR4 especially TLR7 and TLR8 have been examined for cancer therapy in passive immunotherapy approaches. The closely related TLR7 and TLR8 contribute to antitumor responses by affecting immune cells, tumor cells, and the tumor microenvironment and can be activated by nucleoside analogue structures. All TLRs have been used as stand-alone immunotherapeutics or cancer vaccine adjuvants and can be synergistically combined with the formulations and methods of the present disclosure. For more information see van Duin et al. 2005: Triggering TLR signaling in vaccination. Trends in Immunology, 27 (1): 49-55.

In some aspects, the other anti-cancer therapeutic agent is an angiogenesis inhibitor. Angiogenesis inhibitors prevent the extensive growth of blood vessels (angiogenesis) that tumors require to survive. The angiogenesis promoted by tumor cells to meet their increasing nutrient and oxygen demands for example can be blocked by targeting different molecules. Non-limiting examples of angiogenesis-mediating molecules or angiogenesis inhibitors which can be combined with the present disclosure are soluble VEGF (VEGF isoforms VEGF121 and VEGF165, receptors VEGFR1, VEGFR2 and co-receptors Neuropilin-1 and Neuropilin-2) 1 and NRP-1, angiopoietin 2, TSP-1 and TSP-2, angiostatin and related molecules, endostatin, vasostatin, calreticulin, platelet factor-4, TIMP and CDAI, Meth-1 and Meth-2, IFN-α, -β and -γ, CXCL10, IL-4,-12 and -18, prothrombin (kringle domain-2), antithrombin III fragment, prolactin, VEGI, SPARC, osteopontin, maspin, canstatin, proliferin-related protein, restin and drugs like e.g., bevacizumab, itraconazole, carboxyamidotriazole, TNP-470, CM101, IFN-α, platelet factor-4, suramin, SU5416, thrombospondin, VEGFR antagonists, angiostatic steroids+heparin, cartilage-derived angiogenesis Inhibitory factor, matrix metalloproteinase inhibitors, 2-methoxyestradiol, tecogalan, tetrathiomolybdate, thalidomide, thrombospondin, prolactina VB3 inhibitors, linomide, tasquinimod, For review see Schoenfeld and Dranoff 2011: Anti-angiogenesis immunotherapy. Hum Vaccin. (9): 976-81.

In some aspects, the other anti-cancer therapeutic agent is a virus-based vaccine. There are a number of virus-based cancer vaccines available or under development which can be used in a combined therapeutic approach together with the formulations of the present disclosure. One advantage of the use of such viral vectors is their intrinsic ability to initiate immune responses, with inflammatory reactions occurring as a result of the viral infection creating the danger signal necessary for immune activation. An ideal viral vector should be safe and should not introduce an anti-vector immune response to allow for boosting anti-tumor specific responses. Recombinant viruses such as vaccinia viruses, herpes simplex viruses, adenoviruses, adeno-associated viruses, retroviruses and avipoxviruses have been used in animal tumor models and based on their encouraging results, human clinical trials have been initiated. Especially important virus-based vaccines are virus-like particles (VLPs), small particles that contain certain proteins from the outer coat of a virus. Virus-like particles do not contain any genetic material from the virus and cannot cause an infection but they can be constructed to present tumor antigens on their coat. VLPs can be derived from various viruses such as e.g., the hepatitis B virus or other virus families including Parvoviridae (e.g., adeno-associated virus), Retroviridae (e.g., HIV), and Flaviviridae (e.g., Hepatitis C virus). For a general review see Sorensen and Thompsen 2007: “Virus-based immunotherapy of cancer: what do we know and where are we going?” APMIS 115 (11): 1177-93; virus-like particles against cancer are reviewed in Buonaguro et al. 2011: Developments in virus-like particle-based vaccines for infectious diseases and cancer. Expert Rev Vaccines 10 (11): 1569-83; and in Guillén et al. 2010: Virus-like particles as vaccine antigens and adjuvants: application to chronic disease, cancer immunotherapy and infectious disease preventive strategies. Procedia in Vaccinology 2 (2), 128-133.

In some aspects, the other anti-cancer therapeutic agent is a peptide-based target therapy. Peptides can bind to cell surface receptors or affected extracellular matrix surrounding the tumor. Radionuclides which are attached to these peptides (e.g., RGDs) eventually kill the cancer cell if the nuclide decays in the vicinity of the cell. Especially oligo- or multimers of these binding motifs are of great interest, since this can lead to enhanced tumor specificity and avidity. For non-limiting examples see Yamada 2011: Peptide-based cancer vaccine therapy for prostate cancer, bladder cancer, and malignant glioma. Nihon Rinsho 69 (9): 1657-61.

### III. Lipid Nanoparticles

As described herein, LNPs of the present disclosure exhibit tissue-specific tropism, such that the LNPs can allow for the selective delivery of a payload (e.g., heterologous protein) to cells (e.g., cells within a non-liver tissue). In some aspects, the delivery can occur in vivo (e.g., by administering a LNP described herein to a subject) or ex vivo (e.g., by culturing a LNP described herein with the cells in vitro). A “lipid nanoparticle” (LNP), as used herein, refers to a vesicle, such as a spherical vesicle, having a contiguous lipid bilayer. Lipid nanoparticles can be used in methods by which pharmaceutical therapies are delivered to targeted locations. Non-limiting examples of LNPs include liposomes, bolaamphihiles, solid lipid nanoparticles (SLNs), nanostructured lipid carriers (NLCs), monolayer membrane structures (e.g., archaeosomes and micelles), lipid-like nanoparticles (LLNs), polymeric nanoparticles (PNPs), lipid polymer hybrid nanoparticles (LPNs), or combinations thereof.

In some aspects, the lipid nanoparticle comprises one or more types of lipids. A “lipid,” as used herein, refers to a group of organic compounds that include, but are not limited to, esters of fatty acids, and in some aspects, are characterized by being insoluble in water, but soluble in many organic solvents. Accordingly, unless indicated otherwise, lipids include natural lipids and lipid-like materials (e.g., lipidoids). They are usually divided into at least three classes: (1) “simple lipids,” which include fats and oils as well as waxes; (2) “compound lipids,” which include phospholipids and glycolipids; and (3) “derived lipids” such as steroids. Non-limiting examples of lipids include triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). In some aspects, lipids useful for the present disclosure include an ionizable lipid, cationic lipid, lipidoid, phospholipid, sterol, or combinations thereof.

In some aspects, the one or more types of lipids in the LNP comprises an ionizable lipid. Accordingly, in some aspects, a LNP useful for the present disclosure comprises (i) an ionizable lipid and (ii) a VEE replicon, which comprises a nucleic acid sequence encoding a payload.

Non-limiting examples of ionizable lipids include: ((4-hydroxybutyl)azanediyl)bis(hexane-6,1-diyl)bis(2-hexyldecanoate) (ALC-0315), heptadecan-9-yl 8-((2-hydroxyethyl) (6-oxo-6-(undecyloxy) hexyl)amino)octanoate (SM-102), heptadecan-9-yl 8-((2-hydroxyethyl) (8-(nonyloxy)-8-oxooctyl)amino)octanoate (Lipid 5), di((Z)-non-2-en-1-yl) 9-((4-(dimethylamino)butanoyl)oxy)heptadecanedioate (L319), 3-(didodecylamino)-N1,N1,4tridodecyl-1-piperazineethanamine (KL10), Nl-[2 (didodecylamino)ethyl]-N1,N4,N4-tridodecyl 1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl-4-(dimethylamino)butanoate (DLin-MC3-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N, N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), or any combination thereof.

In some aspects, the one or more types of lipids in the LNP comprises a cationic lipid. In some aspects, a LNP useful for the present disclosure comprises (i) a cationic lipid and (ii) a VEE replicon, which comprises a nucleic acid sequence encoding a payload. In some aspects, the LNP (i.e., comprising a cationic lipid and a VEE replicon) further comprises an ionizable lipid.

A “cationic lipid,” as used herein, refers to any of a number of lipid species that carry a net positive charge at a selected pH, such as a physiological pH. Non-limiting examples of a cationic lipid include: 1,2-dioleoyl-3-trimethylammonium-propane (DOTAP), lipofectamine, N-[1-(2,3-dioleoyloxy) propyl]-N,N,N-trimethylammonium chloride (DOTMA), 1-[2-(oleoyloxy)ethyl]-2-oleyl-3-(2-hydroxyethyl) imidazolinium chloride (DOTEVI), 2,3-dioleyloxy-N-[2 (sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxy ethyl ammonium bromide (DMRIE), N-(1,2-dioleoyloxyprop-3-yl)-N,N-dimethyl-N-hydroxy ethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), or any combination thereof. Additional examples are provided throughout the present disclosure.

In some aspects, the one or more types of lipids in the LNP comprises a lipidoid. Accordingly, in some aspects, a LNP described herein comprises: (i) a lipidoid and (ii) a VEE replicon, which comprises a nucleic acid sequence encoding a payload. In some aspects, the LNP (i.e., comprising a lipidoid and a VEE replicon) further comprises an ionizable lipid, a cationic lipid, or both.

As used herein, the term “lipidoid” refers to a molecule having one or more characteristics of a lipid, e.g., synthetic cationic lipids. In some aspects, the lipidoid can have a series of secondary and tertiary amines, which increases the net positive charge of the LNP. Non-limiting examples of lipid-like materials and/or lipidoids include: 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200), 3,6-bis(4-(bis(2-hydroxydodecyl)amino)butyl) piperazine2,5-dione (cKK-E12), tetrakis(8-methylnonyl) 3,3′,3″,3′″-(((methylazanediyl)bis(propane-3,1diyl))bis(azanetriyl))tetrapropionate (306Oi₁₀), G0-C14, 5A2-SC8, 3,6-bis(4-(bis((9Z,12Z)-2-hydroxyoctadeca9,12-dien-1-yl)amino)butyl) piperazine-2,5-dione (OF-02), (((3,6-dioxopiperazine-2,5-diyl)bis(butane-4,1-diyl))bis(azanetriyl))tetrakis(ethane2,1-diyl) (9Z,9′Z,9″Z,9′″Z,12Z,12′Z, 12″Z,12′″Z)-tetrakis(octadeca-9,12-dienoate) (OF-Deg-Lin), (((3,6-dioxopiperazine-2,5-diyl)bis(butane-4,1-diyl))bis(azanetriyl))tetrakis(butane-4,1-diyl) (9Z,9′Z,9″Z,9′″Z,12Z,12′Z,12″Z,12′″Z)-tetrakis(octadeca-9,12-dienoate) (OF-C4-Deg-Lin), N1,N3,N5-tris(3-(didodecylamino) propyl)benzene1,3,5-tricarboxamide (TT3), Hexa (octan-3-yl) 9,9′,9″,9′″,9″″,9′″″-((((benzene-1,3,5-tricarbonyl) ris (azanediyl))tris(propane-3,1-diyl))tris(azanetriyl)) hexanonanoate (FTT5), PL-1 [disclosed in Nature Communications, 12-7264 (2021), which is hereby incorporated by reference], 98N12-5 [disclosed in Molecular Therapy vol. 17 no. 5 May 2009, which is hereby incorporated by reference], ethyl 5,5-di((Z)-heptadec-8-en-1-yl)-1-(3-(pyrrolidin-1-yl) propyl)-2,5-dihydro-1H-imidazole-2-carboxylate (A2-Iso5-2DC18 (A2)) and A12-Iso5-2DC18 (A12), or any combination thereof.

In some aspects, the lipid comprises a sterol. In some aspects, a LNP useful for the present disclosure comprises (i) a sterol and (ii) a VEE replicon, which comprises a nucleic acid sequence encoding a payload. In some aspects, the LNP (i.e., comprising a sterol and a VEE replicon) further comprises an ionizable lipid, a cationic lipid, a lipidoid, or any combination thereof.

As used herein, a “sterol,” refers to cholesterol or cholesterol analogs that could be used to fill lipid membrane packing defects and provide structural integrity. Non-limiting examples of sterols include: a cholesterol, fecosterol, sitosterol, ergosterol, campesterol, stigmasterol, brassicasterol, tomatidine, tomatine, ursolic acid, alpha-tocopherol, and combinations thereof. In some aspects, the sterol is cholesterol.

In some aspects, the lipid comprises a phospholipid. In some aspects, a LNP useful for the present disclosure comprises (i) a phospholipid and (ii) a VEE replicon, which comprises a nucleic acid sequence encoding a payload. In some aspects, the LNP (i.e., comprising a phospholipid and a VEE replicon) further comprises an ionizable lipid, a cationic lipid, a lipidoid, a sterol, or any combination thereof.

Non-limiting examples of phospholipids include: 1,2-dilinoleoyl-sn-glycero-3phosphocholine (DLPC), 1,2-dimyristoyl-sn-glycerol-phosphocholine (DMPC), 1,2-dioleoyl-sn glycerol-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and any combinations thereof. In some aspects, the phospholipid is selected from the group consisting of 1-myristoyl-2-palmitoyl-sn-glycero-3-phosphocholine (14:0-16:0 PC, MPPC), 1-myristoyl-2stearoyl-sn-glycero-3-phosphocholine (14:0-18:0 PC, MSPC), 1-palmitoyl 2-acetyl-sn-glycero-3-phosphocholine (16:0-02:0 PC), 1-palmitoyl-2-myristoyl-sn-glycero-3-phosphocholine (16:0-14:0 PC, PMPC), 1-palmitoyl-2-stearoyl-sn-glycero-3-phosphocholine (16:0-18:0 PC, PSPC), 1-palmitoyl-2-oleoyl-sn-glycero-3-phosphocholine (16:0-18:1 PC, POPC), 1-palmitoyl-2-linoleoyl-sn-glycero-3-phosphocholine (16:0-18:2 PC, PLPC), 1-palmitoyl-2-arachidonoyl-sn-glycero-3-phosphocholine (16:0-20:4 PC), 1-palmitoyl-2-docosahexaenoyl-sn-glycero-3-phosphocholine (14:0-22:6 PC), 1-stearoyl-2-myristoyl-sn-glycero-3-phosphocholine (18:0-14:0 PC, SMPC), 1-stearoyl-2-palmitoyl-sn-glycero-3-phosphocholine (18:0-16:0 PC, SPPC), 1-stearoyl-2-oleoyl-sn-glycero-3-phosphocholine (18:0-18:1 PC, SOPC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero-3-phosphocholine (18:0-22:6 PC), 1-oleoyl-2-myristoyl-sn-glycero-3-phosphocholine (18:1-14:0 PC, OMPC), 1-oleoyl-2-palmitoyl-sn-glycero-3-phosphocholine (18:1-16:0 PC, OPPC), 1-oleoyl-2-stearoyl-sn-glycero-3-phosphocholine (18:1-18:0 PC, OSPC), 1-palmitoyl-2-oleoyl-sn-glycero-3-phosphoethanolamine (16:0-18:1 PE, POPE), 1-palmitoyl-2-linoleoyl-sn-glycero-3-phosphoethanolamine (16:0-18:2 PE), 1-palmitoyl-2-arachidonoyl-sn-glycero-3-phosphoethanolamine (16:0-20:4 PE), 1-palmitoyl-2-docosahexaenoyl-sn-glycero-3-phosphoethanolamine (16:0-22:6 PE), 1-stearoyl-2-oleoyl-sn-glycero-3-phosphoethanolamine (18:0-18: PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-cholesterylhemisuccinoyl-sn-glycero-3-phosphocholine (OChemsPC), and any combination thereof.

Accordingly, in some aspects, LNPs useful for the present disclosure comprise (i) one or more natural lipids (e.g., natural cationic lipids) and (ii) a VEE replicon, which comprises a nucleic acid sequence encoding a payload. In some aspects, LNPs useful for the present disclosure comprise (i) one or more synthetic lipids (e.g., lipidoid) and (ii) a VEE replicon, which comprises a nucleic acid sequence encoding a payload. In some aspects, LNPs use for the present disclosure comprise (i) both natural lipids (e.g., cationic lipids) and synthetic lipids (e.g., lipidoids), and (ii) a VEE replicon, which comprises a nucleic acid sequence encoding a payload.

Examples of lipids that can be used with the LNPs of the present disclosure include, but are not limited to, N1,N3,N5-tris(3-(didodecylamino) propyl)benzene-1,3,5-tricarboxamide (TT3), N-(2,3-dioleoyloxy) propyl)-N,N,N-trimethylammonium chloride (DOTAP); lipofectamine; 1,2-DiLinoleyloxy-N,N-dimethylaminopropane (DLinDMA), 1,2-Dilinolenyloxy-N,N-dimethylaminopropane (DLenDMA); dioctadecyldimethylammonium (DODMA), Distearyldimethylammonium (DSDMA), N,N-dioleyl-N,N,-dimethylammonium chloride (DODAC); N-(2,3-dioleyloxy) propyl)-N,N,N-trimethylammonium chloride (DOTMA); N—N-distearyl-N,N-dimethylammonium bromide (DDAB); 3-(N—(N′,N′-dimethylaminoethane)-carbamoyl) cholesterol (DC-Chol) and N-(1,2-dimyristyloxprop-3-yl)-N,N-dimethyl-N-hydroxyethyl ammonium bromide (DMRIE).

In some aspects, the lipid is represented by Formula I:

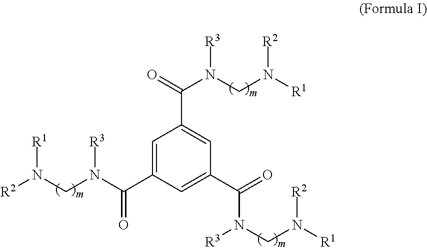

and salts thereof; wherein each R¹ is independently unsubstituted alkyl; each R² is independently unsubstituted alkyl; each R³ is independently hydrogen or substituted or unsubstituted alkyl; and each m is independently 3, 4, 5, 6, 7, or 8. In some aspects, each R¹ is independently unsubstituted alkyl; each R² is independently unsubstituted alkyl; R³ is hydrogen; and each m is 3. In some aspects, at least one R¹ is unsubstituted C₁₋₂₄ alkyl. In some aspects, at least one R¹ is unsubstituted C₁₋₁₈ alkyl. In some aspects, at least one R¹ is unsubstituted C₁₋₁₂ alkyl. In some aspects, at least one R¹ is unsubstituted C6-18 alkyl. In some aspects, at least one R¹ is unsubstituted C6-12 alkyl. In some aspects, at least one R¹ is unsubstituted C8-12 alkyl. In some aspects, at least one R¹ is unsubstituted C10-12 alkyl. In some aspects, at least one R¹ is unsubstituted C11 alkyl.

In some aspects, at least one R² is unsubstituted C₁₋₂₄ alkyl. In some aspects, at least one R² is unsubstituted C₁₋₁₈ alkyl. In some aspects, at least one R² is unsubstituted C₁₋₁₂ alkyl. In some aspects, at least one R² is unsubstituted C6-18 alkyl. In some aspects, at least one R² is unsubstituted C6-12 alkyl. In some aspects, at least one R² is unsubstituted C8-12 alkyl. In some aspects, at least one R² is unsubstituted C10-12 alkyl. In some aspects, at least one R² is unsubstituted C₁₁ alkyl.

In some aspects, at least two R¹ are unsubstituted C₁₋₂₄ alkyl. In some aspects, at least two R¹ are unsubstituted C₁₋₁₈ alkyl. In some aspects, at least two R¹ are unsubstituted C₁₋₁₂ alkyl. In some aspects, at least two R¹ are unsubstituted C6-18 alkyl. In some aspects, at least two R¹ are unsubstituted C6-12 alkyl. In some aspects, at least two R¹ are unsubstituted C8-12 alkyl. In some aspects, at least two R¹ are unsubstituted C10-12 alkyl. In some aspects, at least two R¹ are unsubstituted C₁₁ alkyl.

In some aspects, at least two R² are unsubstituted C₁₋₂₄ alkyl. In some aspects, at least two R² are unsubstituted C₁₋₁₈ alkyl. In some aspects, at least two R² are unsubstituted C₁₋₁₂ alkyl. In some aspects, at least two R² are unsubstituted C6-18 alkyl. In some aspects, at least two R² are unsubstituted C6-12 alkyl. In some aspects, at least two R² are unsubstituted C8-12 alkyl. In some aspects, at least two R² are unsubstituted C10-12 alkyl. In some aspects, at least two R² are unsubstituted C₁₁ alkyl.

In some aspects, all instances of R¹ are unsubstituted C₁₋₂₄ alkyl. In some aspects, all instances of R¹ are unsubstituted C₁₋₁₈ alkyl. In some aspects, all instances of R¹ are unsubstituted C₁₋₁₂ alkyl. In some aspects, all instances of R¹ are unsubstituted C6-18 alkyl. In some aspects, all instances of R¹ are unsubstituted C6-12 alkyl. In some aspects, all instances of R¹ are unsubstituted C8-12 alkyl. In some aspects, all instances of R¹ are unsubstituted C10-12 alkyl. In some aspects, all instances of R¹ are unsubstituted C₁₁ alkyl.

In some aspects, all instances of R² are unsubstituted C₁₋₂₄ alkyl. In some aspects, all instances of R² are unsubstituted C₁₋₁₈ alkyl. In some aspects, all instances of R² are unsubstituted C₁₋₁₂ alkyl. In some aspects, all instances of R² are unsubstituted C6-18 alkyl. In some aspects, all instances of R² are unsubstituted C6-12 alkyl. In some aspects, all instances of R² are unsubstituted C₈₋₁₂ alkyl. In some aspects, all instances of R² are unsubstituted C₁₀₋₁₂ alkyl. In some aspects, all instances of R² are unsubstituted C₁₁ alkyl.

In some aspects, at least one R³ is hydrogen. In some aspects, at least one R³ is substituted or unsubstituted alkyl. In some aspects, at least one R³ is substituted or unsubstituted C₁₋₁₈ alkyl. In some aspects, at least one R³ is substituted or unsubstituted C₁₋₁₂ alkyl. In some aspects, at least one R³ is substituted or unsubstituted C16 alkyl. In some aspects, at least one R³ is substituted or unsubstituted C₁₋₄ alkyl. In some aspects, at least one R³ is substituted or unsubstituted C₂₋₄ alkyl. In some aspects, at least one R³ is substituted or unsubstituted methyl.

In some aspects, at least one R³ is substituted alkyl, wherein the substituted alkyl is substituted with a halogen. In some aspects, at least one R³ is substituted alkyl, wherein the substituted alkyl is substituted with fluorine. In some aspects, at least one R³ is substituted alkyl, wherein the substituted alkyl is substituted with halogenated alkyl.

In some aspects, at least two R³ are hydrogen. In some aspects, at least two R³ are substituted or unsubstituted alkyl. In some aspects, at least two R³ are substituted or unsubstituted C₁₋₁₈ alkyl. In some aspects, at least two R³ are substituted or unsubstituted C₁₋₁₂ alkyl. In some aspects, at least two R³ are substituted or unsubstituted C₁₋₆ alkyl. In some aspects, at least two R³ are substituted or unsubstituted C₁₋₄ alkyl. In some aspects, at least two R³ are substituted or unsubstituted C₂₋₄ alkyl. In some aspects, at least two R³ are substituted or unsubstituted methyl.

In some aspects, at least two R³ are substituted alkyl, wherein the substituted alkyl is substituted with a halogen. In some aspects, at least two R³ are substituted alkyl, wherein the substituted alkyl is substituted with fluorine. In some aspects, at least two R³ are substituted alkyl, wherein the substituted alkyl is substituted with halogenated alkyl.

In some aspects, all instances of R³ are hydrogen. In some aspects, all instances of R³ are substituted or unsubstituted alkyl. In some aspects, all instances of R³ are substituted or unsubstituted C₁₋₁₈ alkyl. In some aspects, all instances of R³ are substituted or unsubstituted C₁₋₁₂ alkyl. In some aspects, all instances of R³ are substituted or unsubstituted C16 alkyl. In some aspects, all instances of R³ are substituted or unsubstituted C₁₋₄ alkyl. In some aspects, all instances of R³ are substituted or unsubstituted C₂₋₄ alkyl. In some aspects, all instances of R³ are substituted or unsubstituted methyl.

In some aspects, all instances of R³ are substituted alkyl, wherein the substituted alkyl is substituted with a halogen. In some aspects, all instances of R³ are substituted alkyl, wherein the substituted alkyl is substituted with fluorine. In some aspects, all instances of R³ are substituted alkyl, wherein the substituted alkyl is substituted with halogenated alkyl.

In some aspects, at least one m is 3. In some aspects, at least one m is 4. In some aspects, at least one m is 5. In some aspects, at least one m is 6. In some aspects, at least one m is 7. In some aspects, at least one m is 8. In some aspects, at least two m are 3. In some aspects, at least two m are 4. In some aspects, at least two m are 5. In some aspects, at least two m are 6. In some aspects, at least two m are 7. In some aspects, at least two m are 8.

In some aspects, all instances of m are 3. In some aspects, all instances of m are 4. In some aspects, all instances of m are 5. In some aspects, all instances of m are 6. In some aspects, all instances of m are 7. In some aspects, all instances of m are 8.

In some aspects of the disclosure, the lipid is TT3, which is represented by:

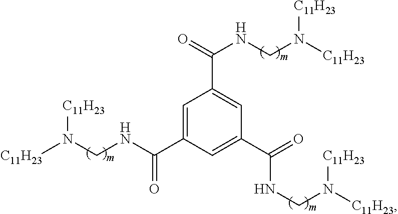

wherein all instances of m=3. The composition, synthesis, and use of Formula I and TT3 are described in WO2016187531A1, which is incorporated herein by reference in its entirety.

Accordingly, in some aspects, LNPs described herein comprise (i) TT3 and (ii) a VEE replicon, which comprises a nucleic acid sequence encoding a payload. “TT3,” as used herein, is capable of forming lipid nanoparticles for delivery of various biologic active agents (e.g., payloads described herein) into the cells. In addition, the present disclosure also demonstrates that an unloaded TT3-LNP can induce immunogenic cell death (ICD) in cancer cells in vivo and in vitro. Immunogenic cell death, as described herein, refers to a form of cell death that can induce an effective immune response through activation of dendritic cells (DCs) and consequent activation of specific T cell response. In some aspects, the cells that undergo immunogenic cell death are tumor cells.

In some aspects, the lipid is DOTAP. Accordingly, in some aspects, LNPs of the present disclosure comprise (i) DOTAP and (ii) a VEE replicon, which comprises a nucleic acid sequence encoding a payload. “DOTAP,” as used herein, is also capable of forming lipid nanoparticles. DOTAP can be used for the highly efficient transfection of DNA including yeast artificial chromosomes (YACs) into eukaryotic cells for transient or stable gene expression, and is also suitable for the efficient transfer of other negatively charged molecules, such as RNA, oligonucleotides, nucleotides, ribonucleoprotein (RNP) complexes, and proteins into research samples of mammalian cells.

In some aspects of the disclosure, the lipid is lipofectamine. In some aspects, LNPs described herein comprise (i) lipofectamine and (ii) a VEE replicon, which comprises a nucleic acid sequence encoding a payload. “Lipofectamine,” as used herein, is a common transfection reagent, produced and sold by Invitrogen, used in molecular and cellular biology. It is used to increase the transfection efficiency of RNA (including mRNA and siRNA) or plasmid DNA into in vitro cell cultures by lipofection. Lipofectamine contains lipid subunits that can form liposomes or lipid nanoparticles in an aqueous environment, which entrap the transfection payload. The RNA-containing liposomes (positively charged on their surface) can fuse with the negatively charged plasma membrane of living cells, due to the neutral co-lipid mediating fusion of the liposome with the cell membrane, allowing nucleic acid cargo molecules to cross into the cytoplasm for replication or expression.

In some aspects, LNPs described herein comprise a single type of lipid (e.g., all lipidoids, e.g., all TT3). In some aspects, LNPs of the present disclosure comprises multiple types of lipids. For instance, in some aspects, LNPs comprise lipidoids (e.g., TT3) along with other lipid ingredients. These typically include other lipid molecules belonging but not limited to phophatidylcholines (PC) (e.g., 1,s-Distearoyl-sn-glycero-3-phophocholine (DSPC), and 1,2-Dioleoyl-sn-glycero-3-phophoethanolamines (DOPE), sterols (e.g., cholesterol), and Polyethylene glycol (PEG)-lipid conjugates (e.g., 1,2-distearoyl-sn-glycero-3-phosphoethanolamine-N-[folate (polyethylene glycol)-2000 (DSPE-PEG2000) and 1,2-dimyristoyl-sn-glycero-3-phosphoethanolamine-N-[methoxy (polyethylene glycol)-2000 (C14-PEG2000)). Table 1 shows the formulation of exemplary LNPs, TT3-LNP and DOTAP-LNP.

TABLE 1

| DOTAP-LNP | DOTAP | DSPC | Cholesterol | DSPE-PEG2000 |
|---|---|---|---|---|
| Molar ratio | 40 | 10 | 48 | 2 |
| TT3-LNP | TT3 | DOPE | Cholesterol | C14-PEG2000 |
| Molar Ratio | 20 | 30 | 40 | 0.75 |

In some aspects, the LNP comprises C14-PEG2000. Accordingly, in some aspects, LNPs useful for the present disclosure comprises (i) C14-PEG2000 and (ii) a VEE replicon, which comprises a nucleic acid sequence encoding a payload. In some aspects, C14-PEG2000 comprises 1,2-dimyristoyl-rac-glycero-3-methoxypolyethylene glycol-2000 (DMG-PEG2000), 1,2-dimyristoyl-sn-glycero-3-phosphoethanolamine-N-[methoxy (polyethylene glycol)-2000] (DMPE-PEG2000), or both. In some aspects, the C14-PEG2000 (or other lipid ingredients disclosed herein) can be embedded in the LNP prior to the encapsulation of the VEE replicon. In some aspects, the C14-PEG2000 (or other lipid ingredients disclosed herein) can be added to the LNP after the encapsulation of the VEE replicon. For example, in some aspects, a VEE replicon is encapsulated in the LNP, and then the C14-PEG2000 (other lipid ingredients disclosed herein) is attached to the LNP using, e.g., micelles.

In some aspects, LNPs useful for the present disclosure comprises a polymeric material. Accordingly, in some aspects, a LNP provided herein comprises (i) a polymeric material and (ii) a VEE replicon, which comprises a nucleic acid sequence encoding a payload. In some aspects, a LNP comprises (i) one or more types of lipids, (ii) a polymeric material, and (iii) a VEE replicon, which comprises a nucleic acid sequence encoding a payload. In some aspects, the one or more types of lipids comprise an ionizable lipid, cationic lipid, lipidoid, phospholipid, sterol, or combinations thereof.

In some aspects, the polymeric material comprises a cationic polymer or a non-cationic polymer, such as those described herein. Non-limiting examples of polymeric materials are disclosed in Jiang et al. Reference Module in Materials Science and Materials Engineering. (2021) and Byun et al. BioChip J (2022), which are hereby incorporated by reference. Non-limiting examples of polymeric materials include: Polyethyleneimine (PEI), Poly (amidoamine) (PAMAM), Poly (β-amino ester) (PBAE), poly (2-N,N-dimethylaminoethyl methacrylate) (PDMAEMA), Poly (amino acid) s (PAAs), chitosan dextran (Raemdonck et al., 2009), cyclodextrin (Singh et al., 2019), cellulose (Kim et al., 2020), hyaluronic acid, polylactic acid (PLA), poly(lactic-co-glycolic acid) (PLGA), and polycaprolactone (PCL).

As is apparent from at least the above disclosure, in some aspects, a LNP useful for the present disclosure comprises a VEE replicon, which comprises a nucleic acid sequence encoding a payload, and one or more of the following components: (i) an ionizable lipid, (ii) a cationic lipid, (iii) a lipid-like material (e.g., lipidoid), (iv) a phospholipid, (v) a sterol, (vi) a pegylated lipid, and (vii) a polymeric material. In some aspects, a LNP described herein comprises a VEE replicon, which comprises a nucleic acid sequence encoding a payload, and one or more of the following: (i) an ionizable lipid, (ii) a cationic lipid, (iii) a phospholipid, (iv) a sterol, and (v) a pegylated lipid. In some aspects, a LNP described herein comprises a VEE replicon, which comprises a nucleic acid sequence encoding a payload, and one or more of the following: (i) a lipid-like material (e.g., lipidoid), (ii) a phospholipid, (iii) a sterol, and (iv) a pegylated lipid. In some aspects, a LNP described herein comprises a VEE replicon, which comprises a nucleic acid sequence encoding a payload, and one or more of the following: (i) a polymeric material, (ii) a phospholipid, (iii) a sterol, and (iv) a pegylated lipid. In some aspects, a LNP described herein comprises a VEE replicon, which comprises a nucleic acid sequence encoding a payload, and one or more of the following: (i) a polymeric material, (ii) a lipid, (iii) a phospholipid, (iv) a sterol, and (v) a pegylated lipid.

Particle size of lipid nanoparticles can affect drug release rate, bio-distribution, mucoadhesion, cellular uptake of water and buffer exchange to the interior of the nanoparticles, and protein diffusion. In some aspects of the disclosure, the diameter of the LNPs ranges from about 30 to about 500 nm. In some aspects of the disclosure, the diameter of the LNPs ranges from about 30 to about 500 nm, about 50 to about 400 nm, about 70 to about 300 nm, about 100 to about 200 nm, about 100 to about 175 nm, or about 100 to about 160 nm. In some aspects of the disclosure, the diameter of the LNPs ranges from 100-160 nm. In some aspects of the disclosure, the diameter of the LNPs can be about 30 nm, about 40 nm, about 50 nm, about 60 nm, about 70 nm, about 80 nm, about 90 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 130 nm, about 140 nm, about 150 nm, or about 160 nm. In some aspects, the lipid nanoparticle has a diameter of about 140 nm.

Zeta potential is a measure of the effective electric charge on the lipid nanoparticle surface. The magnitude of the zeta potential provides information about particle stability. In some aspects, the zeta potential of the nanoparticles described herein ranges from about-20 to about 20 mv. In some aspects of the disclosure, the zeta potential of the LNPs ranges from about 3 to about 6 mv. In some aspects of the disclosure, the zeta potential of the LNPs can be about 3 mv, about 3.1 mv, about 3.2 mv, about 3.3 mv, about 3.4 mv, about 3.5 mv, about 3.6 mv, about 3.7 mv, about 3.8 mv, about 3.9 mv, about 4 mv, about 4.1 mv, about 4.2 mv, about 4.3 mv, about 4.4 mv, about 4.5 mv, about 4.6 mv, about 4.7 mv, about 4.8 mv, about 4.9 mv, about 5 mv, about 5.1 mv, about 5.2 mv, about 5.3 mv, about 5.4 mv, about 5.5 mv, about 5.6 mv, about 5.7 mv, about 5.8 mv, about 5.9 mv, or about 6 mv. In some aspects, the zeta potential of the nanoparticles described herein can be about-6.0 mv, about-5.9 mv, about-5.8 mv, about-5.7 mv, about-5.6 mv, about-5.5 mv, about-5.4 mv, about-5.3 mv, about-5.2 mv, about-5.1 mv, about-5.0 mv, about-4.9 mv, about-4.8 mv, about-4.7 mv, about-4.6 mv, about-4.5 mv, about-4.4 mv, about-4.3 mv, about-4.2 mv, about-4.1 mv, about-4.0 mv, about-3.9 mv, about-3.8 mv, about-3.7 mv, about-3.6 mv, about-3.5 mv, about-3.4 mv, about-3.3 mv, about-3.2 mv, about-3.1 mv, about-3.0 mv, about-2.9 mv, about-2.8 mv, about-2.7 mv, about-2.6 mv, about-2.5 mv, about-2.4 mv, about-2.3 mv, about-2.2 mv, about-2.1 mv, about-2.0 mv, about-1.9 mv, about-1.8 mv, about-1.7 mv, about-1.6 mv, about-1.5 mv, about-1.4 mv, about-1.3 mv, about-1.2 mv, about-1.1 mv, about-1.0 mv, about-0.9 mv, about-0.8 mv, about-0.7 mv, about-0.6 mv, about-0.5 mv, about-0.4 mv, about-0.3 mv, about-0.2 mv, about-0.1 mv, about 0.0 mv, 0.1 about mv, about 0.2 mv, about 0.3 mv, about 0.4 mv, about 0.5 mv, about 0.6 mv, about 0.7 mv, about 0.8 mv, about 0.9 mv, about 1.0 mv, about 1.1 mv, about 1.2 mv, about 1.3 mv, about 1.4 mv, about 1.5 mv, about 1.6 mv, about 1.7 mv, about 1.8 mv, about 1.9 mv, about 2.0 mv, about 2.1 mv, about 2.2 mv, about 2.3 mv, about 2.4 mv, about 2.5 mv, about 2.6 mv, about 2.7 mv, about 2.8 mv, about 2.9 mv, about 3.0 mv, about 3.1 mv, about 3.2 mv, about 3.3 mv, about 3.4 mv, about 3.5 mv, about 3.6 mv, about 3.7 mv, about 3.8 mv, about 3.9 mv, about 4.0 mv, about 4.1 mv, about 4.2 mv, about 4.3 mv, about 4.4 mv, about 4.5 mv, about 4.6 mv, about 4.7 mv, about 4.8 mv, about 4.9 mv, about 5.0 mv, about 5.1 mv, about 5.2 mv, about 5.3 mv, about 5.4 mv, about 5.5 mv, about 5.6 mv, about 5.7 mv, about 5.8 mv, about 5.9 mv, or about 6.0 mv

In some aspects, the disclosure is related to encapsulated polynucleotide (e.g., VEE replicon mRNA) with lipid nanoparticles (LNPs). In some aspects of the disclosure, the mass ratio between the lipid of LNPs and the polynucleotide (e.g., VEE replicon mRNA) ranges from about 1:2 to about 15:1. In some aspects, the mass ratio between the lipid and the polynucleotide (e.g., VEE replicon mRNA) can be about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, about 12:1, about 12.5:1, about 13:1, about 13.5:1, about 14:1, about 14.5:1, or about 15:1. In some aspects of the disclosure, the mass ratio between the lipid and the polynucleotide (e.g., VEE replicon mRNA) is about 10:1.

### IV. VEE Replicon

In addition to the one or more types of lipids (e.g., those described above), LNPs of the present disclosure comprise a VEE replicon, which comprises a nucleic acid sequence encoding a payload. As further described elsewhere in the present disclosure, Applicant has identified that the interaction between the VEE replicon and the one or more lipids (e.g., lipidoids, e.g., TT3) provides the LNPs described herein with certain improved properties, such as tissue-specific tropism, which allows the LNPs to avoid the liver and/or reduce the expression of the payload within the liver, as compared to a non-liver tissue (e.g., spleen and lung).

The VEE virus is a viral pathogen typically carried by mosquitos that causes VEE or encephalomyelitis predominately in equine species. Humans, however, can also contract VEE, and people with weakened immune systems are especially at risk of having severe complications if infected with VEE. The virion of VEE is spherical and possesses a lipid membrane with glycoprotein surface proteins spread around the outer surface. VEE has a genome of approximately 11.45 kb, excluding the 5′-terminal cap and 3′-terminal poly(A) tract, and comprises four nonstructural proteins (nsPs) and five structural proteins. The non-structural proteins include nsP1, nsP2, nsP3, and nsP4, while the structural region encodes proteins C, E3, E2, 6K, and E1. In some aspects, the self-amplifying replicon RNA is a WT replicon RNA derived from VEE. The sequence of the wild-type VEE virus replicon RNA is set forth in SEQ ID NO: 186 (see Table 4).

In some aspects, the VEE replicon useful for the present disclosure comprises a nucleotide sequence that has at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to the sequence set forth in SEQ ID NO: 186 or a fragment thereof. In some aspects, the VEE replicon useful for the present disclosure comprises a nucleotide sequence that has at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to the sequence set forth in SEQ ID NO: 187. In some aspects, the VEE replicon comprises the nucleic acid sequence set forth in SEQ ID NO: 187. In some aspects, the VEE replicon consists of the nucleic acid sequence set forth in SEQ ID NO: 187. In some aspects, the VEE replicon consists essentially of the nucleic acid sequence set forth in SEQ ID NO: 187.

In some aspects, the VEE replicon useful for the present disclosure includes any VEE replicons known in the art. In some aspects, the VEE replicon comprises one or more mutations. For instance, in some aspects, the VEE replicon comprises mutations with the non-structural proteins nsP2 and nsP3, wherein the mutations promote subgenomic expression in human cells. In some aspects, the VEE replicon comprises mutations that allow for greater expression of the encoded payload compared to the wild-type VEE replicon (SEQ ID NO: 186). In some aspects, the self-amplifying replicon RNA, comprises at least one point mutation in a nucleic acid position 3936 and/or 4758 of WT replicon of SEQ ID NO: 186. In some aspects, the self-amplifying replicon RNA, comprises at least one of the following point mutations: guanine to cytosine at position 3936 (G3936C) and adenine to guanine at position 4758 (A4758G) of WT replicon sequence of SEQ ID NO: 186. The G3936C mutation would result in a glycine to arginine change at amino acid residue 1309 (G1309R). The A4758G mutation would result in a serine to glycine change at amino acid residue 1583 (51583G). Additional disclosure regarding such VEE replicons are provided, e.g., in US20200281994A1, which is incorporated herein by reference in its entirety.

Not to be bound by any one theory, in some aspects, the interaction between the VEE replicon and the one or more lipids (e.g., lipidoids, e.g., TT3) results in the LNP not targeting the liver. And, because the LNP does not target the liver, in some aspects, the payload is not expressed in the liver. In some aspects, a LNP described herein (e.g., comprising a VEE replicon, which comprises a nucleic acid sequence encoding a payload) is capable of targeting the liver, but the expression of the payload is reduced compared to the corresponding expression in non-liver tissues.

In some aspects, the VEE replicon is capable of inducing the activation of toll like receptors (TLRs). A subset of TLRs, TLR3, TLR7/8, and TLR9, is involved in antiviral responses by triggering the production of antiviral cytokines such as type I interferons (IFNs). TLR3 responds to double stranded RNA, a replication intermediary for many viruses. TLR7/8 recognize viral single-stranded RNAs, whereas TLR9 recognizes unmethylated CpG motifs within viral DNA. TLRs involved in virus recognition are expressed on endosomal membranes and can be separated according to their requirement for the adaptor protein MyD88: TLR3 activity is MyD88-independent while TLRs7/8/9 depend on MyD88. The activation of TLR3 leads to the production of Type I Interferon (IFN). Type-I interferon signaling through ISGF3 (STAT1/STAT2/IRF9) complex is required for sustained Rip3 activation and necroptosis. Not to be bound by any one theory, in some aspects, the induction of the type I interferon can reduce and/or suppress the expression of the payload within the liver.

### IV.A. Payload

As described herein, VEE replicons useful for the present disclosure comprise a payload. As used herein, the term “payload” refers to any agent that is capable of acting on a target (e.g., a target cell) that is contacted with LNPs described herein. In some aspects, unless indicated otherwise, the term payload can be used interchangeably with the term “biologically active molecules.” Non-limiting examples of payloads that are useful for the present disclosure include: nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), a peptide-conjugated phosphorodiamidate morpholino oligomer (PPMO), or combinations thereof), amino acids (e.g., amino acids comprising a detectable moiety or a toxin or that disrupt translation), polypeptides (e.g., enzymes), lipids, carbohydrates, and small molecules (e.g., small molecule drugs and toxins).

In some aspects, the payload comprises a nucleic acid sequence encoding a protein (e.g., heterologous protein). As will be apparent to those skilled in the arts, any suitable proteins can be encoded in the payloads. In some aspects, the protein comprises a detectable protein (e.g., fluorescent protein). Non-limiting examples of such detectable proteins include: luciferase, wt-GFP, green fluorescent protein (e.g., EGFP, Emerald, Superfolder GFP, Azami Green, mWasabi, TagGFP, TurboGFP, AcGFP, ZsGreen, T-Sapphire, etc.), blue fluorescent protein, (e.g., EBFP, EBFP2, Azurite, mTagBFP, etc.), cyan fluorescent protein (e.g., ECFP, mECFP, Cerulean, mTurquoise, CyPet, AmCyan1, Midori-Ishi Cyan, TagCFP, mTFP1 (Teal), etc.), yellow fluorescent protein (e.g., EYFP, Topaz, Venus, mCitrine, YPet, TagYFP, PhiYFP, ZsYellow1, mBanana, etc.), orange fluorescent protein (e.g., Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, etc.), or red fluorescent protein (e.g., mRuby, mApple, mStrawberry, AsRed2, mRFP1, JRed, mCherry, HcRedl, mRaspberry, dKeima-Tandem, HcRed-Tandem, mPlum, AQ143, etc.).

In some aspects, the payload comprises a nucleic acid sequence encoding a therapeutic protein. A non-limiting example of such a therapeutic protein comprises a cytokine. In some aspects, the cytokine comprises an interleukin (IL)-12 molecule. In some aspects, the IL-12 molecule comprises is IL-12, an IL-12 subunit (e.g., IL-12 beta subunit or IL-12 alpha subunit), or a mutant IL-12 molecule that retains immunomodulatory function. In some aspects, the cytokine is not IL-12. In some aspects, the cytokine is selected from (i) common gamma chain family of cytokines; (ii) IL-1 family of cytokines; (iii) hematopoietic cytokines; (iv) interferons (e.g., type I, type II, or type III); (v) TNF family of cytokines; (vi) IL-17 family of cytokines; (vii) damage-associated molecular patterns (DAMPs); (viii) tolerogenic cytokines; or (ix) combinations thereof. In some aspects, the cytokine comprises IL-2, IL-4, IL-7, IL-10, IL-15, IL-21, IFN-γ, IL-1α, IL-1β, IL-1ra, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoieticn (EPO), Flt-3, IFN-α, IFN-β, IFN-γ, IL-19, IL-20, IL-22, IL-24, TNF-α, TNF-β, BAFF, APRIL, lymphotoxin beta (TNF-γ), IL-17A, IL-17B, IL-17C, IL-17D, IL-17E, IL-17F, IL-25, TSLP, IL-35, IL-27, TGF-β, or combinations thereof. Additional examples of other proteins that can be encoded are provided further below.

Where the payload comprises a nucleic acid sequence encoding an IL-12 protein, in some aspects, the IL-12 protein (e.g., encoded by a nucleic acid molecule described herein) comprises an amino acid sequence that is at least about 70%, at least about 75%, at least about 80%, at least about 85%, at least about 90%, at least about 91%, at least about 92%, at least about 93%, at least about 94% at least about 95%, at least about 96%, at least about 97%, at least about 98%, at least about 99%, or about 100% identical to SEQ ID NO: 182.

In some aspects, the IL-12 molecule comprises IL-12a and/or IL-12β subunits. In some aspects, the IL-12a subunit comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 183.

In some aspects, the IL-12β subunit comprises an amino acid sequence that is at least about 70%, at least about 75%, at least about 80%, at least about 85%, at least about 90%, at least about 91%, at least about 92%, at least about 93%, at least about 94% at least about 95%, at least about 96%, at least about 97%, at least about 98%, at least about 99%, or about 100% identical to SEQ ID NO: 184.

In some aspects, a VEE replicon described herein comprises a nucleic acid sequence which has been codon optimized. Accordingly, in some aspects, the nucleotide sequence encoding an IL-12 protein (e.g., IL-12 p35 subunit, IL-12 p40 subunit, or the heterodimeric IL-12 p70) disclosed herein differs from that of the wild-type nucleotide sequence (e.g., SEQ ID NO: 185).

In some aspects, a VEE replicon comprises a nucleic acid sequence encoding an IL-12β subunit, wherein the nucleic acid sequence has at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the sequence set forth in any one of SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75.

In some aspects, a VEE replicon useful for the present disclosure comprises a nucleic acid sequence encoding an IL-12β subunit, wherein the nucleic acid sequence has (i) at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 51; (ii) at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 52; (iii) at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 53; (iv) at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 54; (v) at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 55; (vi) at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 56; (vii) at least 91%, at least 92%, at least 93%, at least 94%, at least 95%, at least 96%, at least 97%, at least 98%, at least 99%, or 100% sequence identity to the sequence set forth in SEQ ID NO: 57; (viii) at least 92%, at least 93%, at least 94%, at least 95%, at least 96%, at least 97%, at least 98%, at least 99%, or 100% sequence identity to the sequence set forth in SEQ ID NO: 58; (ix) at least 91%, at least 92%, at least 93%, at least 94%, at least 95%, at least 96%, at least 97%, at least 98%, at least 99%, or 100% sequence identity to the sequence set forth in SEQ ID NO: 59; (x) at least 91%, at least 92%, at least 93%, at least 94%, at least 95%, at least 96%, at least 97%, at least 98%, at least 99%, or 100% sequence identity to the sequence set forth in SEQ ID NO: 65, 69, or 74; (xi) at least 91%, at least 92%, at least 93%, at least 94%, at least 95%, at least 96%, at least 97%, at least 98%, at least 99%, or 100% sequence identity to the sequence set forth in SEQ ID NO: 66, 70, or 75; (xii) at least 97%, at least 98%, at least 99%, or 100% sequence identity to the sequence set forth in SEQ ID NO: 62; (xiii) at least 99% or 100% sequence identity to the sequence set forth in SEQ ID NO: 63; or (xiv) at least 98%, at least 99%, or 100% sequence identity to the sequence set forth in SEQ ID NO: 64.

In some aspects, the nucleic acid molecule encoding the IL-12β subunit comprises the sequence set forth in SEQ ID NO: 51. In some aspects, the nucleic acid molecule encoding the IL-12β subunit comprises the sequence set forth in SEQ ID NO: 52. In some aspects, the nucleic acid molecule encoding the IL-12β subunit comprises the sequence set forth in SEQ ID NO: 53. In some aspects, the nucleic acid molecule encoding the IL-12β subunit comprises the sequence set forth in SEQ ID NO: 54. In some aspects, the nucleic acid molecule encoding the IL-12β subunit comprises the sequence set forth in SEQ ID NO: 55. In some aspects, the nucleic acid molecule encoding the IL-12β subunit comprises the sequence set forth in SEQ ID NO: 56 In some aspects, the nucleic acid molecule encoding the IL-12β subunit comprises the sequence set forth in SEQ ID NO: 57. In some aspects, the nucleic acid molecule encoding the IL-12β subunit comprises the sequence set forth in SEQ ID NO: 58. In some aspects, the nucleic acid molecule encoding the IL-12β subunit comprises the sequence set forth in SEQ ID NO: 59. In some aspects, the nucleic acid molecule encoding the IL-12β subunit comprises the sequence set forth in SEQ ID NO: 65. In some aspects, the nucleic acid molecule encoding the IL-12 subunit comprises the sequence set forth in SEQ ID NO: 66. In some aspects, the nucleic acid molecule encoding the IL-12β subunit comprises the sequence set forth in SEQ ID NO: 67. In some aspects, the nucleic acid molecule encoding the IL-12β subunit comprises the sequence set forth in SEQ ID NO: 68. In some aspects, the nucleic acid molecule encoding the IL-12β subunit comprises the sequence set forth in SEQ ID NO: 69. In some aspects, the nucleic acid molecule encoding the IL-12β subunit comprises the sequence set forth in SEQ ID NO: 70. In some aspects, the nucleic acid molecule encoding the IL-12β subunit comprises the sequence set forth in SEQ ID NO: 71. In some aspects, the nucleic acid molecule encoding the IL-12β subunit comprises the sequence set forth in SEQ ID NO: 72. In some aspects, the nucleic acid molecule encoding the IL-12β subunit comprises the sequence set forth in SEQ ID NO: 73. In some aspects, the nucleic acid molecule encoding the IL-12β subunit comprises the sequence set forth in SEQ ID NO: 74. In some aspects, the nucleic acid molecule encoding the IL-12β subunit comprises the sequence set forth in SEQ ID NO: 75. In some aspects, the nucleic acid molecule encoding the IL-12β subunit comprises the sequence set forth in SEQ ID NO: 62. In some aspects, the nucleic acid molecule encoding the IL-12β subunit comprises the sequence set forth in SEQ ID NO: 63. In some aspects, the nucleic acid molecule encoding the IL-12β subunit comprises the sequence set forth in or SEQ ID NO: 64. In some aspects, the nucleic acid molecule encoding the IL-12β subunit comprises the sequence set forth in SEQ ID NO: 60. In some aspects, the nucleic acid molecule encoding the IL-12β subunit comprises the sequence set forth in SEQ ID NO: 61.

In some aspects, a VEE replicon comprises a nucleic acid sequence encoding an IL-12 p35 subunit (also referred to herein as IL-12a subunit), wherein the nucleic acid sequence has at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the sequence set forth in any one of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125.

In some aspects, a VEE replicon useful for the present disclosure comprises a nucleic acid sequence encoding the IL-12 p35 subunit, wherein the nucleic acid sequence has (i) at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 101; (ii) at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 102; (iii) at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 103; (iv) at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 104; (v) at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 105; (vi) at least 88%, at least 89%, at least 90%, at least 91%, at least 92%, at least 93%, at least 94%, at least 95%, at least 96%, at least 97%, at least 98%, at least 99%, or 100% sequence identity to the sequence set forth in SEQ ID NO: 106; (vii) at least 91%, at least 92%, at least 93%, at least 94%, at least 95%, at least 96%, at least 97%, at least 98%, at least 99%, or 100% sequence identity to the sequence set forth in SEQ ID NO: 107; (viii) at least 91%, at least 92%, at least 93%, at least 94%, at least 95%, at least 96%, at least 97%, at least 98%, at least 99%, or 100% sequence identity to the sequence set forth in SEQ ID NO: 108; (ix) at least 92%, at least 93%, at least 94%, at least 95%, at least 96%, at least 97%, at least 98%, at least 99%, or 100% sequence identity to the sequence set forth in SEQ ID NO: 109; (x) at least 91%, at least 92%, at least 93%, at least 94%, at least 95%, at least 96%, at least 97%, at least 98%, at least 99%, or 100% sequence identity to the sequence set forth in SEQ ID NO: 115, 119, or 124; (xi) at least 92%, at least 93%, at least 94%, at least 95%, at least 96%, at least 97%, at least 98%, at least 99%, or 100% sequence identity to the sequence set forth in SEQ ID NO: 116, 120, or 125; (xii) at least 98%, at least 99%, or 100% sequence identity to the sequence set forth in SEQ ID NO: 112; (xiii) at least 98%, at least 99%, or 100% sequence identity to the sequence set forth in SEQ ID NO: 113; or (xiv) at least 98%, at least 99%, or 100% sequence identity to the sequence set forth in SEQ ID NO: 114.

In some aspects, the nucleic acid molecule encoding the IL-12α subunit comprises the sequence set forth in SEQ ID NO: 101. In some aspects, the nucleic acid molecule encoding the IL-12α subunit comprises the sequence set forth in SEQ ID NO: 102. In some aspects, the nucleic acid molecule encoding the IL-12α subunit comprises the sequence set forth in SEQ ID NO: 103. In some aspects, the nucleic acid molecule encoding the IL-12α subunit comprises the sequence set forth in SEQ ID NO: 104. In some aspects, the nucleic acid molecule encoding the IL-12α subunit comprises the sequence set forth in SEQ ID NO: 105. In some aspects, the nucleic acid molecule encoding the IL-12α subunit comprises the sequence set forth in SEQ ID NO: 106. In some aspects, the nucleic acid molecule encoding the IL-12α subunit comprises the sequence set forth in SEQ ID NO: 107. In some aspects, the nucleic acid molecule encoding the IL-12α subunit comprises the sequence set forth in SEQ ID NO: 108. In some aspects, the nucleic acid molecule encoding the IL-12α subunit comprises the sequence set forth in SEQ ID NO: 109. In some aspects, the nucleic acid molecule encoding the IL-12α subunit comprises the sequence set forth in SEQ ID NO: 115. In some aspects, the nucleic acid molecule encoding the IL-12α subunit comprises the sequence set forth in SEQ ID NO: 116. In some aspects, the nucleic acid molecule encoding the IL-12β subunit comprises the sequence set forth in SEQ ID NO: 117. In some aspects, the nucleic acid molecule encoding the IL-12β subunit comprises the sequence set forth in SEQ ID NO: 118. In some aspects, the nucleic acid molecule encoding the IL-12β subunit comprises the sequence set forth in SEQ ID NO: 119]. In some aspects, the nucleic acid molecule encoding the IL-12β subunit comprises the sequence set forth in SEQ ID NO: 120. In some aspects, the nucleic acid molecule encoding the IL-12β subunit comprises the sequence set forth in SEQ ID NO: 121. In some aspects, the nucleic acid molecule encoding the IL-12β subunit comprises the sequence set forth in SEQ ID NO: 122. In some aspects, the nucleic acid molecule encoding the IL-12β subunit comprises the sequence set forth in SEQ ID NO: 123. In some aspects, the nucleic acid molecule encoding the IL-12 subunit comprises the sequence set forth in SEQ ID NO: 124. In some aspects, the nucleic acid molecule encoding the IL-12β subunit comprises the sequence set forth in SEQ ID NO: 125. In some aspects, the nucleic acid molecule encoding the IL-12α subunit comprises the sequence set forth in SEQ ID NO: 112. In some aspects, the nucleic acid molecule encoding the IL-12α subunit comprises the sequence set forth in SEQ ID NO: 113. In some aspects, the nucleic acid molecule encoding the IL-12α subunit comprises the sequence set forth in SEQ ID NO: 114. In some aspects, the nucleic acid molecule encoding the IL-12β subunit comprises the sequence set forth in SEQ ID NO: 110. In some aspects, the nucleic acid molecule encoding the IL-12β subunit comprises the sequence set forth in SEQ ID NO: 111.

In some aspects, the nucleic acid molecule encoding the IL-12 p40 subunit and the nucleic acid molecule encoding the IL-12 p35 subunit can be conjugated to each other. For instance, in some aspects, the present disclosure provides an isolated polynucleotide comprising a first nucleic acid and a second nucleic acid, wherein the first nucleic acid encodes the IL-12 p40 subunit and the second nucleic acid encodes the IL-12 p35 subunit. In some aspects, the IL-12α subunit and the IL-12β subunit are linked by a linker. In some aspects, the linker comprises an amino acid linker of at least about 2, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 amino acids. In some aspects, the linker comprises a (GS) linker. In some aspects, the GS linker has a formula of (Gly3Ser)n or S(Gly3Ser)n, wherein n is a positive integer selected from the group consisting of 1, 2, 3, 4, 5, 6, 7, 8, 9, 10, 11, 12, 13, 14, 15, 16, 17, 18, 19, 20, 30, 40, 50, 60, 70, 80, or 100. In some aspects, the (Gly3Ser)n linker is (Gly3Ser)3 or (Gly3Ser)4.

In some aspects, a VEE replicon comprises a nucleic acid sequence encoding a chemokine. Non-limiting examples of chemokines include, CCL14, CCL19, CCL20, CCL21, CCL25, CCL27, CXCL12, CXCL13, CXCL-8, CCL2, CCL3, CCL4, CCL5, CCL11, and CXCL10. In some aspects, a VEE replicon comprises a nucleic acid sequence encoding a growth factor. As used herein, the term “growth factors” refers to a naturally occurring substance capable of signaling between cells and stimulating cellular growth. While cytokines may be growth factors, certain types of cytokines may also have an inhibitory effect on cell growth, thus differentiating the two terms. Non-limiting examples of growth factors include Adrenomedullin (AM), Angiopoietin (Ang), Autocrine motility factor, Bone morphogenetic proteins (BMPs), Ciliary neurotrophic factor (CNTF), Leukemia inhibitory factor (LIF), Interleukin-6 (IL-6), Macrophage colony-stimulating factor (m-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Fetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukin-1 (IL-1), IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

In some aspects, a VEE replicon useful for the present disclosure comprises a nucleic acid sequence encoding a ligand binding protein. Non-limiting examples of ligand binding proteins include a chimeric antigen receptor (CAR), T cell receptor (TCR), chimeric antibody-T cell receptor (caTCR), chimeric signaling receptor (CSR), T cell receptor mimic (TCR mimic), and combinations thereof.

In some aspects, a VEE replicon comprises a nucleic acid sequence encoding a CAR. In some aspects, the CAR is designed as a standard CAR. In a “standard CAR”, the different components (e.g., the extracellular targeting domain, transmembrane domain, and intracellular signaling/activation domain) are linearly constructed as a single fusion protein. In some aspects, the CAR is designed as a first generation CAR. “First generation” CARs are composed of an extracellular binding domain, a hinge region, a transmembrane domain, and one or more intracellular signaling domains. All first generation CARs contain the CD3ζ chain domain as the intracellular signaling domain. In some aspects, the CAR is designed as a second generation CAR. “Second generation” CARs additionally contain a costimulatory domain (e.g., CD28 or 4-1BB). In some aspects, the CAR is designed as a third generation CAR. “Third generation” CARs are similar to the second generation CARs except that they contain multiple costimulatory domains (e.g., CD28-4-1BB or CD28-OX40). In some aspects, the CAR is designed as a fourth generation CAR. “Fourth generation” CARs (also known as TRUCKs or armored CARs) additionally contain additional factors that can further improve function. For example, in some aspects, the fourth generation CARs additionally contain cytokines which can be released upon CAR signaling in the targeted tumor tissue. In some aspects, the fourth generation CARs comprise one or more additional elements such as homing and suicide genes, which can help further regulate the activity of the CAR. In some aspects, the CAR is designed as a split CAR. In a “split CAR” system, one or more components of the CAR (e.g., extracellular targeting domain, transmembrane domain, and intracellular signaling/activation domain) are split into two or more parts such that it is dependent on multiple inputs that promote assembly of the intact functional receptor. In some aspects, the CAR is designed as a switchable CAR. With a “switchable CAR,” the CAR can be switched (e.g., transiently) on (on-switch CAR) or off (off-switch CAR) in the presence of a stimulus. Additional examples of CARs that can be used with the present disclosure are described, e.g., in US 2020/0172879 A1 and US 2019/0183932 A1, each of which is incorporated herein by reference in its entirety.

In some aspects, a VEE replicon described herein comprises a nucleic acid sequence encoding an antibody or an antigen-binding fragment thereof (collectively referred to herein as “antibody”). The antibody can be derived from natural sources, or partly or wholly synthetically produced. In some aspects, the antibody is a monoclonal antibody. In some aspects, the monoclonal antibody is an IgG antibody. In some aspects, the monoclonal antibody is an IgG1, IgG2, IgG3, or IgG4. In some aspects, the antibody is a polyclonal antibody. In some aspects, the antigen-binding fragment is selected from Fab, Fab′, and F(ab′)₂, F(ab1)₂, Fv, dAb, and Fd fragments. In some aspects, the antigen-binding fragment is an scFv or (scFv) 2 fragment. In some aspects, the antibody or antigen-binding fragment is a NANOBODY® (single-domain antibody). In some aspects, the antibody or antigen-binding fragment is a bispecific or multi-specific antibody.

### IV.B. Additional Components

In some aspects, VEE replicons useful for the present disclosure comprises a nucleic acid sequence with one or more of the following additional components.

### Terminal Architecture Modifications: UTRs

Untranslated regions (UTRs) of a gene are transcribed but not translated. The 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the RNA of the present disclosure to enhance the stability of the molecule. The specific features can also be incorporated to ensure controlled down-regulation of the transcript in case they are misdirected to undesired organs sites.

### 5′ UTR and Translation Initiation

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

5′UTR secondary structures involved in elongation factor binding can interact with other RNA binding molecules in the 5′UTR or 3′UTR to regulate gene expression. For example, the elongation factor EIF4A2 binding to a secondarily structured element in the 5′UTR is necessary for microRNA mediated repression (Meijer H A et al., Science, 2013, 340, 82-85, herein incorporated by reference in its entirety). The different secondary structures in the 5′UTR can be incorporated into the flanking region to either stabilize or selectively destablize mRNAs in specific tissues or cells.

By engineering the features typically found in abundantly expressed genes of specific target organs, one can enhance the stability and protein production of the nucleic acids or mRNA of the disclosure. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of a nucleic acid molecule, such as a mmRNA, in hepatic cell lines or liver. Likewise, use of 5′ UTR from other tissue-specific mRNA to improve expression in that tissue is possible—for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences can be incorporated into the 5′ (or 3′ UTR) UTRs. For example, introns or portions of introns sequences can be incorporated into the flanking regions of the nucleic acids or mRNA of the disclosure. Incorporation of intronic sequences can increase protein production as well as mRNA levels.

In some aspects of the disclosure, at least one fragment of IRES sequences from a GTX gene can be included in the 5′UTR. As a non-limiting example, the fragment can be an 18 nucleotide sequence from the IRES of the GTX gene. As another non-limiting example, an 18 nucleotide sequence fragment from the IRES sequence of a GTX gene can be tandemly repeated in the 5′UTR of a polynucleotide described herein. The 18 nucleotide sequence can be repeated in the 5′UTR at least one, at least twice, at least three times, at least four times, at least five times, at least six times, at least seven times, at least eight times, at least nine times or more than ten times.

Nucleotides can be mutated, replaced and/or removed from the 5′ (or 3′) UTRs. For example, one or more nucleotides upstream of the start codon can be replaced with another nucleotide. The nucleotide or nucleotides to be replaced can be 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more than 60 nucleotides upstream of the start codon. As another example, one or more nucleotides upstream of the start codon can be removed from the UTR.

### 5′UTR, 3′UTR and Translation Enhancer Elements (TEEs)

In some aspects, the 5′UTR of the nucleotide sequence encoding IL-12 comprises at least one translational enhancer polynucleotide, translation enhancer element, translational enhancer elements (collectively referred to as “TEE”s). In some aspects, the TEE is located between the transcription promoter and the start codon. In some aspects, the RNA with at least one TEE in the 5′UTR comprises a cap at the 5′UTR. In some aspects, the at least one TEE can be located in the 5′UTR of nucleotide sequence encoding IL-12 undergoing cap-dependent or cap-independent translation.

The term “translational enhancer element” or “translation enhancer element” (herein collectively referred to as “TEE”) refers to sequences that increase the amount of polypeptide or protein produced from an mRNA.

In one aspect, TEEs are conserved elements in the UTR which can promote translational activity of a nucleic acid such as, but not limited to, cap-dependent or cap-independent translation. The conservation of these sequences has been previously shown by Panek et al (Nucleic Acids Research, 2013, 1-10; herein incorporated by reference in its entirety) across 14 species including humans.

In some aspects, the nucleotide sequence encoding IL-12 has at least one TEE that has at least about 50%, at least about 55%, at least about 60%, at least about 65%, at least about 70%, at least about 75%, at least about 80%, at least about 85%, at least about 90%, at least about 95% or at least about 99% identity with the disclosed in U.S. Application Number 2014/0147454, which is hereby incorporated by reference in its entirety. In some aspects, the RNA includes at least one TEE that has at least about 50%, at least about 55%, at least about 60%, at least about 65%, at least about 70%, at least about 75%, at least about 80%, at least about 85%, at least about 90%, at least about 95% or at least about 99% identity with the TEEs described in US Patent Publication Nos. US20090226470, US20070048776, US20130177581 and US20110124100, International Patent Publication No. WO1999024595, WO2012009644, WO2009075886 and WO2007025008, European Patent Publication No. EP2610341A1 and EP2610340A1, U.S. Pat. Nos. 6,310,197, 6,849,405, 7,456,273, 7,183,395, each of which is herein incorporated by reference in its entirety.

In some aspects, the 5′UTR of the nucleotide sequence encoding IL-12 can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some aspects, the TEE sequences in the 5′UTR of the RNA are the same or different TEE sequences. In some aspects, the TEE sequences are in a pattern such as ABABAB or AABBAABBAABB or ABCABCABC or variants thereof repeated once, twice, or more than three times. In these patterns, each letter, A, B, or C represent a different TEE sequence at the nucleotide level.

In some aspects, the spacer separating two TEE sequences includes other sequences known in the art which regulate the translation of the RNA such as, but not limited to, miR sequences described herein (e.g., miR binding sites and miR seeds). In some aspects, each spacer used to separate two TEE sequences includes a different miR sequence or component of a miR sequence (e.g., miR seed sequence).

In some aspects, the TEE used in the 5′UTR of the nucleotide sequence encoding IL-12 of the present disclosure is an IRES sequence such as, but not limited to, those described in U.S. Pat. No. 7,468,275 and International Patent Publication No. WO2001055369, each of which is herein incorporated by reference in its entirety.

In some aspects, the TEEs described herein are located in the 5′UTR and/or the 3′UTR of the nucleotide sequence encoding IL-12. In some aspects, the TEEs located in the 3′UTR are the same and/or different than the TEEs located in and/or described for incorporation in the 5′UTR.

In some aspects, the 3′UTR of the nucleotide sequence encoding IL-12 can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some aspects, the TEE sequences in the 3′UTR of the nucleotide sequence encoding IL-12 of the present disclosure is the same or different TEE sequences. The TEE sequences is in a pattern such as ABABAB or AABBAABBAABB or ABCABCABC or variants thereof repeated once, twice, or more than three times. In these patterns, each letter, A, B, or C represent a different TEE sequence at the nucleotide level.

In some aspects, the 3′UTR includes a spacer to separate two TEE sequences. In some aspects, the spacer is a 15 nucleotide spacer and/or other spacers known in the art. In some aspects, the 3′UTR can include a TEE sequence-spacer module repeated at least once, at least twice, at least 3 times, at least 4 times, at least 5 times, at least 6 times, at least 7 times, at least 8 times and at least 9 times or more than 9 times in the 3′UTR.

In some aspects, the spacer separating two TEE sequences includes other sequences known in the art which regulate the translation of the nucleotide sequence encoding IL-12, such as, but not limited to, miR sequences described herein (e.g., miR binding sites and miR seeds). In some aspects, each spacer used to separate two TEE sequences includes a different miR sequence or component of a miR sequence (e.g., miR seed sequence).

Incorporating microRNA Binding Sites

In some aspects, the nucleotide sequence encoding IL-12 further comprises a sensor sequence. Sensor sequences include, for example, microRNA binding sites, transcription factor binding sites, structured mRNA sequences and/or motifs, artificial binding sites engineered to act as pseudo-receptors for endogenous nucleic acid binding molecules. Non-limiting examples, of polynucleotides comprising at least one sensor sequence are described U.S. Application No. 2014/0147454, which is hereby incorporated by reference in its entirety.

In some aspects, microRNA (miRNA) profiling of the target cells or tissues is conducted to determine the presence or absence of miRNA in the cells or tissues.

MicroRNAs (or miRNA) are 19-25 nucleotide long noncoding RNAs that bind to the 3′UTR of nucleic acid molecules and down-regulate gene expression either by reducing nucleic acid molecule stability or by inhibiting translation. In some aspects, the RNA comprises one or more microRNA target sequences, microRNA sequences, or microRNA seeds. Such sequences can correspond to any known microRNA such as those taught in US Publication US2005/0261218 and US Publication US2005/0059005, the contents of which are incorporated herein by reference in their entirety. As a non-limiting example, known microRNAs, their sequences and seed sequences in human genome are described in U.S. Application No. 2014/0147454, which is herein incorporated by reference in its entirety.

A microRNA sequence comprises a “seed” region, i.e., a sequence in the region of positions 2-8 of the mature microRNA, which sequence has perfect Watson-Crick complementarity to the miRNA target sequence. A microRNA seed comprises positions 2-8 or 2-7 of the mature microRNA. In some aspects, a microRNA seed comprises 7 nucleotides (e.g., nucleotides 2-8 of the mature microRNA), wherein the seed-complementary site in the corresponding miRNA target is flanked by an adenine (A) opposed to microRNA position 1. In some aspects, a microRNA seed comprises 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K, Johnston W K, Garrett-Engele P, Lim L P, Bartel D P; Mol Cell. 2007 Jul. 6; 27 (1): 91-105. The bases of the microRNA seed have complete complementarity with the target sequence. By engineering microRNA target sequences into the 3′UTR of nucleic acids or mRNA of the disclosure one can target the molecule for degradation or reduced translation, provided the microRNA in question is available. This process will reduce the hazard of off target effects upon nucleic acid molecule delivery. Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is herein incorporated by reference in its entirety).

For example, if the mRNA is not intended to be delivered to the liver but ends up there, then miR-122, a microRNA abundant in liver, can inhibit the expression of the gene of interest if one or multiple target sites of miR-122 are engineered into the 3′UTR of the modified nucleic acids, enhanced modified RNA or ribonucleic acids. Introduction of one or multiple binding sites for different microRNA can be engineered to further decrease the longevity, stability, and protein translation of a modified nucleic acids, enhanced modified RNA or ribonucleic acids. As used herein, the term “microRNA site” refers to a microRNA target site or a microRNA recognition site, or any nucleotide sequence to which a microRNA binds or associates. It should be understood that “binding” can follow traditional Watson-Crick hybridization rules or can reflect any stable association of the microRNA with the target sequence at or adjacent to the microRNA site.

Conversely, for the purposes of the nucleotide sequence encoding IL-12 of the present disclosure, microRNA binding sites can be engineered out of (i.e. removed from) sequences in which they naturally occur in order to increase protein expression in specific tissues. For example, miR-122 binding sites can be removed to improve protein expression in the liver.

In some aspects, the nucleotide sequence encoding IL-12 includes at least one miRNA-binding site in the 3′UTR in order to direct cytotoxic or cytoprotective mRNA therapeutics to specific cells such as, but not limited to, normal and/or cancerous cells (e.g., HEP3B or SNU449).

Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, microRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific microRNAs are involved in immunogenicity, autoimmunity, the immune-response to infection, inflammation, as well as unwanted immune response after gene therapy and tissue/organ transplantation. Immune cells specific microRNAs also regulate many aspects of development, proliferation, differentiation and apoptosis of hematopoietic cells (immune cells). For example, miR-142 and miR-146 are exclusively expressed in the immune cells, particularly abundant in myeloid dendritic cells. It was demonstrated in the art that the immune response to exogenous nucleic acid molecules was shut-off by adding miR-142 binding sites to the 3′UTR of the delivered gene construct, enabling more stable gene transfer in tissues and cells. miR-142 efficiently degrades the exogenous mRNA in antigen presenting cells and suppresses cytotoxic elimination of transduced cells (Annoni A et al., blood, 2009, 114, 5152-5161; Brown B D, et al., Nat med. 2006, 12 (5), 585-591; Brown B D, et al., blood, 2007, 110 (13): 4144-4152, each of which is herein incorporated by reference in its entirety).

Many microRNA expression studies are conducted in the art to profile the differential expression of microRNAs in various cancer cells/tissues and other diseases. Some microRNAs are abnormally over-expressed in certain cancer cells and others are under-expressed. For example, microRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lympho nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia lymphoma (WO2008/073915, US2009/0092974, US2012/0316081, US2012/0283310, WO2010/018563, the content of each of which is incorporated herein by reference in their entirety.)

At least one microRNA site can be engineered into the 3′ UTR of the nucleotide sequence encoding IL-12. In some aspects, at least two, at least three, at least four, at least five, at least six, at least seven, at least eight, at least nine, at least ten or more microRNA sites can be engineered into the 3′ UTR of the nucleotide sequence encoding IL-12. In some aspects, the microRNA sites incorporated into the nucleotide sequence encoding IL-12 are the same or different microRNA sites. In some aspects, the microRNA sites incorporated into the nucleotide sequence encoding IL-12 targets the same or different tissues in the body. As a non-limiting example, through the introduction of tissue-, cell-type-, or disease-specific microRNA binding sites in the 3′ UTR of a mRNA, the degree of expression in specific cell types (e.g., hepatocytes, myeloid cells, endothelial cells, cancer cells, etc.) can be reduced.

In some aspects, a microRNA site is engineered near the 5′ terminus of the 3′UTR, about halfway between the 5′ terminus and 3′ terminus of the 3′UTR and/or near the 3′ terminus of the 3′UTR. In some aspects, a microRNA site is engineered near the 5′ terminus of the 3′UTR and about halfway between the 5′ terminus and 3′ terminus of the 3′UTR. In some aspects, a microRNA site is engineered near the 3′ terminus of the 3′UTR and about halfway between the 5′ terminus and 3′ terminus of the 3′UTR. In some aspects, a microRNA site is engineered near the 5′ terminus of the 3′UTR and near the 3′ terminus of the 3′UTR.

In some aspects, a mRNA comprises microRNA binding region sites that either have 100% identity to known seed sequences or have less than 100% identity to seed sequences. The seed sequence can be partially mutated to decrease microRNA binding affinity and as such result in reduced down modulation of that mRNA transcript. In essence, the degree of match or mis-match between the target mRNA and the microRNA seed can act as a rheostat to more finely tune the ability of the microRNA to modulate protein expression. In addition, mutation in the non-seed region of a microRNA binding site can also impact the ability of a microRNA to modulate protein expression.

### RNA Motifs for RNA Binding Proteins (RBPs)

RNA binding proteins (RBPs) can regulate numerous aspects of co- and post-transcription gene expression such as, but not limited to, RNA splicing, localization, translation, turnover, polyadenylation, capping, modification, export and localization. RNA-binding domains (RBDs), such as, but not limited to, RNA recognition motif (RR) and hnRNP K-homology (KH) domains, typically regulate the sequence association between RBPs and their RNA targets (Ray et al. Nature 2013. 499:172-177; herein incorporated by reference in its entirety). In some aspects, the canonical RBDs bind short RNA sequences. In some aspects, the canonical RBDs recognize RNA structure.

Non limiting examples of RNA binding proteins and related nucleic acid and protein sequences are described in U.S. Application No. 2014/0147454, which is herein incorporated by reference in its entirety.

In some aspects, to increase the stability of the mRNA of interest, an mRNA encoding HuR is co-transfected or co-injected along with the mRNA of interest into the cells or into the tissue. These proteins can also be tethered to the mRNA of interest in vitro and then administered to the cells together. Poly A tail binding protein, PABP interacts with eukaryotic translation initiation factor eIF4G to stimulate translational initiation. Co-administration of mRNAs encoding these RBPs along with the mRNA drug and/or tethering these proteins to the mRNA drug in vitro and administering the protein-bound mRNA into the cells can increase the translational efficiency of the mRNA. The same concept can be extended to co-administration of mRNA along with mRNAs encoding various translation factors and facilitators as well as with the proteins themselves to influence RNA stability and/or translational efficiency.

In some aspects, the nucleotide sequence encoding IL-12 comprises at least one RNA-binding motif such as, but not limited to a RNA-binding domain (RBD).

In some aspects, the first region of linked nucleosides and/or at least one flanking region comprises at least on RBD. In some aspects, the first region of linked nucleosides comprises a RBD related to splicing factors and at least one flanking region comprises a RBD for stability and/or translation factors.

### Other Regulatory Elements in 3′UTR

In addition to microRNA binding sites, other regulatory sequences in the 3′-UTR of natural mRNA, which regulate mRNA stability and translation in different tissues and cells, can be removed or introduced into RNA. Such cis-regulatory elements can include, but are not limited to, Cis-RNP (Ribonucleoprotein)/RBP (RNA binding protein) regulatory elements, AU-rich element (AUE), structured stem-loop, constitutive decay elements (CDEs), GC-richness and other structured mRNA motifs (Parker B J et al., Genome Research, 2011, 21, 1929-1943, which is herein incorporated by reference in its entirety). For example, CDEs are a class of regulatory motifs that mediate mRNA degradation through their interaction with Roquin proteins. In particular, CDEs are found in many mRNAs that encode regulators of development and inflammation to limit cytokine production in macrophage (Leppek K et al., 2013, Cell, 153, 869-881, which is herein incorporated by reference in its entirety).

In some aspects, the RNA is auxotrophic. As used herein, the term “auxotrophic” refers to mRNA that comprises at least one feature that triggers, facilitates or induces the degradation or inactivation of the mRNA in response to spatial or temporal cues such that protein expression is substantially prevented or reduced. Such spatial or temporal cues include the location of the mRNA to be translated such as a particular tissue or organ or cellular environment. Also contemplated are cues involving temperature, pH, ionic strength, moisture content and the like.

### 3′ UTR and the AU Rich Elements

3′UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids or mRNA of the disclosure. When engineering specific nucleic acids or mRNA, one or more copies of an ARE can be introduced to make nucleic acids or mRNA of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids or mRNA of the disclosure and protein production can be assayed at various time points post-transfection. For example, cells can be transfected with different ARE-engineering molecules and by using an ELISA kit to the relevant protein and assaying protein produced at about 6 hr, about 12 hr, about 24 hr, about 48 hr, and/or about 7 days post-transfection.

### 3′ UTR and Triple Helices

In some aspects, the nucleotide sequence encoding IL-12 comprises a triple helix on the 3′ end of the nucleic acid, enhanced nucleotide sequence encoding IL-12 or ribonucleic acid. In some aspects, the 3′ end of the nucleotide sequence encoding IL-12 include a triple helix alone or in combination with a Poly-A tail.

In some aspects, the nucleotide sequence encoding IL-12 comprises at least a first and a second U-rich region, a conserved stem loop region between the first and second region and an A-rich region. In some aspects, the first and second U-rich region and the A-rich region associate to form a triple helix on the 3′ end of the nucleic acid. This triple helix can stabilize the nucleic acid, enhance the translational efficiency of the nucleic acid and/or protect the 3′ end from degradation. Exemplary triple helices include, but are not limited to, the triple helix sequence of metastasis-associated lung adenocarcinoma transcript 1 (MALAT1), MEN-β and polyadenylated nuclear (PAN) RNA (See Wilusz et al., Genes & Development 2012 26:2392-2407; herein incorporated by reference in its entirety).

### Stem Loop

In some aspects, the nucleotide sequence encoding IL-12 includes a stem loop such as, but not limited to, a histone stem loop. In some aspects, the stem loop is a nucleotide sequence that is about 25 or about 26 nucleotides in length such as, but not limited to, SEQ ID NOs: 7-17 as described in International Patent Publication No. WO2013103659, herein incorporated by reference in its entirety. The histone stem loop can be located 3′ relative to the coding region (e.g., at the 3′ terminus of the coding region). As a non-limiting example, the stem loop can be located at the 3′ end of a nucleic acid described herein.

In some aspects, the nucleotide sequence encoding IL-12, which comprises the histone stem loop can be stabilized by the addition of at least one chain terminating nucleoside. Not wishing to be bound by theory, the addition of at least one chain terminating nucleoside can slow the degradation of a nucleic acid and thus can increase the half-life of the nucleic acid.

In some aspects, the chain terminating nucleoside is one described in International Patent Publication No. WO2013103659, herein incorporated by reference in its entirety. In some aspects, the chain terminating nucleosides are 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or α-O-methylnucleoside.

In some aspects, the nucleotide sequence encoding IL-12 includes a histone stem loop, a polyA tail sequence and/or a 5′ cap structure. In some aspects, the histone stem loop is before and/or after the polyA tail sequence. The nucleic acids comprising the histone stem loop and a polyA tail sequence can include a chain terminating nucleoside described herein.

In some aspects, the nucleotide sequence encoding IL-12 comprises a histone stem loop and a 5′ cap structure. The 5′ cap structure can include, but is not limited to, those described herein and/or known in the art.

### 5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

Modifications to the RNA of the present disclosure can generate a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′ mppp-G; which can equivalently be designated 3′ O-Me-m7G (5′)ppp(5′) G). The 3′-O atom of the other, unmodified, guanine becomes linked to the 5′-terminal nucleotide of the capped nucleic acid molecule (e.g., an mRNA or mmRNA). The N7- and 3′-O-methylated guanine provides the terminal moiety of the capped nucleic acid molecule (e.g., mRNA or mmRNA).

Another exemplary cap is mCAP, which is similar to ARCA but has a 2′-β-methyl group on guanosine (i.e., N7,2′-O-dimethyl-guanosine-5′-triphosphate-5′-guanosine, m7Gm-ppp-G)

In some aspects, the cap is a dinucleotide cap analog. In some aspects, the dinucleotide cap analog is modified at different phosphate positions with a boranophosphate group or a phosphoroselenoate group such as the dinucleotide cap analogs described in U.S. Pat. No. 8,519,110, the contents of which are herein incorporated by reference in its entirety.

In some aspects, the cap is a cap analog is a N7-(4-chlorophenoxyethyl) substituted dicucleotide form of a cap analog known in the art and/or described herein. Non-limiting examples of a N7-(4-chlorophenoxyethyl) substituted dinucleotide form of a cap analog include a N7-(4-chlorophenoxyethyl)-G (5′)ppp(5′) G and a N7-(4-chlorophenoxyethyl)-m3′-OG (5′)ppp(5′) G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In some aspects, a cap analog of the present disclosure is a 4-chloro/bromophenoxyethyl analog.

While cap analogs allow for the concomitant capping of a nucleic acid molecule in an in vitro transcription reaction, up to about 20% of transcripts remain uncapped. This, as well as the structural differences of a cap analog from an endogenous 5′-cap structures of nucleic acids produced by the endogenous, cellular transcription machinery, can lead to reduced translational competency and reduced cellular stability. Accordingly, in some aspects, the methods provided herein (see, e.g., Examples 1-3) are capable of increasing the capping efficiency of the produced IL-12 expressing nucleotides described herein. In some aspects, with the methods provided herein, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100% of the polynucleotides are capped. In some aspects, with the methods provided herein, at least about 50% of the polynucleotides are capped. In some aspects, at least about 60% of the polynucleotides are capped. In some aspects, at least about 70% of the polynucleotides are capped. In some aspects, at least about 80% of the polynucleotides are capped. In some aspects, at least about 85% of the polynucleotides are capped. In some aspects, at least about 90% of the polynucleotides are capped. In some aspects, at least about 95% of the polynucleotides are capped. In some aspects, about 100% of the polynucleotides are capped. In some aspects, at least about 80% to about 100% of the polynucleotides are capped.

In some aspects, providing an RNA with a 5′-cap or 5′-cap analog is achieved by in vitro transcription of a DNA template in the presence of said 5′-cap or 5′-cap analog, wherein said 5′-cap is co-transcriptionally incorporated into the generated RNA strand,

In some aspects, RNA can be generated, for example, by in vitro transcription, and the 5′-cap can be attached to the RNA post-transcriptionally using capping enzymes, for example, capping enzymes of vaccinia virus. In some aspects, the nucleotide sequence encoding IL-12 is capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures of the present disclosure are those which, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. This cap results in a higher translational-competency and cellular stability and a reduced activation of cellular pro-inflammatory cytokines, as compared, e.g., to other 5′ cap analog structures known in the art. Cap structures include 7mG(5′)ppp(5′)N,pN2p, 7 mG(5′)ppp(5′) NlmpNp, 7mG(5′)-ppp(5′)NlmpN2mp and m(7)Gpppm(3)(6,6,2′)Apm(2′)Apm(2′)Cpm(2)(3,2′)Up.

In some aspects, 5′ terminal caps include endogenous caps or cap analogs. In some aspects, a 5′ terminal cap comprises a guanine analog. Useful guanine analogs include inosine, N1-methyl-guanosine, 2′ fluoro-guanosine, 7-deaza-guanosine, 8-oxo-guanosine, 2-amino-guanosine, LNA-guanosine, and 2-azido-guanosine.

In some aspects, the 5′ cap comprises a 5′ to 5′ triphosphate linkage. In some aspects, the 5′ cap comprises a 5′ to 5′ triphosphate linkage including thiophosphate modification. In some aspects, the 5′ cap comprises a 2′-0 or 3′-O-ribose-methylated nucleotide. In some aspects, the 5′ cap comprises a modified guanosine nucleotide or modified adenosine nucleotide. In some aspects, the 5′ cap comprises 7-methylguanylate. Exemplary cap structures include m7G(5′)ppp(5′)G, m7,2′O-mG(5′)ppSp(5′)G, m7G(5′)ppp(5′)2′O-mG, and m7,3′O-mG(5′)ppp(5′)2′O-mA.

In some aspects, the nucleotide sequence encoding IL-12 comprises a modified 5′ cap. A modification on the 5′ cap can increase the stability of mRNA, increase the half-life of the mRNA, and could increase the mRNA translational efficiency. In some aspects, the modified 5′ cap comprises one or more of the following modifications: modification at the 2′ and/or 3′ position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

The 5′ cap structure that can be modified includes, but is not limited to, the caps described in U.S. Application No. 2014/0147454 and WO2018/160540 which is incorporated herein by reference in its entirety.

### IRES Sequences

In some aspects, the nucleotide sequence encoding IL-12 comprises an internal ribosome entry site (IRES). First identified as a feature Picorna virus RNA, IRES plays an important role in initiating protein synthesis in absence of the 5′ cap structure. An IRES can act as the sole ribosome binding site, or can serve as one of multiple ribosome binding sites of an mRNA. Nucleic acids or mRNA containing more than one functional ribosome binding site can encode several peptides or polypeptides that are translated independently by the ribosomes (“multicistronic nucleic acid molecules”). When nucleic acids or mRNA are provided with an IRES, further optionally provided is a second translatable region. Examples of IRES sequences that can be used according to the disclosure include without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Terminal Architecture Modifications: Poly-A Tails

During RNA processing, a long chain of adenine nucleotides (poly-A tail) is normally added to a messenger RNA (mRNA) molecules to increase the stability of the molecule. Immediately after transcription, the 3′ end of the transcript is cleaved to free a 3′ hydroxyl. Then poly-A polymerase adds a chain of adenine nucleotides to the RNA. The process, called polyadenylation, adds a poly-A tail that is between 100 and 250 residues long.

In some aspects, the length of the 3′ tail is greater than about 30 nucleotides in length. In some aspects, the poly-A tail is greater than about 35 nucleotides in length. In some aspects, the length is at least about 40 nucleotides. In some aspects, the length is at least about 45 nucleotides. In some aspects, the length is at least about 55 nucleotides. In some aspects, the length is at least about 60 nucleotides. In some aspects, the length is at least 70 nucleotides. In some aspects, the length is at least about 80 nucleotides. In some aspects, the length is at least about 90 nucleotides. In some aspects, the length is at least about 100 nucleotides. In some aspects, the length is at least about 120 nucleotides. In some aspects, the length is at least about 140 nucleotides. In some aspects, the length is at least about 160 nucleotides. In some aspects, the length is at least about 180 nucleotides. In some aspects, the length is at least about 200 nucleotides. In some aspects, the length is at least about 250 nucleotides. In some aspects, the length is at least about 300 nucleotides. In some aspects, the length is at least about 350 nucleotides. In some aspects, the length is at least about 400 nucleotides. In some aspects, the length is at least about 450 nucleotides. In some aspects, the length is at least about 500 nucleotides. In some aspects, the length is at least about 600 nucleotides. In some aspects, the length is at least about 700 nucleotides. In some aspects, the length is at least about 800 nucleotides. In some aspects, the length is at least about 900 nucleotides. In some aspects, the length is at least about 1000 nucleotides. In some aspects, the length is at least about 1100 nucleotides. In some aspects, the length is at least about 1200 nucleotides. In some aspects, the length is at least about 1300 nucleotides. In some aspects, the length is at least about 1400 nucleotides. In some aspects, the length is at least about 1500 nucleotides. In some aspects, the length is at least about 1600 nucleotides. In some aspects, the length is at least about 1700 nucleotides. In some aspects, the length is at least about 1800 nucleotides. In some aspects, the length is at least about 1900 nucleotides. In some aspects, the length is at least about 2000 nucleotides. In some aspects, the length is at least about 2500 nucleotides. In some aspects, the length is at least about 3000 nucleotides.

In some aspects, the nucleotide sequence encoding IL-12 is designed to include a poly A-G Quartet. The G-quartet is a cyclic hydrogen bonded array of four guanine nucleotides that can be formed by G-rich sequences in both DNA and RNA. In this aspect, the G-quartet is incorporated at the end of the poly-A tail. The resultant nucleic acid or mRNA can be assayed for stability, protein production and other parameters including half-life at various time points. It has been discovered that the polyA-G quartet results in protein production equivalent to at least 75% of that seen using a poly-A tail of 120 nucleotides alone.

In some aspects, the nucleotide sequence encoding IL-12 comprises a polyA tail and is stabilized by the addition of a chain terminating nucleoside. In some aspects, the nucleotide sequence encoding IL-12 with a polyA tail further comprise a 5′ cap structure.

In some aspects, the nucleotide sequence encoding IL-12 comprises a polyA-G Quartet. In some aspects, the nucleotide sequence encoding IL-12 with a polyA-G Quartet further comprises a 5′ cap structure.

In some aspects, the nucleotide sequence encoding IL-12, which comprise a poly A tail or a polyA-G Quartet is stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other modified nucleosides known in the art and/or described herein.

### Modified Nucleosides

In some aspects, the nucleotide sequence encoding IL-12 comprises one or more modified nucleosides. In some aspects, the one or more modified nucleosides comprises 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

In some aspects, one or more uridine in the nucleotide sequence encoding IL-12 is replaced by a modified nucleoside. In some aspects, the modified nucleoside replacing uridine is pseudouridine (ψ), N1-methyl-pseudouridine (m1ψ) or 5-methyl-uridine (m5U).

In some aspects, the nucleotide sequence encoding IL-12 comprises a nucleotide sequence encoding IL-12 as described in U.S. Application Number 2014/0147454, International Application WO2018160540, International Application WO2015/196118, or International Application WO2015/089511, which are incorporated herein by reference in their entirety.

### Cytotoxic Nucleosides

In some aspects, the nucleotide sequence encoding IL-12 comprises one or more cytotoxic nucleosides. For example, cytotoxic nucleosides can be incorporated into polynucleotides such as bifunctional nucleotide sequence encoding IL-12s or mRNAs. Cytotoxic nucleoside anti-cancer agents include, but are not limited to, adenosine arabinoside, cytarabine, cytosine arabinoside, 5-fluorouracil, fludarabine, floxuridine, FTORAFUR® (a combination of tegafur and uracil), tegafur ((RS)-5-fluoro-1-(tetrahydrofuran-2-yl)pyrimidine-2,4(1H,3H)-dione), and 6-mercaptopurine.

A number of cytotoxic nucleoside analogues are in clinical use, or have been the subject of clinical trials, as anticancer agents. Examples of such analogues include, but are not limited to, cytarabine, gemcitabine, troxacitabine, decitabine, tezacitabine, 2′-deoxy-2′-methylidenecytidine (DMDC), cladribine, clofarabine, 5-azacytidine, 4′-thio-aracytidine, cyclopentenylcytosine and 1-(2-C-cyano-2-deoxy-beta-D-arabino-pentofuranosyl)-cytosine. Another example of such a compound is fludarabine phosphate. These compounds can be administered systemically and can have side effects which are typical of cytotoxic agents such as, but not limited to, little or no specificity for tumor cells over proliferating normal cells.

A number of prodrugs of cytotoxic nucleoside analogues are also reported in the art. Examples include, but are not limited to, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester). In general, these prodrugs can be converted into the active drugs mainly in the liver and systemic circulation and display little or no selective release of active drug in the tumor tissue. For example, capecitabine, a prodrug of 5′-deoxy-5-fluorocytidine (and eventually of 5-fluorouracil), is metabolized both in the liver and in the tumor tissue. A series of capecitabine analogues containing “an easily hydrolysable radical under physiological conditions” has been claimed by Fujiu et al. (U.S. Pat. No. 4,966,891) and is herein incorporated by reference. The series described by Fujiu includes N4 alkyl and aralkyl carbamates of 5′-deoxy-5-fluorocytidine and the implication that these compounds will be activated by hydrolysis under normal physiological conditions to provide 5′-deoxy-5-fluorocytidine.

A series of cytarabine N4-carbamates has been by reported by Fadl et al (Pharmazie. 1995, 50, 382-7, herein incorporated by reference in its entirety) in which compounds were designed to convert into cytarabine in the liver and plasma. WO 2004/041203, herein incorporated by reference in its entirety, discloses prodrugs of gemcitabine, where some of the prodrugs are N4-carbamates. These compounds were designed to overcome the gastrointestinal toxicity of gemcitabine and were intended to provide gemcitabine by hydrolytic release in the liver and plasma after absorption of the intact prodrug from the gastrointestinal tract. Nomura et al (Bioorg Med. Chem. 2003, 11, 2453-61, herein incorporated by reference in its entirety) have described acetal derivatives of 1-(3-C-ethynyl-β-D-ribo-pentofaranosyl) cytosine which, on bioreduction, produced an intermediate that required further hydrolysis under acidic conditions to produce a cytotoxic nucleoside compound.

Cytotoxic nucleotides which can be chemotherapeutic also include, but are not limited to, pyrazolo[3,4-D]-pyrimidines, allopurinol, azathioprine, capecitabine, cytosine arabinoside, fluorouracil, mercaptopurine, 6-thioguanine, acyclovir, ara-adenosine, ribavirin, 7-deaza-adenosine, 7-deaza-guanosine, 6-aza-uracil, 6-aza-cytidine, thymidine ribonucleotide, 5-bromodeoxyuridine, 2-chloro-purine, and inosine, or combinations thereof.

### V. Pharmaceutical Compositions

In some aspects, the disclosure relates to a pharmaceutical composition comprising the polynucleotide, vector, and/or lipid nanoparticle described herein. In some aspects of the disclosure, the pharmaceutical composition further comprises a pharmaceutically acceptable carrier (excipient). “Acceptable”, as used herein, means that the carrier must be compatible with the active ingredient of the composition and not deleterious to the subject to be treated. In some aspects, the carrier is capable of stabilizing the active ingredient. Pharmaceutically acceptable excipients (carriers) include buffers, which are well known in the art. See, e.g., Remington: The Science and Practice of Pharmacy 20th Ed. (2000) Lippincott Williams and Wilkoins, Ed. K. E. Hoover.

The pharmaceutical compositions to be used for in vivo administration must be sterile. This is readily accomplished by, for example, filtration through sterile filtration membranes. The lipid nanoparticles can be placed into a container having a sterile access port, for example, an intravenous solution bag or vial having a stopper pierceable by a hypodermic injection needle.

In some aspects of the disclosure, the pharmaceutical composition can be formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration. In some aspects of the disclosure, the pharmaceutical composition can be formulated for intratumoral injection. Intratumoral injection, as used herein, refers to direct injections into the tumor. A high concentration of composition can be achieved in situ, while using small amounts of drugs. Local delivery of immunotherapies allows multiple combination therapies, while preventing significant system exposure and off-target toxicities.

In some aspects of the disclosure, the pharmaceutical composition can be formulated for intramuscular injection, intravenous injection, or subcutaneous injection.

In some aspects of the disclosure, the pharmaceutical composition comprises pharmaceutically acceptable carriers, buffer agents, excipients, salts, or stabilizers in the form of lyophilized formulations or aqueous solutions. See, e.g., Remington: The Science and Practice of Pharmacy 20^{th} Ed. (2000) Lippincott Williams and Wilkins, Ed. K. E. Hoover. Acceptable carriers and excipients, or stabilizers are nontoxic to recipients at the dosages and concentrations used, and comprises buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

In some aspects, the pharmaceutical composition described herein comprises lipid nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545, which are hereby incorporated by reference in their entirety. Liposomes with enhanced circulation time are disclosed in U.S. Pat. No. 5,013,556, which is hereby incorporated by reference in its entirety. In some aspects, liposomes can be generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some aspects of the disclosure, the pharmaceutical composition is formulated in sustained-release format. Suitable examples of sustained-release preparations include semipermeable matrices of solid hydrophobic polymers containing the lipid nanoparticles which matrices are in the form of shaped articles, e.g., films or microcapsules. Examples of sustained-release matrices include, but are not limited to, polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPROM DEPOT™ (injectable microspheres composed of lactic acid-glycolic acid copolymer and leuprolide acetate), sucrose acetate isobutyrate, and poly-D-(−)-3-hydroxybutyric acid.

In some aspects, suitable surface-active agents include, but are not limited to, non-ionic agents, such as polyoxyethylenesorbitans (e.g., TWEEN™ 20, 40, 60, 80 or 85) and other sorbitans (e.g., SPAN™ 20, 30, 60, 80, or 85). In some aspects, compositions with a surface-active agent comprise between 0.05 and 5% surface-active agent. In some aspects the composition comprises 0.1 and 2.5%. It will be appreciated that other ingredients can be added, for example mannitol or other pharmaceutically acceptable vehicles, if necessary.

In some aspects, the pharmaceutical composition is in unit dosage forms such as tablets, pills, capsules, powders, granules, solutions or suspensions, or suppositories, for oral, parenteral, or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogenous mixture of a compound of the present disclosure, or a non-toxic pharmaceutically acceptable salt thereof. When referring to these preformulation compositions as homogeneous, it is meant that the active ingredient is dispersed evenly throughout the composition so that the composition can be readily subdivided into equally effective unit dosage forms such as tablets, pills, and capsules. This solid preformulation composition is then subdivided into unit dosage forms of the type described above containing from about 0.1 to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

Suitable emulsions can be prepared using commercially available fat emulsions, such as INTRALIPID™, LIPOSYN™, INFONUTROL™, LIPOFUNDIN™, and LIPIPHYSAN™. The active ingredient can be either dissolved in a pre-mixed emulsion composition or alternatively it can be dissolved in an oil (e.g., soybean oil, safflower oil, cottonseed oil, sesame oil, corn oil, or almond oil) and an emulsion formed upon mixing with a phospholipid (e.g., egg phospholipids, soybean phospholipids, or soybean lecithin) and water. It will be appreciated that other ingredients can be added, for example glycerol or glucose, to adjust tonicity of the emulsion. Suitable emulsions will typically contain up to about 20% oil, for example, between about 5 and about 20%. The fat emulsion can comprise fat droplets having a suitable size and can have a pH in the range of about 5.5 to about 8.0.

Pharmaceutical compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as set out above. In some aspects, the composition is administered by the oral or nasal respiratory route for local or systemic effect.

Compositions in pharmaceutically acceptable solvents can be nebulized by use of gases. Nebulized solutions can be breathed directly from the nebulizing device or the nebulizing device can be attached to a face mask, tent or intermittent positive pressure breathing machine. Solution, suspension, or powder compositions can be administered from devices which deliver the formulation in an appropriate manner.

### VI. Kits for Use in Therapy

The present disclosure also provides kits for use in immunotherapy against a disease or disorder, such as a cancer (e.g., melanoma, lung cancer, colorectal cancer, or renal-cell cancer), and/or treating or reducing the risk for the disease or disorder (e.g., cancer). In some aspects, the kit includes one or more containers comprising a composition described herein.

In some aspects, the kit comprises instructions for use in accordance with any of the methods described herein. For example, the included instructions can comprise a description of administration of the pharmaceutical composition described herein to treat, delay the onset, or alleviate a target disease. In some aspects, the instructions comprise a description of administering the composition described herein to a subject at risk of the target disease/disorder (e.g., cancer).

In some aspects, the instructions comprise dosage information, dosing schedule, and route of administration. In some aspects, the containers are unit doses, bulk packages (e.g., multi-dose packages) or sub-unit doses. In some aspects, the instructions are written instructions on a label or package insert (e.g., a paper sheet included in the kit). In some aspects, the instructions are machine-readable instructions (e.g., instructions carried on a magnetic or optical storage disk).

In some aspects, the label or package insert indicates that the composition disclosed herein is used for treating, delaying the onset, and/or alleviating a disease or disorder associated with cancer, such as those described herein. Instructions can be provided for practicing any of the methods described herein.

In some aspects, the kits described herein are in suitable packaging. In some aspects, suitable packing comprises vials, bottles, jars, flexible packaging (e.g., seal Mylar or plastic bags), or combinations thereof. In some aspects, the packaging comprises packages for use in combination with a specific device such as an inhaler, nasal administration device (e.g., an atomizer), or an infusion device such as a minipump. In some aspects, the kit comprises a sterile access port (for example the container can be an intravenous solution bag or a vial having a stopper pierceable by a hypodermic injection needle). In some aspects, the container can also have a sterile access port (for example the container can be an intravenous solution bag or a vial having a stopper pierceable by a hypodermic injection needle). In some aspects, at least one active agent is a composition as described herein.

In some aspects, the kits further comprise additional components such as buffers and interpretive information. In some aspects, the kit comprises a container and a label or package insert(s) on or associated with the container. In some aspects, the disclosure provides articles of manufacture comprising the contents of the kits described herein.

### General Techniques

The practice of the present disclosure will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Giffiths, and D. G. Newell, eds., 1993-8) J. Wiley and Sons; Method of Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987): PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanette and J. D. Capra, eds., Harwood Academic Publishers, 1995). Without further elaboration, it is believed that one skilled in the art can, based on the above description, utilize the present disclosure to its fullest extent. All publications cited herein (including those listed above and elsewhere in the present disclosure) are incorporated by reference in their entirety.

### EXAMPLES

### Example 1: Construction of LNP Comprising a VEE Replicon

To construct the LNPs described herein, the following materials and methods were used:

### Template Preparation

Template DNA was linearized using a BspQI restriction enzyme. Following linearization, the DNA was buffer exchanged on a filter for 6 diavolumes. This DNA was then IVT'd using T7 RNA polymerase and NTPs. Following IVT, the remaining DNA was digested using DNaseI. The mRNA was then buffer exchanged prior to liquid chromatography. Following this, the mRNA was buffer exchanged again and then assayed for quality control purposes.

Preparation of Conventional BL-1 LNP Formulations with T-Junction

The lipid materials were each weighed out and dissolved in ethanol. The ethanol phase was prepared by mixing all the lipid materials according to composition ratio in the form below. The aqueous phase was prepared by diluting the RNA molecule (e.g., a mRNA with a VEE replicon or a control mRNA without any VEE) with 20 mM Citrate Buffer (pH 4.0), 300 mM NaCl and water. The LNPs were afforded by mixing the ethanol phase and aqueous phase of the LNPs through T-junction mixing at the flow rate ratio of 3:1 (aqueous phase:ethanol phase) and followed by an immediate in-line dilution with TBS buffer (pH 4.0) via T-junction mixing at the flow rate ratio of 1:1 (LNP phase:PEG phase). Table 2 below provides the relative amount of each of the components included in the LNP composition.

TABLE 2
BL-1 LNP Composition

| Components | Exemplary Amount | Weight Ratio |
|---|---|---|
| mRNA | 1.0 mg | 1.0 |
| BL-1 | 12.7 mg | 12.7 |
| DSPC | 2.8 mg | 2.8 |
| Cholesterol | 2.8 mg | 2.8 |
| Beta-sitosterol | 2.8 mg | 2.8 |
| DMG-PEG-2k | 1.5 mg | 1.5 |

Preparation of Post-PEG Micelle TT3 LNP Formulation with T-Junction

The lipid materials were each weighed out and dissolved in ethanol. The ethanol phase was prepared by mixing all the lipid materials except from DMG-PEG-2K, according to composition ratio in the form below. The aqueous phase was prepared by diluting the RNA molecule (e.g., mRNA with a VEE replicon or a control mRNA without any VEE replicon) with 20 mM Citrate Buffer (pH 4.0), 300 mM NaCl and water. PEG micelle phase was prepared by adding the corresponding amount of DMG-PEG-2K into TBS buffer and mixing thoroughly via vortex. Finally, the post-PEG micelles TT3 LNPs were afforded by first mixing the ethanol phase and aqueous phase of the LNPs through a T-junction mixing at the flow rate ratio of 3:1 (aqueous phase:ethanol phase), and followed by an immediate in-line dilution with the PEG micelle phase via T-junction mixing at the flow rate ratio of 1:1 (LNP phase:PEG phase). Table 3 below provides the relative amount of each of the components (both as exemplary amount and weight ratio) included in the LNP composition.

TABLE 3
TT3 LNP Composition

| Components | Exemplary Amount | Weight Ratio |
|---|---|---|
| mRNA | 1.0 mg | 1.0 |
| TT3 | 10 mg | 10.0 |
| DOPE | 8.0 mg | 8.0 |
| Cholesterol | 5.6 mg | 5.6 |
| DMG-PEG-2k | 4.1 mg | 4.1 |

TABLE 3A

| | Weight Ratio |
|---|---|
| TT3 LNP Composition Component | |
| mRNA | 1.0 |
| TT3 | 10.0 |
| DOPE | 8.0 |
| Cholesterol | 5.6 |
| PEG-DMG-2k Micelle | |
| DMG-PEG-2k | 4.1 (relative to mRNA above, which |
| | is not part of the micelle) |

### Buffer Exchange and Freeze/Thaw of BL-1/TT3 LNPs

The afforded BL-1/TT3 LNPs were buffer exchanged and concentrated via tangential flow filtration. Subsequently, 40% sucrose (W/V) in TBS stock solution was added into all the prepared BL-1/TT3 LNPs to make a final solution of LNPs in 10% sucrose. The final RNA concentrations of LNPs were measured by dissociating the LNPs with 2% TE+Triton and further detected with Quant-it RiboGreen assay. LNPs were aliquot into 50 μg/tube aliquots and put the at −80° C. for freezing. Before dosing with LNPs, LNPs were thawed at room temperature.

### Example 2: Analysis of Liver Expression

To assess the ability of the LNPs constructed in Example 1 to target the liver, a biodistribution analysis was conducted in mice. Briefly, animals received an administration of the following and protein expression was assessed using a bioluminescence assay: (1) PBS (negative control), (2) LNP loaded with a control mRNA without any VEE replicon (RNA-no VEE), or (3) LNP loaded with a mRNA with a VEE replicon (RNA-VEE).

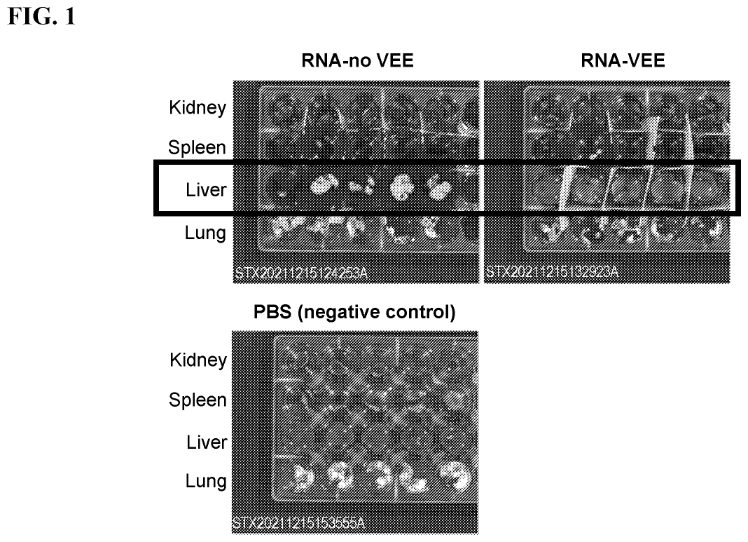

As shown in FIG. 1, in animals that received the RNA-no VEE, there was significant protein expression within the liver. In contrast, in animals that received the RNA-VEE, there was nearly no detectable expression of the protein observed in the liver. The protein expression in other tissues (e.g., spleen and lung) were comparable among the animals from the RNA-no VEE and RNA-VEE groups, demonstrating the specificity of the effect observed in the liver.

The above results demonstrate that the LNPs described herein can selectively avoid and/or reduce expression in the liver, suggesting that the LNPs could allow for greater tolerability (e.g., decreased liver toxicity).

### Example 3: Assessment of Liver Toxicity

To further assess in vivo effect, LNPs of the present disclosure will be administered to animals (e.g., mice) as described in Example 2. Then, liver toxicity will be assessed in the animals via various methods. For instance, in some aspects, the level of liver enzyme (ALT/AST) will be measured, e.g., in the blood of the animals. In some aspects, the general toxicity of the animals will be assessed (e.g., activity, food consumption, body score index, hydration status, or combinations thereof). In some aspects, microscopic assessment of the liver will be conducted to detect any LNP-related impact on the liver.

### Example 4: Construction of Additional Lipid Nanoparticles

To further assess the VEE replicons described herein, additional LNPs were constructed to comprise different lipids described herein (e.g., ionizable lipids, cationic lipids, or lipid-like materials, e.g., lipidoids) as described in Example 1. In total, nine different LNPs were constructed (referred to herein as LNP1-LNP9) and used to encapsulate a mRNA with a VEE replicon (described herein) or a control mRNA without any VEE replicon. The ability of these LNPs to induce payload expression was assessed as described in the subsequent examples.

### Example 5: Analysis of Liver Expression with Lipid Nanoparticles Loaded with a RNA with a VEE Replicon

To further assess the ability of the VEE replicons described herein to selectively avoid and/or reduce expression in the liver, B6.Cg Gt (ROSA) 26Sortm14 (CAG-tdTomato) Hze/J (Ai reporter) mice received a single intravenous administration of LNP1 or LNP2. Both LNP1 and LNP2 were loaded with 20 μg of a VEE replicon comprising a nucleic acid sequence encoding nuclear localization signal (NLS)-Cre. Control animals received an administration of phosphate buffered saline solution (PBS). The Cre gene encodes the Cre protein that is a site-specific DNA recombinase that can catalyze the recombination of DNA between specific sites in a DNA molecule. These sites are known as loxP sequences and contain specific binding sites for Cre that surround a directional core sequence where recombination can occur. The Cre induces tdTomato fluorescence which is indicative of Cre-mediated recombination in the liver tissue. About 72 hours (hrs) after LNP administration, tdTomato fluorescence expression within the liver of the mice was measured using fluorescent IVIS imaging.

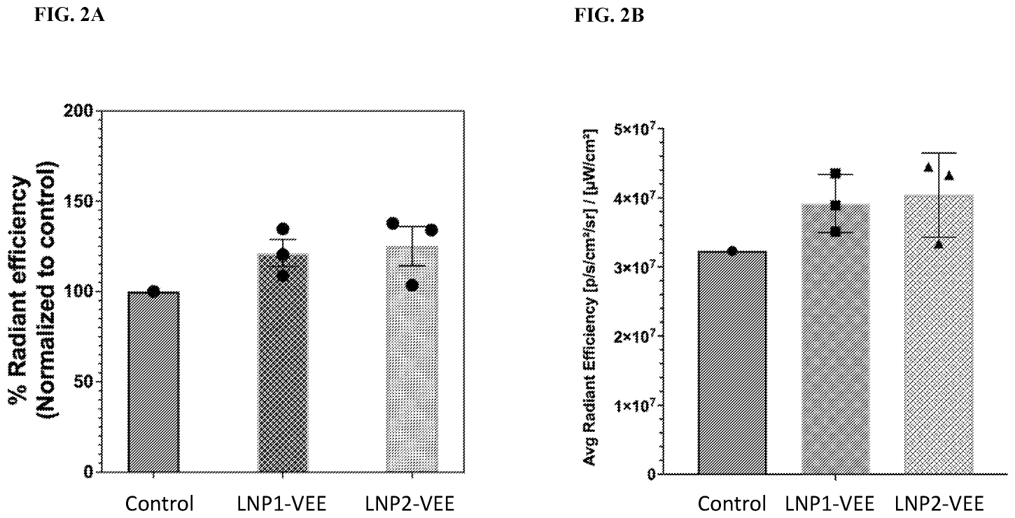

As shown in FIGS. 2A and 2B, the fluorescent signal observed in the liver of mice that received either the LNP1 and LNP2 were not significantly from that observed in the control animals. Additionally, as between animals that received the LNP1 and LNP2, there was also no apparent differences in fluorescence expression in the liver. These results highlight the ability of the VEE replicons described herein to selectively avoid and/or reduce expression in the liver. The results further suggests that liver-avoiding properties of the VEE replicons is independent of at least any differences between LNP1 and LNP2.

### Example 6: Analysis of Liver Expression with Lipid Nanoparticles Loaded with a RNA without VEE Replicon

To confirm that the LNPs described herein can indeed target the liver, B6.Cg Gt (ROSA) 26Sortm14 (CAG-tdTomato) Hze/J (Ai reporter) mice received a single intravenous administration of LNP3, LNP4, LNP5, or LNP6. Each of the LNPs were loaded with 20 μg of an mRNA without any VEE replicon and encoding nuclear localization signal (NLS)-Cre (RNA-no VEE). Control animals received an administration of PBS. About 72 hrs after LNP administration, tdTomato fluorescence expression within the liver of the mice was again measured using fluorescent IVIS imaging. As described in Example 4, it was expected that td-Tomato fluorescence expression within the liver would be indicative of Cre-mediated recombination in the tissue.

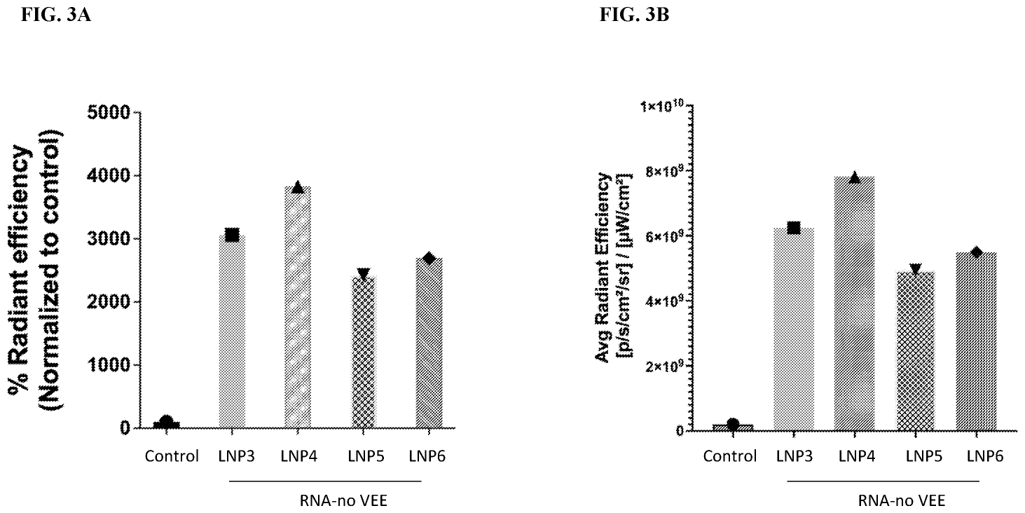

As shown in FIGS. 3A and 3B, in the control animals, there was no detectable fluorescence signal observed in the liver of mice from the control group. In contrast, strong fluorescence signal was observed in the liver of mice that received an administration of one of the tested LNPs (each loaded with the RNA-no VEE).

To further analyze, LNP7 (also loaded with a mRNA without any VEE replicon, wherein the mRNA encodes NLS-Cre; RNA-VEE) was intravenously administered to the tdTomato reporter mice. For comparison, some of the animals received the LNP3 used above (i.e., loaded with a mRNA without any VEE replicon and encoding NLS-Cre). Control animals were treated with PBS alone. Again, about 72 hours after LNP administration, td-Tomato fluorescence was quantified using fluorescent IVIS imaging.

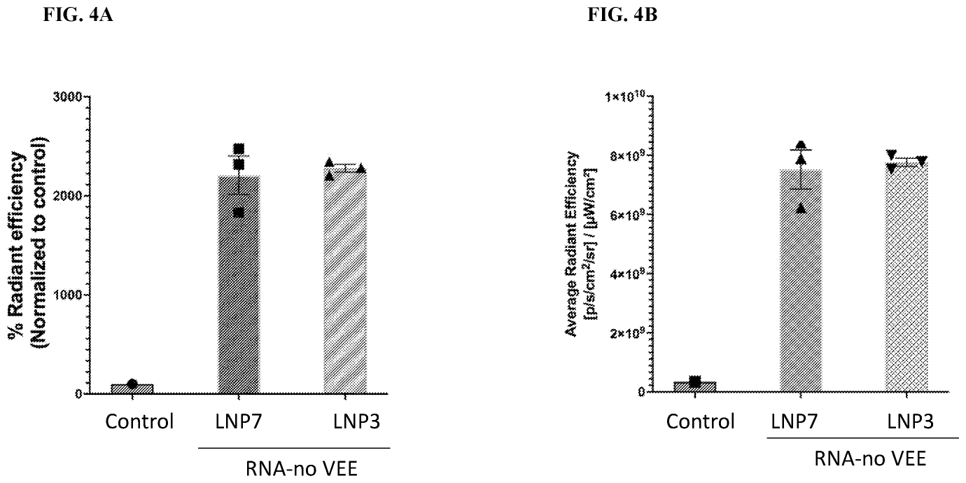

As shown in FIGS. 4A and 4B, compared to the control animals, significant fluorescent signal was observed in the liver of animals that received LNP7. The fluorescence signal was comparable to that observed in mice treated with LNP3.

The results provided in this example collectively demonstrate that the LNPs described herein can indeed target the liver, suggesting that the liver avoidance and/or reduced liver expression observed in the earlier examples is specific to the VEE replicon itself.

### Example 7: Analysis of Firefly Luciferase Expression in the Liver after Administration of Lipid Nanoparticles Loaded with a RNA with a VEE Replicon or a RNA without any VEE Replicon

To confirm the above-described results in a different fluorescence model, C57BL/6J mice received a single administration of one of the following LNPs: LNP8, LNP9, and LNP2. LNP9 and LNP2 were administered to the animals intravenously. LNP8 was administered to the animals intravenously or intratumorally. Each of the LNPs (i.e., LNP8, LNP9, and LNP2) was loaded with 40 μg of a mRNA that includes a VEE replicon and encoding FLuc, a firefly luciferase gene encoding an enzyme that catalyzes the oxygenation of d-luciferin to oxyluciferin, a reaction with produces visible light (530 nm-640 nm). Control animals received an administration of PBS. About 24 hours after the administration, luciferase expression in the liver was measured using bioluminescence IVIS imaging.

As observed earlier with the td-Tomato reporter mice, there was no significant difference in luciferase expression in the liver of C57BL/6J mice that received an administration of LNP2, as compared to the control animals. Similar results were observed in C57BL/6J mice that received either LNP8 or LNP9. Additionally, the route of administration (at least as between intravenous and intratumoral administration) did not appear to have any effect, as no significant differences in luciferase expression was observed in animals that received the LNP8 intravenously or intratumorally.

Again, to confirm that the lipid nanoparticles themselves can indeed target the liver, LNP2, LNP8, and LNP9 were loaded this time with 10 μg of a mRNA without any VEE replicon and encoding firefly luciferase. Then, a single dose of the LNPs was intravenously administered to C57BL/6J mice, and about 24 hours later, luciferase expression in the liver was measured using bioluminescence IVIS imaging.

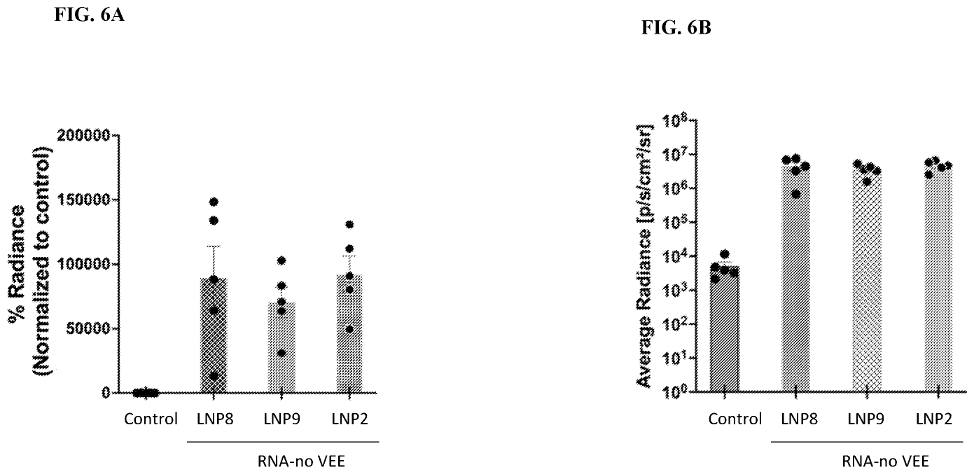

As shown in FIGS. 6A and 6B, the bioluminescence signal in the liver for the LNP groups, including LNP8, LNP9, and LNP2, was significantly higher than that in the controls, indicating specific expression of FLuc-mod RNA in the liver for each LNP tested.

The above results confirm that the LNPs described herein are capable of targeting the liver. The above results also help confirm the liver-avoiding properties of the VEE replicons described herein.

### Example 8: Comparison of Liver Specific Delivery of Lipid Nanoparticles Loaded with Either a RNA with a Vee Replicon or a RNA without any VEE Replicon

To provide a direct comparison between a mRNA without any VEE replicon (RNA-no VEE) and a mRNA with a VEE replicon (RNA-VEE) on liver specificity, C57BL/6J mice received a single intravenous administration of LNP8 loaded with 2 mg/kg of either (1) a RNA-no VEE encoding firefly luciferase or (2) a RNA-VEE encoding firefly luciferase. Then, about 48 hours after administration, bioluminescence IVIS imaging was used to measure luciferase expression in the liver of the animals.

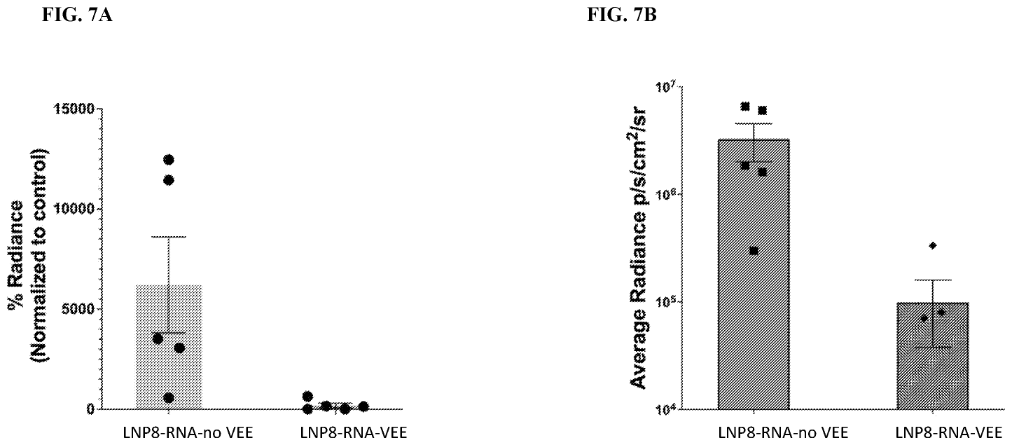

As shown in FIGS. 7A and 7B, the bioluminescence signal in the liver for the RNA-no VEE group was significantly higher than that in the RNA-VEE replicon group. This result further confirms that RNA-no VEE allows expression in the liver while the RNA-VEE prevents expression in the liver. Additionally, the result also further demonstrates that the liver avoiding properties of the VEE replicons described herein are independent of the lipidnanoparticles used.

### Example 9: Lipid Nanoparticles Comprising a RNA with a VEE Replicon and an Ionizable Lipid

To assess the liver avoidance property of the VEE replicons described herein, a lipid nanoparticle comprising an ionizable lipid will be constructed to make a lipid nanoparticle, which will be then used to encapsulate a mRNA with a VEE replicon and encoding a payload (RNA-VEE). The ionizable lipid will be selected from the following: ((4-hydroxybutyl)azanediyl)bis(hexane-6,1-diyl)bis(2-hexyldecanoate) (ALC-0315), heptadecan-9-yl 8-((2-hydroxyethyl)(6-oxo-6-(undecyloxy)hexyl)amino)octanoate (SM-102), heptadecan-9-yl 8-((2-hydroxyethyl)(8-(nonyloxy)-8-oxooctyl)amino)octanoate (Lipid 5), di((Z)-non-2-en-1-yl) 9-((4-(dimethylamino)butanoyl)oxy)heptadecanedioate (L319), 3-(didodecylamino)-N1,N1,4tridodecyl-1-piperazineethanamine (KL10), Nl-[2 (didodecylamino)ethyl]-N1,N4,N4-tridodecyl 1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl-4-(dimethylamino)butanoate (DLin-MC3-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), or any combination thereof.

The above LNP (i.e., comprising an ionizable lipid and a RNA-VEE) will be administered in vivo and then the expression of the encoded payload will be assessed in the liver (e.g., as described in Examples 4-7). In some aspects, liver toxicity will also be assessed in the animals (e.g., as described in Example 3).

### Example 10: Lipid Nanoparticles Comprising a RNA with a VEE Replicon and a Cationic Lipid

To assess the liver avoidance property of the VEE replicons described herein, a lipid nanoparticle comprising a cationic lipid will be constructed to make a lipid nanoparticle, which will be then used to encapsulate a mRNA with a VEE replicon and encoding a payload (RNA-VEE). The cationic lipid will be selected from the following: 1,2-dioleoyl-3-trimethylammonium-propane (DOTAP), lipofectamine, N-[1-(2,3-dioleoyloxy) propyl]-N,N,N-trimethylammonium chloride (DOTMA), 1-[2-(oleoyloxy)ethyl]-2-oleyl-3-(2-hydroxyethyl) imidazolinium chloride (DOTEVI), 2,3-dioleyloxy-N-[2 (sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxy ethyl ammonium bromide (DMRIE), N-(1,2-dioleoyloxyprop-3-yl)-N,N-dimethyl-N-hydroxy ethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), or any combination thereof.

The above LNP (i.e., comprising a cationic lipid and a RNA-VEE) will be administered in vivo and then the expression of the encoded payload will be assessed in the liver (e.g., as described in Examples 4-7). In some aspects, liver toxicity will also be assessed in the animals (e.g., as described in Example 3).

### Example 10: Lipid Nanoparticles Comprising a RNA with a VEE Replicon and a Lipid-Like Material

To assess the liver avoidance property of the VEE replicons described herein, a lipid nanoparticle comprising a lipid-like material will be constructed to make a lipid-like nanoparticle (LLN), which will be then used to encapsulate a mRNA with a VEE replicon and encoding a payload (RNA-VEE). The lipid-like material will be selected from the following: 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200), 3,6-bis(4-(bis(2-hydroxydodecyl)amino)butyl) piperazine2,5-dione (cKK-E12), tetrakis(8-methylnonyl) 3,3′,3″,3′″-(((methylazanediyl)bis(propane-3,1diyl))bis(azanetriyl))tetrapropionate (306Oi10), G0-C14, 5A2-SC8, 3,6-bis(4-(bis((9Z,12Z)-2-hydroxyoctadeca9,12-dien-1-yl)amino)butyl) piperazine-2,5-dione (OF-02), (((3,6-dioxopiperazine-2,5-diyl)bis(butane-4,1-diyl))bis(azanetriyl)) tetrakis(ethane2,1-diyl) (9Z,9′Z,9″Z,9′″Z,12Z,12′Z, 12″Z, 12′″Z)-tetrakis(octadeca-9,12-dienoate) (OF-Deg-Lin), (((3,6-dioxopiperazine-2,5-diyl)bis(butane-4,1-diyl))bis(azanetriyl)) tetrakis(butane-4,1-diyl) (9Z,9′Z,9″Z,9′″Z,12Z,12′Z, 12″Z, 12′″Z)-tetrakis(octadeca-9,12-dienoate) (OF-C4-Deg-Lin), N1,N3,N5-tris(3-(didodecylamino) propyl)benzene1,3,5-tricarboxamide (TT3), Hexa (octan-3-yl) 9,9′,9″,9′″,9″″,9′″″-((((benzene-1,3,5-tricarbonyl) ris (azanediyl))tris(propane-3,1-diyl))tris(azanetriyl)) hexanonanoate (FTT5), PL-1 [disclosed in Nature Communications, 12-7264 (2021), which is hereby incorporated by reference], 98N12-5 [disclosed in Molecular Therapy vol. 17 no. 5 May 2009, which is hereby incorporated by reference], ethyl 5,5-di((Z)-heptadec-8-en-1-yl)-1-(3-(pyrrolidin-1-yl) propyl)-2,5-dihydro-1H-imidazole-2-carboxylate (A2-Iso5-2DC18 (A2)) and A12-Iso5-2DC18 (A12), or any combination thereof.

The above LLP (i.e., comprising a lipid-like material and a RNA-VEE) will be administered in vivo and then the expression of the encoded payload will be assessed in the liver (e.g., as described in Examples 4-7). In some aspects, liver toxicity will also be assessed in the animals (e.g., as described in Example 3).

### Example 12: Lipid Nanoparticles Comprising a RNA with a VEE Replicon and a Polymeric Material

To assess the liver avoidance property of the VEE replicons described herein, a lipid nanoparticle comprising a polymeric material will be constructed to make a polymeric nanoparticle (PNP), which will be then used to encapsulate a mRNA with a VEE replicon and encoding a payload (RNA-VEE). The polymeric material will be selected from the following: polyethyleneimine (PEI), poly (amidoamine) (PAMAM), poly (β-amino ester) (PBAE), poly (2-N,N-dimethylaminoethyl methacrylate) (PDMAEMA), poly (amino acid)s (PAAs), chitosan dextran, cyclodextrin, cellulose, hyaluronic acid, polylactic acid (PLA), poly(lactic-co-glycolic acid) (PLGA), and polycaprolactone (PCL).

The above PNP (i.e., comprising a polymeric material and a RNA VEE) will be administered in vivo and then the expression of the encoded payload will be assessed in the liver (e.g., as described in Examples 4-7). In some aspects, liver toxicity will also be assessed in the animals (e.g., as described in Example 3).

TABLE 4
Exemplary VEE Replicon Sequences

| | |
|---|---|
| Wild-type VEE | ATGGGCGGCGCATGAGAGAAGCCCAGACCAATTACCTACCCAAAATGGA |
| Replicon Sequence | GAAAGTTCACGTTGACATCGAGGAAGACAGCCCATTCCTCAGAGCTTTG |
| (SEQ ID NO: 186) | CAGCGGAGCTTCCCGCAGTTTGAGGTAGAAGCCAAGCAGGTCACTGATA |
| | ATGACCATGCTAATGCCAGAGCGTTTTCGCATCTGGCTTCAAAACTGAT |
| | CGAAACGGAGGTGGACCCATCCGACACGATCCTTGACATTGGAAGTGCG |
| | CCCGCCCGCAGAATGTATTCTAAGCACAAGTATCATTGTATCTGTCCGA |
| | TGAGATGTGCGGAAGATCCGGACAGATTGTATAAGTATGCAACTAAGCT |
| | GAAGAAAAACTGTAAGGAAATAACTGATAAGGAATTGGACAAGAAAATG |
| | AAGGAGCTGGCCGCCGTCATGAGCGACCCTGACCTGGAAACTGAGACTA |
| | TGTGCCTCCACGACGACGAGTCGTGTCGCTACGAAGGGCAAGTCGCTGT |
| | TTACCAGGATGTATACGCGGTTGACGGACCGACAAGTCTCTATCACCAA |
| | GCCAATAAGGGAGTTAGAGTCGCCTACTGGATAGGCTTTGACACCACCC |
| | CTTTTATGTTTAAGAACTTGGCTGGAGCATATCCATCATACTCTACCAA |
| | CTGGGCCGACGAAACCGTGTTAACGGCTCGTAACATAGGCCTATGCAGC |
| | TCTGACGTTATGGAGCGGTCACGTAGAGGGATGTCCATTCTTAGAAAGA |
| | AGTATTTGAAACCATCCAACAATGTTCTATTCTCTGTTGGCTCGACCAT |
| | CTACCACGAGAAGAGGGACTTACTGAGGAGCTGGCACCTGCCGTCTGTA |
| | TTTCACTTACGTGGCAAGCAAAATTACACATGTCGGTGTGAGACTATAG |
| | TTAGTTGCGACGGGTACGTCGTTAAAAGAATAGCTATCAGTCCAGGCCT |
| | GTATGGGAAGCCTTCAGGCTATGCTGCTACGATGCACCGCGAGGGATTC |
| | TTGTGCTGCAAAGTGACAGACACATTGAACGGGGAGAGGGTCTCTTTTC |
| | CCGTGTGCACGTATGTGCCAGCTACATTGTGTGACCAAATGACTGGCAT |
| | ACTGGCAACAGATGTCAGTGCGGACGACGCGCAAAAACTGCTGGTTGGG |
| | CTCAACCAGCGTATAGTCGTCAACGGTCGCACCCAGAGAAACACCAATA |
| | CCATGAAAAATTACCTTTTGCCCGTAGTGGCCCAGGCATTTGCTAGGTG |
| | GGCAAAGGAATATAAGGAAGATCAAGAAGATGAAAGGCCACTAGGACTA |
| | CGAGATAGACAGTTAGTCATGGGGTGTTGTTGGGCTTTTAGAAGGCACA |
| | AGATAACATCTATTTATAAGCGCCCGGATACCCAAACCATCATCAAAGT |
| | GAACAGCGATTTCCACTCATTCGTGCTGCCCAGGATAGGCAGTAACACA |
| | TTGGAGATCGGGCTGAGAACAAGAATCAGGAAAATGTTAGAGGAGCACA |
| | AGGAGCCGTCACCTCTCATTACCGCCGAGGACGTACAAGAAGCTAAGTG |
| | CGCAGCCGATGAGGCTAAGGAGGTGCGTGAAGCCGAGGAGTTGCGCGCA |
| | GCTCTACCACCTTTGGCAGCTGATGTTGAGGAGCCCACTCTGGAGGCAG |
| | ACGTCGACTTGATGTTACAAGAGGCTGGGGCCGGCTCAGTGGAGACACC |
| | TCGTGGCTTGATAAAGGTTACCAGCTACGATGGCGAGGACAAGATCGGC |
| | TCTTACGCTGTGCTTTCTCCGCAGGCTGTACTCAAGAGTGAAAAATTAT |
| | CTTGCATCCACCCTCTCGCTGAACAAGTCATAGTGATAACACACTCTGG |
| | CCGAAAAGGGCGTTATGCCGTGGAACCATACCATGGTAAAGTAGTGGTG |
| | CCAGAGGGACATGCAATACCCGTCCAGGACTTTCAAGCTCTGAGTGAAA |
| | GTGCCACCATTGTGTACAACGAACGTGAGTTCGTAAACAGGTACCTGCA |
| | CCATATTGCCACACATGGAGGAGCGCTGAACACTGATGAAGAATATTAC |
| | AAAACTGTCAAGCCCAGCGAGCACGACGGCGAATACCTGTACGACATCG |
| | ACAGGAAACAGTGCGTCAAGAAAGAACTAGTCACTGGGCTAGGGCTCAC |
| | AGGCGAGCTGGTGGATCCTCCCTTCCATGAATTCGCCTACGAGAGTCTG |
| | AGAACACGACCAGCCGCTCCTTACCAAGTACCAACCATAGGGGTGTATG |
| | GCGTGCCAGGATCAGGCAAGTCTGGCATCATTAAAAGCGCAGTCACCAA |
| | AAAAGATCTAGTGGTGAGCGCCAAGAAAGAAAACTGTGCAGAAATTATA |
| | AGGGACGTCAAGAAAATGAAAGGGCTGGACGTCAATGCCAGAACTGTGG |
| | ACTCAGTGCTCTTGAATGGATGCAAACACCCCGTAGAGACCCTGTATAT |
| | TGACGAAGCTTTTGCTTGTCATGCAGGTACTCTCAGAGCGCTCATAGCC |
| | ATTATAAGACCTAAAAAGGCAGTGCTCTGCGGGGATCCCAAACAGTGCG |
| | GTTTTTTTAACATGATGTGCCTGAAAGTGCATTTTAACCACGAGATTTG |
| | CACACAAGTCTTCCACAAAAGCATCTCTCGCCGTTGCACTAAATCTGTG |
| | ACTTCGGTCGTCTCAACCTTGTTTTACGACAAAAAAATGAGAACGACGA |
| | ATCCGAAAGAGACTAAGATTGTGATTGACACTACCGGCAGTACCAAACC |
| | TAAGCAGGACGATCTCATTCTCACTTGTTTCAGAGGGTGGGTGAAGCAG |
| | TTGCAAATAGATTACAAAGGCAACGAAATAATGACGGCAGCTGCCTCTC |
| | AAGGGCTGACCCGTAAAGGTGTGTATGCCGTTCGGTACAAGGTGAATGA |
| | AAATCCTCTGTACGCACCCACCTCAGAACATGTGAACGTCCTACTGACC |
| | CGCACGGAGGACCGCATCGTGTGGAAAACACTAGCCGGCGACCCATGGA |
| | TAAAAACACTGACTGCCAAGTACCCTGGGAATTTCACTGCCACGATAGA |
| | GGAGTGGCAAGCAGAGCATGATGCCATCATGAGGCACATCTTGGAGAGA |
| | CCGGACCCTACCGACGTCTTCCAGAATAAGGCAAACGTGTGTTGGGCCA |
| | AGGCTTTAGTGCCGGTGCTGAAGACCGCTGGCATAGACATGACCACTGA |
| | ACAATGGAACACTGTGGATTATTTTGAAACGGACAAAGCTCACTCAGCA |
| | GAGATAGTATTGAACCAACTATGCGTGAGGTTCTTTGGACTCGATCTGG |
| | ACTCCGGTCTATTTTCTGCACCCACTGTTCCGTTATCCATTAGGAATAA |
| | TCACTGGGATAACTCCCCGTCGCCTAACATGTACGGGCTGAATAAAGAA |
| | GTGGTCCGTCAGCTCTCTCGCAGGTACCCACAACTGCCTCGGGCAGTTG |
| | CCACTGGAAGAGTCTATGACATGAACACTGGTACACTGCGCAATTATGA |
| | TCCGCGCATAAACCTAGTACCTGTAAACAGAAGACTGCCTCATGCTTTA |
| | GTCCTCCACCATAATGAACACCCACAGAGTGACTTTTCTTCATTCGTCA |
| | GCAAATTGAAGGGCAGAACTGTCCTGGTGGTCGGGGAAAAGTTGTCCGT |
| | CCCAGGCAAAATGGTTGACTGGTTGTCAGACCGGCCTGAGGCTACCTTC |
| | AGAGCTCGGCTGGATTTAGGCATCCCAGGTGATGTGCCCAAATATGACA |
| | TAATATTTGTTAATGTGAGGACCCCATATAAATACCATCACTATCAGCA |
| | GTGTGAAGACCATGCCATTAAGCTTAGCATGTTGACCAAGAAAGCTTGT |
| | CTGCATCTGAATCCCGGCGGAACCTGTGTCAGCATAGGTTATGGTTACG |
| | CTGACAGGGCCAGCGAAAGCATCATTGGTGCTATAGCGCGGCAGTTCAA |
| | GTTTTCCCGGGTATGCAAACCGAAATCCTCACTTGAAGAGACGGAAGTT |
| | CTGTTTGTATTCATTGGGTACGATCGCAAGGCCCGTACGCACAATTCTT |
| | ACAAGCTTTCATCAACCTTGACCAACATTTATACAGGTTCCAGACTCCA |
| | CGAAGCCGGATGTGCACCCTCATATCATGTGGTGCGAGGGGATATTGCC |
| | ACGGCCACCGAAGGAGTGATTATAAATGCTGCTAACAGCAAAGGACAAC |
| | CTGGCGGAGGGGTGTGCGGAGCGCTGTATAAGAAATTCCCGGAAAGCTT |
| | CGATTTACAGCCGATCGAAGTAGGAAAAGCGCGACTGGTCAAAGGTGCA |
| | GCTAAACATATCATTCATGCCGTAGGACCAAACTTCAACAAAGTTTCGG |
| | AGGTTGAAGGTGACAAACAGTTGGCAGAGGCTTATGAGTCCATCGCTAA |
| | GATTGTCAACGATAACAATTACAAGTCAGTAGCGATTCCACTGTTGTCC |
| | ACCGGCATCTTTTCCGGGAACAAAGATCGACTAACCCAATCATTGAACC |
| | ATTTGCTGACAGCTTTAGACACCACTGATGCAGATGTAGCCATATACTG |
| | CAGGGACAAGAAATGGGAAATGACTCTCAAGGAAGCAGTGGCTAGGAGA |
| | GAAGCAGTGGAGGAGATATGCATATCCGACGACTCTTCAGTGACAGAAC |
| | CTGATGCAGAGCTGGTGAGGGTGCATCCGAAGAGTTCTTTGGCTGGAAG |
| | GAAGGGCTACAGCACAAGCGATGGCAAAACTTTCTCATATTTGGAAGGG |
| | ACCAAGTTTCACCAGGCGGCCAAGGATATAGCAGAAATTAATGCCATGT |
| | GGCCCGTTGCAACGGAGGCCAATGAGCAGGTATGCATGTATATCCTCGG |
| | AGAAAGCATGAGCAGTATTAGGTCGAAATGCCCCGTCGAAGAGTCGGAA |
| | GCCTCCACACCACCTAGCACGCTGCCTTGCTTGTGCATCCATGCCATGA |
| | CTCCAGAAAGAGTACAGCGCCTAAAAGCCTCACGTCCAGAACAAATTAC |
| | TGTGTGCTCATCCTTTCCATTGCCGAAGTATAGAATCACTGGTGTGCAG |
| | AAGATCCAATGCTCCCAGCCTATATTGTTCTCACCGAAAGTGCCTGCGT |
| | ATATTCATCCAAGGAAGTATCTCGTGGAAACACCACCGGTAGACGAGAC |
| | TCCGGAGCCATCGGCAGAGAACCAATCCACAGAGGGGACACCTGAACAA |
| | CCACCACTTATAACCGAGGATGAGACCAGGACTAGAACGCCTGAGCCGA |
| | TCATCATCGAAGAGGAAGAAGAGGATAGCATAAGTTTGCTGTCAGATGG |
| | CCCGACCCACCAGGTGCTGCAAGTCGAGGCAGACATTCACGGGCCGCCC |
| | TCTGTATCTAGCTCATCCTGGTCCATTCCTCATGCATCCGACTTTGATG |
| | TGGACAGTTTATCCATACTTGACACCCTGGAGGGAGCTAGCGTGACCAG |
| | CGGGGCAACGTCAGCCGAGACTAACTCTTACTTCGCAAAGAGTATGGAG |
| | TTTCTGGCGCGACCGGTGCCTGCGCCTCGAACAGTATTCAGGAACCCTC |
| | CACATCCCGCTCCGCGCACAAGAACACCGTCACTTGCACCCAGCAGGGC |
| | CTGCTCGAGAACCAGCCTAGTTTCCACCCCGCCAGGCGTGAATAGGGTG |
| | ATCACTAGAGAGGAGCTCGAGGCGCTTACCCCGTCACGCACTCCTAGCA |
| | GGTCGGTCTCGAGAACCAGCCTGGTCTCCAACCCGCCAGGCGTAAATAG |
| | GGTGATTACAAGAGAGGAGTTTGAGGCGTTCGTAGCACAACAACAATGA |
| | CGGTTTGATGCGGGTGCATACATCTTTTCCTCCGACACCGGTCAAGGGC |
| | ATTTACAACAAAAATCAGTAAGGCAAACGGTGCTATCCGAAGTGGTGTT |
| | GGAGAGGACCGAATTGGAGATTTCGTATGCCCCGCGCCTCGACCAAGAA |
| | AAAGAAGAATTACTACGCAAGAAATTACAGTTAAATCCCACACCTGCTA |
| | ACAGAAGCAGATACCAGTCCAGGAAGGTGGAGAACATGAAAGCCATAAC |
| | AGCTAGACGTATTCTGCAAGGCCTAGGGCATTATTTGAAGGCAGAAGGA |
| | AAAGTGGAGTGCTACCGAACCCTGCATCCTGTTCCTTTGTATTCATCTA |
| | GTGTGAACCGTGCCTTTTCAAGCCCCAAGGTCGCAGTGGAAGCCTGTAA |
| | CGCCATGTTGAAAGAGAACTTTCCGACTGTGGCTTCTTACTGTATTATT |
| | CCAGAGTACGATGCCTATTTGGACATGGTTGACGGAGCTTCATGCTGCT |
| | TAGACACTGCCAGTTTTTGCCCTGCAAAGCTGCGCAGCTTTCCAAAGAA |
| | ACACTCCTATTTGGAACCCACAATACGATCGGCAGTGCCTTCAGCGATC |
| | CAGAACACGCTCCAGAACGTCCTGGCAGCTGCCACAAAAAGAAATTGCA |
| | ATGTCACGCAAATGAGAGAATTGCCCGTATTGGATTCGGCGGCCTTTAA |
| | TGTGGAATGCTTCAAGAAATATGCGTGTAATAATGAATATTGGGAAACG |
| | TTTAAAGAAAACCCCATCAGGCTTACTGAAGAAAACGTGGTAAATTACA |
| | TTACCAAATTAAAAGGACCAAAAGCTGCTGCTCTTTTTGCGAAGACACA |
| | TAATTTGAATATGTTGCAGGACATACCAATGGACAGGTTTGTAATGGAC |
| | TTAAAGAGAGACGTGAAAGTGACTCCAGGAACAAAACATACTGAAGAAC |
| | GGCCCAAGGTACAGGTGATCCAGGCTGCCGATCCGCTAGCAACAGCGTA |
| | TCTGTGCGGAATCCACCGAGAGCTGGTTAGGAGATTAAATGCGGTCCTG |
| | CTTCCGAACATTCATACACTGTTTGATATGTCGGCTGAAGACTTTGACG |
| | CTATTATAGCCGAGCACTTCCAGCCTGGGGATTGTGTTCTGGAAACTGA |
| | CATCGCGTCGTTTGATAAAAGTGAGGACGACGCCATGGCTCTGACCGCG |
| | TTAATGATTCTGGAAGACTTAGGTGTGGACGCAGAGCTGTTGACGCTGA |
| | TTGAGGCGGCTTTCGGCGAAATTTCATCAATACATTTGCCCACTAAAAC |
| | TAAATTTAAATTCGGAGCCATGATGAAATCTGGAATGTTCCTCACACTG |
| | TTTGTGAACACAGTCATTAACATTGTAATCGCAAGCAGAGTGTTGAGAG |
| | AACGGCTAACCGGATCACCATGTGCAGCATTCATTGGAGATGACAATAT |
| | CGTGAAAGGAGTCAAATCGGACAAATTAATGGCAGACAGGTGCGCCACC |
| | TGGTTGAATATGGAAGTCAAGATTATAGATGCTGTGGTGGGCGAGAAAG |
| | CGCCTTATTTCTGTGGAGGGTTTATTTTGTGTGACTCCGTGACCGGCAC |
| | AGCGTGCCGTGTGGCAGACCCCCTAAAAAGGCTGTTTAAGCTTGGCAAA |
| | CCTCTGGCAGCAGACGATGAACATGATGATGACAGGAGAAGGGCATTGC |
| | ATGAAGAGTCAACACGCTGGAACCGAGTGGGTATTCTTTCAGAGCTGTG |
| | CAAGGCAGTAGAATCAAGGTATGAAACCGTAGGAACTTCCATCATAGTT |
| | ATGGCCATGACTACTCTAGCTAGCAGTGTTAAATCATTCAGCTACCTGA |
| | GAGGGGCCCCTATAACTCTCTACGGCTAACCTGAATGGACTACGACATA |
| | GTCTAGTCCGCCAAGTCTAGCATATGGGCGCGCCCTCAGCATCGATTGA |
| | ATTGGCCACCATGGTGAGCAAGGGCGAGGAGGATAACATGGCCATCATC |
| | AAGGAGTTCATGCGCTTCAAGGTGCACATGGAGGGCTCCGTGAACGGCC |
| | ACGAGTTCGAGATCGAGGGCGAGGGCGAGGGCCGCCCCTACGAGGGCAC |
| | CCAGACCGCCAAGCTGAAGGTGACCAAGGGTGGCCCCCTGCCCTTCGCC |
| | TGGGACATCCTGTCCCCTCAGTTCATGTACGGCTCCAAGGCCTACGTGA |
| | AGCACCCCGCCGACATCCCCGACTACTTGAAGCTGTCCTTCCCCGAGGG |
| | CTTCAAGTGGGAGCGCGTGATGAACTTCGAGGACGGCGGCGTGGTGACC |
| | GTGACCCAGGACTCCTCCCTGCAGGACGGCGAGTTCATCTACAAGGTGA |
| | AGCTGCGCGGCACCAACTTCCCCTCCGACGGCCCCGTAATGCAGAAGAA |
| | GACCATGGGCTGGGAGGCCTCCTCCGAGCGGATGTACCCCGAGGACGGC |
| | GCCCTGAAGGGCGAGATCAAGCAGAGGCTGAAGCTGAAGGACGGCGGCC |
| | ACTACGACGCTGAGGTCAAGACCACCTACAAGGCCAAGAAGCCCGTGCA |
| | GCTGCCCGGCGCCTACAACGTCAACATCAAGTTGGACATCACCTCCCAC |
| | AACGAGGACTACACCATCGTGGAACAGTACGAACGCGCCGAGGGCCGCC |
| | ACTCCACCGGCGGCATGGACGAGCTGTACAAGTAGGAATTGGCAAGCTG |
| | CTTACATAGAACTCGCGGCGATTGGCATGCCGCCTTAAAATTTTTATTT |
| | TATTTTTCTTTTCTTTTCCGAATCGGATTTTGTTTTTAATATTTCAAAA |
| | AAAAAAAAAAAAAAAAAAAAAACGCGTCGAGGGGAATTAATTCTTGAAG |
| | ACGAAAGGGCCAGGTGGCACTTTTCGGGGAAATGTGCGCGGAACCCCTA |
| | TTTGTTTATTTTTCTAAATACATTCAAATATGTATCCGCTCATGAGACA |
| | ATAACCCTGATAAATGCTTCAATAATATTGAAAAAGGAAGAGTATGAGT |
| | ATTCAACATTTCCGTGTCGCCCTTATTCCCTTTTTTGCGGCATTTTGCC |
| | TTCCTGTTTTTGCTCACCCAGAAACGCTGGTGAAAGTAAAAGATGCTGA |
| | AGATCAGTTGGGTGCACGAGTGGGTTACATCGAACTGGATCTCAACAGC |
| | GGTAAGATCCTTGAGAGTTTTCGCCCCGAAGAACGTTTTCCAATGATGA |
| | GCACTTTTAAAGTTCTGCTATGTGGCGCGGTATTATCCCGTGTTGACGC |
| | CGGGCAAGAGCAACTCGGTCGCCGCATACACTATTCTCAGAATGACTTG |
| | GTTGAGTACTCACCAGTCACAGAAAAGCATCTTACGGATGGCATGACAG |
| | TAAGAGAATTATGCAGTGCTGCCATAACCATGAGTGATAACACTGCGGC |
| | CAACTTACTTCTGACAACGATCGGAGGACCGAAGGAGCTAACCGCTTTT |
| | TTGCACAACATGGGGGATCATGTAACTCGCCTTGATCGTTGGGAACCGG |
| | AGCTGAATGAAGCCATACCAAACGACGAGCGTGACACCACGATGCCTGT |
| | AGCAATGGCAACAACGTTGCGCAAACTATTAACTGGCGAACTACTTACT |
| | CTAGCTTCCCGGCAACAATTAATAGACTGGATGGAGGCGGATAAAGTTG |
| | CAGGACCACTTCTGCGCTCGGCCCTTCCGGCTGGCTGGTTTATTGCTGA |
| | TAAATCTGGAGCCGGTGAGCGTGGGTCTCGCGGTATCATTGCAGCACTG |
| | GGGCCAGATGGTAAGCCCTCCCGTATCGTAGTTATCTACACGACGGGGA |
| | GTCAGGCAACTATGGATGAACGAAATAGACAGATCGCTGAGATAGGTGC |
| | CTCACTGATTAAGCATTGGTAACTGTCAGACCAAGTTTACTCATATATA |
| | CTTTAGATTGATTTAAAACTTCATTTTTAATTTAAAAGGATCTAGGTGA |
| | AGATCCTTTTTGATAATCTCATGACCAAAATCCCTTAACGTGAGTTTTC |
| | GTTCCACTGAGCGTCAGACCCCGTAGAAAAGATCAAAGGATCTTCTTGA |
| | GATCCTTTTTTTCTGCGCGTAATCTGCTGCTTGCAAACAAAAAAACCAC |
| | CGCTACCAGCGGTGGTTTGTTTGCCGGATCAAGAGCTACCAACTCTTTT |
| | TCCGAAGGTAACTGGCTTCAGCAGAGCGCAGATACCAAATACTGTCCTT |
| | CTAGTGTAGCCGTAGTTAGGCCACCACTTCAAGAACTCTGTAGCACCGC |
| | CTACATACCTCGCTCTGCTAATCCTGTTACCAGTGGCTGCTGCCAGTGG |
| | CGATAAGTCGTGTCTTACCGGGTTGGACTCAAGACGATAGTTACCGGAT |
| | AAGGCGCAGCGGTCGGGCTGAACGGGGGGTTCGTGCACACAGCCCAGCT |
| | TGGAGCGAACGACCTACACCGAACTGAGATACCTACAGCGTGAGCATTG |
| | AGAAAGCGCCACGCTTCCCGAAGGGAGAAAGGCGGACAGGTATCCGGTA |
| | AGCGGCAGGGTCGGAACAGGAGAGCGCACGAGGGAGCTTCCAGGGGGAA |
| | ACGCCTGGTATCTTTATAGTCCTGTCGGGTTTCGCCACCTCTGACTTGA |
| | GCGTCGATTTTTGTGATGCTCGTCAGGGGGGCGGAGCCTATGGAAAAAC |
| | GCCAGCAACGCGAGCTCTAATACGACTCACTATAG |
| VEE Replicon | gctcttctaagTAATACGACTCACTATAATGGGCGGCGCATGAGAGAAG |
| Sequence (SEQ ID | CCCAGACCAATTACCTACCCAAAATGGAGAAAGTTCACGTTGACATCGA |
| NO: 187) | GGAAGACAGCCCATTCCTCAGAGCTTTGCAGCGGAGCTTCCCGCAGTTT |
| | GAGGTAGAAGCCAAGCAGGTCACTGATAATGACCATGCTAATGCCAGAG |
| | CGTTTTCGCATCTGGCTTCAAAACTGATCGAAACGGAGGTGGACCCATC |
| | CGACACGATCCTTGACATTGGAAGTGCGCCCGCCCGCAGAATGTATTCT |
| | AAGCACAAGTATCATTGTATCTGTCCGATGAGATGTGCGGAAGATCCGG |
| | ACAGATTGTATAAGTATGCAACTAAGCTGAAGAAAAACTGTAAGGAAAT |
| | AACTGATAAGGAATTGGACAAGAAAATGAAGGAGCTCGCCGCCGTCATG |
| | AGCGACCCTGACCTGGAAACTGAGACTATGTGCCTCCACGACGACGAGT |
| | CGTGTCGCTACGAAGGGCAAGTCGCTGTTTACCAGGATGTATACGCGGT |
| | TGACGGACCGACAAGTCTCTATCACCAAGCCAATAAGGGAGTTAGAGTC |
| | GCCTACTGGATAGGCTTTGACACCACCCCTTTTATGTTTAAGAACTTGG |
| | CTGGAGCATATCCATCATACTCTACCAACTGGGCCGACGAAACCGTGTT |
| | AACGGCTCGTAACATAGGCCTATGCAGCTCTGACGTTATGGAGCGGTCA |
| | CGTAGAGGGATGTCCATTCTTAGAAAGAAGTATTTGAAACCATCCAACA |
| | ATGTTCTATTCTCTGTTGGCTCGACCATCTACCACGAGAAGAGGGACTT |
| | ACTGAGGAGCTGGCACCTGCCGTCTGTATTTCACTTACGTGGCAAGCAA |
| | AATTACACATGTCGGTGTGAGACTATAGTTAGTTGCGACGGGTACGTCG |
| | TTAAAAGAATAGCTATCAGTCCAGGCCTGTATGGGAAGCCTTCAGGCTA |
| | TGCTGCTACGATGCACCGCGAGGGATTCTTGTGCTGCAAAGTGACAGAC |
| | ACATTGAACGGGGAGAGGGTCTCTTTTCCCGTGTGCACGTATGTGCCAG |
| | CTACATTGTGTGACCAAATGACTGGCATACTGGCAACAGATGTCAGTGC |
| | GGACGACGCGCAAAAACTGCTGGTTGGGCTCAACCAGCGTATAGTCGTC |
| | AACGGTCGCACCCAGAGAAACACCAATACCATGAAAAATTACCTTTTGC |
| | CCGTAGTGGCCCAGGCATTTGCTAGGTGGGCAAAGGAATATAAGGAAGA |
| | TCAAGAAGATGAAAGGCCACTAGGACTACGAGATAGACAGTTAGTCATG |
| | GGGTGTTGTTGGGCTTTTAGAAGGCACAAGATAACATCTATTTATAAGC |
| | GCCCGGATACCCAAACCATCATCAAAGTGAACAGCGATTTCCACTCATT |
| | CGTGCTGCCCAGGATAGGCAGTAACACATTGGAGATCGGGCTGAGAACA |
| | AGAATCAGGAAAATGTTAGAGGAGCACAAGGAGCCGTCACCTCTCATTA |
| | CCGCCGAGGACGTACAAGAAGCTAAGTGCGCAGCCGATGAGGCTAAGGA |
| | GGTGCGTGAAGCCGAGGAGTTGCGCGCAGCTCTACCACCTTTGGCAGCT |
| | GATGTTGAGGAGCCCACTCTGGAAGCCGATGTCGACTTGATGTTACAAG |
| | AGGCTGGGGCCGGCTCAGTGGAGACACCTCGTGGCTTGATAAAGGTTAC |
| | CAGCTACGATGGCGAGGACAAGATCGGCTCTTACGCTGTGCTTTCTCCG |
| | CAGGCTGTACTCAAGAGTGAAAAATTATCTTGCATCCACCCTCTCGCTG |
| | AACAAGTCATAGTGATAACACACTCTGGCCGAAAAGGGCGTTATGCCGT |
| | GGAACCATACCATGGTAAAGTAGTGGTGCCAGAGGGACATGCAATACCC |
| | GTCCAGGACTTTCAAGCTCTGAGTGAAAGTGCCACCATTGTGTACAACG |
| | AACGTGAGTTCGTAAACAGGTACCTGCACCATATTGCCACACATGGAGG |
| | AGCGCTGAACACTGATGAAGAATATTACAAAACTGTCAAGCCCAGCGAG |
| | CACGACGGCGAATACCTGTACGACATCGACAGGAAACAGTGCGTCAAGA |
| | AAGAACTAGTCACTGGGCTAGGGCTCACAGGCGAGCTGGTGGATCCTCC |
| | CTTCCATGAATTCGCCTACGAGAGTCTGAGAACACGACCAGCCGCTCCT |
| | TACCAAGTACCAACCATAGGGGTGTATGGCGTGCCAGGATCAGGCAAGT |
| | CTGGCATCATTAAAAGCGCAGTCACCAAAAAAGATCTAGTGGTGAGCGC |
| | CAAGAAAGAAAACTGTGCAGAAATTATAAGGGACGTCAAGAAAATGAAA |
| | GGGCTGGACGTCAATGCCAGAACTGTGGACTCAGTGCTCTTGAATGGAT |
| | GCAAACACCCCGTAGAGACCCTGTATATTGACGAAGCTTTTGCTTGTCA |
| | TGCAGGTACTCTCAGAGCGCTCATAGCCATTATAAGACCTAAAAAGGCA |
| | GTGCTCTGCGGGGATCCCAAACAGTGCGGTTTTTTTAACATGATGTGCC |
| | TGAAAGTGCATTTTAACCACGAGATTTGCACACAAGTCTTCCACAAAAG |
| | CATCTCTCGCCGTTGCACTAAATCTGTGACTTCGGTCGTCTCAACCTTG |
| | TTTTACGACAAAAAAATGAGAACGACGAATCCGAAAGAGACTAAGATTG |
| | TGATTGACACTACCGGCAGTACCAAACCTAAGCAGGACGATCTCATTCT |
| | CACTTGTTTCAGAGGGTGGGTGAAGCAGTTGCAAATAGATTACAAAGGC |
| | AACGAAATAATGACGGCAGCTGCCTCTCAAGGGCTGACCCGTAAAGGTG |
| | TGTATGCCGTTCGGTACAAGGTGAATGAAAATCCTCTGTACGCACCCAC |
| | CTCAGAACATGTGAACGTCCTACTGACCCGCACGGAGGACCGCATCGTG |
| | TGGAAAACACTAGCCGGCGACCCATGGATAAAAACACTGACTGCCAAGT |
| | ACCCTGGGAATTTCACTGCCACGATAGAGGAGTGGCAAGCAGAGCATGA |
| | TGCCATCATGAGGCACATCTTGGAGAGACCGGACCCTACCGACGTCTTC |
| | CAGAATAAGGCAAACGTGTGTTGGGCCAAGGCTTTAGTGCCGGTGCTGA |
| | AGACCGCTGGCATAGACATGACCACTGAACAATGGAACACTGTGGATTA |
| | TTTTGAAACGGACAAAGCTCACTCAGCAGAGATAGTATTGAACCAACTA |
| | TGCGTGAGGTTCTTTGGACTCGATCTGGACTCCGGTCTATTTTCTGCAC |
| | CCACTGTTCCGTTATCCATTAGGAATAATCACTGGGATAACTCCCCGTC |
| | GCCTAACATGTACGGGCTGAATAAAGAAGTGGTCCGTCAGCTCTCTCGC |
| | AGGTACCCACAACTGCCTCGGGCAGTTGCCACTGGAAGAGTCTATGACA |
| | TGAACACTGGTACACTGCGCAATTATGATCCGCGCATAAACCTAGTACC |
| | TGTAAACAGAAGACTGCCTCATGCTTTAGTCCTCCACCATAATGAACAC |
| | CCACAGAGTGACTTTTCTTCATTCGTCAGCAAATTGAAGGGCAGAACTG |
| | TCCTGGTGGTCGGGGAAAAGTTGTCCGTCCCAGGCAAAATGGTTGACTG |
| | GTTGTCAGACCGGCCTGAGGCTACCTTCAGAGCTCGGCTGGATTTAGGC |
| | ATCCCAGGTGATGTGCCCAAATATGACATAATATTTGTTAATGTGAGGA |
| | CCCCATATAAATACCATCACTATCAGCAGTGTGAAGACCATGCCATTAA |
| | GCTTAGCATGTTGACCAAGAAAGCTTGTCTGCATCTGAATCCCGGCGGA |
| | ACCTGTGTCAGCATAGGTTATGGTTACGCTGACAGGGCCAGCGAAAGCA |
| | TCATTGGTGCTATAGCGCGGCAGTTCAAGTTTTCCCGGGTATGCAAACC |
| | GAAATCCTCACTTGAAGAGACGGAAGTTCTGTTTGTATTCATTGGGTAC |
| | GATCGCAAGGCCCGTACGCACAATCCTTACAAGCTTTCATCAACCTTGA |
| | CCAACATTTATACAGGTTCCAGACTCCACGAAGCCGGATGTGCACCCTC |
| | ATATCATGTGGTGCGAGGGGATATTGCCACGGCCACCGAAGGAGTGATT |
| | ATAAATGCTGCTAACAGCAAAGGACAACCTGGCGGAGGGGTGTGCGGAG |
| | CGCTGTATAAGAAATTCCCGGAAAGCTTCGATTTACAGCCGATCGAAGT |
| | AGGAAAAGCGCGACTGGTCAAAGGTGCAGCTAAACATATCATTCATGCC |
| | GTAGGACCAAACTTCAACAAAGTTTCGGAGGTTGAAGGTGACAAACAGT |
| | TGGCAGAGGCTTATGAGTCCATCGCTAAGATTGTCAACGATAACAATTA |
| | CAAGTCAGTAGCGATTCCACTGTTGTCCACCGGCATCTTTTCCGGGAAC |
| | AAAGATCGACTAACCCAATCATTGAACCATTTGCTGACAGCTTTAGACA |
| | CCACTGATGCAGATGTAGCCATATACTGCAGGGACAAGAAATGGGAAAT |
| | GACTCTCAAGGAAGCAGTGGCTAGGAGAGAAGCAGTGGAGGAGATATGC |
| | ATATCCGACGACTCTTCAGTGACAGAACCTGATGCAGAGCTGGTGAGGG |
| | TGCATCCGAAGAGTTCTTTGGCTGGAAGGAAGGGCTACAGCACAAGCGA |
| | TGGCAAAACTTTCTCATATTTGGAAGGGACCAAGTTTCACCAGGCGGCC |
| | AAGGATATAGCAGAAATTAATGCCATGTGGCCCGTTGCAACGGAGGCCA |
| | ATGAGCAGGTATGCATGTATATCCTCGGAGAAAGCATGAGCAGTATTAG |
| | GTCGAAATGCCCCGTCGAAGAGTCGGAAGCCTCCACACCACCTAGCACG |
| | CTGCCTTGCTTGTGCATCCATGCCATGACTCCAGAAAGAGTACAGCGCC |
| | TAAAAGCCTCACGTCCAGAACAAATTACTGTGTGCTCATCCTTTCCATT |
| | GCCGAAGTATAGAATCACTGGTGTGCAGAAGATCCAATGCTCCCAGCCT |
| | ATATTGTTCTCACCGAAAGTGCCTGCGTATATTCATCCAAGGAAGTATC |
| | TCGTGGAAACACCACCGGTAGACGAGACTCCGGAGCCATCGGCAGAGAA |
| | CCAATCCACAGAGGGGACACCTGAACAACCACCACTTATAACCGAGGAT |
| | GAGACCAGGACTAGAACGCCTGAGCCGATCATCATCGAAGAGGAAGAAG |
| | AGGATAGCATAAGTTTGCTGTCAGATGGCCCGACCCACCAGGTGCTGCA |
| | AGTCGAGGCAGACATTCACGGGCCGCCCTCTGTATCTAGCTCATCCTGG |
| | TCCATTCCTCATGCATCCGACTTTGATGTGGACAGTTTATCCATACTTG |
| | ACACCCTGGAGGGAGCTAGCGTGACCAGCGGGGCAACGTCAGCCGAGAC |
| | TAACTCTTACTTCGCAAAGAGTATGGAGTTTCTGGCGCGACCGGTGCCT |
| | GCGCCTCGAACAGTATTCAGGAACCCTCCACATCCCGCTCCGCGCACAA |
| | GAACACCGTCACTTGCACCCAGCAGGGCCTGCTCGAGAACCAGCCTAGT |
| | TTCCACCCCGCCAGGCGTGAATAGGGTGATCACTAGAGAGGAGCTCGAG |
| | GCGCTTACCCCGTCACGCACTCCTAGCAGGTCGGTCTCGAGAACCAGCC |
| | TGGTCTCCAACCCGCCAGGCGTAAATAGGGTGATTACAAGAGAGGAGTT |
| | TGAGGCGTTCGTAGCACAACAACAATGACGGTTTGATGCGGGTGCATAC |
| | ATCTTTTCCTCCGACACCGGTCAAGGGCATTTACAACAAAAATCAGTAA |
| | GGCAAACGGTGCTATCCGAAGTGGTGTTGGAGAGGACCGAATTGGAGAT |
| | TTCGTATGCCCCGCGCCTCGACCAAGAAAAAGAAGAATTACTACGCAAG |
| | AAATTACAGTTAAATCCCACACCTGCTAACAGAAGCAGATACCAGTCCA |
| | GGAAGGTGGAGAACATGAAAGCCATAACAGCTAGACGTATTCTGCAAGG |
| | CCTAGGGCATTATTTGAAGGCAGAAGGAAAAGTGGAGTGCTACCGAACC |
| | CTGCATCCTGTTCCTTTGTATTCATCTAGTGTGAACCGTGCCTTTTCAA |
| | GCCCCAAGGTCGCAGTGGAAGCCTGTAACGCCATGTTGAAAGAGAACTT |
| | TCCGACTGTGGCTTCTTACTGTATTATTCCAGAGTACGATGCCTATTTG |
| | GACATGGTTGACGGAGCTTCATGCTGCTTAGACACTGCCAGTTTTTGCC |
| | CTGCAAAGCTGCGCAGCTTTCCAAAGAAACACTCCTATTTGGAACCCAC |
| | AATACGATCGGCAGTGCCTTCAGCGATCCAGAACACGCTCCAGAACGTC |
| | CTGGCAGCTGCCACAAAAAGAAATTGCAATGTCACGCAAATGAGAGAAT |
| | TGCCCGTATTGGATTCGGCGGCCTTTAATGTGGAATGCTTCAAGAAATA |
| | TGCGTGTAATAATGAATATTGGGAAACGTTTAAAGAAAACCCCATCAGG |
| | CTTACTGAAGAAAACGTGGTAAATTACATTACCAAATTAAAAGGACCAA |
| | AAGCTGCTGCTCTTTTTGCGAAGACACATAATTTGAATATGTTGCAGGA |
| | CATACCAATGGACAGGTTTGTAATGGACTTAAAGAGAGACGTGAAAGTG |
| | ACTCCAGGAACAAAACATACTGAAGAACGGCCCAAGGTACAGGTGATCC |
| | AGGCTGCCGATCCGCTAGCAACAGCGTATCTGTGCGGAATCCACCGAGA |
| | GCTGGTTAGGAGATTAAATGCGGTCCTGCTTCCGAACATTCATACACTG |
| | TTTGATATGTCGGCTGAAGACTTTGACGCTATTATAGCCGAGCACTTCC |
| | AGCCTGGGGATTGTGTTCTGGAAACTGACATCGCGTCGTTTGATAAAAG |
| | TGAGGACGACGCCATGGCTCTGACCGCGTTAATGATTCTGGAAGACTTA |
| | GGTGTGGACGCAGAGCTGTTGACGCTGATTGAGGCGGCTTTCGGCGAAA |
| | TTTCATCAATACATTTGCCCACTAAAACTAAATTTAAATTCGGAGCCAT |
| | GATGAAATCTGGAATGTTCCTCACACTGTTTGTGAACACAGTCATTAAC |
| | ATTGTAATCGCAAGCAGAGTGTTGAGAGAACGGCTAACCGGATCACCAT |
| | GTGCAGCATTCATTGGAGATGACAATATCGTGAAAGGAGTCAAATCGGA |
| | CAAATTAATGGCAGACAGGTGCGCCACCTGGTTGAATATGGAAGTCAAG |
| | ATTATAGATGCTGTGGTGGGCGAGAAAGCGCCTTATTTCTGTGGAGGGT |
| | TTATTTTGTGTGACTCCGTGACCGGCACAGCGTGCCGTGTGGCAGACCC |
| | CCTAAAAAGGCTGTTTAAGCTTGGCAAACCTCTGGCAGCAGACGATGAA |
| | CATGATGATGACAGGAGAAGGGCATTGCATGAAGAGTCAACACGCTGGA |
| | ACCGAGTGGGTATTCTTTCAGAGCTGTGCAAGGCAGTAGAATCAAGGTA |
| | TGAAACCGTAGGAACTTCCATCATAGTTATGGCCATGACTACTCTAGCT |
| | AGCAGTGTTAAATCATTCAGCTACCTGAGAGGGGCCCCTATAACTCTCT |
| | ACGGCTAACCTGAATGGACTACGACATAGTCTAGTCCGCCAAGGCCACC |
| | GATATCTATGTTACGTGCAAAGGTGATTGTCACCCCCCGAAAGACCATA |
| | TTGTGACACACCCTCAGTATCACGCCCAAACATTTACAGCCGCGGTGTC |
| | AAAAACCGCGTGGACGTGGTTAACATCCCTGCTGGGAGGATCAGCCGTA |
| | ATTATTATAATTGGCTTGGTGCTGGCTACTATTGTGGCCATGTACGTGC |
| | TGACCAACCAGAAACATAATTGAATACAGCAGCAATTGGCAAGCTGCTT |
| | ACATAGAACTCGCGGCGATTGGCATGCCGCCTTAAAATTTTTATTTTAT |
| | TTTTCTTTTCTTTTCCGAATCGGATTTTGTTTTTAATATTTCAAAAAAA |
| | AAAAAAAAAAAAAAAAAAAAAAAAAAAAAAAAAAaaagaagagcGCAGC |
| | TCTGGCCCGTGTCTCAAAATCTCTGATGTTACATTGCACAAGATAAAAA |
| | TATATCATCATGAACAATAAAACTGTCTGCTTACATAAACAGTAATACA |
| | AGGGGTGTTATGAGCCATATTCAACGGGAAACGTCGAGGCCGCGATTAA |
| | ATTCCAACATGGATGCTGATTTATATGGGTATAAATGGGCTCGCGATAA |
| | TGTCGGGCAATCAGGTGCGACAATCTATCGCTTGTATGGGAAGCCCGAT |
| | GCGCCAGAGTTGTTTCTGAAACATGGCAAAGGTAGCGTTGCCAATGATG |
| | TTACAGATGAGATGGTCAGACTAAACTGGCTGACGGAATTTATGCCTCT |
| | TCCGACCATCAAGCATTTTATCCGTACTCCTGATGATGCATGGTTACTC |
| | ACCACTGCGATCCCCGGAAAAACAGCATTCCAGGTATTAGAAGAATATC |
| | CTGATTCAGGTGAAAATATTGTTGATGCGCTGGCAGTGTTCCTGCGCCG |
| | GTTGCATTCGATTCCTGTTTGTAATTGTCCTTTTAACAGCGATCGCGTA |
| | TTTCGTCTCGCTCAGGCGCAATCACGAATGAATAACGGTTTGGTTGATG |
| | CGAGTGATTTTGATGACGAGCGTAATGGCTGGCCTGTTGAACAAGTCTG |
| | GAAAGAAATGCATAAACTTTTGCCATTCTCACCGGATTCAGTCGTCACT |
| | CATGGTGATTTCTCACTTGATAACCTTATTTTTGACGAGGGGAAATTAA |
| | TAGGTTGTATTGATGTTGGACGAGTCGGAATCGCAGACCGATACCAGGA |
| | TCTTGCCATCCTATGGAACTGCCTCGGTGAGTTTTCTCCTTCATTACAG |
| | AAACGGCTTTTTCAAAAATATGGTATTGATAATCCTGATATGAATAAAT |
| | TGCAGTTTCATTTGATGCTCGATGAGTTTTTCTAATCAGAATTGGTTAA |
| | TTGGTTGTAACACTGGCAGAGCATTACGCTGACTTGACGGGACGGCGCA |
| | AGCTCATGACCAAAATCCCTTAACGTGAGTTACGCGTCGTTCCACTGAG |
| | CGTCAGACCCCGTAGAAAAGATCAAAGGATCTTCTTGAGATCCTTTTTT |
| | TCTGCGCGTAATCTGCTGCTTGCAAACAAAAAAACCACCGCTACCAGCG |
| | GTGGTTTGTTTGCCGGATCAAGAGCTACCAACTCTTTTTCCGAAGGTAA |
| | CTGGCTTCAGCAGAGCGCAGATACCAAATACTGTTCTTCTAGTGTAGCC |
| | GTAGTTAGGCCACCACTTCAAGAACTCTGTAGCACCGCCTACATACCTC |
| | GCTCTGCTAATCCTGTTACCAGTGGCTGCTGCCAGTGGCGATAAGTCGT |
| | GTCTTACCGGGTTGGACTCAAGACGATAGTTACCGGATAAGGCGCAGCG |
| | GTCGGGCTGAACGGGGGGTTCGTGCACACAGCCCAGCTTGGAGCGAACG |
| | ACCTACACCGAACTGAGATACCTACAGCGTGAGCTATGAGAAAGCGCCA |
| | CGCTTCCCGAAGGGAGAAAGGCGGACAGGTATCCGGTAAGCGGCAGGGT |
| | CGGAACAGGAGAGCGCACGAGGGAGCTTCCAGGGGGAAACGCCTGGTAT |
| | CTTTATAGTCCTGTCGGGTTTCGCCACCTCTGACTTGAGCGTCGATTTT |
| | TGTGATGCTCGTCAGGGGGGCGGAGCCTATGGAAAAACGCCAGCAACGC |
| | GGCCTTTTTACGGTTCCTGGCCTTTTGCTGGCCTTTTGCTCACAT |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A method of reducing or avoiding the expression of a heterologous protein in the liver of a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the heterologous protein.

2. The method of claim 1, wherein reducing the expression comprises (i) reducing the amount of heterologous protein that is expressed in the liver, (ii) reducing the duration of the expression of the heterologous protein in the liver, or (iii) both (i) and (ii), as compared to that of a reference subject (e.g., a subject who received a corresponding lipid nanoparticle but where the replicon is not a VEE replicon).

3. The method of claim 2, wherein after the administration, the amount of heterologous protein that is expressed in the liver is reduced by at least about 5%, at least about 10%, at least about 20%, at least about 30%, at least about 40%, at least about 50%, at least about 60%, at least about 70%, at least about 80%, at least about 90%, or about 100%, as compared to that of the reference subject.

4. The method of claim 3, wherein after the administration, the liver does not express the heterologous protein.

5. The method of claim 2 or 3, wherein after the administration, the duration of the expression of the heterologous protein in the liver is reduced by at least about 5%, at least about 10%, at least about 20%, at least about 30%, at least about 40%, at least about 50%, at least about 60%, at least about 70%, at least about 80%, at least about 90%, or about 100%, as compared to that of the reference subject.

6. The method of any one of claims 1 to 5, wherein after the administration, the heterologous protein is expressed in a non-liver tissue of the subject, wherein the non-liver tissue is selected from spleen, lung, tumor, or combinations thereof.

7. The method of claim 6, wherein after the administration, the amount of heterologous protein that is expressed in the non-liver tissue is at least about 90%, at least about 91%, at least about 92%, at least about 93%, at least about 94%, at least about 95%, at least about 96%, at least about 97%, at least about 98%, at least about 99%, or at least about 100% or more, as compared to that observed in the non-liver tissue of the reference subject.

8. The method of claim 6 or 7, wherein after the administration, the duration of the expression of the heterologous protein in the non-liver tissue is at least about 90%, at least about 91%, at least about 92%, at least about 93%, at least about 94%, at least about 95%, at least about 96%, at least about 97%, at least about 98%, at least about 99%, or at least about 100%, as compared to that observed in the non-liver tissue of the reference subject.

9. A method of selectively expressing a heterologous protein in a non-liver tissue of a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the heterologous protein.

10. The method of claim 9, wherein after the administration, the amount of heterologous protein that is expressed in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold greater than the corresponding amount observed in the liver of the subject.

11. The method of claim 9 or 10, wherein after the administration, the duration of the expression of the heterologous protein in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding duration observed in the liver of the subject.

12. The method of any one of claims 9 to 11, wherein the non-liver tissue comprises spleen, lung, or both.

13. A method of treating a disease or disorder in a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding the heterologous protein, and wherein after the administration, the heterologous protein is preferentially expressed in a non-liver tissue of the subject.

14. The method of claim 13, wherein after the administration, the amount of heterologous protein that is expressed in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding amount observed in the liver of the subject.

15. The method of claim 13 or 14, wherein after the administration, the duration of the expression of the heterologous protein in the non-liver tissue is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding duration observed in the liver of the subject.

16. The method of any one of claims 13 to 15, wherein the non-liver tissue comprises spleen, lung, or both.

17. The method of any one of claims 13 to 16, wherein the disease or disorder comprises a cancer, inflammatory disorders, monogenic disorders, neurological disorders, psychiatric disorders, or combinations thereof.

18. The method of any one of claim 17, wherein the cancer comprises a melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, head and neck cancer, or combinations thereof.

19. A method of increasing tolerability of a lipid nanoparticle-based therapy in a subject in need thereof, comprising administering to the subject a lipid nanoparticle, which comprises (i) one or more types of lipids, and (ii) a replicon derived from a Venezuelan equine encephalitis (VEE) virus (“VEE replicon”), wherein the VEE replicon comprises a nucleic acid sequence encoding a heterologous protein.

20. The method of claim 19, wherein the tolerability of the lipid nanoparticle-based therapy is increased in the subject by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or greater than the corresponding tolerability observed in a reference subject (e.g., a subject who received a corresponding lipid nanoparticle but where the replicon is not a VEE replicon).

21. The method of claim 20, wherein the increased tolerability of the lipid nanoparticle-based therapy is associated with decreased liver toxicity.

22. The method of claim 21, wherein after the administration, the subject exhibits decreased liver toxicity compared to the corresponding liver toxicity observed in the reference subject.

23. The method of claim 22, wherein the liver toxicity in the subject is decreased by at least about 5%, at least about 10%, at least about 20%, at least about 30%, at least about 40%, at least about 50%, at least about 60%, at least about 70%, at least about 80%, at least about 90%, or about 100%, as compared to that of the reference subject.

24. The method of any one of claims 19 to 23, wherein after the administration, the subject exhibits decreased expression of the heterologous protein in the liver as compared to the corresponding expression observed in the liver of the reference subject.

25. The method of claim 24, wherein after the administration, the amount of heterologous protein that is expressed in the liver of the subject is reduced by at least about 5%, at least about 10%, at least about 20%, at least about 30%, at least about 40%, at least about 50%, at least about 60%, at least about 70%, at least about 80%, at least about 90%, or about 100%, as compared to that observed in the liver of the reference subject.

26. The method of claim 24 or 25, wherein after the administration, the duration of the expression of the heterologous protein in the liver of the subject is reduced by at least about 5%, at least about 10%, at least about 20%, at least about 30%, at least about 40%, at least about 50%, at least about 60%, at least about 70%, at least about 80%, at least about 90%, or about 100%, as compared to that observed in the liver of the reference subject.

27. The method of any one of claims 19 to 26, wherein the amount of heterologous protein that is expressed in the non-liver tissue of the subject is at least about 90%, at least about 91%, at least about 92%, at least about 93%, at least about 94%, at least about 95%, at least about 96%, at least about 97%, at least about 98%, at least about 99%, or at least about 100% or more, as compared to that observed in the non-liver tissue of the reference subject.

28. The method of any one of claims 19 to 27, wherein the duration of the expression of the heterologous protein in the non-liver tissue of the subject is at least about 90%, at least about 91%, at least about 92%, at least about 93%, at least about 94%, at least about 95%, at least about 96%, at least about 97%, at least about 98%, at least about 99%, or at least about 100%, as compared to that of the reference subject.

29. A method of producing a lipid nanoparticle-based therapy that is associated with decreased liver toxicity, comprising combining (i) a lipid nanoparticle, which comprises one or more types of lipids, and (ii) a replicon derived from a Venezuelan equine encephalitis (VEE) virus (“VEE replicon”), wherein the VEE replicon comprises a nucleic acid sequence encoding a heterologous protein.

30. The method of any one of claims 1 to 29, wherein the VEE replicon has a nucleotide sequence as set forth in SEQ ID NO: 187.

31. The method of any one of claims 1 to 30, wherein the heterologous protein comprises a cytokine, an antibody or antigen-binding fragment thereof, chimeric antigen receptor, or combination thereof.

32. The method of claim 31, wherein the cytokine comprises an interleukin (IL)-12 protein.

33. The method of claim 32, wherein the nucleic acid sequence of the VEE replicon comprises: (i) a nucleotide sequence encoding a IL-12 beta subunit and having at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the sequence set forth in SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, or SEQ ID NO: 75; (ii) a nucleotide sequence encoding a IL-12 alpha subunit and having at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, or SEQ ID NO: 125; or (iii) both (i) and (ii).

34. The method of claim 33, wherein the cytokine does not comprise an IL-12 protein.

35. The method of any one of claims 1 to 34, wherein the one or more types of lipid comprises an ionizable lipid, a cationic lipid, a lipidoid, a phospholipid, a sterol, or combinations thereof.

36. The method of claim 35, wherein the one or more types of lipid comprise TT3, 1,2-Dioleoyl-sn-glycero-3-phosphoethanolamine (DOPE), cholesterol, C14-PEG2000, or a combination thereof.

37. The method of claim 36, wherein the one or more types of lipid is TT3.

38. The method of claim 36 or 37, wherein the C14-PEG2000 comprises 1,2-dimyristoyl-rac-glycero-3-methoxypolyethylene glycol-2000 (DMG-PEG2000), 1,2-dimyristoyl-sn-glycero-3-phosphoethanolamine-N-[methoxy (polyethylene glycol)-2000] (DMPE-PEG2000), or both.

39. The method of any one of claims 36 to 38, wherein the C14-PEG2000 is embedded in the lipid nanoparticle.

40. The method of any one of claims 36 to 38, wherein the C14-PEG2000 has been added after the isolated polynucleotide has been encapsulated in the lipid nanoparticle.

41. The method of any one of claims 1 to 40, wherein the wherein the lipid nanoparticle has a diameter of about 30-500 nm.

42. The method of any one of claims 1 to 41, wherein the lipid nanoparticle has a diameter of about 50-400 nm.

43. The method of any one of claims 1 to 42, wherein the lipid nanoparticle has a diameter of about 70-300 nm.

44. The method of any one of claims 1 to 43, wherein the lipid nanoparticle has a diameter of about 100-200 nm.

45. The method of any one of claims 1 to 44, wherein the lipid nanoparticle has a diameter of about 100-175 nm.

46. The method of any one of claims 1 to 45, wherein the lipid nanoparticle has a diameter of about 100-160 nm.

47. The method of any one of claims 1 to 46, wherein the one or more types of lipid and the VEE replicon have a mass ratio of about 1:2 to about 15:1.

48. The method of claim 47, wherein the one or more types of lipid and the VEE replicon have a mass ratio of 1:2, 1:1.5, 1:1.2, 1:1.1, 1:1, 1.1:1, 1.2:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, or 15:1.

49. The method of any one of claims 1 to 48, wherein the VEE replicon comprises a 5′-cap.

50. The method of any one of claims 1 to 49, wherein the VEE replicon further comprises a regulatory element.

51. The method of claim 50, wherein the regulatory element is selected from the group consisting of at least one translation enhancer element (TEE), a translation initiation sequence, at least one microRNA binding site or seed thereof, a 3′ tailing region of linked nucleosides, an AU rich element (ARE), a post transcription control modulator, a 5′ UTR, a 3′ UTR, and combinations thereof.

52. The method of claim 51, wherein the 3′ tailing region of linked nucleosides comprises a poly-A tail, a polyA-G quartet, or a stem loop sequence.

53. The method of any one of claims 1 to 52, wherein the VEE replicon comprises at least one modified nucleoside.

54. The method of claim 53, wherein the at least one modified nucleoside is selected from the group consisting of 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

55. The method of any one of claims 1 to 54, wherein the one or more types of lipid and the VEE replicon have a mass ratio of about 10:1.

56. The method of any one of claims 1 to 55, wherein the lipid nanoparticle is administered to the subject via intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration.

57. The method of any one of claims 1 to 56, further comprising administering to the subject at least one additional therapeutic agent.

58. The method of claim 57, wherein the at least one additional therapeutic agent comprises a chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof.

59. The method of claim 58, wherein the immune checkpoint inhibitor comprises an anti-PD-1 antibody, anti-PD-L1 antibody, anti-LAG-3 antibody, anti-CTLA-4 antibody, anti-GITR antibody, anti-TIM3 antibody, or any combination thereof.
